(19) United States
(12) Patent Application Publication
(10) Pub. No.: US 2023/0037671 A1
(43) Pub. Date: Feb. 9, 2023

(54) Bioengineered Microbial Spores

(71) Applicant: The Trustees of Columbia University in the City of New York, New York, NY (US)

(72) Inventor: Juan Manuel de Jesus Flores Quijano, New York, NY (US)

(21) Appl. No.: 17/384,110

(22) Filed: Jul. 23, 2021

Related U.S. Application Data
(60) Provisional application No. 63/056,297, filed on Jul. 24, 2020.

(51) Int. Cl. C12N 15/11; C12N 9/22; C12N 15/75; C12N 1/20

(52) U.S. Cl. CPC C12N 15/11; C12N 9/22; C12N 2800/80; C12N 1/20; C12N 2310/20; C12N 15/75

(57) ABSTRACT

Phenotypically modified bioengineered microbial spores are provided. The microbial spores may be used in various spore-based technologies such as probiotics, biomaterials and vaccines.

### CROSS REFERENCE TO RELATED APPLICATIONS

This application claims priority to U.S. Provisional Application No. 63/056,297 filed on Jul. 24, 2020, which is incorporated herein by reference in its entirety.

### SEQUENCE LISTING

A sequence listing, filed as the ASCII text file “01001-008655-US1_SL.txt”, created on Jul. 9, 2021 and having a file size of 276 kilobytes, is incorporated herein by reference in its entirety.

### FIELD OF THE INVENTION

The present invention relates to genetically intact bioengineered microbial spores such as bacterial spores.

### BACKGROUND

Bioengineering continues to offer solutions to problems in agriculture, industry, medicine, and environment. Numerous technologies have been developed through genetic edition, such as metabolic engineering,¹ bacterial spore-based vaccines,² cell-based therapies,^{3,4} herbicide-resistant crops,⁵ and biosensors,⁶ among others.⁷ Nonetheless, despite continuing advances, the full potential of genome editing remains unexploited. This is in part due to policies in place that vary in a case by case manner,⁸ and also due to consumers' preferences.^{9,10}

To promote broader adoption of bioengineered organisms derived from genome editing, several biosafety tools have been developed over the past decade. One of these tools includes the use of RNA molecules to generate temporary physiological changes in an organism of interest, thus completely avoiding genome edition.¹¹ However, these RNA-based technologies have limitations including short endurance of the phenotype induced and the inability to express exogenous genes. Another approach is biocontainment, which consists of synthetic gene circuits that make the survival of an organism dependent on laboratory conditions or specific stimuli.¹²⁻¹⁴ However, organisms programmed for biocontainment are based on genetic edition. Furthermore, the death of such an organism in the environment presents the risk of releasing its DNA, which can later be absorbed by other microorganisms that are naturally competent,¹⁵ thus raising new concerns.

Genetic editing of B. subtilis (spores) has allowed scientists to create spore-based technologies such as probiotics, biomaterials, and vaccines that offer unique solutions due to the qualities that spores posses. For example, spores can be stored for long periods of time, they are easy and cheap to produce, and they are easily engineered, mainly through the expression of proteins on their spore coats. However, concerns on the safety of products that contain synthetic DNA may keep consumers away from these technologies.

The present disclosure offers a solution to this problem by establishing a method to engineer spores where the final product is spores with engineered phenotypes, but wild type genetics. In the context of bioengineering with synthetic gene circuits, our approach introduces a bioengineering tool that leverages differential expression patterns in different compartments of an organism. The synthetic gene circuits are programmed to behave differently in different cellular compartments by responding to natural differences in expression patterns,^{18,19} without relying on the addition of chemicals or promoter inducers.

### SUMMARY

The present disclosure provides for a method of producing a modified microbial spore. The method may comprise: (a) introducing at least one vector into a spore-forming microorganism, wherein the at least one vector encodes (i) a sequence-specific endonuclease system which targets the at least one vector, and (ii) an exogenous gene product, wherein the endonuclease system is operably linked to a spore-specific promoter, and (b) culturing the spore-forming microorganism to generate a modified microbial spore.

The at least one vector may further encode an exonuclease.

The at least one vector may comprise a vector encoding the sequence-specific endonuclease system and the exogenous gene product. The at least one vector may comprise two vectors encoding the sequence-specific endonuclease system and the exogenous gene product on different vectors.

The spore-forming microorganism may be a spore-forming bacterium or a spore-forming fungus. The spore-forming bacterium may be Bacillus subtilis, Bacillus firmus, Bacillus megaterium, Bacillus amyloliquefaciens, Bacillus licheniformis, or Bacillus pumilus.

The sequence-specific endonuclease system may comprise an RNA-guided endonuclease. In one embodiment, the RNA-guided endonuclease comprises a CRISPR/Cas system. In one embodiment, the RNA-guided DNA endonuclease is a Cas enzyme or a variant thereof, such as Cas9. In one embodiment, the at least one vector encodes a Cas enzyme and a guide RNA (gRNA). In one embodiment, the at least one vector encodes a Cas enzyme and two gRNAs.

In certain embodiments, the spore-specific promoter is PsspA.

In certain embodiments, the exonuclease is D15.

The at least one vector may be one or more plasmids.

The present disclosure provides for a phenotypically modified microbial spore. The spore may comprise an exogenous gene product (e.g., associated with the spore coat), where the microbial spore is free of exogenous genes, or substantially free of exogenous genes. The spore may not be genetically engineered.

The microbial spore may be a bacterial spore or a fungal spore.

The exogenous gene product may be a protein. The exogenous gene product may be an enzyme.

Also encompassed by the present disclosure is a composition comprising the microbial spore.

### BRIEF DESCRIPTION OF THE DRAWINGS

The patent or application file contains at least one drawing executed in color. Copies of this patent or application publication with color drawing(s) will be provided by that Office upon request and payment of the necessary fee.

The following drawings form part of the present specification and are included to further demonstrate certain aspects of the present disclosure, which can be better understood by reference to one or more of these drawings in combination with the detailed description of specific embodiments presented herein.

FIGS. 1A-1C. Schematization of the method to engineer spores while leaving the genome intact. (FIG. 1A) Self-digestion circuit. Cas9g1/g2=Cas9+one or two sgRNAs, respectively; D15=D15 exonuclease; PsspA=sporulation specific promoter; TS=endonuclease target site; T=transcriptional terminator; ori=replication origin; FPs=genes encoding foreign proteins. (FIG. 1B) Events derived from the plasmid during sporulation of B. subtilis. Circles=foreign proteins. (FIG. 1C) Final product: engineered spores with intact genomes.

FIGS. 2A-2F. Stability of plasmids during growth and sporulation. (FIGS. 2A, 2B) Bacterial growth in LB media. (FIGS. 2C, 2D) Adaptation to DSM. (FIGS. 2E, 2F) Sporulation in DSM. pBb is the backbone vector from which the rest of the plasmids built in this study are derived. Cas9g1/Cas9g2=Cas9+one or two sgRNAs, respectively. D15=D15 exonuclease. GFP=CotB-GFP fusion protein for expression of GFP in the spore coat. KinA=histidine kinase that induces sporulation artificially when overexpressed. Plasmid pLS1 was used as a control to assess whether our experiment was able to detect plasmid instability. *=controls where plasmid self-digestion was not expected. **=control where plasmid loss was expected.

FIGS. 3A-3D. GFP-coated spores. FIGS. 3A, 3B: pCas9g1-D15-GFP spores. FIGS. 3C, 3D: Mix of pCas9g1-D15-GFP and WT spores. FIGS. 3A, 3C: GFP imaging with confocal microscopy. FIGS. 3B, 3D: Optical microscopy. Red arrows in FIGS. 3C and 3D indicate spores without GFP fluorescence, which correspond to the WT strain.

FIGS. 4A-4B. Atomic force microscopy of WT and pCas9g1-D15-KinA spores. FIG. 4A: WT spores. FIG. 4B: KinA spores. Spores shown have an average diameter of ˜0.65 μm, and average length of 1.66 μm. Scale bar, 0.5 μm.

FIGS. 5A-5C. Germination of spores in LB or Valine media, and growth in LB-agar with or without antibiotic. Cm=chloramphenicol.

FIG. 6. Map of plasmid pV1.

FIG. 7. Map of plasmid pV1RX.

FIG. 8. Map of plasmid pBb.

FIG. 9. Map of plasmid pV1-PsspA.

FIG. 10. Map of plasmid pCas9g1-cons.

FIG. 11. Map of plasmid pCas9g2-cons.

FIG. 12. Map of plasmid pCas9g1-D15-cons.

FIG. 13. Map of plasmid pCas9g2-D15-cons.

FIG. 14. Map of plasmid pCas9g1.

FIG. 15. Map of plasmid pCas9g2.

FIG. 16. Map of plasmid pCas9g1-D15.

FIG. 17. Map of plasmid pCas9g2-D15.

FIG. 18. Map of plasmid pCas9g1-D15-mut.

FIG. 19. Map of plasmid pCas9g2-D15-mut.

FIG. 20. Map of plasmid pCas9g1-D15-GFP.

FIG. 21. Map of plasmid pCas9g2-D15-GFP.

FIG. 22. Map of plasmid pCas9g1-D15-KinA.

FIG. 23. Map of plasmid pCas9g2-D15-KinA.

FIGS. 24A-24D. GFP-coated spores. FIGS. 24A, 24C: GFP imaging with confocal microscopy. FIGS. 24B, 24D: Optical microscopy. FIGS. 24A, 24B: Strain pCas9g1-D15-GFP. FIGS. 24C, 24D: Strain pCas9g2-D15-GFP.

FIGS. 25A-25C. Germination of spore strains corresponding to mutated plasmids that were retained. Growth in LB-agar with or without antibiotic. Cm=chloramphenicol.

### DETAILED DESCRIPTION

The present disclosure provides for methods to produce phenotypically engineered microbial spores with wild type genetics. For example, the spores may contain exogenous proteins on their spore coats. The engineered microbial spores may be used in various spore-based technologies in agriculture, medicine, and industry such as probiotics, biomaterials (e.g., energy converting materials, living material systems, etc.) and vaccines.

In certain embodiments, B. subtilis spores produced by the methods express recombinant proteins while maintaining wild type genetics. The methods may utilize plasmids which self-digest following sporulation, after expression of the desired protein(s) is complete. In certain embodiments, the methods can produce recombinant protein-expressing spores that contain no detectable plasmid or chromosome alteration.

The present disclosure provides for a method of producing a modified microbial spore. The method may comprise: (a) introducing at least one vector into a spore-forming microorganism, wherein the at least one vector encodes (i) a sequence-specific endonuclease system which targets the at least one vector, and (ii) an exogenous gene product, wherein the endonuclease system is operably linked to a spore-specific promoter, and (b) culturing the spore-forming microorganism to generate a modified microbial spore.

The at least one vector may further encode an exonuclease, such as D15.

The at least one vector may comprise a vector encoding the sequence-specific endonuclease system and the exogenous gene product. The at least one vector may comprise two vector encoding the sequence-specific endonuclease system and the exogenous gene product on two different vectors.

The spore-forming microorganism may be a spore-forming bacterium or a spore-forming fungus. For example, the spore-forming bacterium may be Bacillus subtilis, Bacillus firmus, Bacillus megaterium, Bacillus amyloliquefaciens, Bacillus licheniformis, or Bacillus pumilus.

The sequence-specific endonuclease system may comprise an RNA-guided endonuclease, such as a CRISPR/Cas system. The RNA-guided DNA endonuclease may be a Cas enzyme or a variant thereof, such as Cas9. The at least one vector may encode a Cas enzyme and at least one guide RNA (gRNA) (e.g., a gRNA, two gRNAs, three gRNAs, etc.) which targets the at least one vector.

The at least one vector may be a plasmid.

Also encompassed by the present disclosure is a phenotypically modified microbial spore, comprising an exogenous gene product, wherein the microbial spore is substantially free of exogenous genes.

The microbial spore may be a bacterial spore or a fungal spore.

The exogenous gene product may be a protein, a polypeptide, or a peptide. The exogenous gene product may be an enzyme.

The present disclosure also provides for a composition comprising the present modified microbial spore.

The present compositions may be used in probiotics for human consumption, antibody production for therapeutic use, vaccines (e.g., oral vaccines for livestock), production of recombinant proteins for biological research, environmental remediation, fertilizer additives, etc.

The exogenous gene product (e.g., a protein) may be expressed cytoplasmically in a microbial cell, e.g., in soluble form, during vegetative growth of the cell. The exogenous gene product, while made as a soluble material, becomes associated with spores during the sporulation process. In certain embodiments, the exogenous gene product may be expressed as a fusion product with a spore coat protein which will be displayed on the spore surface. The term “associated” as used herein may refer to the physical relationship of exogenous gene product to a spore, or a portion thereof. During sporulation, the exogenous gene product is produced during vegetative phase of cell growth and is physically associated with the spore. The exogenous gene product may be displayed on the spore's surface, or may be entrained or entrapped within the spore coat.

Microorganisms or microbes include, but are not limited to, the two prokaryotic domains, Bacteria and Archaea, as well as eukaryotic fungi and protists. By way of example, the microorganisms may include Proteobacteria (such as Pseudomonas, Enterobacter, Stenotrophomonas, Burkholderia, Rhizobium, Herbaspirillum, Pantoea, Serratia, Rahnella, Azospirillum, Azorhizobium, Azotobacter, Duganella, Delftia, Bradyrhizobiun, Sinorhizobium and Halomonas), Firmicutes (such as Bacillus, Paenibacillus, Lactobacillus, Mycoplasma, and Acetobacterium), Actinobacteria (such as Streptomyces, Rhodococcus, Microbacterium, and Curtobacterium), and the fungi Ascomycota (such as Trichoderma, Ampelomyces, Coniothyrium, Paecoelomyces, Penicillium, Cladosporium, Hypocrea, Beauveria, Metarhizium, Verticullium, Cordyceps, Pichea, and Candida, Basidiomycota (such as Coprinus, Corticium, and Agaricus) and Oomycota (such as Pythium, Mucor, and Mortierella).

Bacteria from the genus Bacillus include, but are not limited to, Bacillus subtilis, Bacillus firmus, Bacillus megaterium, Bacillus amyloliquefaciens, Bacillus brevis, Bacillus clausii, Bacillus lentus, Bacillus uniflagellatus, B. vallismortis, Bacillus lateropsorus, Bacillus laterosporus BOD, Bacillus megaterium, Bacillus polymyxa, Bacillus licheniformis, Bacillus pumilus, and Bacillus sterothermophilus, Bacillus simplex, Bacillus sphaericus, Bacillus stearothermophilus, Bacillus thuringiensis, Bacillus coagulans, Bacillus thermophilus, Bacillus mycoides, Bacillus cereus, Bacillus circulans, or any combination thereof. In certain embodiments, the bacterial spore is Bacillus thurengiensis var. aizawai, Bacillus licheniformis var. endoparasiticus, or Bacillus thuringiensis var. kurstaki.

The microbial spore may be a bacterial spore or a fungal spore. Bacterial spores may include endospores and akinetes. Fungal spores may include statismospores, ballistospores, autospores, aplanospores, zoospores, mitospores, megaspores, microspores, meiospores, chlamydospores, urediniospores, teliospores, oospores, carpospores, tetraspores, sporangiospores, zygospores, basidiospores, asco spores, and asciospores.

As used herein, the term “phenotype” refers to the observable characteristics of a individual cell (e.g., a microorganism), cell culture, organism, or group of organisms. In some embodiments, the phenotypically modified spore has at least one heterologous trait. As used herein, the term “heterologous trait” refers to a phenotype imparted to a modified spore, not by an exogenous DNA, heterologous polynucleotide or heterologous nucleic acid.

As used herein, the term “endogenous” or “endogenous gene,” refers to the naturally occurring gene, in the location in which it is naturally found within the host cell.

As used herein, the term “exogenous” may refer to a substance coming from some source other than its native source. For example, the terms “exogenous protein,” or “exogenous gene” refer to a protein or gene from a non-native source or location, and that have been artificially supplied to a biological system.

As used herein, “promoter” refers to a DNA sequence capable of controlling the expression of a coding sequence or functional RNA. The promoter sequence may contain proximal and more distal upstream elements.

As used herein, the term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter).

As used herein, the phrases “vector”, “recombinant construct”, “expression construct”, “construct”, and “recombinant DNA construct” are used interchangeably. A vector comprises an artificial combination of nucleic acid fragments, e.g., regulatory and coding sequences that are not found together in nature. For example, a vector may comprise regulatory sequences and coding sequences that are derived from different sources, or regulatory sequences and coding sequences derived from the same source, but arranged in a manner different than that found in nature. Such construct may be used by itself or may be used in conjunction with another vector. The choice of vector may be dependent upon the method that will be used to transform host cells as is well known to those skilled in the art. For example, a plasmid vector can be used. Vectors can be plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating.

In certain embodiments, the vector is or is not integrated into the microbial chromosome (e.g., the bacterial chromosome).

Nucleic acids (e.g., vectors) can be delivered to a bacterial cell by transformation, e.g., heat shock, electroporation, etc. In one embodiment, bacterial cells are incubated in a solution containing divalent cations (e.g., calcium chloride) under cold conditions, before being exposed to a heat pulse (heat shock). The sequence-specific nuclease can be introduced into the cell in the form of a protein or in the form of a nucleic acid encoding the sequence-specific nuclease, such as a vector, an mRNA or a cDNA. Nucleic acids can be delivered as part of a larger construct, such as a plasmid or viral vector, or directly, e.g., by electroporation, lipid vesicles, viral transporters, microinjection, and biolistics.

The term “substantially free” of an agent should be understood as meaning free of the agent, or that any amount of the agent present in the spore is so low so as not to have any effect on the spore (e.g., on the phenotype of the spore, and/or on the properties of the spore).

The term “about” in reference to a numeric value refers to ±10% of the stated numeric value. In other words, the numeric value can be in a range of 90% of the stated value to 110% of the stated value.

### Bacterial Spores

In certain embodiments, the microorganism is an endospore-forming bacterium, for example, from the Bacilli and Clostridia genera. Both Bacilli and Clostridia undergo a lifecycle involving growth (multiplication) of cells, conversion of cells to endospores (which occurs when the cells are stressed by, for example, lack of nutrients), activation of the endospores (which occurs when the endospores are re-exposed to nutrients), followed by germination of the endospores and finally outgrowth of new vegetative cells (which occurs under continued exposure to nutrients).

Bacteria may include, but are not limited to, species including spore-forming members of the phylum Firmicutes and spore-forming members of the phylum Actinobacteria as listed in Bergey's Manual of Systematic Bacteriology, Second Edition (2009), hereby incorporated by reference in its entirety. Non-limiting examples of the bacterial species include Bacillus subtilis, Bacillus agri, Bacillus aizawai, Bacillus albolactis, Bacillus alcalophilus, Bacillus altitudinis, Bacillus alvei, Bacillus amyloliquefaciens, Bacillus aneurinolyticus, Bacillus anthracis, Bacillus aquaemaris, Bacillus atrophaeus, Bacillus boronophilus, Bacillus brevis, Bacillus butanolivorans, Bacillus caldolyyicus, Bacillus centrosporus, Bacillus cereus, Bacillus circulans, Bacillus clausii, Bacillus coagulans, Bacillus endoparasiticus, Bacillus endorhythmos, Bacillus firmus, Bacillus flavothermus, Bacillus formis, Bacillus fusiformis, Bacillus globigii, Bacillus infernus, Bacillus kurstaki, Bacillus lacticola, Bacillus lactimorbus, Bacillus lactis, Bacillus laterosporus, Bacillus larvae, Bacillus laterosporus, Bacillus lentimorbus, Bacillus lentus, Bacillus licheniformis, Bacillus medusa, Bacillus metiens, Bacillus megaterium, Bacillus mesentericus, Bacillus mojavensis, Bacillus mucilaginosus, Bacillus mycoides, Bacillus natto, Bacillus nigrificans, Bacillus pantothenicus, Bacillus popilliae, Bacillus polymyxa, Bacillus pseudoanthracis, Bacillus pumilus, Bacillus schlegelii, Bacillus siamensis, Bacillus spp., Bacillus simplex, Bacillus sphaericus, Bacillus sporothermodurans, Bacillus stearothermophilus, Bacillus subtilis, Bacillus thermoglucosidasius, Bacillus thuringiensis, Bacillus unifagellatus, Bacillus vulgatis, Bacillus weihenstephanensis, Clostridium thermocellum, Clostridium ljungdahlii, Clostridium acetobutylicum, Clostridium beijerinckii, Clostridium butyricum, Clostridium ljungdahlii, Clostridium butyricum, Pasteuria penetrans, Pasteuria thornei, Pasteuria nishizawae and a species of the genus Streptomyces.

Bacillus subtilis is a Gram-positive, catalase-positive bacterium commonly found in soil. Members of the genus Bacillus have the ability to form tough, protective endospores, a characteristic which allows the spores of the organism to tolerate extreme environmental conditions, to be heat resistant, and to quantitatively survive lengthy exposure to a wide range of temperatures including freezing and boiling, without loss of viability.

As used herein, “spore” or “spores” may refer to structures produced by bacteria and fungi that are adapted for survival and dispersal. Spores are generally characterized as dormant structures; however, spores are capable of differentiation through the process of germination. Germination is the differentiation of spores into vegetative cells that are capable of metabolic activity, growth, and reproduction. The germination of a single spore results in a single fungal or bacterial vegetative cell. Fungal spores are units of asexual reproduction, and in some cases are necessary structures in fungal life cycles. Bacterial spores are structures for surviving conditions that may ordinarily be nonconductive to the survival or growth of vegetative cells.

The term “Bacillus spore” in relation to Bacillus spore cell relates herein to a spore that may be characterized as a dormant, tough, non-reproductive structure produced by Bacillus bacteria. The primary function of spores is generally to ensure the survival of a bacterium through periods of environmental stress. They are therefore resistant to ultraviolet and gamma radiation, desiccation, lysozyme, temperature, starvation, and chemical disinfectants. Spores are commonly found in soil and water, where they may survive for long periods of time. The spore coat is impermeable to many toxic molecules and may also contain enzymes that are involved in germination. The core has normal cell structures, such as DNA and ribosomes, but is metabolically inactive. When a bacterium detects that environmental conditions are becoming unfavorable it may start the process of sporulation, which takes about eight hours.

### Sporulation Promoter

A sporulation promoter may also be referred to as a sporulation-specific promoter or spore-specific promoter.

The nuclease (e.g., the sequence-specific endonuclease) may be operably linked to, or under the control of, a sporulation promoter which will cause expression of the nuclease in the spore (e.g., the forespore, or the endospore). Sporulation promoters may be a fragment or variant of a promoter which remains transcriptionally active during sporulation.

In certain embodiments, the sporulation promoter is PsspA.

The sporulation promoter may be a native bclA promoter from a B. cereus family member. The sporulation promoter may comprise one or more sigma-K sporulation-specific polymerase promoter sequences. Non-limiting examples of sporulation promoters also include BclA promoter (B. anthracis Sterne), BetA promoter (B. anthracis Sterne), BAS1882 promoter (B. anthracis Sterne), Gene 3572 promoter (B. weihenstephensis KBAB 4), YVTN beta-propeller protein promoter (B. weihenstephensis KBAB 4), Cry1A promoter (B. thuringiensis HD-73), ExsY promoter (B. thuringiensis serovar konkukian str. 97-27), CotY promoter (B. thuringiensis Al Hakam), YjcA promoter (B. thuringiensis serovar kurstaki str. HD73), YjcB promoter (B. thuringiensis serovar kurstaki str. HD73), BxpB promoter (B. thuringiensis Al Hakam), Rhamnose promoter (B. thuringiensis Al Hakam), CotY/CotZ promoter (B. anthracis Sterne), BclC promoter (B. anthracis Sterne), Sigma K promoter (B. anthracis Sterne), InhA promoter (B. thuringiensis Al Hakam), BclA cluster glycosyl transferase operon 1 (B. thuringiensis serovar konkukian str. 97-27), BclA cluster glycosyl transferase operon 2 (B. thuringiensis serovar kurstaki str. HD73), and Glycosyl transferase promoter (B. thuringiensis Al Hakam). See, U.S. Pat. Nos. 11,044,916; 10,988,769; and 10,836,800, the content of each of which is incorporated herein by reference in its entirety.

### Nucleases

A nuclease can be an agent capable of cleaving a phosphodiester bond connecting two nucleotide residues in a nucleic acid molecule. In some embodiments, a nuclease is a protein, e.g., an enzyme, that can bind a nucleic acid molecule and cleave a phosphodiester bond connecting nucleotide residues within the nucleic acid molecule. A nuclease may be an endonuclease, cleaving phosphodiester bonds within a polynucleotide chain, or an exonuclease, cleaving phosphodiester bonds at the end of the polynucleotide chain. In some embodiments, a nuclease is a site-specific or sequence-specific nuclease (e.g., a sequence-specific endonuclease), binding and/or cleaving a specific phosphodiester bond within a specific nucleotide sequence, which is also referred to herein as the “recognition sequence,” the “nuclease target site,” or the “target site.” In some embodiments, a nuclease is a RNA-guided nuclease, which is associated with (e.g., binds to) an RNA (e.g., a guide RNA, “gRNA”) having a sequence that can bind specifically to a target site, thereby providing the sequence specificity of the nuclease. Nucleases may be specific to either single-stranded or double-stranded nucleic acid sequences. Some enzymes have both exonuclease and endonuclease properties. In some embodiments, a nuclease recognizes a single stranded target site, while in other embodiments, a nuclease recognizes a double-stranded target site, for example, a double-stranded DNA target site. The target sites of many naturally occurring nucleases, for example, many naturally occurring DNA restriction nucleases, are well known to those of skill in the art. In many cases, a DNA nuclease, such as EcoRI, HindIII, or BamHI, recognize a palindromic, double-stranded DNA target site of 4 to 10 base pairs in length, and cut each of the two DNA strands at a specific position within the target site.

### Endonucleases

Non-limiting examples of the site-specific endonucleases include a zinc finger nuclease (ZFN), a ZFN dimer, a ZFNickase, a transcription activator-like effector nuclease (TALEN), meganucleases, or a RNA-guided DNA endonuclease (e.g., CRISPR/Cas systems such as CRISPR/Cas9). Meganucleases are endonucleases characterized by their capacity to recognize and cut large DNA sequences (12 base pairs or greater). Any suitable meganuclease may be used in the present methods, including endonucleases in the LAGLIDADG and PI-Sce family.

In some embodiments, the sequence-specific endonuclease is a nickase.

The sequence-specific endonuclease of the methods and compositions described herein can be engineered, chimeric, or isolated from an organism. Endonucleases can be engineered to recognize a specific DNA sequence, by, e.g., mutagenesis. Seligman et al. (2002) Mutations altering the cleavage specificity of a homing endonuclease, Nucleic Acids Research 30: 3870-3879. Combinatorial assembly is a method where protein subunits form different enzymes can be associated or fused. Arnould et al. (2006) Engineering of large numbers of highly specific homing endonucleases that induce recombination to novel DNA targets, Journal of Molecular Biology 355: 443-458. These two approaches, mutagenesis and combinatorial assembly, may be combined to produce an engineered endonuclease with desired DNA recognition sequence.

The term “RNA-guided nuclease” may refer to a nuclease that forms a complex with (e.g., binds or associates with) one or more RNA that is not a target for cleavage. The CRISPR (Clustered Regularly interspaced Short Palindromic Repeats) system exploits RNA-guided DNA-binding and sequence-specific cleavage of target DNA. The guide RNA/Cas combination confers site specificity to the nuclease. A single guide RNA (sgRNA) can be complementary to a target sequence. The Cas (CRISPR-associated) protein binds to the sgRNA and the target DNA to which the sgRNA binds and introduces a double-strand break. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is used interchangeably to refer to guide RNAs that exist as either single molecules or as a complex of two or more molecules. Typically, gRNAs that exist as single RNA species comprise two domains: (1) a first domain that shares homology to a target nucleic acid (e.g., a domain can direct binding of a Cas9 complex to the target); and (2) a second domain that binds a Cas9 protein. In some embodiments, the second domain corresponds to a sequence known as a tracrRNA. In some embodiments, the RNA-programmable nuclease is the (CRISPR-associated system) Cas9 endonuclease, for example Cas9 (Csn1) from Streptococcus pyogenes. The CRISPR/Cas9 system may comprise a Cas9 nuclease and an engineered crRNA/tracrRNA (or single guide RNA).

The terms “gRNA,” “guide RNA” and “CRISPR guide sequence” may be used interchangeably throughout and refer to a nucleic acid comprising a sequence that determines the specificity of a Cas DNA binding protein of a CRISPR/Cas system. A gRNA hybridizes to (complementary to, partially or completely) a target nucleic acid sequence. The gRNA or portion thereof that hybridizes to the target nucleic acid may be between 15-25 nucleotides, 18-22 nucleotides, or 19-21 nucleotides in length. In some embodiments, the gRNA sequence that hybridizes to the target nucleic acid is 15, 16, 17, 18, 19, 20, 21, 22, 23, 24, or 25 nucleotides in length. In some embodiments, the gRNA sequence that hybridizes to the target nucleic acid is between 10-30, or between 15-25, nucleotides in length.

In addition to a sequence that binds to a target nucleic acid, in some embodiments, the gRNA may also comprise a scaffold sequence. Expression of a gRNA encoding both a sequence complementary to a target nucleic acid and scaffold sequence has the dual function of both binding (hybridizing) to the target nucleic acid and recruiting the endonuclease to the target nucleic acid, which may result in site-specific CRISPR activity. In some embodiments, such a chimeric gRNA may be referred to as a single guide RNA (sgRNA). As used herein, a “scaffold sequence,” also referred to as a tracrRNA, refers to a nucleic acid sequence that recruits a Cas endonuclease to a target nucleic acid bound (hybridized) to a complementary gRNA sequence.

In some embodiments, the gRNA sequence does not comprise a scaffold sequence and a scaffold sequence is expressed as a separate transcript. In such embodiments, the gRNA sequence further comprises an additional sequence that is complementary to a portion of the scaffold sequence and functions to bind (hybridize) the scaffold sequence and recruit the endonuclease to the target nucleic acid.

In one embodiment, Cas protein may be a functional derivative of a naturally occurring Cas protein.

Cas9 harbors two independent nuclease domains homologous to HNH and RuvC endonucleases, and by mutating either of the two domains, the Cas9 protein can be converted to a nickase that introduces single-strand breaks (Cong, L. et al. Science 339, 819-823 (2013)).

Cleavage of a target sequence may comprise cleaving one or two strands at the location of the target sequence by the Cas enzyme. The methods and compositions of the present disclosure may be used with the single- or double-strand-inducing version of Cas9, as well as with other RNA-guided DNA nucleases, such as other bacterial Cas9-like systems. The sequence-specific nuclease of the present methods and compositions described herein can be engineered, chimeric, or isolated from an organism. The nuclease can be introduced into the cell in form of a DNA, mRNA and protein.

In some embodiments, the Cas endonuclease is a Cas9 enzyme or variant thereof. In some embodiments, the Cas9 endonuclease is derived from Streptococcus pyogenes, Staphylococcus aureus, Neisseria meningitidis, Streptococcus thermophilus, or Treponema denticola. In some embodiments, the endonuclease is a Cas9 homolog or ortholog.

Alternatively or in addition, the Cas endonuclease is a Cpf1 nuclease. In some embodiments, the host cell expresses a Cpf1 nuclease derived from Provetella spp. or Francisella spp.

In one embodiment, the CRISPR-Cas system for use in the methods provided herein is a Class 2 system. In one embodiment, the CRISPR-Cas system for use in the methods provided herein is a Type II, Type V or Type VI Class 2 system. In one embodiment, the CRISPR-Cas system for use in the methods provided herein is selected from Cas9, Cas12a, Cas12b, Cas12c, Cas12d, Cas12e, Cas13a, Cas13b, Cas13c or homologs, orthologs or paralogs thereof.

In some embodiments, the Cas proteins can comprise one or multiple nuclease domains. A Cas effector protein can target single stranded or double stranded nucleic acid molecules (e.g. DNA or RNA nucleic acids) and can generate double strand or single strand breaks. In some embodiments, the Cas effector proteins are wild-type or naturally occurring Cas proteins. In some embodiments, the Cas effector proteins are mutant Cas proteins, wherein one or more mutations, insertions, or deletions are made in a WT or naturally occurring Cas protein (e.g., a parental Cas protein) to produce a Cas protein with one or more altered characteristics compared to the parental Cas protein.

In some instances, the Cas protein is a wild-type (WT) nuclease. Non-limiting examples of Cas proteins include C2c1, C2c2, C2c3, Cas1, Cas1B, Cas2, Cas3, Cas4, Cas5, Cash, Cas7, Cas8, Cas9 (also known as Csn1 and Csx12), Cas10, Cpf1, Csy1, Csy2, Csy3, Cse1, Cse2, Csc1, Csc2, Csa5, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx100, Csx16, CsaX, Csx3, Csx1, Csx15, Csf1, Csf2, Csf3, Csf4, MAD1-20, SmCsm1, homologes thereof, orthologes thereof, variants thereof, mutants thereof, or modified versions thereof. Suitable nucleic acid guided Cas nucleases (e.g., Cas9) can be from an organism from a genus including, but not limited to: Thiomicrospira, Succinivibrio, Candidatus, Porphyromonas, Acidomonococcus, Prevotella, Smithella, Moraxella, Synergistes, Francisella, Leptospira, Catenibacterium, Kandleria, Clostridium, Dorea, Coprococcus, Enterococcus, Fructobacillus, Weissella, Pediococcus, Corynebacter, Sutterella, Legionella, Treponema, Roseburia, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, Alicyclobacillus, Brevibacilus, Bacillus, Bacteroidetes, Brevibacilus, Carnobacterium, Clostridiaridium, Clostridium, Desulfonatronum, Desulfovibrio, Helcococcus, Leptotrichia, Listeria, Methanomethyophilus, Methylobacterium, Opitutaceae, Paludibacter, Rhodobacter, Sphaerochaeta, Tuberibacillus, and Campylobacter.

In some embodiments, the Cas endonuclease and the nucleic acid encoding the gRNA are provided on the same nucleic acid (e.g., a vector). In some embodiments, the Cas endonuclease and the nucleic acid encoding the gRNA are provided on different nucleic acids (e.g., different vectors). Alternatively or in addition, the Cas endonuclease may be provided or introduced into the cell in protein form.

In further embodiment, the endonuclease is a transcription activator-like effector nuclease (TALEN). TALENs are composed of a TAL effector domain that binds to a specific nucleotide sequence and an endonuclease domain that catalyzes a double strand break at the target site (PCT Patent Publication No. WO2011072246; Miller et al., Nat. Biotechnol. 29, 143-148 (2011); Cermak et al., Nucleic Acid Res. 39, e82 (2011)). Sequence-specific endonucleases may be modular in nature, and DNA binding specificity is obtained by arranging one or more modules. Bibikova et al., Mol. Cell. Biol. 21, 289-297 (2001). Boch et al., Science 326, 1509-1512 (2009).

ZFNs can be composed of two or more (e.g., 2-8, 3-6, 6-8, or more) sequence-specific DNA binding domains (e.g., zinc finger domains) fused to an effector endonuclease domain (e.g., the FokI endonuclease). Porteus et al., Nat. Biotechnol. 23, 967-973 (2005). Kim et al. (2007) Hybrid restriction enzymes: Zinc finger fusions to Fok I cleavage domain, Proceedings of the National Academy of Sciences of USA, 93: 1156-1160. U.S. Pat. No. 6,824,978. PCT Publication Nos. WO1995/09233 and WO1994018313.

### Exonucleases

In certain embodiments, the exonucleases used in the methods, systems, and compositions provided herein include exonucleases with a 5′ to 3′ and/or a 3′ to 5′ exonuclease activity on a double-stranded DNA (dsDNA) substrate or single stranded DNA (ssDNA) substrate. In certain embodiments, the exonuclease will recognize a dsDNA substrate with a blunt end, including a blunt end with a 5′ phosphate group. In certain embodiments, the exonuclease will recognize a dsDNA substrate with an overhang of ssDNA (e.g., a 5′ or 3′ ssDNA region at a terminus of a dsDNA molecule, including ends produced by endonucleases which provide staggered cuts in dsDNA substrates). In certain embodiments, the exonuclease will recognize a dsDNA substrate having an internal break in one strand (e.g., a nicked dsDNA).

In one embodiment, the exonuclease is D15, an exonuclease from the T5 phage.

Exonucleases with 5′ to 3′ exonuclease activity that can be used herein also include a bacteriophage lambda exo protein, an Rac prophage RecE exonuclease protein, an Artemis protein, an Apollo protein, a DNA2 exonuclease protein, an Exo 1 exonuclease protein, a herpesvirus SOX protein, UL12 exonuclease protein, an enterobacterial exonuclease VIII protein, a T7 phage exonuclease protein or a related protein with equivalent 5′ to 3′ exonuclease activity. Exonucleases with 3′ to 5′ exonuclease activity that can be used herein include an E. coli Exonuclease III, a mammalian Trex2 exonuclease protein, a related protein with equivalent 3′ to 5′ exonuclease activity. In certain embodiments, the aforementioned exonucleases will comprise conserved DEDD catalytic residues characteristic of the DEDD/DnaQ superfamily of exonucleases (Bernad et al., 1989). In certain embodiments, the exonuclease can comprise an allelic variant of any of the aforementioned exonucleases.

### Applications of the Present Methods/Compositions

The present disclosure also provides for a composition comprising the present modified spore.

Compositions comprising microbial spores may be suitable for agricultural, horticultural, environmental, probiotic, aquatic, industrial and sanitation uses, among others. The present disclosure provides microbial spore compositions (e.g., bacterial spore compositions) for industrial uses such as agriculture, environmental remediation, composting, methane production, cleaning supplies, oil recovery, wastewater treatment, and direct fed microbials.

Regarding agricultural applications, the present composition may be used to enhance the health of plants. The present compositions/spores may be coated onto seeds or other plant propagative material, such that once sown or planted in an enhanced environment which supports germination of the seed, stimulation of plant growth or biological protection of the seed and resulting plant can be established. In one embodiment, the present composition may be used as, or in, a biopesticide.

The present composition may be for use in foods for human consumption or for animal feed. In certain embodiments, the present disclosure provides for a probiotic composition. In certain embodiments, the animal feed is a direct fed microbial. Probiotics are living microbes that have beneficial effects on humans and/or animals when ingested. The probiotic may comprise the present composition and an acceptable carrier. In certain embodiments, the bacterium is a spore-forming Bacillus species with probiotic effects. Representative examples of such spore-forming bacteria include various spore-forming Bacillus species including, but not limited to, Bacillus species such as Bacillus subtilis, Bacillus licheniformis, Bacillus coagulans, Bacillus pumilis, Bacillus clausii, mixtures thereof, and other microbes as described herein.

An acceptable carrier may be added to the composition. The acceptable carrier may be a liquid carrier, a solid carrier, a water soluble carrier, or any other suitable carrier. In one embodiment, the liquid carrier is milk or a milk replacer. Milk replacers are typically milk substitutes in powdered form that are mixed with water to form a composition that resembles milk. In one embodiment, the liquid carrier is water. Dry carriers include, but are not limited to, animal feed, whey, limestone (calcium carbonate), rice hulls, yeast culture, dried starch, and sodium silico aluminate. In certain embodiments, the water soluble carrier is selected from the group consisting of whey, maltodextrin, sucrose, dextrose, dried starch, and sodium silico aluminate. In other embodiments, the acceptable carrier for a direct fed microbial is selected from the group consisting of vegetable oil, sucrose, silicon dioxide, polysorbate 80, propylene glycol, butylated hydroxyanisole, citric acid, and ethoxyquin. In certain embodiments, the acceptable carrier is selected from the group consisting of animal feed, milk, yogurt, cheese, fermented milk, cereal, fermented cereal, juice, ice-cream, or a formulation for infants or children.

As used herein, “carrier”, “acceptable carrier”, or “pharmaceutical carrier” refers to a diluent, adjuvant, excipient, or vehicle. Such carriers can be sterile liquids, such as water and oils, including those of petroleum, animal, vegetable, or synthetic origin; such as peanut oil, soybean oil, mineral oil, sesame oil, and the like. Water or aqueous solution saline solutions and aqueous dextrose and glycerol solutions are preferably employed as carriers, in some embodiments as injectable solutions. Alternatively, the carrier can be a solid dosage form carrier, including but not limited to one or more of a binder (for compressed pills), a glidant, an encapsulating agent, a flavorant, and a colorant. The choice of carrier can be selected with regard to the intended route of administration and standard pharmaceutical practice. In some aspects, carriers may be granular in structure, such as sand or sand particles. In further aspects, the carriers may be dry, as opposed to a moist or wet carrier. In some aspects, carriers can be nutritive substances and/or prebiotic substances selected from fructooligosaccharides, inulins, isomalto-oligosaccharides, lactitol, lactosucruse, lactulose, pyrodextrines, soy oligosaccharides, transgalacto-oligosaccharides, xylo-oligosaccharides, and vitamins. In some aspects, carriers can be in solid or liquid form. In some aspects, carriers can be zeolites, calcium carbonate, magnesium carbonate, trehalose, chitosan, shellac, albumin, starch, skim-milk powder, sweet-whey powder, maltodextrin, lactose, and inulin. In some aspects, a carrier is water or physiological saline.

In another aspect, the present disclosure relates to a method for treating water in an aquaculture system, comprising contacting a composition comprising a bacterial spore with water in an aquaculture system. In certain embodiments, the aquaculture system comprises an animal.

The present composition may be used for animal feeds. The present disclosure relates to a method for feeding an animal comprising administering a composition comprising a bacterial spore to an animal. In certain embodiments, the animal is selected from the group consisting of poultry, ruminants, calves, pigs, rabbits, horses, crustaceans, mollusks, fish and pets. In another aspect, the present disclosure relates to a method for feeding an animal in an aquaculture system, comprising administering an animal feed comprising a composition comprising a bacterial spore to the animal in the aquaculture system. The animal may be fish, shrimp, or mollusks. In certain embodiments, the aquaculture system comprises a pathogen selected from the group consisting of a Vibrio species, an Aeromonas species and a Flavobacterium species. In certain embodiments, the composition is in the form of granules, briquettes, pellets, tablets, or capsules.

The present composition may be used for wastewater treatment, and the present disclosure relates to a method for treating wastewater comprising adding a composition comprising a microbial spore (e.g., a bacterial spore) to wastewater.

The present composition may be used for environmental remediation. The present disclosure relates to a method for environmental remediation comprising applying a composition comprising a microbial spore (e.g., a bacterial spore) to soil or water. Bacillus species may be used for environmental remediation both in water and land. The bacteria break down chemicals and pollutants via enzyme and acid generation that further break down the pollutant or bind it in a more non-reactive form.

The present disclosure relates to a cleaning product comprising a composition comprising the present spore (e.g., the bacterial spore) and an acceptable carrier. In certain embodiments, the acceptable carrier is selected from the group consisting of a detergent, a soap, and a fragrance. In certain embodiments, the cleaning compounds is for use in bathrooms and transit areas.

The present disclosure provides for a method of producing methane from waste material, comprising applying a composition comprising the present spore (e.g., the bacterial spore) to waste material.

The present disclosure relates to a method of treating animal waste, bedding or litter comprising applying a composition comprising the present spore (e.g., the bacterial spore) to animal waste, bedding or litter.

In another aspect, the present disclosure relates to a method of preparing silage comprising applying a composition comprising the present spore (e.g., the bacterial spore) to silage.

The present disclosure relates to a method for microbial enhanced oil recovery (MEOR), comprising applying a composition comprising the present spore (e.g., the bacterial spore) to an oil well. In certain embodiments, the bacterial spore is Bacillus subtilis, Bacillus licheniformis, Bacillus mojavensis, and other microbes described herein. U.S. Pat. No. 9,610,333.

The present composition may be a pharmaceutical composition. The present disclosure relates to a method of treating or preventing a disease/condition in a subject susceptible to or afflicted with the disease/condition. The method may comprise administering to the subject an effective amount of the present composition (comprising a microbial spore such as a bacterial spore or a fungal spore). In certain embodiments, the disease is a bacterial disease, a fungal disease or a viral disease. In certain embodiments, the disease is necrotic enteritis. In certain embodiments, the subject is a human. In certain embodiments, the subject is a non-human animal. In certain embodiments, the subject is a chicken, a turkey or a duck. In certain embodiments, the subject is selected from the group consisting of fish, shrimp, or mollusks. In certain embodiments, the subject is infected with Clostridium perfringens. In certain embodiments, treating or preventing the disease results in one or more of reducing mortality, reducing lesion number, and increasing weight gain compared to a subject that is not administered the composition.

The present modified spore may be used as a vehicle for heterologous antigen delivery and vaccination. In one embodiment, heterologous antigens displayed on the spore surface as a fusion product with spore coat proteins can be used to elicit protective immune responses. The composition may comprise the microbial spore and an adjuvant. An adjuvant may be administered together with an antigen which nonspecifically enhances the immune response to that antigen. Adjuvants may be insoluble and undegradable substances (e.g., inorganic gels such as aluminum hydroxide), or water-in-oil emulsions such as incomplete Freund's adjuvant. Generally, adjuvants may retard the destruction of antigen and allow the persistence of low but effective levels of antigen in the tissues and also nonspecifically activate the lymphoid system by provoking an inflammatory response. One adjuvant is Freund's complete adjuvant having mycobacteria suspended in a water-in-oil emulsion. For example, the adjuvant may be cholera toxin, a non-toxic variant of Escherichia coli labile toxin, and a portion or a derivative thereof.

The present modified spore may be used as a vehicle for drug and/or enzyme delivery where the drug and/or enzyme is the exogenous gene product (which may be associated with the spore coat).

The composition may be formulated to be administered by one or more of the following routes: intravenous, intramuscular, intraperitoneal, intradermal, intrapulmonary, intravaginal, rectal, oral, buccal, sublingual, intranasal, intraocular, and subcutaneous.

In certain embodiments, the composition is in the form of a dry powder. In certain embodiments, the composition is in the form of an emulsion. In certain embodiments, the compositions may be prepared in the forms of granules, briquettes, pellets, tablets, or capsules.

The present disclosure provides a kit comprising: a vector described herein; or a composition described herein. The kit may comprise instructions for using the vector, or the composition, in a method described herein. In a related embodiment, the kit further includes an applicator for the composition, for example, a spray bottle, a nasal sprayer, a fluid dropper, an oral inhaler, a nasal inhaler, or a syringe.

An embodiment provides a vaccination kit that includes a unit dose of the composition according to any of embodiments herein, a container, and instructions for use.

The following examples of specific aspects for carrying out the present invention are offered for illustrative purposes only, and are not intended to limit the scope of the present invention in any way.

### Example 1

Developments in genome editing offer potential solutions to challenges in agriculture, industry, medicine, and the environment. However, many technologies remain unexploited due to limitations in the use of genetically altered organisms. In this study, we use B. subtilis spores to explore the possibility of bioengineering organisms while leaving their genome intact. Taking advantage of the differential expression between the mother cell and the fore-spore compartments during sporulation, we created plasmids programmed to modify the spore phenotype from the mother cell compartment, but to “self-digest” in the forespore. At the end of sporulation, the mother cell undergoes lysis and releases the phenotypically engineered, genetically unaltered spores. Using this approach, we demonstrated the potential to express foreign proteins in B. subtilis spores without genome alterations by producing spores expressing GFP in their protective coats, where approximately 90% of the spore population had no detectable plasmid or chromosome alterations. In a separate demonstration, we programmed KinA overexpression during vegetative growth to artificially induce sporulation, and also obtained spores with nearly 90% of them free of detectable plasmid. Artificial induction of sporulation could potentially simplify the bioprocess for industrial spore production, as it reduces the number of steps involved. Overall, these findings demonstrate the potential to create genetically intact bioengineered organisms.

In this work, we investigated the possibility of creating genetically intact bioengineered organisms by introducing synthetic gene circuits in plasmids that are deleted once the phenotype is completed. Plasmid DNA digestion in vivo has been previously performed efficiently in the bacteria E. coli, although with different purposes that resulted in the final product carrying genome alterations.^{16,17} However, such studies revealed that plasmid DNA digestion in vivo is possible. In contrast to these studies in which plasmid DNA digestion circuitry is introduced separately by a phage¹⁶ or by integration into the chromosome,¹⁷ here we developed a DNA digestion circuit that is self-contained in the target plasmid that also carries the phenotype genes.

When the self-digestion circuit and phenotype genes are self-contained in the same plasmid, it becomes important to avoid DNA self-digestion before expression of the phenotype. The process of sporulation in Bacillus offers an opportunity to exert control over self-digestion and phenotype expression due to differential expression in the mother cell and the fore spore compartments during sporulation.^{18,19} Using B. subtilis spores as a model organism, we exploited differential expression by building plasmids that behave differently in each compartment by leveraging spore-specific promoters. The plasmids modify the spore from the mother cell, while they suffer programmed self-digestion inside the fore-spore. This programmed self-digestion is triggered when the spore-specific promoters on the plasmid become activated during sporulation. The promoters drive the expression of DNA nuclease genes which are also on the plasmid. Then, the DNA nucleases target the plasmid itself, cut it, and digest it. At the end of sporulation, the mother cell undergoes lysis and releases the genetically intact bioengineered spores.

In the context of bioengineering with synthetic gene circuits, our approach introduces a bioengineering tool that leverages differential expression patterns in different compartments of an organism. The synthetic gene circuits we developed in this work are programmed to behave differently in different cellular compartments by responding to natural differences in expression patterns,^{18,19} without relying on the addition of chemicals or promoter inducers. The present method can also be used in other organisms by harnessing differential expression of genes in different compartments and tissues. Here, this capability is exploited to modify the spore from the mother cell and self-digest the plasmid in the forespore upon activation of certain spore-specific promoters.

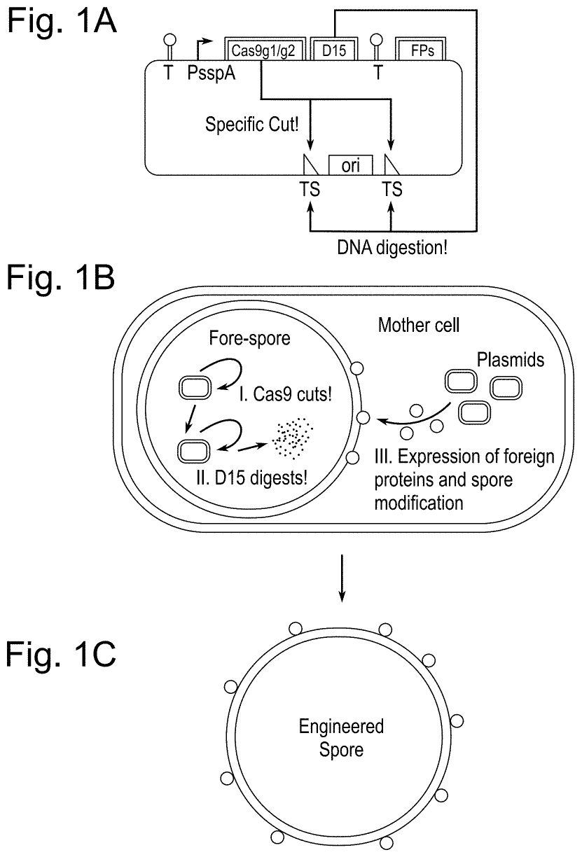

Our approach is summarized in FIGS. 1A-1C. The plasmids we designed contain a synthetic self-digestion gene circuit (FIG. 1A) that consists of PsspA (a sporulation-specific promoter²⁰), Cas9g1 or Cas9g2 (a combination of the Cas9 gene and one or two different sgRNAs, respectively), and D15 (the exonuclease from the T5 phage²¹). Transcriptional terminators isolate the self-digestion circuit from the rest of the genetic elements in the plasmid such as the origin of replication or other expression cassettes. Target sites, sequences meant to be targeted by Cas9g1 or Cas9g2, are located in the plasmid itself, close to the replication origin. Some plasmids additionally contain expression cassettes encoding foreign proteins, devised to modify the spore phenotype, and these are positioned outside the self-digestion circuit. The promoter PsspA starts being active at time point T3 of sporulation, and only inside the forespore.²⁰ By the stage T3, mother cell and fore-spore are already well-defined compartments, which prevents Cas9 and D15 from digesting the plasmid in the mother cell. In the forespore, Cas9g1 or Cas9g2 recognize the target sites and linearize the plasmid, which is later digested into single nucleotides by D15. Because the plasmid in the mother cell is intact, the foreign proteins are expressed constitutively to modify the spore (FIG. 1B). By the end of sporulation, the mother cell is lysed, and the engineered spores are released and purified. The final product is engineered spores with intact genomes (FIG. 1C).

Using the self-digestion circuit, we created plasmids to either produce spores expressing GFP in their protective coats, or overexpress KinA, a histidine kinase that was previously reported to induce sporulation artificially. We first determined plasmid stability during vegetative growth and sporulation, and then we analyzed plasmid self-digestion once sporulation was completed by quantifying average plasmid copy numbers per cell or per spore, respectively. Then we analyzed the presence of plasmids in colonies generated from individual spores upon germination by colony PCR. The results of these analyses showed that for both GFP expressing and KinA overexpressing spores, approximately 90% of the spores contained no detectible plasmid. We further investigated those cases in which plasmids were retained, and found that the plasmids contained mutations in key elements of the self-digestion program.

### Results and Discussion

Evaluation of Plasmid Self-Digestion at the Population Level Through qPCR.

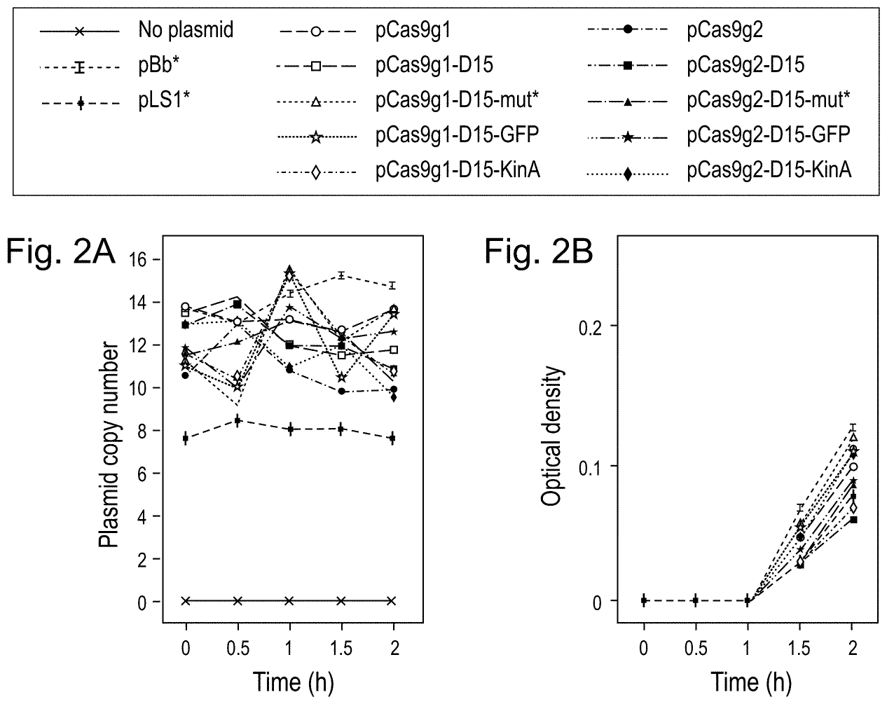

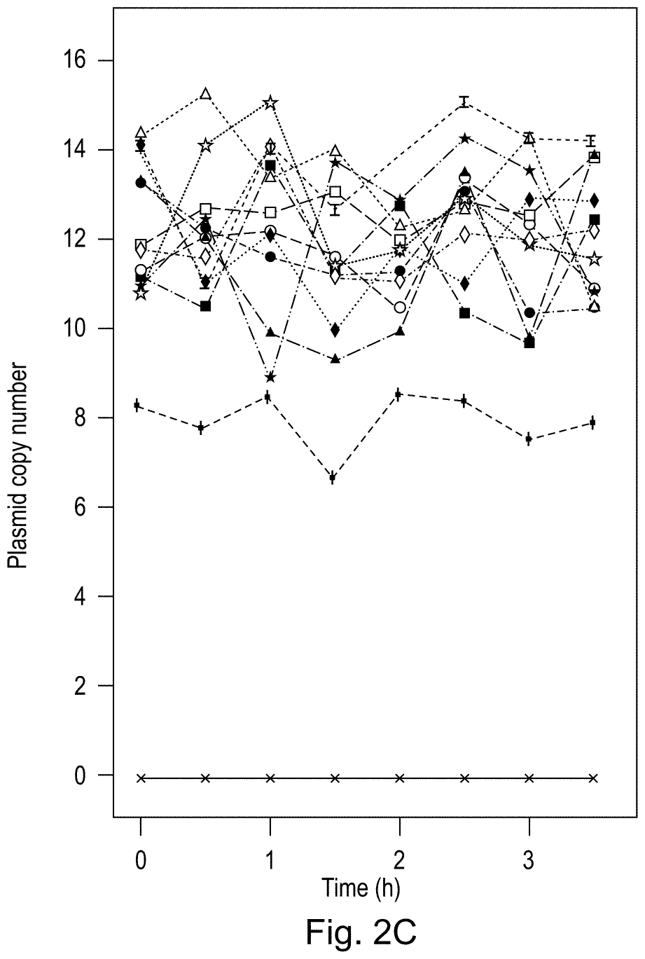

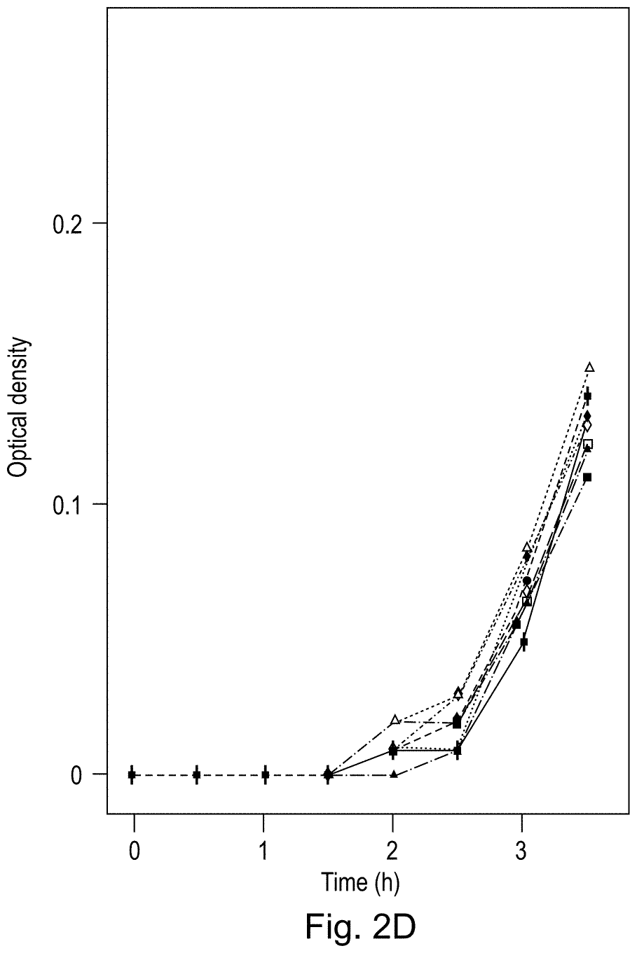

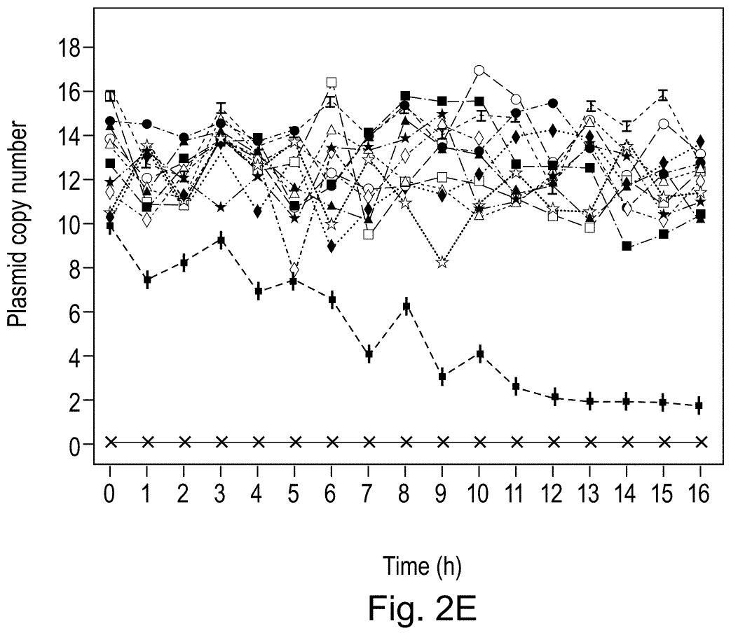

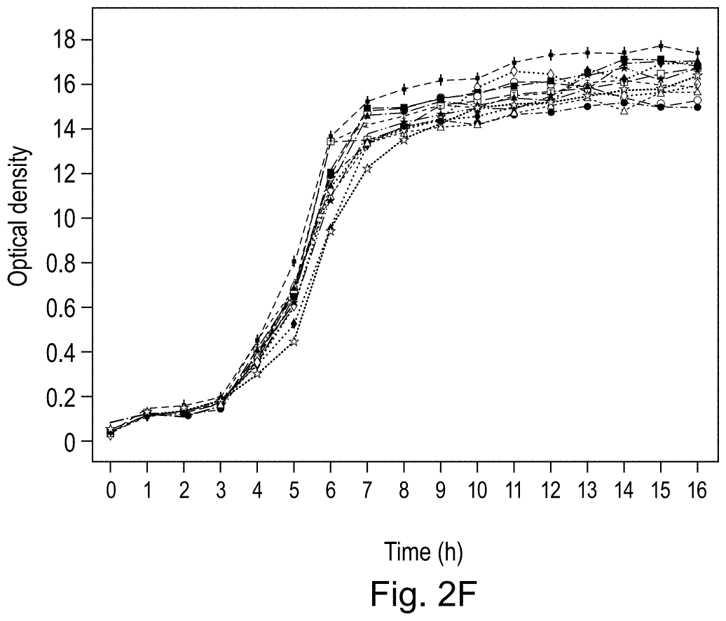

To evaluate the success of the programmed self-digestion of the plasmids, we first quantified plasmid DNA in spores at the population level. Reports of quantification of plasmid DNA in spores is limited to a small number of published studies.²²⁻²⁴ These studies show that while some plasmids are present in all spores,^{23,24} others such as pLS1,²⁴ are unstable such that only a small fraction of the spore population retained it. Plasmid stability in the mother cell is particularly important because in all the reported engineered spore phenotypes, it is the spore outer layers that get modified by proteins expressed in the mother cell. Therefore, we first analyzed whether our plasmids were stable during vegetative growth and in the mother cell during sporulation. After transforming B. subtilis with our plasmids, bacteria was grown in LB media for 2 h, followed by growth in sporulation media (DSM) for 3 h, followed by sporulation in DSM for 24 h (without antibiotic in the three cases). Plasmid copy number (PCN) was quantified through each stage as described in the methods section, and we observed that the plasmids were stable in the mother cell through all three stages, with the exception of plasmid pLS1 (FIGS. 2A-2F). The loss of pLS1 was anticipated in agreement with previous reports that show this plasmid to be unstable during vegetative growth in the absence of antibiotic as selective pressure, while most of it is lost during sporulation independently of the presence or absence of antibiotic,²⁴ although the molecular mechanism by which this occurs remains obscure. In this experiment, we used pLS1 as a control that allowed us to test if our experiment could detect plasmid instability.

After plasmid stability experiments, we transformed B. subtilis with the various plasmids and generated the different spore strains. These strains were analyzed through qPCR, and the average PCN per spore was determined and compared among strains to determine if plasmid self-digestion occurred. Three positive controls were used in this case: pBb (the Backbone plasmid used to build all the plasmids generated in this study), and pCas9g1-D15-mut and pCas9g2-D15-mut (plasmids for which the promoter driving expression of the DNA nucleases was deleted). Our results showed that a significantly reduced PCN per spore was detected in strains pCas9g1-D15, pCas9g2-D15, and variants from both (Table 1), while positive controls kept a higher PCN per spore. Strains that only contained the endonuclease showed no reduction in PCN per spore, suggesting that plasmid self-digestion occurred as a result of the coupled activity of endonuclease and exonuclease.

TABLE 1
Average Plasmid Copy Number per Spore

| | | Plasmid to |
|---|---|---|
| | Components for | chromo- |
| | self-digestion or | some |
| Plasmid | generation of a phenotype | ratio |
| pBb^{a} | | 6.51 |
| pLS1^{b} | | 0.02 |
| pCas9g1 | Cas9, and one sgRNA | 5.33 |
| pCas9g1- | Cas9, one sgRNA, and D15 | 0.11 |
| D15 | | |
| pCas9g1- | Cas9 and D15 with no | 6.38 |
| D15-mut^{a} | promoter, and one sgRNA | |
| pCas9g1- | Cas9, one sgRNA, | 0.21 |
| D15-GFP | D15, and GFP | |
| pCas9g1- | Cas9, one sgRNA, KinA | 0.36 |
| D15-KinA | | |
| pCas9g2 | Cas9, and two sgRNAs | 4.13 |
| pCas9g2- | Cas9, two sgRNAs, and | 0.16 |
| D15 | D15 | |
| pCas9g2- | Cas9 and D15 with no | 6.21 |
| D15-mut^{a} | promoter, and two sgRNAs | |
| pCas9g2- | Cas9, two sgRNAs, | 0.15 |
| D15-GFP | D15, and GFP | |
| pCas9g2- | Cas9, two sgRNA, KinA | 0.17 |
| D15-KinA | | |

^{a}Controls where plasmid self-digestion was not expected.
^{b}Control where plasmid loss was expected.

### Evaluation of Plasmid Elimination at the Individual Cell Level Through Colony PCR.

Although the experiments with qPCR report a substantial reduction in average PCN values, it does not allow the determination of the presence and absence of plasmids in individual spores. To determine and count the number of individual plasmid-free spores, we germinated spores, allowing them to replicate any plasmids retained, and then analyzed colonies for plasmid presence/absence through colony PCR. To achieve this, we heat-activated spores, germinated them in LB or valine media as previously described,²⁵ and plated them on LB-agar for development of individual colonies. The valine media was used because it allows germination of superdormant spores, which germinate poorly otherwise, even in the presence of other nutrients.²⁵ Germinating superdormant spores was important given that it is unknown if the presence of plasmid DNA in Bacillus affects the rate at which superdormant spores are generated. Thus, germinating superdormant spores assures a more precise way of counting plasmid-free spores.

Colony PCR was performed with primers targeting a conserved region among all our designer plasmids, and we found that nearly 90% of bacterial colonies derived from strains pCas9g1-D15, pCas9g2-D15, and variants from both, had no detectable plasmid (Table 2). On the other hand, plasmid was detected in all of the colonies derived from positive control strains (pBb, pCas9g1-D15-mut, and pCas9g2-D15-mut). Similarly, plasmid was detected in all the colonies examined that derived from plasmids that contained the Cas9 endonuclease, but not the D15 exonuclease (pCas9g1 and pCas9g2).

TABLE 2
Ratio of Spores That Tested
Negative for Plasmid

| | Negative colonies | |
|---|---|---|
| Plasmid | LB | Valine |
| pBb^{a} | 0/100 | 0/100 |
| pLS1^{b} | 96/100 | 99/100 |
| pCas9g1 | 0/100 | 0/100 |
| pCas9g1-D15 | 89/100 | 88/100 |
| pCas9g1-D15-mut^{a} | 0/100 | 0/100 |
| pCas9g1-D15-GFP | 89/100 | 89/100 |
| pCas9g1-D15-KinA | 88/100 | 84/100 |
| pCas9g2 | 0/100 | 0/100 |
| pCas9g2-D15 | 92/100 | 86/100 |
| pCas9g2-D15-mut^{a} | 0/100 | 0/100 |
| pCas9g2-D15-GFP | 88/100 | 91/100 |
| pCas9g2-D15-KinA | 87/100 | 86/100 |

^{a}Controls where plasmid self-digestion was not expected.
^{b}Control where plasmid loss was expected.

In contrast with our results, previous studies have achieved targeted plasmid digestion in vivo in E. coli with the use of the endonuclease Cas9 alone, without an additional exonuclease, ^{16,17} indicating that the elements necessary for successful DNA digestion in vivo may vary from host to host.

Another notable difference between our study and those performed in E. coli is the origin of the DNA digestion. While in our study the plasmids are programmed to undergo self-digestion (cis-acting), the studies mentioned above contain the DNA digestion program in a DNA molecule distinct from the one being targeted (trans-acting). Therefore, their synthetic gene circuits continue to express the Cas9 indefinitely, while in our study, the expression of Cas9 decreases as the amount or the integrity of the target plasmids decrease. This could be part of the reason why the plasmid is retained in some spores, although we later demonstrate that various mutations arise during sporulation, and such may be the cause for plasmid retention. Note that in the studies in E. coli previously mentioned, the resulting bacteria cannot be classified as to have a wild type genome anymore.

In agreement with our strategy for having plasmids undergo self-digestion during sporulation, data from positive controls in qPCR and colony PCR experiments suggest that plasmid loss did not occur throughout the processes of germination or growth for colony formation. Indeed, studies show that plasmid replication during germination tends to be more robust than chromosome replication.^{24,26}

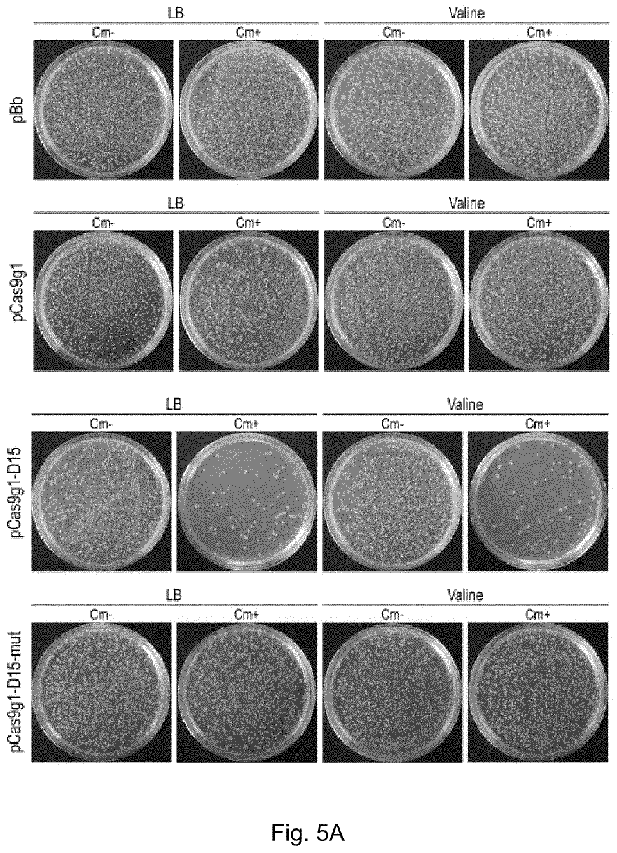

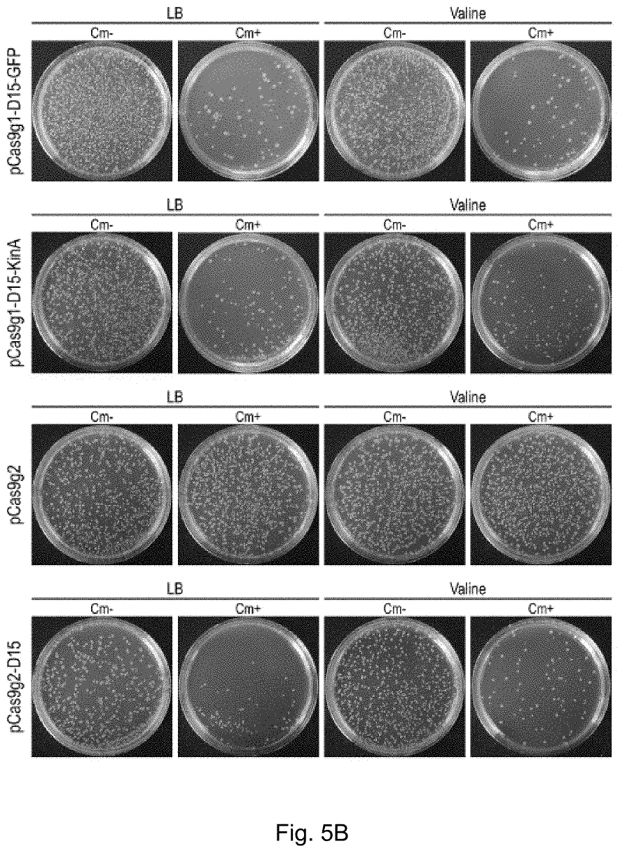

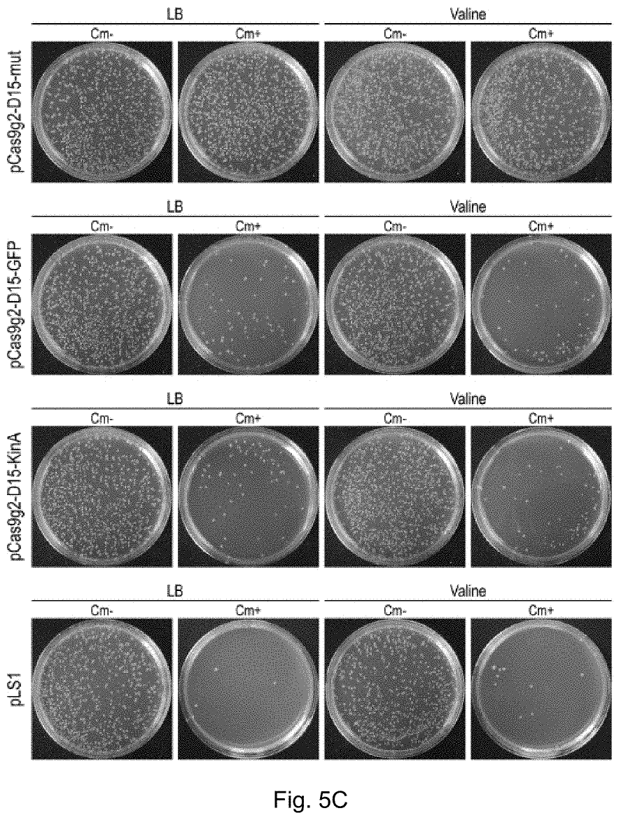

To further support the idea that plasmid loss occurred during sporulation, we performed another experiment in which we compared germination and colony formation in media with or without antibiotic, for which the plasmids provide resistance (FIGS. 5A-5C). Thus, we would have expected that if plasmid loss occurred during germination, positive controls would have shown fewer colonies in media with antibiotic than in media without it. However, only strains pCas9g1-D15, pCas9g2-D15, and GFP and KinA derivatives showed fewer colonies, in agreement with qPCR results, suggesting that plasmid loss occurred during sporulation.

Our analysis thus far focused on quantification of plasmids, but it could also be possible that cells contain fragments of plasmid DNA integrated into the chromosome. Although processes such as plasmid-chromosome recombination are known to be rare,²⁷ we nevertheless developed a strategy to test plasmid integration into the chromosome (described in the Methods section). Because the prevalence of such chromosome alterations are small, it is difficult to quantify the precise fraction of cells that contain such alterations. Our tests done with six different plasmids containing the self-digestion circuit showed that out of the six hundred colonies tested, only five colonies had detectible plasmid integration in the chromosome. (Tables 6-7).

TABLE 6
Plasmid integration into the chromosome

| Plasmid | Colonies with recombination |
|---|---|
| pCas9g1-D15 | 0/100 |
| pCas9g1-D15-GFP | 1/100 |
| pCas9g1-D15-KinA | 1/100 |
| pCas9g2-D15 | 1/100 |
| pCas9g2-D15-GFP | 0/100 |
| pCas9g2-D15-KinA | 2/100 |

TABLE 7
Primers used for identification of plasmid
integration events through reverse PCR

| Oligo | Target | Oligo sequence (5′→3′) | Plasmid(s) |
|---|---|---|---|
| R001 | 1 | CCTGAATAGAGTTCATAAACAATCC | All |
| R002 | | TTCAGGAATTGTCAGATAGGCC | |
| R003 | 1A | AACAATCCTGCATGATAACCATC | |
| R004 | | GTCAGATAGGCCTAATGACTG | |
| R005 | 2 | TGCAGGGTAAAATTTATATCCTTCT | All |
| R006 | | TTAGTGACAAGGGTGATAAACTC | |
| R007 | 2A | ATATCCTTCTTGTTTTATGTTTCGG | |
| R008 | | AGGGTGATAAACTCAAATACAGC | |
| R009 | 3 | TACGGTGTAAACCTTCCTCCA | All |
| R010 | | TACACCGTAAAAGGTTAATCTCC | |
| R011 | 3A | CCTCCAAATCAGACAAACGTTTC | |
| R012 | | GGTTAATCTCCTATGGTGGTTTG | |
| R013 | 4 | CATTGCTCTCCTCCAGTTGC | All |
| R014 | | AATGAAACATGGCATTCAGTCAC | |
| R015 | 4A | CAGTTGCACATTGGACAAAGC | |
| R016 | | GTCACAAAAGGTTGTTGCTGAAG | |
| R017 | 5 | CCTCTCGGTTATGAGTTAGTTC | All |
| R018 | | AAGAACGAAGTCGAGATCAGG | |
| R019 | 5A | GTTATGAGTTAGTTCAAATTCGTTC | |
| R020 | | GTCGAGATCAGGGAATGAGTT | |
| R021 | 6 | AACTCGGTCGCCGCATACA | All |
| R022 | | TTGCCCGGCGTCAACACG | |
| R023 | 6A | CCGCATACACTATTCTCAGAATG | |
| R024 | | ACACGGGATAATACCGCGC | |
| R025 | 7 | TAGTTATCTACACGACGGGGA | All |
| R026 | | ACGATACGGGAGGGCTTAC | |
| R027 | 7A | AGTCAGGCAACTATGGATGAAC | |
| R028 | | CCATCTGGCCCCAGTGCT | |
| R029 | 8 | GGATAAGGCGCAGCGGTC | All |
| R030 | | GGTAACTATCGTCTTGAGTCC | |
| R031 | 8A | AGCGGTCGGACTGAACGG | |
| R032 | | GTCCAACCCGGAAAGACATG | |
| R033 | 9 | AGCTATGAGATGGAGAAAGCC | All |
| R034 | | TACCCAGGCTTAACCTTCTTC | |
| R035 | 9A | GCCATAACCATGAGTGATAACAC | |
| R036 | | CTTCTTCGGCTTTTTCACGAG | |
| R037 | 10 | GCGTGTTCAGACTAGAACATC | All |
| R038 | | CCCTTATCACCTAGAACAATCGT | |
| R039 | 10A | ACCAGAGTATAAAGGTAATCGTG | |
| R040 | | ATCGTAGTTCTGGCAGAGTAG | |
| R041 | 11 | TGCTATTGCTGCTGTAGGTCA | All |
| R042 | | CCACACAGTAGGTAGGTAAATC | |
| R043 | 11A | CAAGATGTGTTAGATAAGTTCACG | |
| R044 | | ATCAACCAGAATCAAGTTTCGGA | |
| R045 | 12 | GATTGAGGGAGATTTAAATCCTG | All |
| R046 | | CCAAATAGATTAAGCGCAAATCC | |
| R047 | 12A | TCCTGATAATAGTGATGTGGACA | |
| R048 | | CCGCTTTATCAGTAGAATCTACC | |
| R049 | 13 | TTATATTGATGGGGGAGCTAGC | All |
| R050 | | CATCAATATAACCTGCATATCCG | |
| R051 | 13A | GGGGAGCTAGCCAAGAAGA | |
| R052 | | CCTGCATATCCGTTTTTTGATTG | |
| R053 | 14 | CCAGCATTTCTTTCAGGTGAAC | All |
| R054 | | GGTTTTCGCATTCCTTCAGTAAC | |
| R055 | 14A | GAACAGAAGAAAGCCATTGTTGA | |
| R056 | | AACATATTTGACCTTTGTCAATTCG | |
| R057 | 15 | GTCAAAGTAATGGGGCGGCA | All |
| R058 | | TGTTCATGTAAACTATCGCCTTG | |
| R059 | 15A | CGGCATAAGCCAGAAAATATCG | |
| R060 | | CCTTGTCCAGACACTTGTGC | |
| R061 | 16 | GGTTGAAACTCGCCAAATCAC | All |
| R062 | | AATTGGCGTTTGATAAAACCAGC | |
| R063 | 16A | AAATCACTAAGCATGTGGCACAA | |
| R064 | | GCTTTATCAAGTTCACTCAAACC | |
| R065 | 17 | CACAGTGCGCAAAGTATTGTC | All |
| R066 | | CTGTGGCAAAATCTCGCCC | |
| R067 | 17A | TCCATGCCCCAAGTCAATATTG | |
| R068 | | CTCGCCCTTTATCCCAGACA | |
| R069 | 18 | GCTAGTCATTATGAAAAGTTGAAGG | All |
| R070 | | CACATATTTGCTTGGCAGAGC | |
| R071 | 18A | GTTGAAGGGTAGTCCAGAAGA | |
| R072 | | GCAGAGCCAGCTCATTTCC | |
| R073 | 19 | GAGATCTGCAGTGACTGGGA | pCas9g1-D15 |
| R074 | | ATCTCTAGAAGCTCCAGTCAC | |
| R075 | 19A | CCTGGCGACTAGTCTTGGA | |
| R076 | | TCACTGCAGATCTCTAGAAGC | |
| R077 | 20 | GAGATCAGGTCAGACGCGAT | pCas9g2-D15 |
| R078 | | ATCTCTAGAAGCTCCAGTCAC | |
| R079 | 20A | GATTTCCTGGGTGCTCGAG | |
| R080 | | TCACTGCAGATCTCTAGAAGC | |
| R081 | 21 | CTCCATCTGGATTTGTTCAGAAC | pCas9g2-D, pCas9g2- |
| R082 | | AGTTCTGAGGTCATTACTGGATC | D15- |
| R083 | 21A | AGAACGCTCGGTTGCCGC | GFP, pCas9g2-D15- |
| R084 | | TGGATCTATCAACAGGAGTCC | KinA |
| R085 | 22 | GCAAGCTTGACAAGTATTTCTTC | pCas9g1-D15-GFP, |
| R086 | | TGCCATCTTAACCGGATAAGG | pCas9g2- |
| R087 | 22A | GGAGGGCAAACTACTAGAGATG | D15-GFP |
| R088 | | GATAAGGAGATCATTGAGGAAGC | |
| R089 | 23 | CGTGCTTGAGGCGGACGA | pCas9g1-D15-GFP, |
| R090 | | CACCGGTGTTTGTTCCTCGA | pCas9g2- |
| R091 | 23A | ACGACCTGTCAGAAGTGTTCA | D15-GFP |
| R092 | | CGATCTTAAGGTCCGGTTCG | |
| R093 | 24 | GCAGTAGCGACTACCAGAG | pCas9g1-D15-GFP, |
| R094 | | TGAGCTTTTGCTGCTCTGGTA | pCas9g2- |
| R095 | 24A | ACCAGAGTAGTCGTAGCCC | D15-GFP |
| R096 | | GGTAGTCGCTACTCTTCAGG | |
| R097 | 25 | GGAGGACGGAAACATTCTGG | pCas9g1-D15-GFP, |
| R098 | | TCGATTCCTTTGAGTTCAATACG | pCas9g2- |
| R099 | 25A | TTCTGGGCCACAAACTGGAAT | D15-GFP |
| R100 | | TACGGTTTACCAGGGTGTCG | |
| R101 | 26 | TTGGTGAGAATCCAGGGGTC | pCas9g1-D15-GFP |
| R102 | | GTTCTGAACAAATCCAGATGGAG | |
| R103 | 26A | GGGGTCCCCAATAATTACGAT | |
| R104 | | GGAGTTCTGAGGTCATTACTG | |
| R105 | 27 | CGCCGAATGAAGCGGATGA | pCas9g2-D15-GFP |
| R106 | | TCCACCACCGCTTTCCCAT | |
| R107 | 27A | ATGAAATTGCTCATCGTCTCGTG | |
| R108 | | ATGGCTCGAGCACCCAGG | |
| R109 | 28 | GACAGCCGAATTCGGATTCC | pCas9g1-D15-KinA, |
| R110 | | AAGCTTGCCATCTTAACCGGA | pCas9g2- |
| R111 | 28A | GGATTCCCGCGTATATCTTGA | D15-KinA |
| R112 | | GATAAGGAGATCATTGAGGAAGC | |
| R113 | 29 | GAACCTGCAAGCCCGGAAT | pCas9g1-D15-KinA, |
| R114 | | TCGCAGTTTAGGGATTGATGG | pCas9g2- |
| R115 | 29A | GGAATCGACTACATATATAACGG | D15-KinA |
| R116 | | GATGGCCTGTTTCTTCTTCAAG | |
| R117 | 30 | TTGTCAAGCAATCCTGGTTCAC | pCas9g1-D15-KinA, |
| R118 | | TCTCTTTTACATCCTCGTGATCG | pCas9g2- |
| R119 | 30A | CCTTTCAGAACAGGGTCATCTA | D15-KinA |
| R120 | | TCGCAAGGATGCAGCTGATC | |
| R121 | 31 | ACCGAAGATGAGCATTCTGTTC | pCas9g1-D15-KinA, |
| R122 | | TATGATAATGTCTACTGTTCCCC | pCas9g2- |
| R123 | 31A | GTTCATGTTACTGTCAAAGACGA | D15-KinA |
| R124 | | CCCATCAGGCATTGATTCAAC | |
| R125 | 32 | ACATGTGGACAGCCATCCTG | pCas9g1-D15-KinA, |
| R126 | | ATGTATAACTCCCTGATGGTTTTC | pCas9g2- |
| R127 | 32A | ATCCTGAAAAAGGCACAGCGT | D15-KinA |
| R128 | | TGTCACCATTAATCCAAGCCC | |
| RR129 | 33 | GTCATAGCTGTTTCCTGTGTG | pUC57 |
| RR130 | | CAAGCTTGGCGTAATCATGGT | |
| RR131 | 33A | TGTGTGAAATTGTTATCCGCTCA | |
| RR132 | | TGCAGAGGCCTGCATGCAA | |
| RR133 | 34 | CCGCTTACCGGATACCTGT | pUC57 |
| RR134 | | GAAGCTCCCTCGTGCGCT | |
| RR135 | 34A | TGTCCGCCTTTCTCCCTTC | |
| RR136 | | CAGGCGTTTCCCCCTGGA | |
| RR137 | 35 | GAACGAAAACTCACGTTAAGGG | pUC57 |
| RR138 | | ACGGGGTCTGACGCTCAG | |
| RR139 | 35A | GGGATTTTGGTCATGAGATTATC | |
| RR140 | | GGATCTCAAGAAGATCCTTTGAT | |
| RR141 | 36 | AGCGGTTAGCTCCTTCGGT | pUC57 |
| RR142 | | CCCAACGATCAAGGCGAGTT | |
| RR143 | 36A | GGTCCTCCGATCGTTGTCA | |
| RR144 | | GGCTTCATTCAGCTCCGGTT | |
| RR145 | 37 | CACCTGACGTCTAAGAAACCAT | pUC57 |
| RR146 | | ACATTTCCCCGAAAAGTGCCA | |
| RR147 | 37A | AGGCGTATCACGAGGCCC | |
| RR148 | | TAGGGGTTCCGCGCACATTT | |
| RR149 | 38 | GAAAGGGGGATGTGCTGCAA | pUC57 |
| RR150 | | CTTCGCTATTACGCCAGCTG | |
| RR151 | 38A | TCGGTGCGGGCCTCTTC | |
| RR152 | | GCAAGGCGATTAAGTTGGGTA | |

Targets labeled with an “A” (1A, 2A, 3A . . . ) correspond to nested PCRs, which are performed when increased specificity is needed.
The sequences of oligo R001-oligo RR152 correspond to SEQ ID Nos: 73-224, respectively.

### Bioengineered Spores: GFP Expressing Spores and Artificial Sporulation.

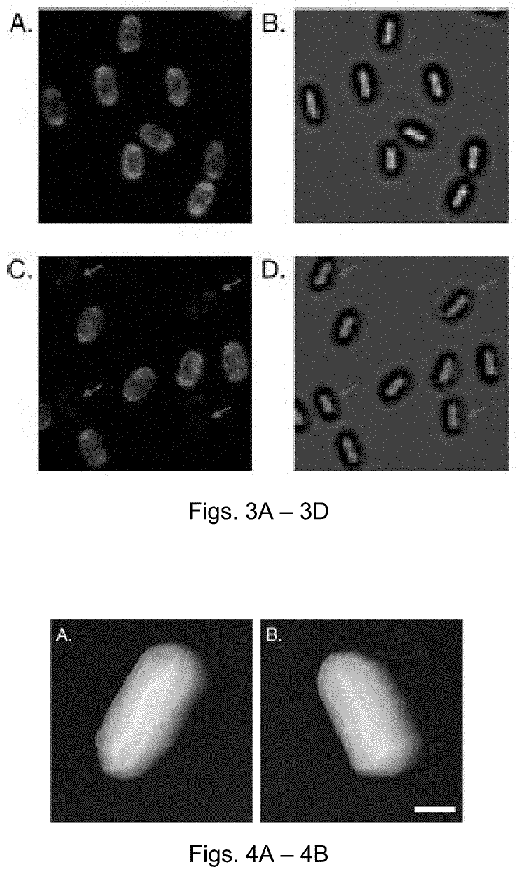

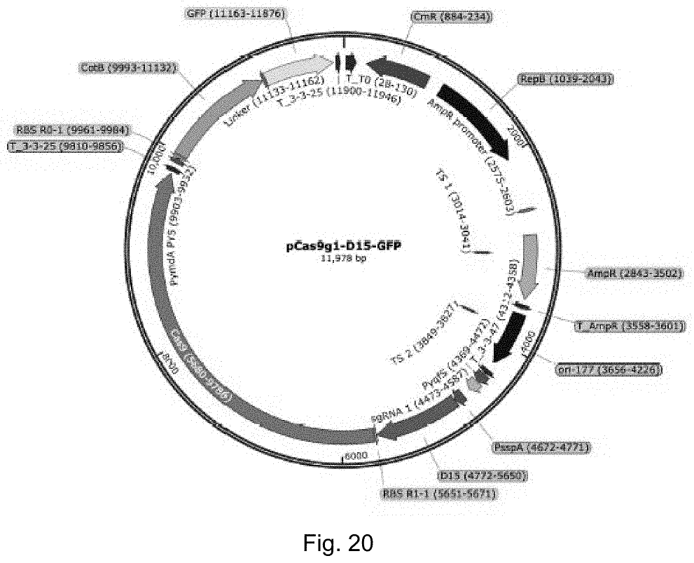

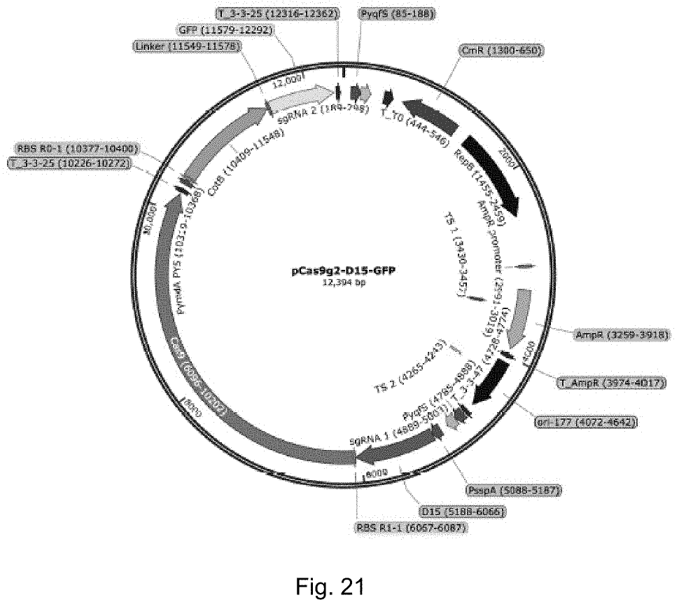

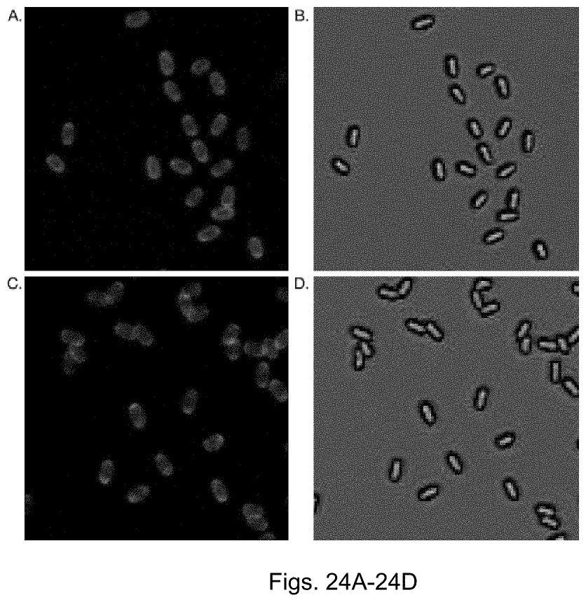

Spores of Bacillus can be bioengineered, primarily through the expression of proteins and peptides on the spore coat.²⁸ Using our plasmids that self-digest in the fore-spore, we explored the possibility of expressing GFP on the coat of spores without altering their genome. To achieve this goal, we took advantage of a fusion protein (CotB-GFP) reported in a previous study, in which the spore coat protein CotB was used to localize GFP on the spore coat.²⁹ We paired a CotB-GFP expression cassette to our circuit for plasmid self-digestion (FIGS. 20-21, Supporting DNA Sequences) and produced spores. We examined these spores through qPCR and PCR as described in the previous sections and found that nearly 90% of them had no plasmid (Tables 1-2). We also studied these spores for green fluorescence through confocal microscopy and found that all expressed GFP on their coats (FIGS. 3A-3D, FIGS. 24A-24D). Thus, while the majority of these spores have plasmids self-digested, they are expressing GFP in their coats. Therefore, these experiments demonstrate the potential for engineering organisms without genome alterations, while taking advantage of the customizability that comes with designer plasmids.

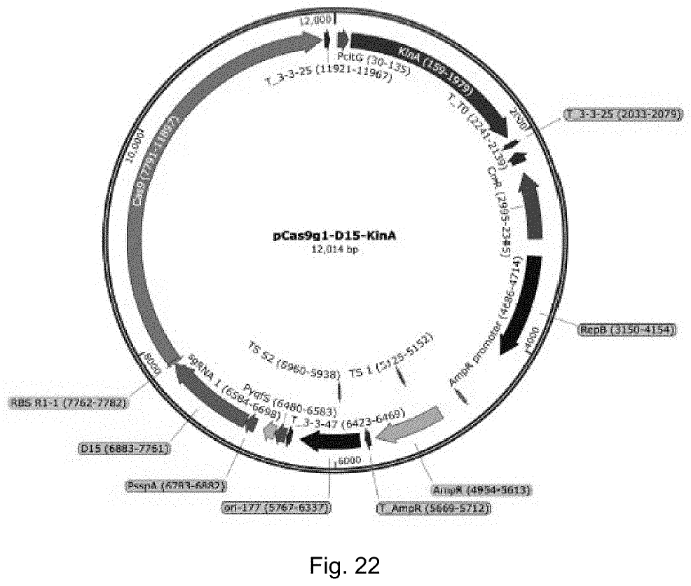

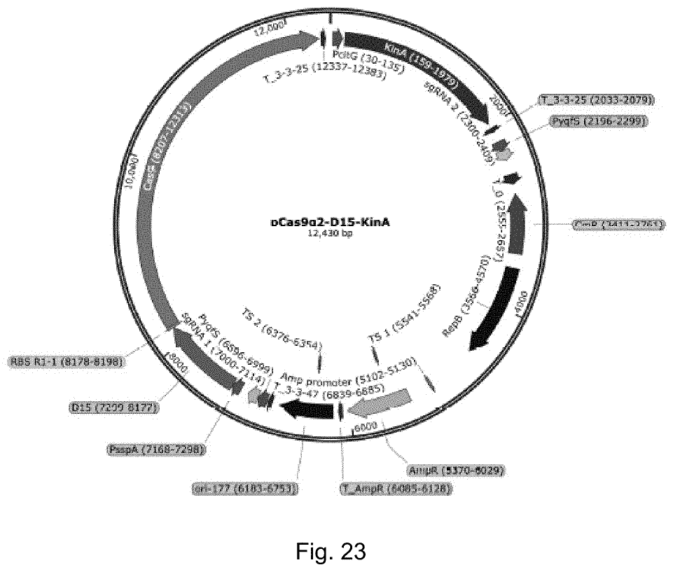

In a separate bioengineering example, we used the self-digesting plasmids containing a synthetic gene circuit to artificially induce sporulation. Previous findings showed that overexpression of KinA in B. subtilis can induce sporulation even in the absence of chemical or physical sporulation inducers.³⁰ On the basis of this finding, we built a gene circuit to overexpress KinA for constitutive sporulation and assembled it together with the circuit for plasmid self-digestion (FIGS. 22-23, Supporting DNA Sequences).

Using the KinA circuit (pCas9g1-D15-KinA and pCas9g2-D15-KinA), we were able to obtain spores in LB media with a sporulation yield comparable to the original study³⁰ (Table 3). We found that approximately 90% of the spores produced with the KinA circuit had no detectable plasmid (Tables 1 and 2).

TABLE 3
Sporulation Induction

| | | CFU/mL | | |
|---|---|---|---|---|
| Plasmid | Medium | Viable cells | Spores | Efficiency |
| no plasmid | LB | 8.3 × 10⁸ | 1.1 × 10⁴ | 1.3 × 10⁻⁵ |
| | DSM | 5.0 × 10⁸ | 4.1 × 10⁸ | 8.2 × 10⁻¹ |
| pBb | LB | 7.8 × 10⁸ | 1.4 × 10⁴ | 1.8 × 10⁻⁵ |
| | DSM | 4.2 × 10⁸ | 2.7 × 10⁸ | 6.4 × 10⁻¹ |
| pCas9g1-D15-KinA | LB | 3.0 × 10⁸ | 1.2 × 10⁸ | 4.0 × 10⁻¹ |
| | DSM | 1.9 × 10⁸ | 0.8 × 10⁸ | 4.2 × 10⁻¹ |
| pCas9g2-D15-KinA | LB | 3.6 × 10⁸ | 1.9 × 10⁸ | 5.2 × 10⁻¹ |
| | DSM | 2.2 × 10⁸ | 1.3 × 10⁸ | 5.9 × 10⁻¹ |

Besides qPCR and colony PCR, we analyzed spores of the strain pCas9g1-D15-KinA through atomic force microscopy and observed that they possessed similar size, and similar morphology to wild type spores, characterized by wrinkles on the spore surface and overall spore dimensions (FIGS. 4A-4B). Thus, we were able to produce spores through artificial sporulation, with the majority without detectible genome alterations, and these appeared to be morphologically similar to the WT spores.

### Isolation and Sequencing of Plasmids Retained.

To better understand why the plasmid was retained in nearly 10% of the spores from strains pCas9g1-D15, pCas9g2-D15, and their derivatives, we isolated the retained plasmids (from strains germinated with LB) and analyzed each plasmid by DNA sequencing. We detected that several mutations emerged on DNA fragments that are key for the functioning of the self-digestion gene circuit such as the sequence being targeted by Cas9, or the Cas9 gene itself (Table 4).

TABLE 4
Mutations in Retained Plasmids^{a}

| | TS mutation | TS | truncated |
|---|---|---|---|
| | and | complete | Cas9 |
| Plasmid | Indels | deletion | gene |
| pCas9g1-D15 | 6/11 | 3/11 | 2/11 |
| pCas9g1-D15-GFP | 9/11 | 1/11 | 1/11 |
| pCas9g1-D15-KinA | 11/12 | 1/12 | 0/12 |
| pCas9g2-D15 | 8/8 | 0/8 | 0/8 |
| pCas9g2-D15-GFP | 11/12 | 1/12 | 0/12 |
| pCas9g2-D15-KinA | 9/13 | 3/13 | 1/13 |

^{a}Data represents the number of plasmids of a species with mutations of a certain category over the total plasmids retained of that species.

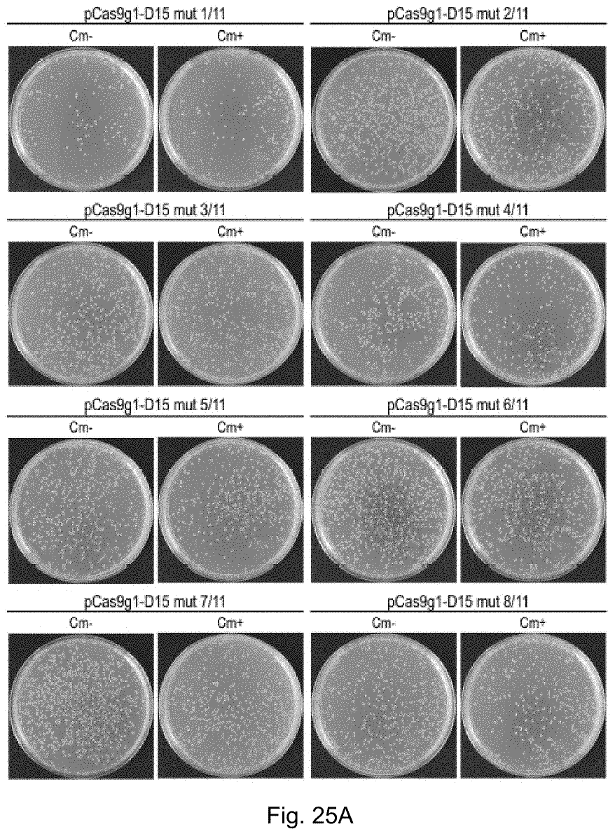

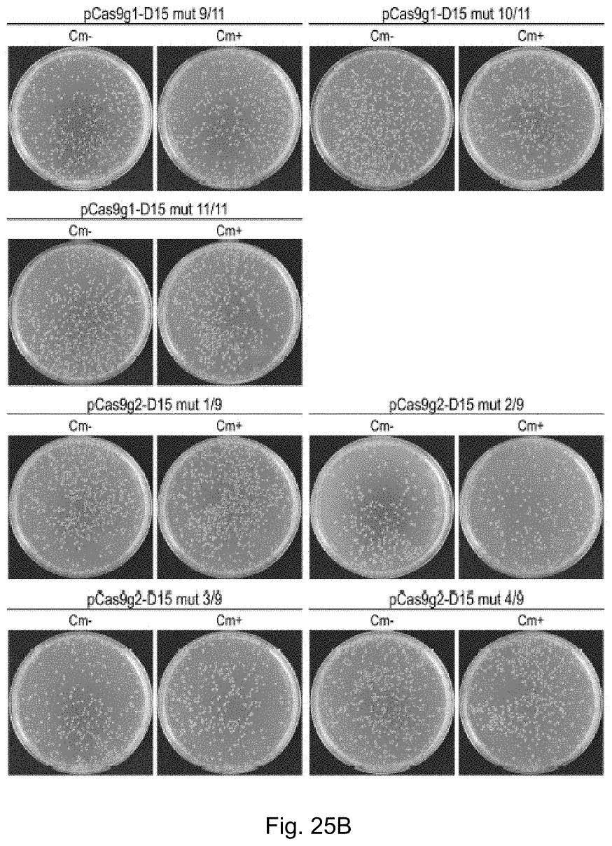

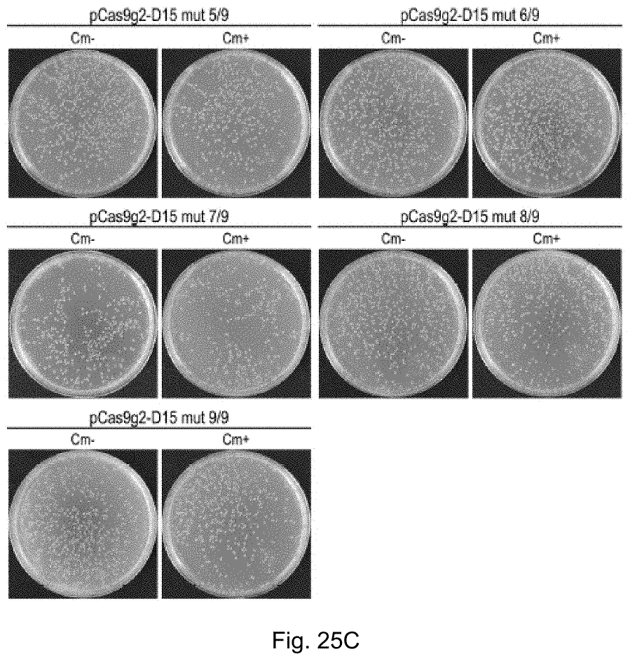

To confirm whether mutations in the plasmids were responsible for the failure of plasmid self-digestion, we used the isolated plasmids to transform wild type bacteria and performed sporulation. We analyzed the newly generated spores by comparing germination on antibiotic-free media vs media containing antibiotic and observed a similar amount of colonies in both media (FIGS. 25A-25C). This resistance to antibiotic indicates that plasmid self-digestion did not occur, a result of the mutations that arose as shown in Table 4.

Several factors may have played a role for the mutations shown in Table 4. In general, double-stranded breaks are known to be recognized by DNA repair and recombination machineries.³¹⁻³⁴ Additionally, CRISPR-Cas systems have also been reported to have interactions with DNA repair machineries,³⁵⁻³⁸ although the precise mechanisms are not fully understood. We also find in the literature that different enzymes play important roles on ensuring the integrity of the DNA before entry into sporulation. For example, Sda prevents entry into sporulation in response to DNA damage, or when there is only one copy of DNA, which would generate anucleate, unviable spores.³¹ SirA on the other hand, prevents that the chromosome copy number exceeds two.³¹ Distinctly, DisA delays entry into sporulation if the chromosome is compromised by DNA damage, although it is not clear if it has specificity for chromosomic DNA or if it can also affect plasmid DNA.³¹ Further understanding on how Cas9 interacts with the diverse molecular pathways in B. subtilis during sporulation could be a key factor to redesigning our strategy, while emerging Cas9-based gene editing techniques continue to rely on the repair of double-stranded breaks by the host.^{35,39,40}

Our results show that it is possible to eliminate plasmids while expressing phenotypes in most of the spores. At the population level, approximately 90% of the spores produced with our approach were free from detectible plasmids and plasmid integration into the chromosome. While we identified mutations of the self-digestion circuitry and plasmid integration as impediments to make our plasmids fully disappear from the spore population, previous studies involving DNA digestion in the bacteria E. coli show that it is possible to eliminate plasmids in an entire population.^{16,17} One of these studies used engineered phages to infect bacteria and cause the elimination of native plasmids that provide antibiotic resistance,¹⁶ and another study used gene circuits inserted in the genome that can eliminate plasmids by induction, as a strategy to protect industrial secrets contained in DNA sequences.¹⁷ One of the reasons behind the disparity in efficiency of plasmid elimination in E. coli vs sporulating B. subtilis could be the differences in the bacterial host, for example, the activity of host DNA repair machineries and DNA exonucleases, and possible involvement of unknown anti-CRISPR-Cas systems.⁴¹ A better understanding of these mechanisms could help find ways to enhance plasmid elimination further in the spore system, which could benefit the use of bioengineering concepts in various applications without causing genetic alterations.

In contrast to the biocontainment approaches that lower the viability of organisms in the natural environment, our approach to the bioengineering of spores of Bacillus subtilis offers the possibility to release bioengineered spores to the environment without introducing synthetic or foreign DNA sequences. This feature could be particularly useful in the case of spores, because several spore-based industrial products are intended to be released to the environment, such as spore-based biopesticides that have long been used in the wild type form.⁴² Spore bioengineering could also benefit other applications of spores in vaccines, probiotics, energy converting materials,⁴³ and living material systems. Remarkably, the only way to produce these spores is to use the source bacteria that are transformed by the plasmid containing the synthetic gene circuit. If the first generation of spores derived from this source germinates, subsequent generations of bacteria and the spores will not retain the bioengineered features of the first generation of spores. This characteristic may provide advantages in the commercialization of bioengineered spore technologies by preventing the replication of the bioengineered features in the subsequent generations of spores. Furthermore, if accidental germination degrades the function of the spore-based products, the synthetic gene circuit could potentially be used to block the expression of key spore receptors to cause deficiency in germination.⁴⁴

### Materials and Methods

### Strains, Media, and Plasmid Construction.

For cloning purposes, we used Mix & Go Z-competent E. coli strain Zymo 5a from Zymo Research (cat. no. T3007). We used Gibson Assembly for the construction of the plasmids and confirmed the DNA sequence by Sanger sequencing.

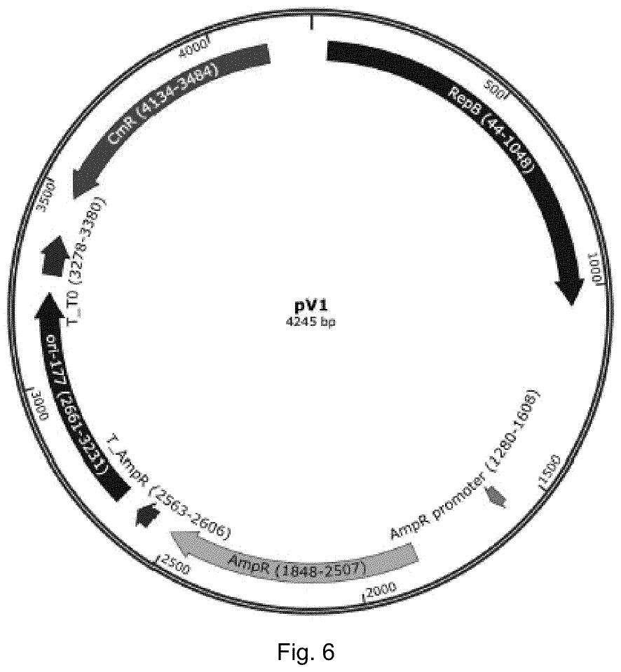

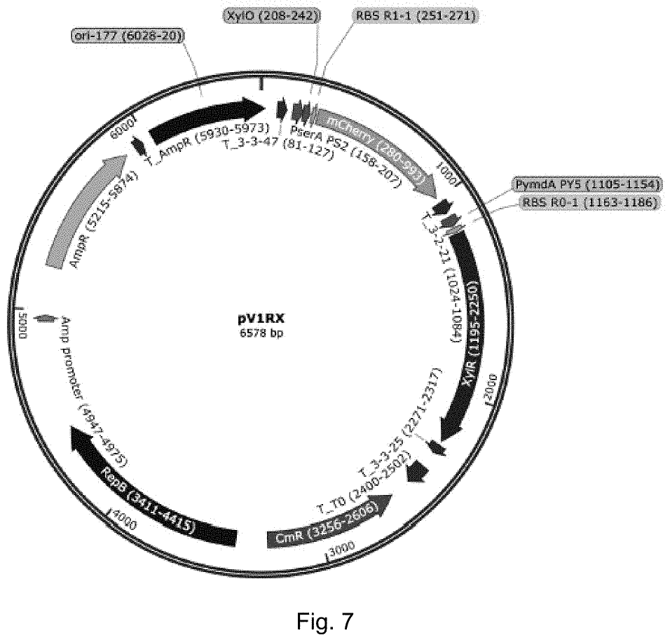

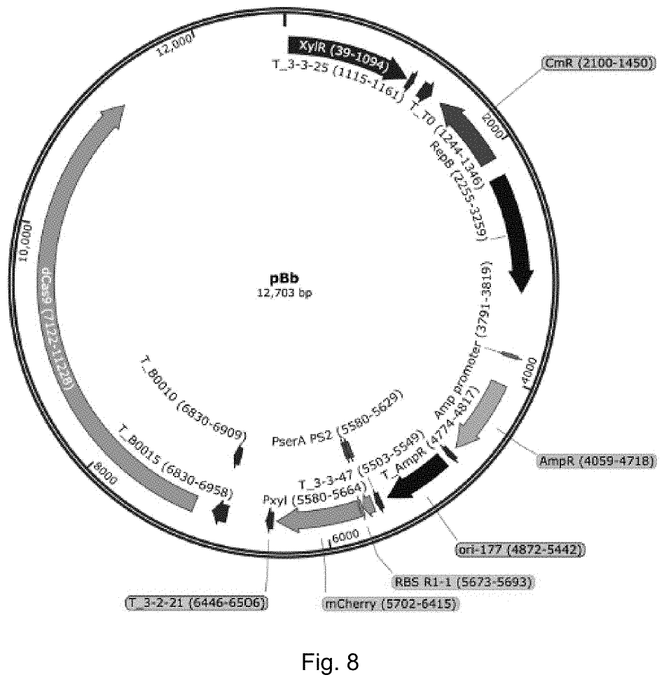

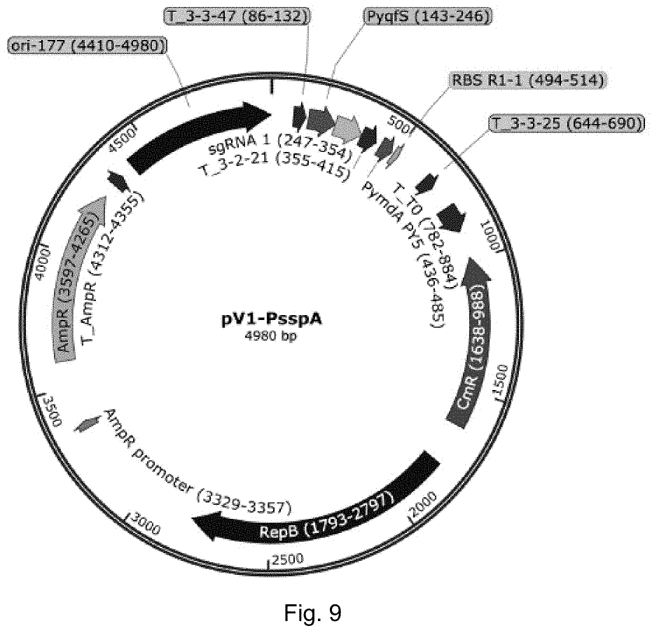

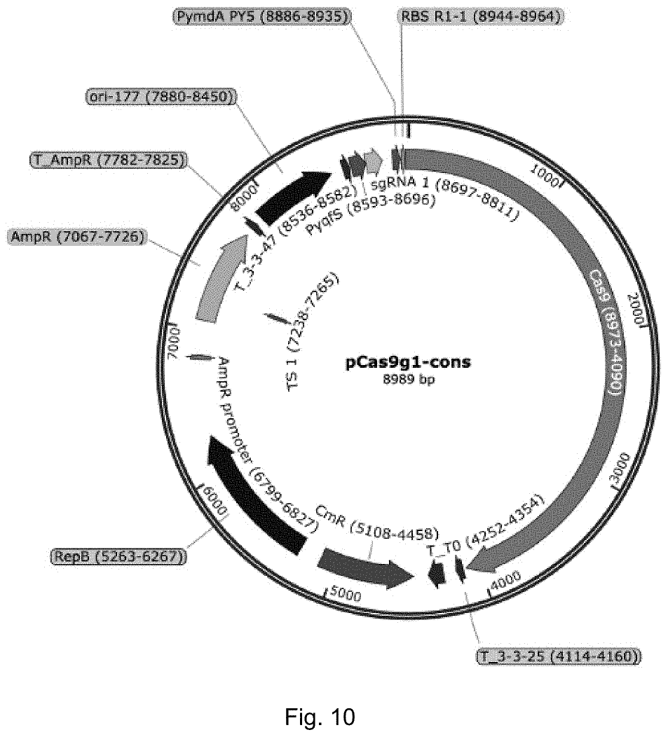

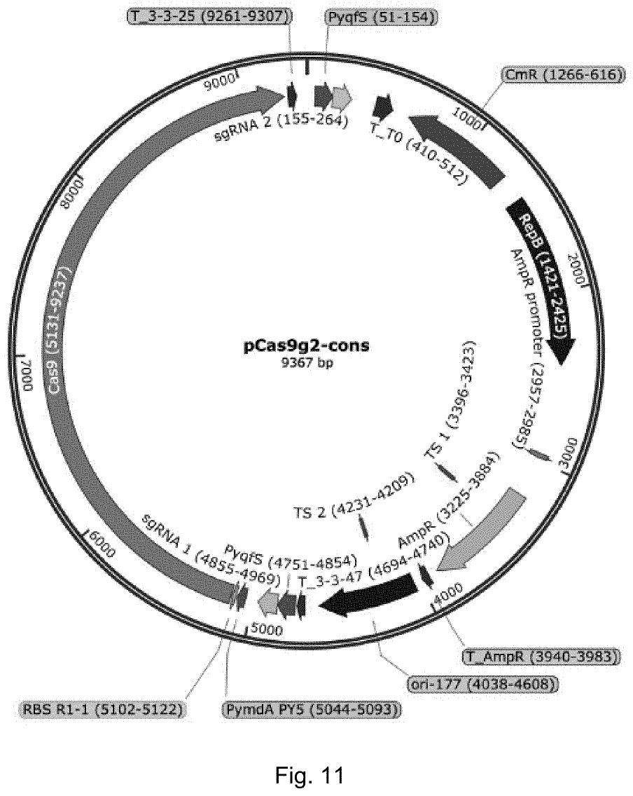

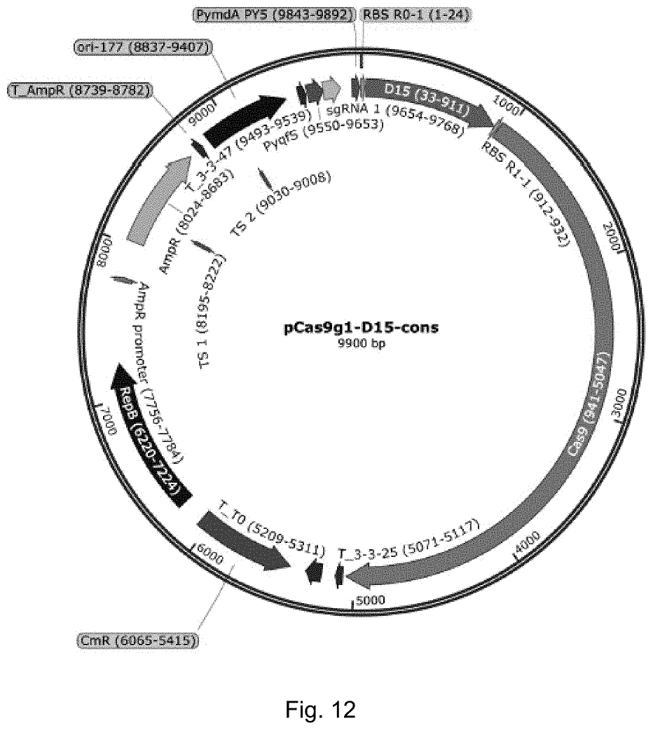

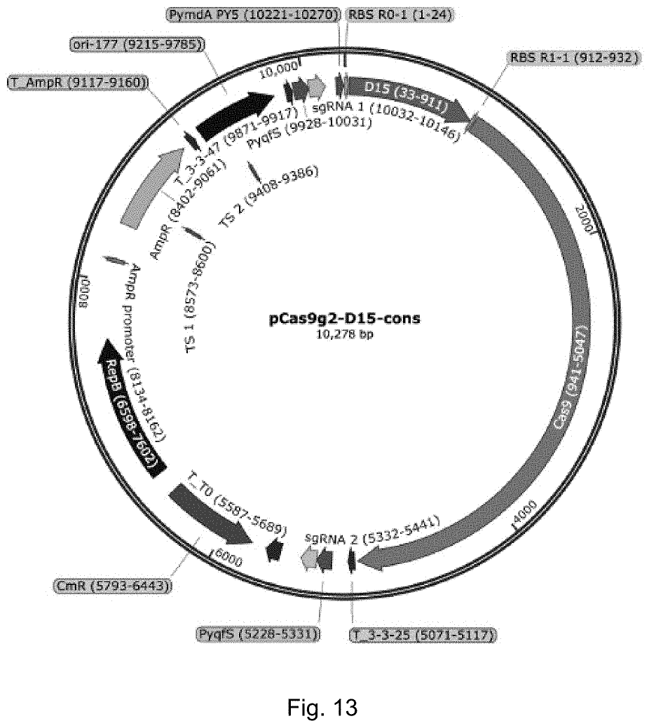

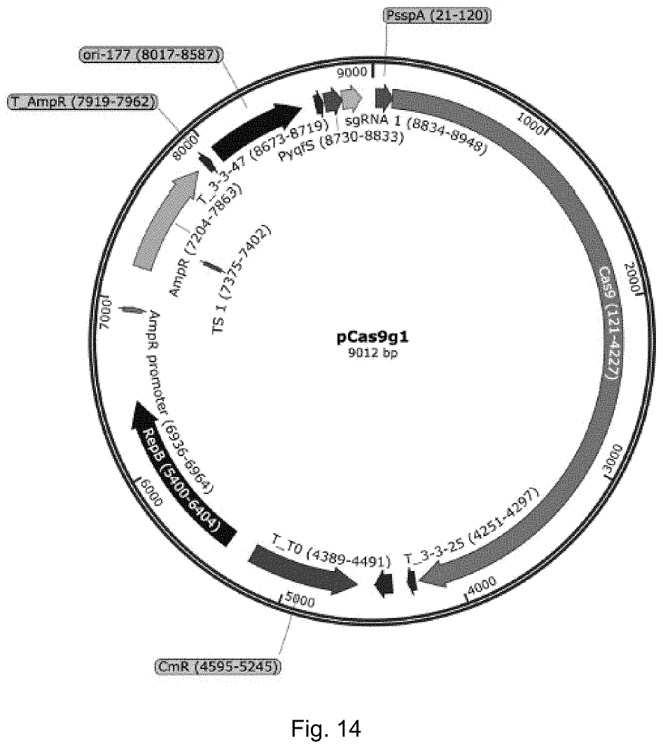

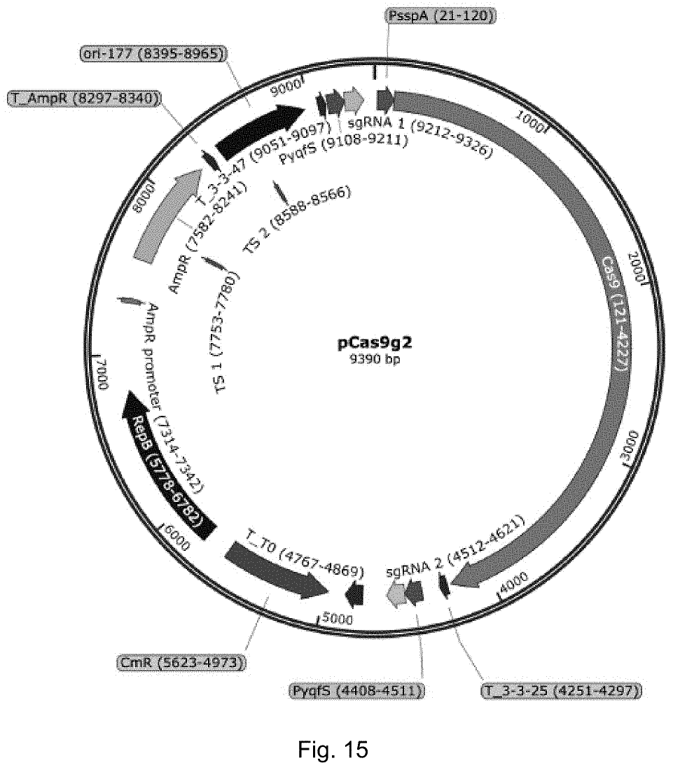

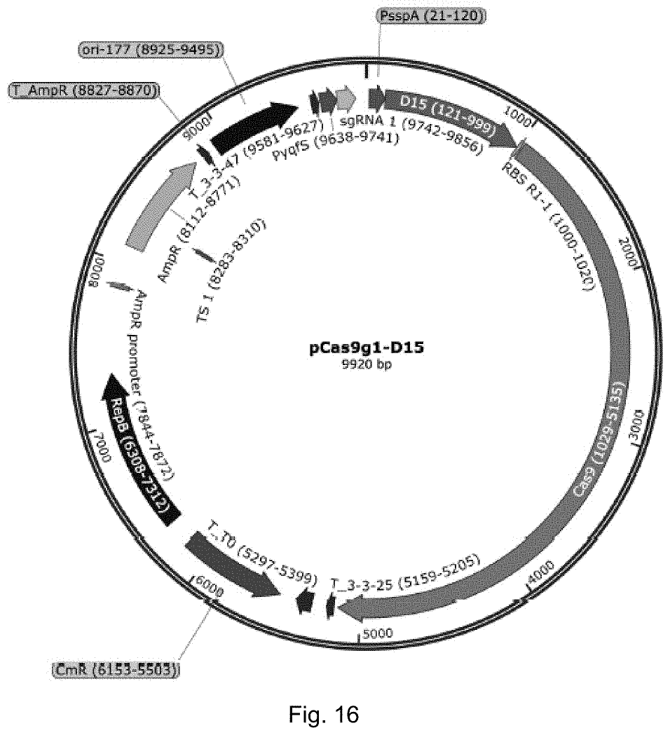

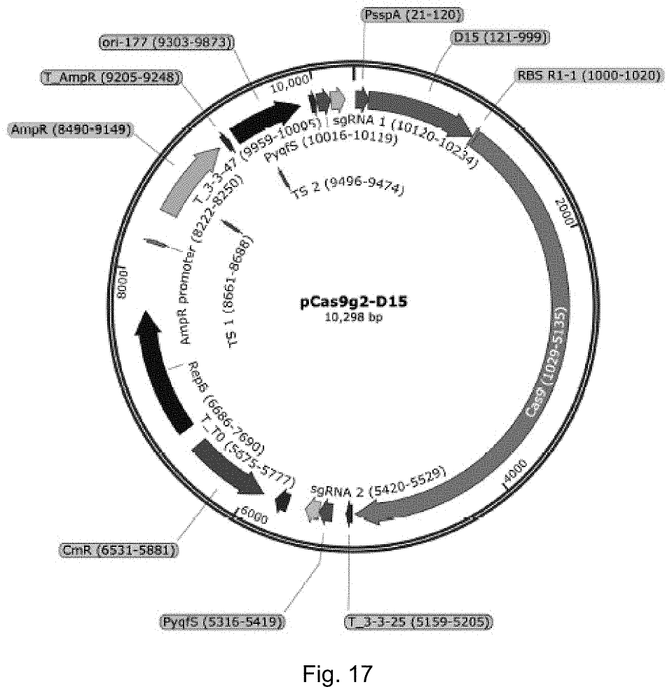

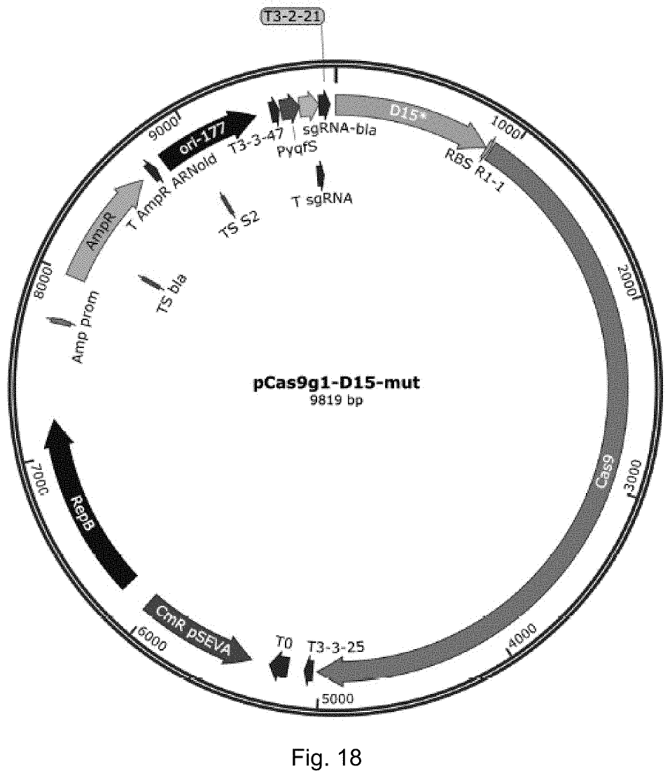

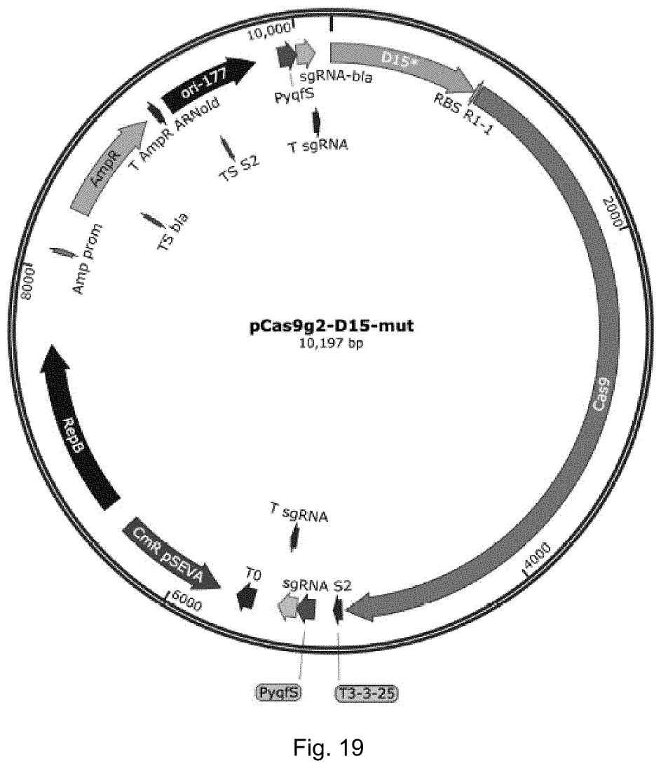

Transformation of B. subtilis 168 was performed through electroporation as previously described,⁴⁵ followed by growth at 37° C. on LB plates containing 25 μg/mL chloramphenicol. We used plasmid pHY300PLK46 to build pBb, which we used as the backbone to build all our designer plasmids. Details for genetic constructions can be found in Table 5. Plasmid maps can be found in FIGS. 6-23. Plasmid DNA sequences can be found in Supporting DNA Sequences. Several synthetic genetic parts shown in the plasmid maps were taken from a library of parts for B. subtilis previously reported.⁴⁷

TABLE 5
Primers used for the construction of the plasmids in our study

| Oligo | | | | |
|---|---|---|---|---|
| (SEQ | | Target | | Resulting |
| ID No.) | Oligo sequence (5′→3′) | DNA | Source | plasmid |
| 001 | ttgtccttttccgctgcata | pHY300PLK | Takara Bio - | pV1 |
| | AACAATATGGCCCGTTTG | | #3060 | |
| 002 | gccagggttttcccagtcac | | | |
| | TGCAGATCTCTAGAAGCT | | | |
| 003 | caagcttctagagatctgca | pSEVA3b61 | Addgene - | |
| | GTGACTGGGAAAACCCT | | #58310 | |
| 004 | aacaaacgggccatattgtt | | | |
| | TATGCAGCGGAAAAGGAC | | | |
| 005 | tatggaaaaacgctttgccc | gB1 | Gene | pV1RX |
| | CACACAGGCTCTTGAACAAG | | fragment | |
| 006 | AACACCATTGACTCCTTCATC | | synthesis | |
| 007 | GCAGCCAAGTGAATACACTG | gB2 | Gene | |
| 008 | Actgcagatctctagaagct | | fragment | |
| | ATGCTGAATGACTTCTTACAGC | | synthesis | |
| 009 | tgtaagaagtcattcagcat | pV1 | This study | |
| | AGCTTCTAGAGATCTGCA | | | |
| 010 | cttgttcaagagcctgtgtg | | | |
| | GGGCAAAGCGTTTTTCC | | | |
| 011 | tacggttatccacagaatca | pV1RX | This study | pBb |
| | GAGGGCAAACTACTAGAGA | | | |
| 012 | ccaagtaatcgtgaatgtcg | | | |
| | ACCGGATAAGGAGATCATTG | | | |
| 013 | caatgatctccttatccggt | pJWV102- | Addgene - | |
| | CGACATTCACGATTACTTGG | PL-dCas9 | #85588 | |
| 014 | atctctagtagtttgccctc | | | |
| | TGATTCTGTGGATAACCGTA | | | |
| 015 | gcttgtcagggggcg | gB20 | Gene | pV1- |
| | GAGCCTATGGAAAAACGCTTTG | | fragment | PsspA |
| 016 | ccctttaagagcgtcatgta | | synthesis | |
| | AACTTCATTGTAAATTCTGAAGAAATA | | | |
| | CTTGTCAAG | | | |
| 017 | tcagaatttacaatgaagtt | gB20 | Gene | |
| | TACATGACGCTCTTAAAGGGG | | fragment | |
| 018 | tcgccagggttttc | | synthesis | |
| | CCAGTCACTGCAGATCTCT | | | |
| 019 | AGCTTCTAGAGATCTGCA | pV1 | This study | |
| 020 | GGGCAAAGCGTTTTTCC | | | |
| 021 | AATAGGCTTAGATATCGGCACA | pCas9 | Addgene - | pCas9g1- |
| 022 | TCAGTCACCTCCTAGCTG | | #42876 | cons |
| 023 | GAAACACGCATTGATTTGAGT | pV1- | This study | |
| 024 | CCCATCCGACGCTATTTGT | PsspA | | |
| 025 | gactggagcttctagagatc | gB26 | Gene | pCas9g2- |
| | AGGTCAGACGCGATTTCC | | fragment | cons |
| 026 | gggttttcccagtcactgca | | synthesis | |
| | TGACCCAGACACCTATTCC | | | |
| 027 | aggaataggtgtctgggtca | pCas9g1- | This study | |
| | TGCAGTGACTGGGAAAACC | cons | | |
| 028 | caggaaatcgcgtctgacct | | | |
| | GATCTCTAGAAGCTCCAGTCAC | | | |
| 029 | tacaatgaagtttacatgac | gB27 | Gene | pCas9g1- |
| | GATTAACTAATAAGGAGGGCAAAC | | fragment | D15-cons |
| 030 | ctaaaaccccctttaagagc | | synthesis | |
| | TTATCATTGTTCCGCGATCTC | | | |
| 031 | agatcgcggaacaatgataa | pCas9g1- | This study | |
| | GCTCTTAAAGGGGGTTTTAGA | cons | | |
| 032 | gccctccttattagttaatc | | | |
| | GTCATGTAAACTTCATTGTAAATTCTGA | | | |
| | AG | | | |
| 033 | tacaatgaagtttacatgac | gB27 | Gene | pCas9g2- |
| | GATTAACTAATAAGGAGGGCAAAC | | fragment | D15-cons |
| 034 | ctaaaaccccctttaagagc | | synthesis | |
| | TTATCATTGTTCCGCGATCTC | | | |
| 035 | agatcgcggaacaatgataa | pCas9g2- | This study | |
| | GCTCTTAAAGGGGGTTTTAGA | cons | | |
| | gccctccttattagttaatc | | | |
| 036 | GTCATGTAAACTTCATTGTAAATTCTGA | | | |
| | AG | | | |
| 037 | gttttggtccctcaatgatc | gB27 | Gene | pCas9g1 |
| | TCCTTATCCGGTTAAGATGG | | fragment | |
| 038 | ttgtgccgatatcta | | synthesis | |
| | AGCCTATTGAGTATTTCTTATCCAT | | | |
| 039 | ATGGATAAGAAATACTCAATAGGCT | pCas9g1- | This study | |
| 040 | CCATCTTAACCGGATAAGGA | cons | | |
| 041 | gttttggtccctcaatgatc | gB27 | Gene | pCas9g2 |
| | TCCTTATCCGGTTAAGATGG | | fragment | |
| 042 | ttgtgccgatatcta | | synthesis | |
| | AGCCTATTGAGTATTTCTTATCCAT | | | |
| 043 | ATGGATAAGAAATACTCAATAGGCT | pCas9g2- | This study | |
| 044 | CCATCTTAACCGGATAAGGA | cons | | |
| 045 | gttttggtccctcaatgatc | gB28 | Gene | pCas9g1- |
| | TCCTTATCCGGTTAAGATGG | | fragment | D15 |
| 046 | cctcttcctcctcgatgaat | | synthesis | |
| | TTGCCCCAGGATTTACTCAT | | | |
| 047 | ATGAGTAAATCCTGGGGCAA | pCas9g1- | This study | |
| 048 | CCATCTTAACCGGATAAGGA | D15-cons | | |
| 049 | gttttggtccctcaatgatc | gB28 | Gene | pCas9g2- |
| | TCCTTATCCGGTTAAGATGG | | fragment | D15 |
| 050 | cctcttcctcctcgatgaat | | synthesis | |
| | TTGCCCCAGGATTTACTCAT | | | |
| 051 | ATGAGTAAATCCTGGGGCAA | pCas9g2- | This study | |
| 052 | CCATCTTAACCGGATAAGGA | D15-cons | | |
| 053 | ccttatccggttaagatggc | pCas9g1- | This study | pCas9g1- |
| | GAGTAAATCCTGGGGCAAA | D15 | | D15-mut |
| 054 | atttgccccaggatttactc | | | |
| | GCCATCTTAACCGGATAAGG | | | |
| 055 | ccttatccggttaagatggc | pCas9g2- | This study | pCas9g2- |
| | GAGTAAATCCTGGGGCAAA | D15 | | D15-mut |
| 056 | atttgccccaggatttactc | | | |
| | GCCATCTTAACCGGATAAGG | | | |
| 057 | gagcgtctcagcttctagag | pCas9g1- | This study | pCas9g1- |
| | GTGACTGGGAAAACCCTG | D15 | | D15-GFP |
| 058 | cggataaggagatcattgag | | | |
| | GAAGCTATACTGTAAAGTACAAGCA | | | |
| 059 | gtactttacagtatagcttc | gB21 | Gene | |
| | CTCAATGATCTCCTTATCCGG | | fragment | |
| 060 | gccagggttttcccagtcac | | synthesis | |
| | CTCTAGAAGCTGAGACGCT | | | |
| 061 | gagcgtctcagcttctagag | pCas9g2- | This study | pCas9g2- |
| | TAGAGATCTGCAGTGACTGG | D15 | | D15-GFP |
| 062 | cggataaggagatcattgag | | | |
| | GAAGCTATACTGTAAAGTACAAGCA | | | |
| 063 | gtactttacagtatagcttc | gB21 | Gene | |
| | CTCAATGATCTCCTTATCCGG | | fragment | |
| 064 | ccagtcactgcagatctcta | | synthesis | |
| | CTCTAGAAGCTGAGACGCT | | | |
| 065 | ttaagatggcaagcttgaca | B. subtilis | Ref. 30 | pCas9g1- |
| | GCCGAATTCGGATTCCCG | MF2352 | | D15-KinA |
| 066 | ttgtaaagctcgtccatccc | chromosome | | |
| | CGGGCATGCTTATTTTTTTGGA | | | |
| 067 | caaaaaaataagcatgcccg | pCas9g1- | This study | |
| | GGGATGGACGAGCTTTAC | D15-GFP | | |
| 068 | cgcgggaatccgaattcggc | | | |
| | TGTCAAGCTTGCCATCTTAAC | | | |
| 069 | ttaagatggcaagcttgaca | B. subtilis | Ref. 30 | pCas9g2- |
| | GCCGAATTCGGATTCCCG | MF2352 | | D15-KinA |
| 070 | ttgtaaagctcgtccatccc | chromosome | | |
| | CGGGCATGCTTATTTTTTTGGA | | | |
| 071 | caaaaaaataagcatgcccg | pCas9g2- | This study | |
| | GGGATGGACGAGCTTTAC | D15-GFP | | |
| 072 | cgcgggaatccgaattcggc | | | |
| | TGTCAAGCTTGCCATCTTAAC | | | |

Lowercases represent oligo tails (for construction through Gibson Assembly)

### Sporulation, Purification, and Determination of Sporulation Efficiency.

For sporulation, freshly transformed bacterial colonies were grown in shaking antibiotic-free LB for 2 h at 37° C. One volume of this culture was then centrifuged and resuspended in 200 volumes of antibiotic-free DSM and incubated for 3 h in shaking at 37° C. Finally, one volume of this culture was centrifuged and resuspended in 20 volumes of antibiotic-free DSM and incubated for 24 h to let cells sporulate.

Spore purification was carried out following two different methods. Method 1: Spores were collected by centrifugation, resuspended in resuspension buffer (50 mM Tris, 50 mg/L lysozyme, pH 7.5), centrifuged, washed with 0.05% SDS, and then centrifuged and washed with double distilled water (ddH2O) three times. Method 2: Spores were treated as in Method 1 followed by centrifugation and resuspension in 0.5% sodium hypochlorite for 10 min at room temperature. Immediately after 10 min, 3 volumes of 5 mg/L sodium thiosulfate were added and the sample was shortly vortexed. The resulting sample was washed twice with ddH2O and then treated with DNase I at 37° C. followed by heat inactivation at 75° C. The resulting sample was washed with ddH2O twice.

Spores obtained by artificial sporulation induction were purified through Method 1, and sporulation efficiency was determined as previously reported in the literature.⁴⁸ GFP spores were purified using Method 1, omitting the SDS step. Spores for qPCR, spores that were later germinated for colony PCR, and spores used for the antibiotic-based plate assay were purified through Method 2.

qPCR-Based Assay for Plasmid Quantification.

Spores obtained with the purification Method 2 were lysed using the OmniLyse Cell Lysis Kit (Claremont Biosolutions, LLC. cat. no. 01.341.24) for 60 seconds in the static mode. The resulting sample was used for absolute quantification of plasmid through qPCR following a method previously reported.⁴⁹ We used primers QPCRCH1

(5′-ACTGCCCGTTACTGGTATAAAG-3′; SEQ ID No: 243)

### and QPCRCH2

(5′-TGCACCTTGCTTTGGTTTATG-3′; SEQ ID No: 244)

to target the chromosome, and primers QPCRP1 (5′-GGCGGTGATCACTGATGAATA-3′; SEQ ID No: 245) and QPCRP2 (5′-GCTGTCTCTCCACTGTCAAATA-3′; SEQ ID No: 246) to target a conserved sequence among all the plasmids used in our study. Neither of the two different sets of primers amplified the opposite target.

### Colony PCR-Based Assay for Plasmid Detection.

Spores obtained with purification Method 2 were plated on LB-agar without antibiotic after having being diluted such that every resulting colony would be the product of a single spore. Full colonies were picked and resuspended with water individually. Colonies were analyzed through colony PCR with primers targeting the plasmid. Amplification or no amplification was counted as the presence or absence of plasmid, correspondingly. One hundred colonies were analyzed for every strain.

### Antibiotic-Based Assay for Plasmid Detection.

Spores obtained by purification Method 2 were plated on LB-agar with or without antibiotic.

Isolation of Plasmid after Sporulation, and Sequencing.

We used the QIAprep Spin Miniprep Kit from QIAGEN to isolate plasmid DNA from B. subtilis. In addition to the protocol of the manufacturer, we added 1 mg/mL lysozyme in the step in which a bacteria pellet is resuspended with buffer P1. This bacterial resuspension was incubated at 37° C. for 10 min. After that, we followed the protocol of the manufacturer.

### GFP-Coated Spores.

GFP-coated spores were obtained with purification Method 1 (skipping the use of 0.05% SDS), placed on a glass bottom dish, and dried by evaporation through incubation at room temperature overnight. A Zeiss LSM 800 microscope was used for confocal microscopy using the 63× objective. GFP was excited with a laser at 488 nm, and its emission was detected in the range of 500-520 nm. Because autofluorescence has been previously observed in wild type spores in long periods of GFP excitation,²⁹ we limited exposure time to 2-5 s.

### Atomic Force Microscopy.

Atomic force microscopy was carried out with a multimode SPM (Veeco Instruments) operated in tapping mode as previously described.⁵⁰

Detection of Plasmid Integration into the Chromosome.

The detection of plasmid integration events was performed in accordance to a previously described protocol known as inverse PCR.⁵¹ Genomic DNA of B. subtilis was extracted and treated with restriction enzyme CviQI or DpnII to produce fragments of about 3000 bp or less. Then, DNA fragments were religated with T4 ligase, and used for PCR. A second, nested PCR was performed, and amplified DNA fragments were sequenced. Several sets of primers were used to target different locations along the plasmids (Table 7). Integration was confirmed or discarded by alignment of the sequenced DNA against the genome of B. subtilis strain 168, using the tool blastn from the NCBI.

### REFERENCES

(1) King, Z. A., Lloyd, C. J., Feist, A. M., and Palsson, B. O. (2015) Next-generation genome-scale models for metabolic engineering. Curr. Opin. Biotechnol. 35, 23-29.
(2) Karauzum, H., Updegrove, T. B., Kong, M., Wu, I., Datta, S. K., and Ramamurthi, K. S. (2018) Vaccine display on artificial bacterial spores enhances protective efficacy against Staphylococcus aureus infection. FEMS Microbiol. Lett. 365, 1-9.
(3) Liu, C., Zhang, L., Liu, H., and Cheng, K. (2017) Delivery strategies of the CRISPR-Cas9 gene-editing system for therapeutic applications. J. Controlled Release 266, 17-26.
(4) Naldini, L. (2011) Ex vivo gene transfer and correction for cell-based therapies. Nat. Rev. Genet. 12, 301-315.
(5) Owen, M. D. K., and Zelaya, I. A. (2005) Herbicide-resistant crops and weed resistance to herbicides. Pest Manage. Sci. 61, 301-311.
(6) Su, L., Jia, W., Hou, C., and Lei, Y. (2011) Microbial biosensors: A review. Biosens. Bioelectron. 26, 1788-1799.
(7) Donohoue, P. D., Barrangou, R., and May, A. P. (2017) Advances in Industrial Biotechnology Using CRISPR-Cas Systems. Trends Biotechnol. 32, 134-146.
(8) Strauss, S. S., and Sax, J. K. (2016) Ending event-based regulation of GMO crops. Nat. Biotechnol. 34, 474-477.
(9) Lefebvre, S., Cook, L. A., and Griffiths, M. A. (2019) Consumer perceptions of genetically modified foods: a mixed-method approach. J. Consum. Mark. 36, 113-123.
(10) Wunderlich, S., and Gatto, K. A. (2015) Consumer perception of genetically modified organisms and sources of information. Adv. Nutr. 6, 842-851.
(11) Mitter, N., Worrall, E. A., Robinson, K. E., Li, P., Jain, R. G., Taochy, C., Fletcher, S. J., Carroll, B. J., Lu, G. Q., and Xu, Z. P. (2017) Clay nanosheets for topical delivery of RNAi for sustained protection against plant viruses. Nat. Plants 3, 16207.
(12) Hosseini, S., Curilovs, A., and Cutting, S. M. (2018) Biological containment of genetically modified Bacillus subtilis. Appl. Environ. Microbiol. 84, 1-15.
(13) Lee, J. W., Chan, C. T. Y., Slomovic, S., and Collins, J. J. (2018) Next-generation biocontainment systems for engineered organisms. Nat. Chem. Biol. 14, 530-537.
(14) Maselko, M., Heinsch, S. C., Chacón, J. M., Harcombe, W. R., and Smanski, M. J. (2017) Engineering species-like barriers to sexual reproduction. Nat. Commun. 8, 883.
(15) Mell, J. C., and Redfield, R. J. (2014) Natural competence and the evolution of DNA uptake specificity. J. Bacteriol. 196, 1471-1483.
(16) Kim, J.-S., Cho, D.-H., Park, M., Chung, W.-J., Shin, D., Ko, K. S., and Kweon, D.-H. (2016) CRISPR/Cas9-Mediated Re-Sensitization of Antibiotic-Resistant Escherichia coli Harboring Extended-Spectrum β-Lactamases. J. Microbiol. Biotechnol. 26, 394-401.
(17) Caliando, B. J., and Voigt, C. A. (2015) Targeted DNA degradation using a CRISPR device stably carried in the host genome. Nat. Commun. 6, 6989.
(18) Piggot, P. J., and Hibert, D. W. (2004) Compartmentalization of Gene Expression during Bacillus subtilis Spore Formation. Microbiol. Mol. Biol. Rev. 68, 234-262.
(19) Steil, L., Serrano, M., Henriques, A. O., and Volker, U. (2005) Genome-wide analysis of temporally regulated and compartment specific gene expression in sporulating cells of Bacillus subtilis. Microbiology 151, 399-420.
(20) Regan, G., Itaya, M., and Piggot, P. J. (2012) Coupling of sigma-G Activation to Completion of Engulfment during Sporulation of Bacillus subtilis Survives Large Perturbations to DNA Translocation and Replication. J. Bacteriol. 194, 6264-6271.
(21) Sayers, J. R., and Eckstein, F. (1991) A single-strand specific endonuclease activity copurifies with overexpressed T5 D15 exonuclease. Nucleic Acids Res. 19, 4127-4132.
(22) Mason, J. M., and Setlow, P. (1987) Different Small, Acid-Soluble Proteins of the α/β Type Have Interchangeable Roles in the Heat and UV Radiation Resistance of Bacillus subtilis Spores. J. Bacteriol. 169, 3633-3637.
(23) Mason, J. M., Hackett, R. H., and Setlow, P. (1988) Regulation of Expression of Genes Coding for Small, Acid-Soluble Proteins of Bacillus subtilis Spores: Studies Using lacZ Gene Fusions. J. Bacteriol. 170, 239-244.
(24) Turgeon, N., Laflamme, C., Ho, J., and Duchaine, C. (2008) Evaluation of the plasmid copy number in B. cereus spores, during germination, bacterial growth and sporulation using real-time PCR. Plasmid 60, 118-124.
(25) Ghosh, S., and Setlow, P. (2009) Isolation and Characterization of Superdormant Spores of Bacillus Species. J. Bacteriol. 191, 1787-1797.
(26) Yoshikawa, H., Ogasawara, N., and Seiki, M. (1980) Initiation of DNA Replication in Bacillus subtilis. Mol. Gen. Genet. 179, 265-272.
(27) Khasanov, F. K., Zvingila, D. J., Zainullin, A. A., Prozorov, A. A., and Bashkirov, V. I. (1992) Homologous recombination in Bacillus subtilis requires approximately 70 bp of homology. Mol. Gen. Genet. 234, 494-497.
(28) Chen, H., Ullah, J., and Jia, J. (2017) Progress in Bacillus subtilis Spore Surface Display Technology towards Environment, Vaccine Development, and Biocatalysis. J. Mol. Microbiol. Biotechnol. 27, 159-167.
(29) Imamura, D., Kuwana, R., Takamatsu, H., and Watabe, K. (2010) Localization of proteins to different layers and regions of Bacillus subtilis spore coats. J. Bacteriol. 192, 518-524.
(30) Eswaramoorthy, P., Duan, D., Dinh, J., Dravis, A., Devi, S. N., and Fujita, M. (2010) The threshold level of the sensor histidine kinase KinA governs entry into sporulation in Bacillus subtilis. J. Bacteriol. 192, 3870-3882.
(31) Lenhart, J. S., Schroeder, J. W., Walsh, B. W., and Simmons, L. A. (2012) DNA Repair and Genome Maintenance in Bacillus subtilis. Microbiol. Mol. Biol. Rev. 76, 530-564.
(32) Wigley, D. B. (2013) Bacterial DNA repair: recent insights into the mechanism of RecBCD, AddAB and AdnAB. Nat. Rev. Microbiol. 11, 9-13.
(33) Li, X., and Heyer, W. D. (2008) Homologous recombination in DNA repair and DNA damage tolerance. Cell Res. 18, 99-113.
(34) Shuman, S., and Glickman, M. S. (2007) Bacterial DNA repair by non-homologous end joining. Nat. Rev. Microbiol. 5, 852-861.
(35) Cubbon, A., Ivancic-Bace, I., and Bolt, E. L. (2018) CRISPR-Cas immunity, DNA repair and genome stability. Biosci. Rep. 38, 1-10.
(36) Bernheim, A., Bikard, D., Touchon, M., and Rocha, E. P. C. (2019) A matter of background: DNA repair pathways as a possible cause for the sparse distribution of CRISPR-Cas systems in bacteria. Philos. Trans. R. Soc. B Biol. Sci. 374, 1-10.
(37) Levy, A., Goren, M. G., Yosef, I., Auster, O., Manor, M., Amitai, G., Edgar, R., Qimron, U., and Sorek, R. (2015) CRISPR adaptation biases explain preference for acquisition of foreign DNA. Nature 520, 505-510.
(38) Babu, M., Beloglazova, N., Flick, R., Graham, C., Skarina, T., Nocek, B., Gagarinova, A., Pogoutse, O., Brown, G., Binkowski, A., Phanse, S., Joachimiak, A., Koonin, E. V., Savchenko, A., Emili, A., Greenblatt, J., Edwards, A. M., and Yakunin, A. F. (2011) A dual function of the CRISPR-Cas system in bacterial antivirus immunity and DNA repair. Mol. Microbiol. 79, 484-502.
(39) Pickar-Oliver, A., and Gersbach, C. A. (2019) The next generation of CRISPR-Cas technologies and applications. Nat. Rev. Mol. Cell Biol. 20, 490-507.
(40) Mougiakos, I., Bosma, E. F., Ganguly, J., van der Oost, J., and van Kranenburg, R. (2018) Hijacking CRISPR-Cas for high-throughput bacterial metabolic engineering: advances and prospects. Curr. Opin. Biotechnol. 50, 146-157.
(41) Hwang, S., and Maxwell, K. L. (2019) Meet the Anti-CRISPRs: Widespread Protein Inhibitors of CRISPR-Cas Systems. Cris. J. 2, 23-30.
(42) Rosas-Garcia, N. (2009) Biopesticide Production from Bacillus thuringiensis: An Environmentally Friendly Alternative. Recent Pat. Biotechnol. 3, 28-36.
(43) Chen, X., Mahadevan, L., Driks, A., and Sahin, O. (2014) Bacillus spores as building blocks for stimuli-responsive materials and nanogenerators. Nat. Nanotechnol. 9, 137-141.
(44) Paidhungat, M., and Setlow, P. (2000) Role of Ger proteins in nutrient and nonnutrient triggering of spore germination in Bacillus subtilis. J. Bacteriol. 182, 2513-2519.
(45) Xue, G., Johnson, J. S., and Dalrymple, B. P. (1999) High osmolarity improves the electro-transformation efficiency of the gram positive bacteria Bacillus subtilis and Bacillus licheniformis. J. Microbiol. Methods 34, 183-191.
(46) Ishiwa, H., and Shibahara-Sone, H. (1986) New shuttle vectors for Escherichia coli and Bacillus subtilis I. Construction and characterization of plasmid pHY460 with twelve unique cloning sites. Jpn. J. Genet. 61, 515-528.
(47) Guiziou, S., Sauveplane, V., Chang, H., Clert, C., Declerck, N., Jules, M., and Bonnet, J. (2016) A part toolbox to tune genetic expression in Bacillus subtilis. Nucleic Acids Res. 44, 7495-7508.
(48) Eswaramoorthy, P., Dravis, A., Devi, S. N., Vishnoi, M., Dao, H. A., and Fujita, M. (2011) Expression level of a chimeric kinase governs entry into sporulation in Bacillus subtilis. J. Bacteriol. 193, 6113-6122.
(49) Lee, C., Kim, J., Shin, S. G., and Hwang, S. (2006) Absolute and relative QPCR quantification of plasmid copy number in Escherichia coli. J. Biotechnol. 123, 273-280.
(50) Ghosh, S., Setlow, B., Wahome, P. G., Cowan, A. E., Plomp, M., Malkin, A. J., and Setlow, P. (2008) Characterization of spores of Bacillus subtilis that lack most coat layers. J. Bacteriol. 190, 6741-6748.
(51) Boulin, T., and Bessereau, J. (2007) Mos1-mediated insertional mutagenesis in Caenorhabditis elegans. Nat. Protoc. 2, 1276-1287.

### Supporting DNA Sequences

>pV1
(SEQ ID No: 225)
ttgtccttttccgctgcataaacaatatggcccgtttgtt
GAACTACTCTTTAATAAAATAATTTTTCCGTTCCC
AATTCCACATTGCAATAATAGAAAATCCATCTTCATCGGCTTTTTCGTCATCATCTGT
ATGAATCAAATCGCCTTCTTCTGTGTCATCAAGGTTTAATTTTTTATGTATTTCTTTTA
ACAAACCACCATAGGAGATTAACCTTTTACGGTGTAAACCTTCCTCCAAATCAGACA
AACGTTTCAAATTCTTTTCTTCATCATCGGTCATAAAATCCGTATCCTTTACAGGATA
TTTTGCAGTTTCGTCAATTGCCGATTGTATATCCGATTTATATTTATTTTTCGGTCGAA
TCATTTGAACTTTTACATTTGGATCATAGTCTAATTTCATTGCCTTTTTCCAAAATTG
AATCCATTGTTTTTGATTCACGTAGTTTTCTGTATTCTTAAAATAAGTTGGTTCCACA
CATACCAATACATGCATGTGCTGATTATAAGAATTATCTTTATTATTTATTGTCACTT
CCGTTGCACGCATAAAACCAACAAGATTTTTATTAATTTTTTTATATTGCATCATTCG
GCGAAATCCTTGAGCCATATCTGACAAACTCTTATTTAATTCTTCGCCATCATAAAC
ATTTTTAACTGTTAATGTGAGAAACAACCAACGAACTGTTGGCTTTTGTTTAATAACT
TCAGCAACAACCTTTTGTGACTGAATGCCATGTTTCATTGCTCTCCTCCAGTTGCACA
TTGGACAAAGCCTGGATTTACAAAACCACACTCGATACAACTTTCTTTCGCCTGTTTC
ACGATTTTGTTTATACTCTAATATTTCAGCACAATCTTTTACTCTTTCAGCCTTTTTAA
ATTCAAGAATATGCAGAAGTTCAAAGTAATCAACATTAGCGATTTTCTTTTCTCTCC
ATGGTCTCACTTTTCCACTTTTTGTCTTGTCCACTAAAACCCTTGATTTTTCATCTGAA
TAAATGCTACTATTAGGACACATAATATTAAAAGAAACCCCCATCTATTTAGTTATT
TGTTTGGTCACTTATAACTTTAACAGATGGGGTTTTTCTGTGCAACCAATTTTAAGGG
TTTTCCAATACTTTAAAACACATACATACCAACACTTCAACGCACCTTTCAGCAACT
AAAATAAAAATGACGTTATTTCTATATGTATCAAGATAAGAAAGAACAAGTTCAAA
ACCATCAAAAAAAGACACCTTTTCAGGTGCTTTTTTTATTTTATAAACTCATTCCCTG
ATCTCGACTTCGTTCTTTTTTTACCTCTCGGTTATGAGTTAGTTCAAATTCGTTCTTTT
TAGGTTCTAAATCGTGTTTTTCTTGGAATTGTGCTGTTTTATCCTTTACCTTGTCTACA
AACCCCTTAAAAACGTTTTTAAAGGCTTTTAAGCGTCTGTACGTTCCTTAAGGAATT
ATTCCTTAGTGCTTTCTAGGTTAATGTCATGATAATAATGGTTTCTTAGACGTCAGGT
GGCACTTTTCGGGGAAATGTCCGCGGAACCCCTATTTGTTTATTTTTCTAAATACATT
CAAATATGTATCCGCTCATGAGACAATAACCCTGATAAATGCTTCAATAATATTGAA
AAAGGAAGAGTATGAGTATTCAACATTTCCGTGTCGCCCTTATTCCCTTTTTTGCGGC
ATTTTGCCTTCCTGTTTTTGCTCACCCAGAAACGCTGGTGAAAGTAAAAGATGCTGA
AGATCAGTTGGGTGCACGAGTGGGTTACATCGAACTGGATCTCAACAGCGGTAAGA
TCCTTGAGAGTTTTCGCCCCGAAGAACGTTTTCCAATGATGAGCACTTTTAAAGTTCT
GCTATGTGGCGCGGTATTATCCCGTGTTGACGCCGGGCAAGAGCAACTCGGTCGCCG
CATACACTATTCTCAGAATGACTTGGTTGAGTACTCACCAGTCACAGAAAAGCATCT
TACGGATGGCATGACAGTAAGAGAATTATGCAGTGCTGCCATAACCATGAGTGATA
ACACTGCGGCCAACTTACTTCTGACAACGATCGGAGGACCGAAGGAGCTAACCGCT
TTTTTGCACAACATGGGGGATCATGTAACTCGCCTTGATCGTTGGGAACCGGAGCTG
AATGAAGCCATACCAAACGACGAGCGTGACACCACGATGCCTGCAGCAATGGCAAC
AACGTTGCGCAAACTATTAACTGGCGAACTACTTACTCTAGCTTCCCGGCAACAATT
AATAGACTGGATGGAGGCGGATAAAGTTGCAGGACCACTTCTGCGCTCGGCCCTTCC
GGCTGGCTGGTTTATTGCTGATAAATCTGGAGCCGGTGAGCGTGGGTCTCGCGGTAT
CATTGCAGCACTGGGGCCAGATGGTAAGCCCTCCCGTATCGTAGTTATCTACACGAC
GGGGAGTCAGGCAACTATGGATGAACGAAATAGACAGATCGCTGAGATAGGTGCCT
CACTGATTAAGCATTGGTAACTGTCAGACCAAGTTTACTCATATATACTTTAGATTG
ATTTAAAACTTCATTTTTAATTTAAAAGGATCTAGGTGAAGATCCTTTTTGATAATCT
CATGACCAAAATCCCTTAACGTGAGTTTTCGTTCCACTGAGCGTCAGACCCCTTAAT
AAGATGATCTTCTTGAGATCGTTTTGGTCTGCGCGTAATCTCTTGCTCTGAAAACGA
AAAAACCGCCTTGCAGGGAGGTTTTTCGAAGGTTCTCTGAGCTACCAACTCTTTGAA
CCGAGGTAACTGGCTTGCAGGAGCGCAGTCACCAAAACTTGTCCTTTCAGTTTAGCC
TTAACCGGCGCATGACTTCAAGACTAACTCCTCTAAATCAATTACCAGTGGCTGCTG
CCAGTGGTGCTTTTGCATGTCTTTCCGGGTTGGACTCAAGACGATAGTTACCGGATA
AGGCGCAGCGGTCGGACTGAACGGGGGGTTCGTGCATACAGTCCAGCTTGGAGCGA
ACTGCCTACCCGGAACTGAGTGTCAGGCGTGGAATGAGACAAACGCGGCCATAACA
GCGGAATGACACCGGTAAACCGAAAGGCAGGAACAGGAGAGCGCACGAGGGAGCC
GCCAGGGGGAAACGCCTGGTATCTTTATAGTCCTGTCGGGTTTCGCCACCACTGATT
TGAGCGTCAGATTTCGTGATGCTTGTCAGGGGGCGGAGCCTATGGAAAAACGCTTTG
CCcaagcttctagagatctgcagtgactgggaaaaccctggc
GACTAGTCTTGGACTCCTGTTGATAGATCC
AGTAATGACCTCAGAACTCCATCTGGATTTGTTCAGAACGCTCGGTTGCCGCCGGGC
GTTTTTTATTGGTGAGAATCCAGGGGTCCCCAATAATTACGATTTAAATCCTTCAAA
CTTCCCAAAGGCGAGCCCTAGTGACATTAGAAAACCGACTGTAAAAAGTACAGTCG
GCATTATCTCATATTATAAAAGCCAGTCATTAGGCCTATCTGACAATTCCTGAATAG
AGTTCATAAACAATCCTGCATGATAACCATCACAAACAGAATGATGTACCTGTAAA
GATAGCGGTAAATATATTGAATTACCTTTATTAATGAATTTTCCTGCTGTAATAATGG
GTAGAAGGTAATTACTATTATTATTGATATTTAAGTTAAACCCAGTAAATGAAGTCC
ATGGAATAATAGAAAGAGAAAAAGCATTTTCAGGTATAGGTGTTTTGGGAAACAAT
TTCCCCGAACCATTATATTTCTCTACATCAGAAAGGTATAAATCATAAAACTCTTTG
AAGTCATTCTTTACAGGAGTCCAAATACCAGAGAATGTTTTAGATACACCATCAAAA
ATTGTATAAAGTGGCTCTAACTTATCCCAATAACCTAACTCTCCGTCGCTATTGTAAC
CAGTTCTAAAAGCTGTATTTGAGTTTATCACCCTTGTCACTAAGAAAATAAATGCAG
GGTAAAATTTATATCCTTCTTGTTTTATGTTTCGGTATAAAACACTAATATCAATTTC
TGTGGTTATACTAAAAGTCGTTTGTTGGTTCAAATAATGATTAAATATCTCTTTTCTC
TTCCAATTGTCTAAATCAATTTTATTAAAGTTCATTTGATATGCCTCCTAAATTTTTAT
CTAAAGTGAATTTAGGAGGCTTACTTGTCTGCTTTCTTCATTAGAATCAATCCTTTTT
TAAAAGTCAATCCCGTTTGTTGAAGACTT
>pV1RX
(SEQ ID No: 226)
tatggaaaaacgctttgccccacacaggctcttgaacaag
GGATAACAATTTGTCATAGCTGTATTCGTTAC
AAGGAGGATTTTCGAAAAAACACCCTAACGGGTGTTTTTTTATAGCTGGTCTCCCTT
GCTTGTACTTTACAGTATGCCATACGAAACTGCGTCAATACACGTTGACACTCTTTTG
TAGTTATGTTAAATTATCAGATCGCCATCATTAGTTTGTTTAAACAACAAACTAATA
CTAGAGGCTCTTAAAGGGGGTTTTAGATACCAGAGATGGTGAGCAAGGGCGAGGAG
GATAACATGGCCATCATCAAGGAGTTCATGCGCTTCAAGGTGCACATGGAGGGCTC
CGTGAACGGCCACGAGTTCGAGATCGAGGGCGAGGGCGAGGGCCGCCCCTACGAG
GGCACCCAGACCGCCAAGCTGAAGGTGACCAAGGGTGGCCCCCTGCCCTTCGCCTG
GGACATCCTGTCCCCTCAGTTCATGTACGGCTCCAAGGCCTACGTGAAGCACCCCGC
CGACATCCCCGACTACTTGAAGCTGTCCTTCCCCGAGGGCTTCAAGTGGGAGCGCGT
GATGAACTTCGAGGACGGCGGCGTGGTGACCGTGACCCAGGACTCCTCCCTGCAGG
ACGGCGAGTTCATCTACAAGGTGAAGCTGCGCGGCACCAACTTCCCCTCCGACGGC
CCCGTAATGCAGAAGAAGACCATGGGCTGGGAGGCCTCCTCCGAGCGGATGTACCC
CGAGGACGGCGCCCTGAAGGGCGAGATCAAGCAGAGGCTGAAGCTGAAGGACGGC
GGCCACTACGACGCTGAGGTCAAGACCACCTACAAGGCCAAGAAGCCCGTGCAGCT
GCCCGGCGCCTACAACGTCAACATCAAGTTGGACATCACCTCCCACAACGAGGACT
ACACCATCGTGGAACAGTACGAACGCGCCGAGGGCCGCCACTCCACCGGCGGCATG
GACGAGCTGTACAAGTAATAGCTGCCACCTTTCTGAACTTTGGTAATGCGTCTCGGT
ACCAAATTCCAGAAAAGAGGCCTCCCGAAAGGGGGGCCTTTTTTCGTTTTGGTCCCT
CAATGATCTCCTTATCCGGTTAAGATGGCAAGCTTGACAAGTATTTCTTCAGAATTT
ACAATGAAGTTTACTAGAGGATTAACTAATAAGGAGGGCAAACTACTAGAGATGAC
GGGCCTGAACAAGAGCACCGTAAgcagccaagtgaat
acactgatgaaggagtcaatggtgttCGAGATCG
GCCAGGGCCAGAGCTCAGGTGGCCGCCGCCCGGTAATGTTAGTCTTCAACAAGAAA
GCGGGCTATAGTGTGGGCATCGACGTCGGCGTAGACTACATCAACGGAATCCTTAC
GGATTTAGAGGGCACGATTGTGTTGGACCAGTATAGACACCTTGAGAGCAACAGCC
CGGAGATCACTAAGGATATCCTTATCGACATGATCCACCATTTCATCACACAGATGC
CTCAGAGCCCTTATGGATTCATCGGCATCGGCATCTGTGTACCGGGTTTAATCGACA
AGGACCAGAAGATCGTGTTTACGCCTAATAGCAATTGGCGTGACATCGATCTGAAG
AGCAGCATCCAGGAGAAATATAACGTAAGCGTGTTCATCGAGAACGAAGCGAACGC
AGGAGCGTACGGCGAGAAGTTGTTCGGTGCAGCGAAGAACCATGACAATATCATCT
ATGTGTCAATTTCAACGGGCATCGGAATTGGCGTGATCATTAATAACCACCTGTACC
GTGGCGTGTCAGGATTTAGCGGTGAGATGGGCCACATGACGATCGATTTCAACGGA
CCGAAGTGTTCATGTGGCAATAGAGGCTGTTGGGAGCTTTACGCGAGCGAGAAAGC
GCTGCTGAAGAGCTTGCAAACGAAGGAGAAGAAACTCAGCTACCAGGACATCATCA
ATCTTGCACACCTTAACGACATTGGCACACTGAACGCGCTGCAGAACTTCGGCTTCT
ACCTGGGCATCGGATTGACAAACATCTTGAACACGTTCAATCCGCAGGCAGTGATCC
TGCGCAACTCAATCATCGAGAGCCACCCGATGGTGCTGAACAGCATGCGCTCAGAG
GTGAGCAGCCGTGTGTACAGCCAGCTCGGAAACTCATACGAGCTGCTTCCGAGCAG
TCTTGGCCAGAACGCTCCTGCGCTTGGCATGAGCAGCATCGTAATCGACCACTTCTT
AGATATGATCACGATGTAATAGTCAAATGTCAGACGAAAATGCCAATTATTGAAGC
GGCTAACGCCGCTTTTTTTGTTTCTGGTCTCCCCATAGTTTAACAAGTGCtgtaagaagt
cattcagcatagcttctagagatctgcagtGACTGGGAAAACCCTGGCGACTAGTCTTGG
ACTCCTGTTGATAGATCCAGTAATGACCTCAGAACTCCATCTGGATTTGTTCAGAACGCT
CGGTTGCCGCCGGGCGTTTTTTATTGGTGAGAATCCAGGGGTCCCCAATAATTACGATTT
AAATCCTTCAAACTTCCCAAAGGCGAGCCCTAGTGACATTAGAAAACCGACTGTAAAAAGT
ACAGTCGGCATTATCTCATATTATAAAAGCCAGTCATTAGGCCTATCTGACAATTCC
TGAATAGAGTTCATAAACAATCCTGCATGATAACCATCACAAACAGAATGATGTAC
CTGTAAAGATAGCGGTAAATATATTGAATTACCTTTATTAATGAATTTTCCTGCTGTA
ATAATGGGTAGAAGGTAATTACTATTATTATTGATATTTAAGTTAAACCCAGTAAAT
GAAGTCCATGGAATAATAGAAAGAGAAAAAGCATTTTCAGGTATAGGTGTTTTGGG
AAACAATTTCCCCGAACCATTATATTTCTCTACATCAGAAAGGTATAAATCATAAAA
CTCTTTGAAGTCATTCTTTACAGGAGTCCAAATACCAGAGAATGTTTTAGATACACC
ATCAAAAATTGTATAAAGTGGCTCTAACTTATCCCAATAACCTAACTCTCCGTCGCT
ATTGTAACCAGTTCTAAAAGCTGTATTTGAGTTTATCACCCTTGTCACTAAGAAAAT
AAATGCAGGGTAAAATTTATATCCTTCTTGTTTTATGTTTCGGTATAAAACACTAATA
TCAATTTCTGTGGTTATACTAAAAGTCGTTTGTTGGTTCAAATAATGATTAAATATCT
CTTTTCTCTTCCAATTGTCTAAATCAATTTTATTAAAGTTCATTTGATATGCCTCCTAA
ATTTTTATCTAAAGTGAATTTAGGAGGCTTACTTGTCTGCTTTCTTCATTAGAATCAA
TCCTTTTTTAAAAGTCAATCCCGTTTGTTGAAGACTTTTGTCCTTTTCCGCTGCATAA
ACAATATGGCCCGTTTGTTGAACTACTCTTTAATAAAATAATTTTTCCGTTCCCAATT
CCACATTGCAATAATAGAAAATCCATCTTCATCGGCTTTTTCGTCATCATCTGTATGA
ATCAAATCGCCTTCTTCTGTGTCATCAAGGTTTAATTTTTTATGTATTTCTTTTAACAA
ACCACCATAGGAGATTAACCTTTTACGGTGTAAACCTTCCTCCAAATCAGACAAACG
TTTCAAATTCTTTTCTTCATCATCGGTCATAAAATCCGTATCCTTTACAGGATATTTT
GCAGTTTCGTCAATTGCCGATTGTATATCCGATTTATATTTATTTTTCGGTCGAATCA
TTTGAACTTTTACATTTGGATCATAGTCTAATTTCATTGCCTTTTTCCAAAATTGAAT
CCATTGTTTTTGATTCACGTAGTTTTCTGTATTCTTAAAATAAGTTGGTTCCACACAT
ACCAATACATGCATGTGCTGATTATAAGAATTATCTTTATTATTTATTGTCACTTCCG
TTGCACGCATAAAACCAACAAGATTTTTATTAATTTTTTTATATTGCATCATTCGGCG
AAATCCTTGAGCCATATCTGACAAACTCTTATTTAATTCTTCGCCATCATAAACATTT
TTAACTGTTAATGTGAGAAACAACCAACGAACTGTTGGCTTTTGTTTAATAACTTCA
GCAACAACCTTTTGTGACTGAATGCCATGTTTCATTGCTCTCCTCCAGTTGCACATTG
GACAAAGCCTGGATTTACAAAACCACACTCGATACAACTTTCTTTCGCCTGTTTCAC
GATTTTGTTTATACTCTAATATTTCAGCACAATCTTTTACTCTTTCAGCCTTTTTAAAT
TCAAGAATATGCAGAAGTTCAAAGTAATCAACATTAGCGATTTTCTTTTCTCTCCAT
GGTCTCACTTTTCCACTTTTTGTCTTGTCCACTAAAACCCTTGATTTTTCATCTGAATA
AATGCTACTATTAGGACACATAATATTAAAAGAAACCCCCATCTATTTAGTTATTTG
TTTGGTCACTTATAACTTTAACAGATGGGGTTTTTCTGTGCAACCAATTTTAAGGGTT
TTCCAATACTTTAAAACACATACATACCAACACTTCAACGCACCTTTCAGCAACTAA
AATAAAAATGACGTTATTTCTATATGTATCAAGATAAGAAAGAACAAGTTCAAAAC
CATCAAAAAAAGACACCTTTTCAGGTGCTTTTTTTATTTTATAAACTCATTCCCTGAT
CTCGACTTCGTTCTTTTTTTACCTCTCGGTTATGAGTTAGTTCAAATTCGTTCTTTTTA
GGTTCTAAATCGTGTTTTTCTTGGAATTGTGCTGTTTTATCCTTTACCTTGTCTACAAA
CCCCTTAAAAACGTTTTTAAAGGCTTTTAAGCGTCTGTACGTTCCTTAAGGAATTATT
CCTTAGTGCTTTCTAGGTTAATGTCATGATAATAATGGTTTCTTAGACGTCAGGTGGC
ACTTTTCGGGGAAATGTCCGCGGAACCCCTATTTGTTTATTTTTCTAAATACATTCAA
ATATGTATCCGCTCATGAGACAATAACCCTGATAAATGCTTCAATAATATTGAAAAA
GGAAGAGTATGAGTATTCAACATTTCCGTGTCGCCCTTATTCCCTTTTTTGCGGCATT
TTGCCTTCCTGTTTTTGCTCACCCAGAAACGCTGGTGAAAGTAAAAGATGCTGAAGA
TCAGTTGGGTGCACGAGTGGGTTACATCGAACTGGATCTCAACAGCGGTAAGATCCT
TGAGAGTTTTCGCCCCGAAGAACGTTTTCCAATGATGAGCACTTTTAAAGTTCTGCT
ATGTGGCGCGGTATTATCCCGTGTTGACGCCGGGCAAGAGCAACTCGGTCGCCGCAT
ACACTATTCTCAGAATGACTTGGTTGAGTACTCACCAGTCACAGAAAAGCATCTTAC
GGATGGCATGACAGTAAGAGAATTATGCAGTGCTGCCATAACCATGAGTGATAACA
CTGCGGCCAACTTACTTCTGACAACGATCGGAGGACCGAAGGAGCTAACCGCTTTTT
TGCACAACATGGGGGATCATGTAACTCGCCTTGATCGTTGGGAACCGGAGCTGAAT
GAAGCCATACCAAACGACGAGCGTGACACCACGATGCCTGCAGCAATGGCAACAAC
GTTGCGCAAACTATTAACTGGCGAACTACTTACTCTAGCTTCCCGGCAACAATTAAT
AGACTGGATGGAGGCGGATAAAGTTGCAGGACCACTTCTGCGCTCGGCCCTTCCGG
CTGGCTGGTTTATTGCTGATAAATCTGGAGCCGGTGAGCGTGGGTCTCGCGGTATCA
TTGCAGCACTGGGGCCAGATGGTAAGCCCTCCCGTATCGTAGTTATCTACACGACGG
GGAGTCAGGCAACTATGGATGAACGAAATAGACAGATCGCTGAGATAGGTGCCTCA
CTGATTAAGCATTGGTAACTGTCAGACCAAGTTTACTCATATATACTTTAGATTGATT
TAAAACTTCATTTTTAATTTAAAAGGATCTAGGTGAAGATCCTTTTTGATAATCTCAT
GACCAAAATCCCTTAACGTGAGTTTTCGTTCCACTGAGCGTCAGACCCCTTAATAAG
ATGATCTTCTTGAGATCGTTTTGGTCTGCGCGTAATCTCTTGCTCTGAAAACGAAAA
AACCGCCTTGCAGGGAGGTTTTTCGAAGGTTCTCTGAGCTACCAACTCTTTGAACCG
AGGTAACTGGCTTGCAGGAGCGCAGTCACCAAAACTTGTCCTTTCAGTTTAGCCTTA
ACCGGCGCATGACTTCAAGACTAACTCCTCTAAATCAATTACCAGTGGCTGCTGCCA
GTGGTGCTTTTGCATGTCTTTCCGGGTTGGACTCAAGACGATAGTTACCGGATAAGG
CGCAGCGGTCGGACTGAACGGGGGGTTCGTGCATACAGTCCAGCTTGGAGCGAACT
GCCTACCCGGAACTGAGTGTCAGGCGTGGAATGAGACAAACGCGGCCATAACAGCG
GAATGACACCGGTAAACCGAAAGGCAGGAACAGGAGAGCGCACGAGGGAGCCGCC
AGGGGGAAACGCCTGGTATCTTTATAGTCCTGTCGGGTTTCGCCACCACTGATTTGA
GCGTCAGATTTCGTGATGCTTGTCAGGGGGCGGAGCC
>pBb
(SEQ ID No: 227)
tacggttatccacagaatcagagggcaaactactagagat
GACGGGCCTGAACAAGAGCACCGTAAGCAGC
CAAGTGAATACACTGATGAAGGAGTCAATGGTGTTCGAGATCGGCCAGGGCCAGAG
CTCAGGTGGCCGCCGCCCGGTAATGTTAGTCTTCAACAAGAAAGCGGGCTATAGTGT
GGGCATCGACGTCGGCGTAGACTACATCAACGGAATCCTTACGGATTTAGAGGGCA
CGATTGTGTTGGACCAGTATAGACACCTTGAGAGCAACAGCCCGGAGATCACTAAG
GATATCCTTATCGACATGATCCACCATTTCATCACACAGATGCCTCAGAGCCCTTAT
GGATTCATCGGCATCGGCATCTGTGTACCGGGTTTAATCGACAAGGACCAGAAGATC
GTGTTTACGCCTAATAGCAATTGGCGTGACATCGATCTGAAGAGCAGCATCCAGGA
GAAATATAACGTAAGCGTGTTCATCGAGAACGAAGCGAACGCAGGAGCGTACGGCG
AGAAGTTGTTCGGTGCAGCGAAGAACCATGACAATATCATCTATGTGTCAATTTCAA
CGGGCATCGGAATTGGCGTGATCATTAATAACCACCTGTACCGTGGCGTGTCAGGAT
TTAGCGGTGAGATGGGCCACATGACGATCGATTTCAACGGACCGAAGTGTTCATGTG
GCAATAGAGGCTGTTGGGAGCTTTACGCGAGCGAGAAAGCGCTGCTGAAGAGCTTG
CAAACGAAGGAGAAGAAACTCAGCTACCAGGACATCATCAATCTTGCACACCTTAA
CGACATTGGCACACTGAACGCGCTGCAGAACTTCGGCTTCTACCTGGGCATCGGATT
GACAAACATCTTGAACACGTTCAATCCGCAGGCAGTGATCCTGCGCAACTCAATCAT
CGAGAGCCACCCGATGGTGCTGAACAGCATGCGCTCAGAGGTGAGCAGCCGTGTGT
ACAGCCAGCTCGGAAACTCATACGAGCTGCTTCCGAGCAGTCTTGGCCAGAACGCT
CCTGCGCTTGGCATGAGCAGCATCGTAATCGACCACTTCTTAGATATGATCACGATG
TAATAGTCAAATGTCAGACGAAAATGCCAATTATTGAAGCGGCTAACGCCGCTTTTT
TTGTTTCTGGTCTCCCCATAGTTTAACAAGTGCTGTAAGAAGTCATTCAGCATAGCTT
CTAGAGATCTGCAGTGACTGGGAAAACCCTGGCGACTAGTCTTGGACTCCTGTTGAT
AGATCCAGTAATGACCTCAGAACTCCATCTGGATTTGTTCAGAACGCTCGGTTGCCG
CCGGGCGTTTTTTATTGGTGAGAATCCAGGGGTCCCCAATAATTACGATTTAAATCC
TTCAAACTTCCCAAAGGCGAGCCCTAGTGACATTAGAAAACCGACTGTAAAAAGTA
CAGTCGGCATTATCTCATATTATAAAAGCCAGTCATTAGGCCTATCTGACAATTCCT
GAATAGAGTTCATAAACAATCCTGCATGATAACCATCACAAACAGAATGATGTACC
TGTAAAGATAGCGGTAAATATATTGAATTACCTTTATTAATGAATTTTCCTGCTGTAA
TAATGGGTAGAAGGTAATTACTATTATTATTGATATTTAAGTTAAACCCAGTAAATG
AAGTCCATGGAATAATAGAAAGAGAAAAAGCATTTTCAGGTATAGGTGTTTTGGGA
AACAATTTCCCCGAACCATTATATTTCTCTACATCAGAAAGGTATAAATCATAAAAC
TCTTTGAAGTCATTCTTTACAGGAGTCCAAATACCAGAGAATGTTTTAGATACACCA
TCAAAAATTGTATAAAGTGGCTCTAACTTATCCCAATAACCTAACTCTCCGTCGCTA
TTGTAACCAGTTCTAAAAGCTGTATTTGAGTTTATCACCCTTGTCACTAAGAAAATA
AATGCAGGGTAAAATTTATATCCTTCTTGTTTTATGTTTCGGTATAAAACACTAATAT
CAATTTCTGTGGTTATACTAAAAGTCGTTTGTTGGTTCAAATAATGATTAAATATCTC
TTTTCTCTTCCAATTGTCTAAATCAATTTTATTAAAGTTCATTTGATATGCCTCCTAAA
TTTTTATCTAAAGTGAATTTAGGAGGCTTACTTGTCTGCTTTCTTCATTAGAATCAAT
CCTTTTTTAAAAGTCAATCCCGTTTGTTGAAGACTTTTGTCCTTTTCCGCTGCATAAA
CAATATGGCCCGTTTGTTGAACTACTCTTTAATAAAATAATTTTTCCGTTCCCAATTC
CACATTGCAATAATAGAAAATCCATCTTCATCGGCTTTTTCGTCATCATCTGTATGAA
TCAAATCGCCTTCTTCTGTGTCATCAAGGTTTAATTTTTTATGTATTTCTTTTAACAAA
CCACCATAGGAGATTAACCTTTTACGGTGTAAACCTTCCTCCAAATCAGACAAACGT
TTCAAATTCTTTTCTTCATCATCGGTCATAAAATCCGTATCCTTTACAGGATATTTTG
CAGTTTCGTCAATTGCCGATTGTATATCCGATTTATATTTATTTTTCGGTCGAATCAT
TTGAACTTTTACATTTGGATCATAGTCTAATTTCATTGCCTTTTTCCAAAATTGAATC
CATTGTTTTTGATTCACGTAGTTTTCTGTATTCTTAAAATAAGTTGGTTCCACACATA
CCAATACATGCATGTGCTGATTATAAGAATTATCTTTATTATTTATTGTCACTTCCGT
TGCACGCATAAAACCAACAAGATTTTTATTAATTTTTTTATATTGCATCATTCGGCGA
AATCCTTGAGCCATATCTGACAAACTCTTATTTAATTCTTCGCCATCATAAACATTTT
TAACTGTTAATGTGAGAAACAACCAACGAACTGTTGGCTTTTGTTTAATAACTTCAG
CAACAACCTTTTGTGACTGAATGCCATGTTTCATTGCTCTCCTCCAGTTGCACATTGG
ACAAAGCCTGGATTTACAAAACCACACTCGATACAACTTTCTTTCGCCTGTTTCACG
ATTTTGTTTATACTCTAATATTTCAGCACAATCTTTTACTCTTTCAGCCTTTTTAAATT
CAAGAATATGCAGAAGTTCAAAGTAATCAACATTAGCGATTTTCTTTTCTCTCCATG
GTCTCACTTTTCCACTTTTTGTCTTGTCCACTAAAACCCTTGATTTTTCATCTGAATAA
ATGCTACTATTAGGACACATAATATTAAAAGAAACCCCCATCTATTTAGTTATTTGTT
TGGTCACTTATAACTTTAACAGATGGGGTTTTTCTGTGCAACCAATTTTAAGGGTTTT
CCAATACTTTAAAACACATACATACCAACACTTCAACGCACCTTTCAGCAACTAAAA
TAAAAATGACGTTATTTCTATATGTATCAAGATAAGAAAGAACAAGTTCAAAACCAT
CAAAAAAAGACACCTTTTCAGGTGCTTTTTTTATTTTATAAACTCATTCCCTGATCTC
GACTTCGTTCTTTTTTTACCTCTCGGTTATGAGTTAGTTCAAATTCGTTCTTTTTAGGT
TCTAAATCGTGTTTTTCTTGGAATTGTGCTGTTTTATCCTTTACCTTGTCTACAAACCC
CTTAAAAACGTTTTTAAAGGCTTTTAAGCGTCTGTACGTTCCTTAAGGAATTATTCCT
TAGTGCTTTCTAGGTTAATGTCATGATAATAATGGTTTCTTAGACGTCAGGTGGCACT
TTTCGGGGAAATGTCCGCGGAACCCCTATTTGTTTATTTTTCTAAATACATTCAAATA
TGTATCCGCTCATGAGACAATAACCCTGATAAATGCTTCAATAATATTGAAAAAGGA
AGAGTATGAGTATTCAACATTTCCGTGTCGCCCTTATTCCCTTTTTTGCGGCATTTTG
CCTTCCTGTTTTTGCTCACCCAGAAACGCTGGTGAAAGTAAAAGATGCTGAAGATCA
GTTGGGTGCACGAGTGGGTTACATCGAACTGGATCTCAACAGCGGTAAGATCCTTGA
GAGTTTTCGCCCCGAAGAACGTTTTCCAATGATGAGCACTTTTAAAGTTCTGCTATGT
GGCGCGGTATTATCCCGTGTTGACGCCGGGCAAGAGCAACTCGGTCGCCGCATACA
CTATTCTCAGAATGACTTGGTTGAGTACTCACCAGTCACAGAAAAGCATCTTACGGA
TGGCATGACAGTAAGAGAATTATGCAGTGCTGCCATAACCATGAGTGATAACACTG
CGGCCAACTTACTTCTGACAACGATCGGAGGACCGAAGGAGCTAACCGCTTTTTTGC
ACAACATGGGGGATCATGTAACTCGCCTTGATCGTTGGGAACCGGAGCTGAATGAA
GCCATACCAAACGACGAGCGTGACACCACGATGCCTGCAGCAATGGCAACAACGTT
GCGCAAACTATTAACTGGCGAACTACTTACTCTAGCTTCCCGGCAACAATTAATAGA
CTGGATGGAGGCGGATAAAGTTGCAGGACCACTTCTGCGCTCGGCCCTTCCGGCTGG
CTGGTTTATTGCTGATAAATCTGGAGCCGGTGAGCGTGGGTCTCGCGGTATCATTGC
AGCACTGGGGCCAGATGGTAAGCCCTCCCGTATCGTAGTTATCTACACGACGGGGA
GTCAGGCAACTATGGATGAACGAAATAGACAGATCGCTGAGATAGGTGCCTCACTG
ATTAAGCATTGGTAACTGTCAGACCAAGTTTACTCATATATACTTTAGATTGATTTAA
AACTTCATTTTTAATTTAAAAGGATCTAGGTGAAGATCCTTTTTGATAATCTCATGAC
CAAAATCCCTTAACGTGAGTTTTCGTTCCACTGAGCGTCAGACCCCTTAATAAGATG
ATCTTCTTGAGATCGTTTTGGTCTGCGCGTAATCTCTTGCTCTGAAAACGAAAAAAC
CGCCTTGCAGGGAGGTTTTTCGAAGGTTCTCTGAGCTACCAACTCTTTGAACCGAGG
TAACTGGCTTGCAGGAGCGCAGTCACCAAAACTTGTCCTTTCAGTTTAGCCTTAACC
GGCGCATGACTTCAAGACTAACTCCTCTAAATCAATTACCAGTGGCTGCTGCCAGTG
GTGCTTTTGCATGTCTTTCCGGGTTGGACTCAAGACGATAGTTACCGGATAAGGCGC
AGCGGTCGGACTGAACGGGGGGTTCGTGCATACAGTCCAGCTTGGAGCGAACTGCC
TACCCGGAACTGAGTGTCAGGCGTGGAATGAGACAAACGCGGCCATAACAGCGGAA
TGACACCGGTAAACCGAAAGGCAGGAACAGGAGAGCGCACGAGGGAGCCGCCAGG
GGGAAACGCCTGGTATCTTTATAGTCCTGTCGGGTTTCGCCACCACTGATTTGAGCG
TCAGATTTCGTGATGCTTGTCAGGGGGCGGAGCCTATGGAAAAACGCTTTGCCCCAC
ACAGGCTCTTGAACAAGGGATAACAATTTGTCATAGCTGTATTCGTTACAAGGAGGA
TTTTCGAAAAAACACCCTAACGGGTGTTTTTTTATAGCTGGTCTCCCTTGCTTGTACT
TTACAGTATGCCATACGAAACTGCGTCAATACACGTTGACACTCTTTTGTAGTTATGT
TAAATTATCAGATCGCCATCATTAGTTTGTTTAAACAACAAACTAATACTAGAGGCT
CTTAAAGGGGGTTTTAGATACCAGAGATGGTGAGCAAGGGCGAGGAGGATAACATG
GCCATCATCAAGGAGTTCATGCGCTTCAAGGTGCACATGGAGGGCTCCGTGAACGG
CCACGAGTTCGAGATCGAGGGCGAGGGCGAGGGCCGCCCCTACGAGGGCACCCAG
ACCGCCAAGCTGAAGGTGACCAAGGGTGGCCCCCTGCCCTTCGCCTGGGACATCCT
GTCCCCTCAGTTCATGTACGGCTCCAAGGCCTACGTGAAGCACCCCGCCGACATCCC
CGACTACTTGAAGCTGTCCTTCCCCGAGGGCTTCAAGTGGGAGCGCGTGATGAACTT
CGAGGACGGCGGCGTGGTGACCGTGACCCAGGACTCCTCCCTGCAGGACGGCGAGT
TCATCTACAAGGTGAAGCTGCGCGGCACCAACTTCCCCTCCGACGGCCCCGTAATGC
AGAAGAAGACCATGGGCTGGGAGGCCTCCTCCGAGCGGATGTACCCCGAGGACGGC
GCCCTGAAGGGCGAGATCAAGCAGAGGCTGAAGCTGAAGGACGGCGGCCACTACG
ACGCTGAGGTCAAGACCACCTACAAGGCCAAGAAGCCCGTGCAGCTGCCCGGCGCC
TACAACGTCAACATCAAGTTGGACATCACCTCCCACAACGAGGACTACACCATCGT
GGAACAGTACGAACGCGCCGAGGGCCGCCACTCCACCGGCGGCATGGACGAGCTGT
ACAAGTAATAGCTGCCACCTTTCTGAACTTTGGTAATGCGTCTCGGTACCAAATTCC
AGAAAAGAGGCCTCCCGAAAGGGGGGCCTTTTTTCGTTTTGGTCCCTCcaatgatctcc
ttatccggtcgacattcacgattacttggCTAGCGCTATATGCGTTGATGCAATTTCTA
TGCGCACCCGTTCTCGGAGCACTGTCCGACCGCTTTGGCCGCCGCCCAGTCCTGCTCGC
TTCGCTACTTGGAGCCACTATCGACTACGCGATCATGGCGACCACACCCGTCCTGTGGA
TCTATCGATGCATGCAGCCGGTACCATCACAAGCACTTTGGGACGTTCTCCCTTAGTGC
TTTTTTGATTTCTCATAGGCCGGCCTGTTAGTCATATGGACACTTAAGCTCTAGACGGT
GATCAACACGCTAGCCAGGCATCAAATAAAACGAAAGGCTCAGTCGAAAGACTGGGCCT
TTCGTTTTATCTGTTGTTTGTCGGTGAACGCTCTCTACTAGAGTCACACTGGCTCACCT
TCGGGTGGGCCTTTCTGCGTTTATAGAATTCACTAGTAATTGTGAGGCGGATAACAATT
CTCACTCATTCTACAGTTTATTCTTGACATTGCACTGTCCCCCTGGTATAATAACTAA
TTGTGAGCGCTCACAATTAAGATCCCCGGGGCGGCCGCGAATTCAAAAGATCTAAA
GAGGAGAAAGGATCTATGGATAAGAAATACTCAATAGGCTTAGCTATCGGCACAAA
TAGCGTCGGATGGGCGGTGATCACTGATGAATATAAGGTTCCGTCTAAAAAGTTCAA
GGTTCTGGGAAATACAGACCGCCACAGTATCAAAAAAAATCTTATAGGGGCTCTTTT
ATTTGACAGTGGAGAGACAGCGGAAGCGACTCGTCTCAAACGGACAGCTCGTAGAA
GGTATACACGTCGGAAGAATCGTATTTGTTATCTACAGGAGATTTTTTCAAATGAGA
TGGCGAAAGTAGATGATAGTTTCTTTCATCGACTTGAAGAGTCTTTTTTGGTGGAAG
AAGACAAGAAGCATGAACGTCATCCTATTTTTGGAAATATAGTAGATGAAGTTGCTT
ATCATGAGAAATATCCAACTATCTATCATCTGCGAAAAAAATTGGTAGATTCTACTG
ATAAAGCGGATTTGCGCTTAATCTATTTGGCCTTAGCGCATATGATTAAGTTTCGTG
GTCATTTTTTGATTGAGGGAGATTTAAATCCTGATAATAGTGATGTGGACAAACTAT
TTATCCAGTTGGTACAAACCTACAATCAATTATTTGAAGAAAACCCTATTAACGCAA
GTGGAGTAGATGCTAAAGCGATTCTTTCTGCACGATTGAGTAAATCAAGACGATTAG
AAAATCTCATTGCTCAGCTCCCCGGTGAGAAGAAAAATGGCTTATTTGGGAATCTCA
TTGCTTTGTCATTGGGTTTGACCCCTAATTTTAAATCAAATTTTGATTTGGCAGAAGA
TGCTAAATTACAGCTTTCAAAAGATACTTACGATGATGATTTAGATAATTTATTGGC
GCAAATTGGAGATCAATATGCTGATTTGTTTTTGGCAGCTAAGAATTTATCAGATGC
TATTTTACTTTCAGATATCCTAAGAGTAAATACTGAAATAACTAAGGCTCCCCTATC
AGCTTCAATGATTAAACGCTACGATGAACATCATCAAGACTTGACTCTTTTAAAAGC
TTTAGTTCGACAACAACTTCCAGAAAAGTATAAAGAAATCTTTTTTGATCAATCAAA
AAACGGATATGCAGGTTATATTGATGGGGGAGCTAGCCAAGAAGAATTTTATAAAT
TTATCAAACCAATTTTAGAAAAAATGGATGGTACTGAGGAATTATTGGTGAAACTAA
ATCGTGAAGATTTGCTGCGCAAGCAACGGACCTTTGACAACGGCTCTATTCCCCATC
AAATTCACTTGGGTGAGCTGCATGCTATTTTGAGAAGACAAGAAGACTTTTATCCAT
TTTTAAAAGACAATCGTGAGAAGATTGAAAAAATCTTGACTTTTCGAATTCCTTATT
ATGTTGGTCCATTGGCGCGTGGCAATAGTCGTTTTGCATGGATGACTCGGAAGTCTG
AAGAAACAATTACCCCATGGAATTTTGAAGAAGTTGTCGATAAAGGTGCTTCAGCTC
AATCATTTATTGAACGCATGACAAACTTTGATAAAAATCTTCCAAATGAAAAAGTAC
TACCAAAACATAGTTTGCTTTATGAGTATTTTACGGTTTATAACGAATTGACAAAGG
TCAAATATGTTACTGAAGGAATGCGAAAACCAGCATTTCTTTCAGGTGAACAGAAG
AAAGCCATTGTTGATTTACTCTTCAAAACAAATCGAAAAGTAACCGTTAAGCAATTA
AAAGAAGATTATTTCAAAAAAATAGAATGTTTTGATAGTGTTGAAATTTCAGGAGTT
GAAGATAGATTTAATGCTTCATTAGGTACCTACCATGATTTGCTAAAAATTATTAAA
GATAAAGATTTTTTGGATAATGAAGAAAATGAAGATATCTTAGAGGATATTGTTTTA
ACATTGACCTTATTTGAAGATAGGGAGATGATTGAGGAAAGACTTAAAACATATGC
TCACCTCTTTGATGATAAGGTGATGAAACAGCTTAAACGTCGCCGTTATACTGGTTG
GGGACGTTTGTCTCGAAAATTGATTAATGGTATTAGGGATAAGCAATCTGGCAAAAC
AATATTAGATTTTTTGAAATCAGATGGTTTTGCCAATCGCAATTTTATGCAGCTGATC
CATGATGATAGTTTGACATTTAAAGAAGACATTCAAAAAGCACAAGTGTCTGGACA
AGGCGATAGTTTACATGAACATATTGCAAATTTAGCTGGTAGCCCTGCTATTAAAAA
AGGTATTTTACAGACTGTAAAAGTTGTTGATGAATTGGTCAAAGTAATGGGGCGGCA
TAAGCCAGAAAATATCGTTATTGAAATGGCACGTGAAAATCAGACAACTCAAAAGG
GCCAGAAAAATTCGCGAGAGCGTATGAAACGAATCGAAGAAGGTATCAAAGAATTA
GGAAGTCAGATTCTTAAAGAGCATCCTGTTGAAAATACTCAATTGCAAAATGAAAA
GCTCTATCTCTATTATCTCCAAAATGGAAGAGACATGTATGTGGACCAAGAATTAGA
TATTAATCGTTTAAGTGATTATGATGTCGATGCCATTGTTCCACAAAGTTTCCTTAAA
GACGATTCAATAGACAATAAGGTCTTAACGCGTTCTGATAAAAATCGTGGTAAATCG
GATAACGTTCCAAGTGAAGAAGTAGTCAAAAAGATGAAAAACTATTGGAGACAACT
TCTAAACGCCAAGTTAATCACTCAACGTAAGTTTGATAATTTAACGAAAGCTGAACG
TGGAGGTTTGAGTGAACTTGATAAAGCTGGTTTTATCAAACGCCAATTGGTTGAAAC
TCGCCAAATCACTAAGCATGTGGCACAAATTTTGGATAGTCGCATGAATACTAAATA
CGATGAAAATGATAAACTTATTCGAGAGGTTAAAGTGATTACCTTAAAATCTAAATT
AGTTTCTGACTTCCGAAAAGATTTCCAATTCTATAAAGTACGTGAGATTAACAATTA
CCATCATGCCCATGATGCGTATCTAAATGCCGTCGTTGGAACTGCTTTGATTAAGAA
ATATCCAAAACTTGAATCGGAGTTTGTCTATGGTGATTATAAAGTTTATGATGTTCGT
AAAATGATTGCTAAGTCTGAGCAAGAAATAGGCAAAGCAACCGCAAAATATTTCTT
TTACTCTAATATCATGAACTTCTTCAAAACAGAAATTACACTTGCAAATGGAGAGAT
TCGCAAACGCCCTCTAATCGAAACTAATGGGGAAACTGGAGAAATTGTCTGGGATA
AAGGGCGAGATTTTGCCACAGTGCGCAAAGTATTGTCCATGCCCCAAGTCAATATTG
TCAAGAAAACAGAAGTACAGACAGGCGGATTCTCCAAGGAGTCAATTTTACCAAAA
AGAAATTCGGACAAGCTTATTGCTCGTAAAAAAGACTGGGATCCAAAAAAATATGG
TGGTTTTGATAGTCCAACGGTAGCTTATTCAGTCCTAGTGGTTGCTAAGGTGGAAAA
AGGGAAATCGAAGAAGTTAAAATCCGTTAAAGAGTTACTAGGGATCACAATTATGG
AAAGAAGTTCCTTTGAAAAAAATCCGATTGACTTTTTAGAAGCTAAAGGATATAAG
GAAGTTAAAAAAGACTTAATCATTAAACTACCTAAATATAGTCTTTTTGAGTTAGAA
AACGGTCGTAAACGGATGCTGGCTAGTGCCGGAGAATTACAAAAAGGAAATGAGCT
GGCTCTGCCAAGCAAATATGTGAATTTTTTATATTTAGCTAGTCATTATGAAAAGTT
GAAGGGTAGTCCAGAAGATAACGAACAAAAACAATTGTTTGTGGAGCAGCATAAGC
ATTATTTAGATGAGATTATTGAGCAAATCAGTGAATTTTCTAAGCGTGTTATTTTAGC
AGATGCCAATTTAGATAAAGTTCTTAGTGCATATAACAAACATAGAGACAAACCAA
TACGTGAACAAGCAGAAAATATTATTCATTTATTTACGTTGACGAATCTTGGAGCTC
CCGCTGCTTTTAAATATTTTGATACAACAATTGATCGTAAACGATATACGTCTACAA
AAGAAGTTTTAGATGCCACTCTTATCCATCAATCCATCACTGGTCTTTATGAAACAC
GCATTGATTTGAGTCAGCTAGGAGGTGACTAAGTCGACCTCGAGACTAGTCAAGGTC
GGCAATTCTGCAGTACTAGGACGCCGCCAAGCCAGCTTAAACCCAGCTCAATGAGC
TGGGTTTTTTGTTTGTTAAAAATGAAGAAGAAACTGTGAAGCGTATTTATAGCAAAG
CACTCAAAAGTTTACCTTATGGGTGCTTTTTTCGTGCTTTTTTGAAAAGACAAAAAA
AAGAACCTTGCCAAGCAAGATTCTTTTGATAGCGCTATCGCTGAGCGCCGGTCGCTA
CCATTACCAGTTGGTCTGGTGTCAAAAATAATAATAACCGGGCAGGCCATGTCTGCC
CGTATTTCGCGTAAGGAAATCCATTATGTACTATTTCTGGTGATGAAATCAACGTAA
CATTTAAAGCTGTCAAAGCCAAAGTCATGAGATGGCGTATGGAGCGTAAAGCTGAC
AAGAGCGGTGTTGCGATGATTGAGATGACCTTCCTTGCACCAAGTGAATTGCCTCAA
GAAAGCACTCAATCAAAGATTCTTGTAGATGGAAAAGAACTTGCTGATTTCGCTGAA
AATCGTCAAGACTATCAAATTACCTATAAAGGTCAACGGCCAAAAGTCTCAGTTGA
AGAAAACAATCAAGTAGCTTCAACTGTGGTAGATAGTGGAGAAGATAGCCTTCCAG
TACTTGTTCGCCTCGTTTCAGAAAGTGGAAAACAAGTCAAGGAATACCGTATCCAGT
TGACTAAGGAAAAACCAGTTTCTGCTGTACAAGAAGATCTTCCAAAACTCGAATTTG
TTGAAAAAGATTTGGCCTACAAGACAGTTGAGAAAAAAGATTCAACACTGTATCTA
GGTGAAACTCGTGTAGAACAAGAAGGAAAAGTTGGAAAAGAACGTATCTTTACAGT
GATTAATCCTGATGGAAGTAAGGAAGAAAAACTCCGTGAAGTGGTAGAAGTTCCGA
CAGACCGCATCGTCTTGGTTGGAACCAAACCAGTAGCTCAAGAAGCTAAAAAACCA
CAAGTGTCAGAAAAAGCAGATACAAAACCAATTGATTCAAGTGAAGCTGATCAAAC
TAATAAAGCCCAGTTACCAAATACAGGTAGTGCGGCAAGCCAAGCAGCAGTAGCAG
CAGGTTTAGCCTGCCTCGCGCGTTTCGGTGATGACGGTGAAAACCTCTGACACATGC
AGCTCCCGGAGACGGTCACAGCTTGTCTGTAAGCGGATGCCGGGAGCAGACAAGCC
CGTCAGGGCGCGTCAGCGGGTGTTGGCGGGTGTCGGGGCGCAGCCATGACCCAGTC
ACGTAGCGATAGCGGAGTGTATACTGGCTTAACTATGCGGCATCAGAGCAGATTGT
ACTGAGAGTGCACCATATGCGGTGTGAAATACCGCACAGATGCGTAAGGAGAAAAT
ACCGCATCAGGCGCTCTTCCGCTTCCTCGCTCACTGACTCGCTGCGCTCGGTCGTTCG
GCTGCGGCGAGCGGTATCAGCTCACTCAAAGGCGGTAA
>pV1-PsspA
(SEQ ID No: 228)
GAGCCTATGGAAAAACGCTTTGCCCCACACAGGCTCTTGAACAAGGGATAACAATT
TGTCATAGCTGTATTCGTTACAAGGAGGATTTTCGAAAAAACACCCTAACGGGTGTT
TTTTTATAGCTGGTCTCCCCAAGTGCTGTAAAAAACGCCGAATGAAGCGGATGAAAT
TGCTCATCGTCTCGTGAAAAAGCCGAAGAAGGTTAAGCCTGGGTATAAAAAGAAAA
TGAGCTATGAGATGGAGAAAGCCATAACCATGAGTGATAACACTGGTTTTAGAGCT
AGAAATAGCAAGTTAAAATAAGGCTAGTCCGTTATCAACTTGAAAAAGTGGCACCG
AGTCGGTGCTTTTTTTCTCGGTACCAAATTCCAGAAAAGAGGCCTCCCGAAAGGGGG
GCCTTTTTTCGTTTTGGTCCCTCAATGATCTCCTTATCCGGTTAAGATGGCAAGCTTG
ACAAGTATTTCTTCAGAATTTACAATGAAGTTTACATGACGCTCTTAAAGGGGGTTT
TAGATACCAGAGATGGATAAGAAATACTCAATAGGCTTAGATATCGGCACAAATAG
CGTCGGATGGGCGGTGAAACACGCATTGATTTGAGTCAGCTAGGAGGTGACTGATA
ATCAAATGTCAGACGAAAATGCCAATTATTGAAGCGGCTAACGCCGCTTTTTTTGTT
TCTGGTCTCCCTTGCTTGTACTTTACAGTATAGCTTCTAGAGATCTGCAGTGACTGGA
GCTTCTAGAGATCTGCAGTGACTGGGAAAACCCTGGCGACTAGTCTTGGACTCCTGT
TGATAGATCCAGTAATGACCTCAGAACTCCATCTGGATTTGTTCAGAACGCTCGGTT
GCCGCCGGGCGTTTTTTATTGGTGAGAATCCAGGGGTCCCCAATAATTACGATTTAA
ATCCTTCAAACTTCCCAAAGGCGAGCCCTAGTGACATTAGAAAACCGACTGTAAAA
AGTACAGTCGGCATTATCTCATATTATAAAAGCCAGTCATTAGGCCTATCTGACAAT
TCCTGAATAGAGTTCATAAACAATCCTGCATGATAACCATCACAAACAGAATGATGT
ACCTGTAAAGATAGCGGTAAATATATTGAATTACCTTTATTAATGAATTTTCCTGCTG
TAATAATGGGTAGAAGGTAATTACTATTATTATTGATATTTAAGTTAAACCCAGTAA
ATGAAGTCCATGGAATAATAGAAAGAGAAAAAGCATTTTCAGGTATAGGTGTTTTG
GGAAACAATTTCCCCGAACCATTATATTTCTCTACATCAGAAAGGTATAAATCATAA
AACTCTTTGAAGTCATTCTTTACAGGAGTCCAAATACCAGAGAATGTTTTAGATACA
CCATCAAAAATTGTATAAAGTGGCTCTAACTTATCCCAATAACCTAACTCTCCGTCG
CTATTGTAACCAGTTCTAAAAGCTGTATTTGAGTTTATCACCCTTGTCACTAAGAAA
ATAAATGCAGGGTAAAATTTATATCCTTCTTGTTTTATGTTTCGGTATAAAACACTAA
TATCAATTTCTGTGGTTATACTAAAAGTCGTTTGTTGGTTCAAATAATGATTAAATAT
CTCTTTTCTCTTCCAATTGTCTAAATCAATTTTATTAAAGTTCATTTGATATGCCTCCT
AAATTTTTATCTAAAGTGAATTTAGGAGGCTTACTTGTCTGCTTTCTTCATTAGAATC
AATCCTTTTTTAAAAGTCAATCCCGTTTGTTGAAGACTTTTGTCCTTTTCCGCTGCAT
AAACAATATGGCCCGTTTGTTGAACTACTCTTTAATAAAATAATTTTTCCGTTCCCAA
TTCCACATTGCAATAATAGAAAATCCATCTTCATCGGCTTTTTCGTCATCATCTGTAT
GAATCAAATCGCCTTCTTCTGTGTCATCAAGGTTTAATTTTTTATGTATTTCTTTTAAC
AAACCACCATAGGAGATTAACCTTTTACGGTGTAAACCTTCCTCCAAATCAGACAAA
CGTTTCAAATTCTTTTCTTCATCATCGGTCATAAAATCCGTATCCTTTACAGGATATT
TTGCAGTTTCGTCAATTGCCGATTGTATATCCGATTTATATTTATTTTTCGGTCGAAT
CATTTGAACTTTTACATTTGGATCATAGTCTAATTTCATTGCCTTTTTCCAAAATTGA
ATCCATTGTTTTTGATTCACGTAGTTTTCTGTATTCTTAAAATAAGTTGGTTCCACAC
ATACCAATACATGCATGTGCTGATTATAAGAATTATCTTTATTATTTATTGTCACTTC
CGTTGCACGCATAAAACCAACAAGATTTTTATTAATTTTTTTATATTGCATCATTCGG
CGAAATCCTTGAGCCATATCTGACAAACTCTTATTTAATTCTTCGCCATCATAAACAT
TTTTAACTGTTAATGTGAGAAACAACCAACGAACTGTTGGCTTTTGTTTAATAACTTC
AGCAACAACCTTTTGTGACTGAATGCCATGTTTCATTGCTCTCCTCCAGTTGCACATT
GGACAAAGCCTGGATTTACAAAACCACACTCGATACAACTTTCTTTCGCCTGTTTCA
CGATTTTGTTTATACTCTAATATTTCAGCACAATCTTTTACTCTTTCAGCCTTTTTAAA
TTCAAGAATATGCAGAAGTTCAAAGTAATCAACATTAGCGATTTTCTTTTCTCTCCAT
GGTCTCACTTTTCCACTTTTTGTCTTGTCCACTAAAACCCTTGATTTTTCATCTGAATA
AATGCTACTATTAGGACACATAATATTAAAAGAAACCCCCATCTATTTAGTTATTTG
TTTGGTCACTTATAACTTTAACAGATGGGGTTTTTCTGTGCAACCAATTTTAAGGGTT
TTCCAATACTTTAAAACACATACATACCAACACTTCAACGCACCTTTCAGCAACTAA
AATAAAAATGACGTTATTTCTATATGTATCAAGATAAGAAAGAACAAGTTCAAAAC
CATCAAAAAAAGACACCTTTTCAGGTGCTTTTTTTATTTTATAAACTCATTCCCTGAT
CTCGACTTCGTTCTTTTTTTACCTCTCGGTTATGAGTTAGTTCAAATTCGTTCTTTTTA
GGTTCTAAATCGTGTTTTTCTTGGAATTGTGCTGTTTTATCCTTTACCTTGTCTACAAA
CCCCTTAAAAACGTTTTTAAAGGCTTTTAAGCGTCTGTACGTTCCTTAAGGAATTATT
CCTTAGTGCTTTCTAGGTTAATGTCATGATAATAATGGTTTCTTAGACGTCAGGTGGC
ACTTTTCGGGGAAATGTCCGCGGAACCCCTATTTGTTTATTTTTCTAAATACATTCAA
ATATGTATCCGCTCATGAGACAATAACCCTGATAAATGCTTCAATAATATTGAAAAA
GGAAGAGTATGAGTATTCAACATTTCCGTGTCGCCCTTATTCCCTTTTTTGCGGCATT
TTGCCTTCCTGTTTTTGCTCACCCAGAAACGCTGGTGAAAGTAAAAGATGCTGAAGA
TCAGTTGGGTGCACGAGTGGGTTACATCGAACTGGATCTCAACAGCGGTAAGATCCT
TGAGAGTTTTCGCCCCGAAGAACGTTTTCCAATGATGAGCACTTTTAAAGTTCTGCT
ATGTGGCGCGGTATTATCCCGTGTTGACGCCGGGCAAGAGCAACTCGGTCGCCGCAT
ACACTATTCTCAGAATGACTTGGTTGAGTACTCACCAGTCACAGAAAAGCATCTTAC
GGATGGCATGACAGTAAGAGAATTATGCAGTGCTGCCATAACCATGAGTGATAACA
CTGCGGCCAACTTACTTCTGACAACGATCGGAGGACCGAAGGAGCTAACCGCTTTTT
TGCACAACATGGGGGATCATGTAACTCGCCTTGATCGTTGGGAACCGGAGCTGAAT
GAAGCCATACCAAACGACGAGCGTGACACCACGATGCCTGCAGCAATGGCAACAAC
GTTGCGCAAACTATTAACTGGCGAACTACTTACTCTAGCTTCCCGGCAACAATTAAT
AGACTGGATGGAGGCGGATAAAGTTGCAGGACCACTTCTGCGCTCGGCCCTTCCGG
CTGGCTGGTTTATTGCTGATAAATCTGGAGCCGGTGAGCGTGGGTCTCGCGGTATCA
TTGCAGCACTGGGGCCAGATGGTAAGCCCTCCCGTATCGTAGTTATCTACACGACGG
GGAGTCAGGCAACTATGGATGAACGAAATAGACAGATCGCTGAGATAGGTGCCTCA
CTGATTAAGCATTGGTAACTGTCAGACCAAGTTTACTCATATATACTTTAGATTGATT
TAAAACTTCATTTTTAATTTAAAAGGATCTAGGTGAAGATCCTTTTTGATAATCTCAT
GACCAAAATCCCTTAACGTGAGTTTTCGTTCCACTGAGCGTCAGACCCCTTAATAAG
ATGATCTTCTTGAGATCGTTTTGGTCTGCGCGTAATCTCTTGCTCTGAAAACGAAAA
AACCGCCTTGCAGGGAGGTTTTTCGAAGGTTCTCTGAGCTACCAACTCTTTGAACCG
AGGTAACTGGCTTGCAGGAGCGCAGTCACCAAAACTTGTCCTTTCAGTTTAGCCTTA
ACCGGCGCATGACTTCAAGACTAACTCCTCTAAATCAATTACCAGTGGCTGCTGCCA
GTGGTGCTTTTGCATGTCTTTCCGGGTTGGACTCAAGACGATAGTTACCGGATAAGG
CGCAGCGGTCGGACTGAACGGGGGGTTCGTGCATACAGTCCAGCTTGGAGCGAACT
GCCTACCCGGAACTGAGTGTCAGGCGTGGAATGAGACAAACGCGGCCATAACAGCG
GAATGACACCGGTAAACCGAAAGGCAGGAACAGGAGAGCGCACGAGGGAGCCGCC
AGGGGGAAACGCCTGGTATCTTTATAGTCCTGTCGGGTTTCGCCACCACTGATTTGA
GCGTCAGATTTCGTGATGCTTGTCAGGGGGCGGAGCCTATGGAAAAACGCTTTGCCC
>pCas9g1-cons
(SEQ ID No: 229)
aataggcttagatatcggcacaaatagcgtcggatggg
CGGTGATCACTGATGAATATAAGGTTCCGTCTA
AAAAGTTCAAGGTTCTGGGAAATACAGACCGCCACAGTATCAAAAAAAATCTTATA
GGGGCTCTTTTATTTGACAGTGGAGAGACAGCGGAAGCGACTCGTCTCAAACGGAC
AGCTCGTAGAAGGTATACACGTCGGAAGAATCGTATTTGTTATCTACAGGAGATTTT
TTCAAATGAGATGGCGAAAGTAGATGATAGTTTCTTTCATCGACTTGAAGAGTCTTT
TTTGGTGGAAGAAGACAAGAAGCATGAACGTCATCCTATTTTTGGAAATATAGTAG
ATGAAGTTGCTTATCATGAGAAATATCCAACTATCTATCATCTGCGAAAAAAATTGG
TAGATTCTACTGATAAAGCGGATTTGCGCTTAATCTATTTGGCCTTAGCGCATATGAT
TAAGTTTCGTGGTCATTTTTTGATTGAGGGAGATTTAAATCCTGATAATAGTGATGTG
GACAAACTATTTATCCAGTTGGTACAAACCTACAATCAATTATTTGAAGAAAACCCT
ATTAACGCAAGTGGAGTAGATGCTAAAGCGATTCTTTCTGCACGATTGAGTAAATCA
AGACGATTAGAAAATCTCATTGCTCAGCTCCCCGGTGAGAAGAAAAATGGCTTATTT
GGGAATCTCATTGCTTTGTCATTGGGTTTGACCCCTAATTTTAAATCAAATTTTGATT
TGGCAGAAGATGCTAAATTACAGCTTTCAAAAGATACTTACGATGATGATTTAGATA
ATTTATTGGCGCAAATTGGAGATCAATATGCTGATTTGTTTTTGGCAGCTAAGAATTT
ATCAGATGCTATTTTACTTTCAGATATCCTAAGAGTAAATACTGAAATAACTAAGGC
TCCCCTATCAGCTTCAATGATTAAACGCTACGATGAACATCATCAAGACTTGACTCT
TTTAAAAGCTTTAGTTCGACAACAACTTCCAGAAAAGTATAAAGAAATCTTTTTTGA
TCAATCAAAAAACGGATATGCAGGTTATATTGATGGGGGAGCTAGCCAAGAAGAAT
TTTATAAATTTATCAAACCAATTTTAGAAAAAATGGATGGTACTGAGGAATTATTGG
TGAAACTAAATCGTGAAGATTTGCTGCGCAAGCAACGGACCTTTGACAACGGCTCTA
TTCCCCATCAAATTCACTTGGGTGAGCTGCATGCTATTTTGAGAAGACAAGAAGACT
TTTATCCATTTTTAAAAGACAATCGTGAGAAGATTGAAAAAATCTTGACTTTTCGAA
TTCCTTATTATGTTGGTCCATTGGCGCGTGGCAATAGTCGTTTTGCATGGATGACTCG
GAAGTCTGAAGAAACAATTACCCCATGGAATTTTGAAGAAGTTGTCGATAAAGGTG
CTTCAGCTCAATCATTTATTGAACGCATGACAAACTTTGATAAAAATCTTCCAAATG
AAAAAGTACTACCAAAACATAGTTTGCTTTATGAGTATTTTACGGTTTATAACGAAT
TGACAAAGGTCAAATATGTTACTGAAGGAATGCGAAAACCAGCATTTCTTTCAGGTG
AACAGAAGAAAGCCATTGTTGATTTACTCTTCAAAACAAATCGAAAAGTAACCGTT
AAGCAATTAAAAGAAGATTATTTCAAAAAAATAGAATGTTTTGATAGTGTTGAAATT
TCAGGAGTTGAAGATAGATTTAATGCTTCATTAGGTACCTACCATGATTTGCTAAAA
ATTATTAAAGATAAAGATTTTTTGGATAATGAAGAAAATGAAGATATCTTAGAGGAT
ATTGTTTTAACATTGACCTTATTTGAAGATAGGGAGATGATTGAGGAAAGACTTAAA
ACATATGCTCACCTCTTTGATGATAAGGTGATGAAACAGCTTAAACGTCGCCGTTAT
ACTGGTTGGGGACGTTTGTCTCGAAAATTGATTAATGGTATTAGGGATAAGCAATCT
GGCAAAACAATATTAGATTTTTTGAAATCAGATGGTTTTGCCAATCGCAATTTTATG
CAGCTGATCCATGATGATAGTTTGACATTTAAAGAAGACATTCAAAAAGCACAAGT
GTCTGGACAAGGCGATAGTTTACATGAACATATTGCAAATTTAGCTGGTAGCCCTGC
TATTAAAAAAGGTATTTTACAGACTGTAAAAGTTGTTGATGAATTGGTCAAAGTAAT
GGGGCGGCATAAGCCAGAAAATATCGTTATTGAAATGGCACGTGAAAATCAGACAA
CTCAAAAGGGCCAGAAAAATTCGCGAGAGCGTATGAAACGAATCGAAGAAGGTATC
AAAGAATTAGGAAGTCAGATTCTTAAAGAGCATCCTGTTGAAAATACTCAATTGCA
AAATGAAAAGCTCTATCTCTATTATCTCCAAAATGGAAGAGACATGTATGTGGACCA
AGAATTAGATATTAATCGTTTAAGTGATTATGATGTCGATCACATTGTTCCACAAAG
TTTCCTTAAAGACGATTCAATAGACAATAAGGTCTTAACGCGTTCTGATAAAAATCG
TGGTAAATCGGATAACGTTCCAAGTGAAGAAGTAGTCAAAAAGATGAAAAACTATT
GGAGACAACTTCTAAACGCCAAGTTAATCACTCAACGTAAGTTTGATAATTTAACGA
AAGCTGAACGTGGAGGTTTGAGTGAACTTGATAAAGCTGGTTTTATCAAACGCCAAT
TGGTTGAAACTCGCCAAATCACTAAGCATGTGGCACAAATTTTGGATAGTCGCATGA
ATACTAAATACGATGAAAATGATAAACTTATTCGAGAGGTTAAAGTGATTACCTTAA
AATCTAAATTAGTTTCTGACTTCCGAAAAGATTTCCAATTCTATAAAGTACGTGAGA
TTAACAATTACCATCATGCCCATGATGCGTATCTAAATGCCGTCGTTGGAACTGCTTT
GATTAAGAAATATCCAAAACTTGAATCGGAGTTTGTCTATGGTGATTATAAAGTTTA
TGATGTTCGTAAAATGATTGCTAAGTCTGAGCAAGAAATAGGCAAAGCAACCGCAA
AATATTTCTTTTACTCTAATATCATGAACTTCTTCAAAACAGAAATTACACTTGCAAA
TGGAGAGATTCGCAAACGCCCTCTAATCGAAACTAATGGGGAAACTGGAGAAATTG
TCTGGGATAAAGGGCGAGATTTTGCCACAGTGCGCAAAGTATTGTCCATGCCCCAAG
TCAATATTGTCAAGAAAACAGAAGTACAGACAGGCGGATTCTCCAAGGAGTCAATT
TTACCAAAAAGAAATTCGGACAAGCTTATTGCTCGTAAAAAAGACTGGGATCCAAA
AAAATATGGTGGTTTTGATAGTCCAACGGTAGCTTATTCAGTCCTAGTGGTTGCTAA
GGTGGAAAAAGGGAAATCGAAGAAGTTAAAATCCGTTAAAGAGTTACTAGGGATCA
CAATTATGGAAAGAAGTTCCTTTGAAAAAAATCCGATTGACTTTTTAGAAGCTAAAG
GATATAAGGAAGTTAAAAAAGACTTAATCATTAAACTACCTAAATATAGTCTTTTTG
AGTTAGAAAACGGTCGTAAACGGATGCTGGCTAGTGCCGGAGAATTACAAAAAGGA
AATGAGCTGGCTCTGCCAAGCAAATATGTGAATTTTTTATATTTAGCTAGTCATTATG
AAAAGTTGAAGGGTAGTCCAGAAGATAACGAACAAAAACAATTGTTTGTGGAGCAG
CATAAGCATTATTTAGATGAGATTATTGAGCAAATCAGTGAATTTTCTAAGCGTGTT
ATTTTAGCAGATGCCAATTTAGATAAAGTTCTTAGTGCATATAACAAACATAGAGAC
AAACCAATACGTGAACAAGCAGAAAATATTATTCATTTATTTACGTTGACGAATCTT
GGAGCTCCCGCTGCTTTTAAATATTTTGATACAACAATTGATCGTAAACGATATACGTCT
ACAAAAGAAGTTTTAGATGCCACTCTTATCCATCAATCCATCACTGGTCTTTATgaaaca
cgcattgatttgagtcagctaggaggtgactgaTAATCAAATGTCAGACGAAAATGCCAATT
ATTGAAGCGGCTAACGCCGCTTTTTTTGTTTCTGGTCTCCCTTGCTTGTACTTTACAGTATA
GCTTCTAGAGATCTGCAGTGACTGGAGCTTCTAGAGATCTGCAGTGACTGGGAAAAC
CCTGGCGACTAGTCTTGGACTCCTGTTGATAGATCCAGTAATGACCTCAGAACTCCA
TCTGGATTTGTTCAGAACGCTCGGTTGCCGCCGGGCGTTTTTTATTGGTGAGAATCCA
GGGGTCCCCAATAATTACGATTTAAATCCTTCAAACTTCCCAAAGGCGAGCCCTAGT
GACATTAGAAAACCGACTGTAAAAAGTACAGTCGGCATTATCTCATATTATAAAAG
CCAGTCATTAGGCCTATCTGACAATTCCTGAATAGAGTTCATAAACAATCCTGCATG
ATAACCATCACAAACAGAATGATGTACCTGTAAAGATAGCGGTAAATATATTGAAT
TACCTTTATTAATGAATTTTCCTGCTGTAATAATGGGTAGAAGGTAATTACTATTATT
ATTGATATTTAAGTTAAACCCAGTAAATGAAGTCCATGGAATAATAGAAAGAGAAA
AAGCATTTTCAGGTATAGGTGTTTTGGGAAACAATTTCCCCGAACCATTATATTTCTC
TACATCAGAAAGGTATAAATCATAAAACTCTTTGAAGTCATTCTTTACAGGAGTCCA
AATACCAGAGAATGTTTTAGATACACCATCAAAAATTGTATAAAGTGGCTCTAACTT
ATCCCAATAACCTAACTCTCCGTCGCTATTGTAACCAGTTCTAAAAGCTGTATTTGA
GTTTATCACCCTTGTCACTAAGAAAATAAATGCAGGGTAAAATTTATATCCTTCTTGT
TTTATGTTTCGGTATAAAACACTAATATCAATTTCTGTGGTTATACTAAAAGTCGTTT
GTTGGTTCAAATAATGATTAAATATCTCTTTTCTCTTCCAATTGTCTAAATCAATTTT
ATTAAAGTTCATTTGATATGCCTCCTAAATTTTTATCTAAAGTGAATTTAGGAGGCTT
ACTTGTCTGCTTTCTTCATTAGAATCAATCCTTTTTTAAAAGTCAATCCCGTTTGTTG
AAGACTTTTGTCCTTTTCCGCTGCATAAACAATATGGCCCGTTTGTTGAACTACTCTT
TAATAAAATAATTTTTCCGTTCCCAATTCCACATTGCAATAATAGAAAATCCATCTTC
ATCGGCTTTTTCGTCATCATCTGTATGAATCAAATCGCCTTCTTCTGTGTCATCAAGG
TTTAATTTTTTATGTATTTCTTTTAACAAACCACCATAGGAGATTAACCTTTTACGGT
GTAAACCTTCCTCCAAATCAGACAAACGTTTCAAATTCTTTTCTTCATCATCGGTCAT
AAAATCCGTATCCTTTACAGGATATTTTGCAGTTTCGTCAATTGCCGATTGTATATCC
GATTTATATTTATTTTTCGGTCGAATCATTTGAACTTTTACATTTGGATCATAGTCTA
ATTTCATTGCCTTTTTCCAAAATTGAATCCATTGTTTTTGATTCACGTAGTTTTCTGTA
TTCTTAAAATAAGTTGGTTCCACACATACCAATACATGCATGTGCTGATTATAAGAA
TTATCTTTATTATTTATTGTCACTTCCGTTGCACGCATAAAACCAACAAGATTTTTAT
TAATTTTTTTATATTGCATCATTCGGCGAAATCCTTGAGCCATATCTGACAAACTCTT
ATTTAATTCTTCGCCATCATAAACATTTTTAACTGTTAATGTGAGAAACAACCAACG
AACTGTTGGCTTTTGTTTAATAACTTCAGCAACAACCTTTTGTGACTGAATGCCATGT
TTCATTGCTCTCCTCCAGTTGCACATTGGACAAAGCCTGGATTTACAAAACCACACT
CGATACAACTTTCTTTCGCCTGTTTCACGATTTTGTTTATACTCTAATATTTCAGCAC
AATCTTTTACTCTTTCAGCCTTTTTAAATTCAAGAATATGCAGAAGTTCAAAGTAATC
AACATTAGCGATTTTCTTTTCTCTCCATGGTCTCACTTTTCCACTTTTTGTCTTGTCCA
CTAAAACCCTTGATTTTTCATCTGAATAAATGCTACTATTAGGACACATAATATTAA
AAGAAACCCCCATCTATTTAGTTATTTGTTTGGTCACTTATAACTTTAACAGATGGGG
TTTTTCTGTGCAACCAATTTTAAGGGTTTTCCAATACTTTAAAACACATACATACCAA
CACTTCAACGCACCTTTCAGCAACTAAAATAAAAATGACGTTATTTCTATATGTATC
AAGATAAGAAAGAACAAGTTCAAAACCATCAAAAAAAGACACCTTTTCAGGTGCTT
TTTTTATTTTATAAACTCATTCCCTGATCTCGACTTCGTTCTTTTTTTACCTCTCGGTT
ATGAGTTAGTTCAAATTCGTTCTTTTTAGGTTCTAAATCGTGTTTTTCTTGGAATTGT
GCTGTTTTATCCTTTACCTTGTCTACAAACCCCTTAAAAACGTTTTTAAAGGCTTTTA
AGCGTCTGTACGTTCCTTAAGGAATTATTCCTTAGTGCTTTCTAGGTTAATGTCATGA
TAATAATGGTTTCTTAGACGTCAGGTGGCACTTTTCGGGGAAATGTCCGCGGAACCC
CTATTTGTTTATTTTTCTAAATACATTCAAATATGTATCCGCTCATGAGACAATAACC
CTGATAAATGCTTCAATAATATTGAAAAAGGAAGAGTATGAGTATTCAACATTTCCG
TGTCGCCCTTATTCCCTTTTTTGCGGCATTTTGCCTTCCTGTTTTTGCTCACCCAGAAA
CGCTGGTGAAAGTAAAAGATGCTGAAGATCAGTTGGGTGCACGAGTGGGTTACATC
GAACTGGATCTCAACAGCGGTAAGATCCTTGAGAGTTTTCGCCCCGAAGAACGTTTT
CCAATGATGAGCACTTTTAAAGTTCTGCTATGTGGCGCGGTATTATCCCGTGTTGAC
GCCGGGCAAGAGCAACTCGGTCGCCGCATACACTATTCTCAGAATGACTTGGTTGAG
TACTCACCAGTCACAGAAAAGCATCTTACGGATGGCATGACAGTAAGAGAATTATG
CAGTGCTGCCATAACCATGAGTGATAACACTGCGGCCAACTTACTTCTGACAACGAT
CGGAGGACCGAAGGAGCTAACCGCTTTTTTGCACAACATGGGGGATCATGTAACTC
GCCTTGATCGTTGGGAACCGGAGCTGAATGAAGCCATACCAAACGACGAGCGTGAC
ACCACGATGCCTGCAGCAATGGCAACAACGTTGCGCAAACTATTAACTGGCGAACT
ACTTACTCTAGCTTCCCGGCAACAATTAATAGACTGGATGGAGGCGGATAAAGTTGC
AGGACCACTTCTGCGCTCGGCCCTTCCGGCTGGCTGGTTTATTGCTGATAAATCTGG
AGCCGGTGAGCGTGGGTCTCGCGGTATCATTGCAGCACTGGGGCCAGATGGTAAGC
CCTCCCGTATCGTAGTTATCTACACGACGGGGAGTCAGGCAACTATGGATGAACGA
AATAGACAGATCGCTGAGATAGGTGCCTCACTGATTAAGCATTGGTAACTGTCAGAC
CAAGTTTACTCATATATACTTTAGATTGATTTAAAACTTCATTTTTAATTTAAAAGGA
TCTAGGTGAAGATCCTTTTTGATAATCTCATGACCAAAATCCCTTAACGTGAGTTTTC
GTTCCACTGAGCGTCAGACCCCTTAATAAGATGATCTTCTTGAGATCGTTTTGGTCTG
CGCGTAATCTCTTGCTCTGAAAACGAAAAAACCGCCTTGCAGGGAGGTTTTTCGAAG
GTTCTCTGAGCTACCAACTCTTTGAACCGAGGTAACTGGCTTGCAGGAGCGCAGTCA
CCAAAACTTGTCCTTTCAGTTTAGCCTTAACCGGCGCATGACTTCAAGACTAACTCCT
CTAAATCAATTACCAGTGGCTGCTGCCAGTGGTGCTTTTGCATGTCTTTCCGGGTTGG
ACTCAAGACGATAGTTACCGGATAAGGCGCAGCGGTCGGACTGAACGGGGGGTTCG
TGCATACAGTCCAGCTTGGAGCGAACTGCCTACCCGGAACTGAGTGTCAGGCGTGG
AATGAGACAAACGCGGCCATAACAGCGGAATGACACCGGTAAACCGAAAGGCAGG
AACAGGAGAGCGCACGAGGGAGCCGCCAGGGGGAAACGCCTGGTATCTTTATAGTC
CTGTCGGGTTTCGCCACCACTGATTTGAGCGTCAGATTTCGTGATGCTTGTCAGGGG
GCGGAGCCTATGGAAAAACGCTTTGCCCGAGCCTATGGAAAAACGCTTTGCCCCAC
ACAGGCTCTTGAACAAGGGATAACAATTTGTCATAGCTGTATTCGTTACAAGGAGGA
TTTTCGAAAAAACACCCTAACGGGTGTTTTTTTATAGCTGGTCTCCCCAAGTGCTGTA
AAAAACGCCGAATGAAGCGGATGAAATTGCTCATCGTCTCGTGAAAAAGCCGAAGA
AGGTTAAGCCTGGGTATAAAAAGAAAATGAGCTATGAGATGGAGAAAGCCATAACC
ATGAGTGATAACACTGGTTTTAGAGCTAGAAATAGCAAGTTAAAATAAGGCTAGTC
CGTTATCAACTTGAAAAAGTGGCACCGAGTCGGTGCTTTTTTTCTCGGTACCAAATT
CCAGAAAAGAGGCCTCCCGAAAGGGGGGCCTTTTTTCGTTTTGGTCCCTCAATGATC
TCCTTATCCGGTTAAGATGGCAAGCTTGACAAGTATTTCTTCAGAATTTACAATGAA
GTTTACATGACGCTCTTAAAGGGGGTTTTAGATACCAGAGATGGATAAGAAATACTC
>pCas9g2-cons
(SEQ ID No: 230)
AGGTCAGACGCGATTTCCTGGGTGCTCGAGCCATGGGAAAGCGGTGGTGGAAAAAA
CGCCGAATGAAGCGGATGAAATTGCTCATCGTCTCGTGAAAAAGCCGAAGAAGGTT
AAGCCTGGGTATAAAAAGAAAATGAGCTATGAGATGGAGAAATTGAAGTCATGCGC
CGGTTAGTTTTAGAGCTAGAAATAGCAAGTTAAAATAAGGCTAGTCCGTTATCAACT
TGAAAAAGTGGCACCGAGTCGGTGCTTTTTTTCTCGGTACCAAATTCCAGAAAAGAG
GCCTCCCGAAAGGGGGGCCTTTTTTCGTTTTGGTCCCTCAATGATCCGACAGGAAGT
GGTATTAGCGACTCCTACAAGGAATAGGTGTCTGGGTCATGCAGTGACTGGGAAAA
CCCTGGCGACTAGTCTTGGACTCCTGTTGATAGATCCAGTAATGACCTCAGAACTCC
ATCTGGATTTGTTCAGAACGCTCGGTTGCCGCCGGGCGTTTTTTATTGGTGAGAATCC
AGGGGTCCCCAATAATTACGATTTAAATCCTTCAAACTTCCCAAAGGCGAGCCCTAG
TGACATTAGAAAACCGACTGTAAAAAGTACAGTCGGCATTATCTCATATTATAAAAG
CCAGTCATTAGGCCTATCTGACAATTCCTGAATAGAGTTCATAAACAATCCTGCATG
ATAACCATCACAAACAGAATGATGTACCTGTAAAGATAGCGGTAAATATATTGAAT
TACCTTTATTAATGAATTTTCCTGCTGTAATAATGGGTAGAAGGTAATTACTATTATT
ATTGATATTTAAGTTAAACCCAGTAAATGAAGTCCATGGAATAATAGAAAGAGAAA
AAGCATTTTCAGGTATAGGTGTTTTGGGAAACAATTTCCCCGAACCATTATATTTCTC
TACATCAGAAAGGTATAAATCATAAAACTCTTTGAAGTCATTCTTTACAGGAGTCCA
AATACCAGAGAATGTTTTAGATACACCATCAAAAATTGTATAAAGTGGCTCTAACTT
ATCCCAATAACCTAACTCTCCGTCGCTATTGTAACCAGTTCTAAAAGCTGTATTTGA
GTTTATCACCCTTGTCACTAAGAAAATAAATGCAGGGTAAAATTTATATCCTTCTTGT
TTTATGTTTCGGTATAAAACACTAATATCAATTTCTGTGGTTATACTAAAAGTCGTTT
GTTGGTTCAAATAATGATTAAATATCTCTTTTCTCTTCCAATTGTCTAAATCAATTTT
ATTAAAGTTCATTTGATATGCCTCCTAAATTTTTATCTAAAGTGAATTTAGGAGGCTT
ACTTGTCTGCTTTCTTCATTAGAATCAATCCTTTTTTAAAAGTCAATCCCGTTTGTTG
AAGACTTTTGTCCTTTTCCGCTGCATAAACAATATGGCCCGTTTGTTGAACTACTCTT
TAATAAAATAATTTTTCCGTTCCCAATTCCACATTGCAATAATAGAAAATCCATCTTC
ATCGGCTTTTTCGTCATCATCTGTATGAATCAAATCGCCTTCTTCTGTGTCATCAAGG
TTTAATTTTTTATGTATTTCTTTTAACAAACCACCATAGGAGATTAACCTTTTACGGT
GTAAACCTTCCTCCAAATCAGACAAACGTTTCAAATTCTTTTCTTCATCATCGGTCAT
AAAATCCGTATCCTTTACAGGATATTTTGCAGTTTCGTCAATTGCCGATTGTATATCC
GATTTATATTTATTTTTCGGTCGAATCATTTGAACTTTTACATTTGGATCATAGTCTA
ATTTCATTGCCTTTTTCCAAAATTGAATCCATTGTTTTTGATTCACGTAGTTTTCTGTA
TTCTTAAAATAAGTTGGTTCCACACATACCAATACATGCATGTGCTGATTATAAGAA
TTATCTTTATTATTTATTGTCACTTCCGTTGCACGCATAAAACCAACAAGATTTTTAT
TAATTTTTTTATATTGCATCATTCGGCGAAATCCTTGAGCCATATCTGACAAACTCTT
ATTTAATTCTTCGCCATCATAAACATTTTTAACTGTTAATGTGAGAAACAACCAACG
AACTGTTGGCTTTTGTTTAATAACTTCAGCAACAACCTTTTGTGACTGAATGCCATGT
TTCATTGCTCTCCTCCAGTTGCACATTGGACAAAGCCTGGATTTACAAAACCACACT
CGATACAACTTTCTTTCGCCTGTTTCACGATTTTGTTTATACTCTAATATTTCAGCAC
AATCTTTTACTCTTTCAGCCTTTTTAAATTCAAGAATATGCAGAAGTTCAAAGTAATC
AACATTAGCGATTTTCTTTTCTCTCCATGGTCTCACTTTTCCACTTTTTGTCTTGTCCA
CTAAAACCCTTGATTTTTCATCTGAATAAATGCTACTATTAGGACACATAATATTAA
AAGAAACCCCCATCTATTTAGTTATTTGTTTGGTCACTTATAACTTTAACAGATGGGG
TTTTTCTGTGCAACCAATTTTAAGGGTTTTCCAATACTTTAAAACACATACATACCAA
CACTTCAACGCACCTTTCAGCAACTAAAATAAAAATGACGTTATTTCTATATGTATC
AAGATAAGAAAGAACAAGTTCAAAACCATCAAAAAAAGACACCTTTTCAGGTGCTT
TTTTTATTTTATAAACTCATTCCCTGATCTCGACTTCGTTCTTTTTTTACCTCTCGGTT
ATGAGTTAGTTCAAATTCGTTCTTTTTAGGTTCTAAATCGTGTTTTTCTTGGAATTGT
GCTGTTTTATCCTTTACCTTGTCTACAAACCCCTTAAAAACGTTTTTAAAGGCTTTTA
AGCGTCTGTACGTTCCTTAAGGAATTATTCCTTAGTGCTTTCTAGGTTAATGTCATGA
TAATAATGGTTTCTTAGACGTCAGGTGGCACTTTTCGGGGAAATGTCCGCGGAACCC
CTATTTGTTTATTTTTCTAAATACATTCAAATATGTATCCGCTCATGAGACAATAACC
CTGATAAATGCTTCAATAATATTGAAAAAGGAAGAGTATGAGTATTCAACATTTCCG
TGTCGCCCTTATTCCCTTTTTTGCGGCATTTTGCCTTCCTGTTTTTGCTCACCCAGAAA
CGCTGGTGAAAGTAAAAGATGCTGAAGATCAGTTGGGTGCACGAGTGGGTTACATC
GAACTGGATCTCAACAGCGGTAAGATCCTTGAGAGTTTTCGCCCCGAAGAACGTTTT
CCAATGATGAGCACTTTTAAAGTTCTGCTATGTGGCGCGGTATTATCCCGTGTTGAC
GCCGGGCAAGAGCAACTCGGTCGCCGCATACACTATTCTCAGAATGACTTGGTTGAG
TACTCACCAGTCACAGAAAAGCATCTTACGGATGGCATGACAGTAAGAGAATTATG
CAGTGCTGCCATAACCATGAGTGATAACACTGCGGCCAACTTACTTCTGACAACGAT
CGGAGGACCGAAGGAGCTAACCGCTTTTTTGCACAACATGGGGGATCATGTAACTC
GCCTTGATCGTTGGGAACCGGAGCTGAATGAAGCCATACCAAACGACGAGCGTGAC
ACCACGATGCCTGCAGCAATGGCAACAACGTTGCGCAAACTATTAACTGGCGAACT
ACTTACTCTAGCTTCCCGGCAACAATTAATAGACTGGATGGAGGCGGATAAAGTTGC
AGGACCACTTCTGCGCTCGGCCCTTCCGGCTGGCTGGTTTATTGCTGATAAATCTGG
AGCCGGTGAGCGTGGGTCTCGCGGTATCATTGCAGCACTGGGGCCAGATGGTAAGC
CCTCCCGTATCGTAGTTATCTACACGACGGGGAGTCAGGCAACTATGGATGAACGA
AATAGACAGATCGCTGAGATAGGTGCCTCACTGATTAAGCATTGGTAACTGTCAGAC
CAAGTTTACTCATATATACTTTAGATTGATTTAAAACTTCATTTTTAATTTAAAAGGA
TCTAGGTGAAGATCCTTTTTGATAATCTCATGACCAAAATCCCTTAACGTGAGTTTTC
GTTCCACTGAGCGTCAGACCCCTTAATAAGATGATCTTCTTGAGATCGTTTTGGTCTG
CGCGTAATCTCTTGCTCTGAAAACGAAAAAACCGCCTTGCAGGGAGGTTTTTCGAAG
GTTCTCTGAGCTACCAACTCTTTGAACCGAGGTAACTGGCTTGCAGGAGCGCAGTCA
CCAAAACTTGTCCTTTCAGTTTAGCCTTAACCGGCGCATGACTTCAAGACTAACTCCT
CTAAATCAATTACCAGTGGCTGCTGCCAGTGGTGCTTTTGCATGTCTTTCCGGGTTGG
ACTCAAGACGATAGTTACCGGATAAGGCGCAGCGGTCGGACTGAACGGGGGGTTCG
TGCATACAGTCCAGCTTGGAGCGAACTGCCTACCCGGAACTGAGTGTCAGGCGTGG
AATGAGACAAACGCGGCCATAACAGCGGAATGACACCGGTAAACCGAAAGGCAGG
AACAGGAGAGCGCACGAGGGAGCCGCCAGGGGGAAACGCCTGGTATCTTTATAGTC
CTGTCGGGTTTCGCCACCACTGATTTGAGCGTCAGATTTCGTGATGCTTGTCAGGGG
GCGGAGCCTATGGAAAAACGCTTTGCCCGAGCCTATGGAAAAACGCTTTGCCCCAC
ACAGGCTCTTGAACAAGGGATAACAATTTGTCATAGCTGTATTCGTTACAAGGAGGA
TTTTCGAAAAAACACCCTAACGGGTGTTTTTTTATAGCTGGTCTCCCCAAGTGCTGTA
AAAAACGCCGAATGAAGCGGATGAAATTGCTCATCGTCTCGTGAAAAAGCCGAAGA
AGGTTAAGCCTGGGTATAAAAAGAAAATGAGCTATGAGATGGAGAAAGCCATAACC
ATGAGTGATAACACTGGTTTTAGAGCTAGAAATAGCAAGTTAAAATAAGGCTAGTC
CGTTATCAACTTGAAAAAGTGGCACCGAGTCGGTGCTTTTTTTCTCGGTACCAAATT
CCAGAAAAGAGGCCTCCCGAAAGGGGGGCCTTTTTTCGTTTTGGTCCCTCAATGATC
TCCTTATCCGGTTAAGATGGCAAGCTTGACAAGTATTTCTTCAGAATTTACAATGAA
GTTTACATGACGCTCTTAAAGGGGGTTTTAGATACCAGAGATGGATAAGAAATACTC
AATAGGCTTAGATATCGGCACAAATAGCGTCGGATGGGCGGTGATCACTGATGAAT
ATAAGGTTCCGTCTAAAAAGTTCAAGGTTCTGGGAAATACAGACCGCCACAGTATC
AAAAAAAATCTTATAGGGGCTCTTTTATTTGACAGTGGAGAGACAGCGGAAGCGAC
TCGTCTCAAACGGACAGCTCGTAGAAGGTATACACGTCGGAAGAATCGTATTTGTTA
TCTACAGGAGATTTTTTCAAATGAGATGGCGAAAGTAGATGATAGTTTCTTTCATCG
ACTTGAAGAGTCTTTTTTGGTGGAAGAAGACAAGAAGCATGAACGTCATCCTATTTT
TGGAAATATAGTAGATGAAGTTGCTTATCATGAGAAATATCCAACTATCTATCATCT
GCGAAAAAAATTGGTAGATTCTACTGATAAAGCGGATTTGCGCTTAATCTATTTGGC
CTTAGCGCATATGATTAAGTTTCGTGGTCATTTTTTGATTGAGGGAGATTTAAATCCT
GATAATAGTGATGTGGACAAACTATTTATCCAGTTGGTACAAACCTACAATCAATTA
TTTGAAGAAAACCCTATTAACGCAAGTGGAGTAGATGCTAAAGCGATTCTTTCTGCA
CGATTGAGTAAATCAAGACGATTAGAAAATCTCATTGCTCAGCTCCCCGGTGAGAA
GAAAAATGGCTTATTTGGGAATCTCATTGCTTTGTCATTGGGTTTGACCCCTAATTTT
AAATCAAATTTTGATTTGGCAGAAGATGCTAAATTACAGCTTTCAAAAGATACTTAC
GATGATGATTTAGATAATTTATTGGCGCAAATTGGAGATCAATATGCTGATTTGTTTT
TGGCAGCTAAGAATTTATCAGATGCTATTTTACTTTCAGATATCCTAAGAGTAAATA
CTGAAATAACTAAGGCTCCCCTATCAGCTTCAATGATTAAACGCTACGATGAACATC
ATCAAGACTTGACTCTTTTAAAAGCTTTAGTTCGACAACAACTTCCAGAAAAGTATA
AAGAAATCTTTTTTGATCAATCAAAAAACGGATATGCAGGTTATATTGATGGGGGAG
CTAGCCAAGAAGAATTTTATAAATTTATCAAACCAATTTTAGAAAAAATGGATGGTA
CTGAGGAATTATTGGTGAAACTAAATCGTGAAGATTTGCTGCGCAAGCAACGGACC
TTTGACAACGGCTCTATTCCCCATCAAATTCACTTGGGTGAGCTGCATGCTATTTTGA
GAAGACAAGAAGACTTTTATCCATTTTTAAAAGACAATCGTGAGAAGATTGAAAAA
ATCTTGACTTTTCGAATTCCTTATTATGTTGGTCCATTGGCGCGTGGCAATAGTCGTT
TTGCATGGATGACTCGGAAGTCTGAAGAAACAATTACCCCATGGAATTTTGAAGAA
GTTGTCGATAAAGGTGCTTCAGCTCAATCATTTATTGAACGCATGACAAACTTTGAT
AAAAATCTTCCAAATGAAAAAGTACTACCAAAACATAGTTTGCTTTATGAGTATTTT
ACGGTTTATAACGAATTGACAAAGGTCAAATATGTTACTGAAGGAATGCGAAAACC
AGCATTTCTTTCAGGTGAACAGAAGAAAGCCATTGTTGATTTACTCTTCAAAACAAA
TCGAAAAGTAACCGTTAAGCAATTAAAAGAAGATTATTTCAAAAAAATAGAATGTT
TTGATAGTGTTGAAATTTCAGGAGTTGAAGATAGATTTAATGCTTCATTAGGTACCT
ACCATGATTTGCTAAAAATTATTAAAGATAAAGATTTTTTGGATAATGAAGAAAATG
AAGATATCTTAGAGGATATTGTTTTAACATTGACCTTATTTGAAGATAGGGAGATGA
TTGAGGAAAGACTTAAAACATATGCTCACCTCTTTGATGATAAGGTGATGAAACAGC
TTAAACGTCGCCGTTATACTGGTTGGGGACGTTTGTCTCGAAAATTGATTAATGGTA
TTAGGGATAAGCAATCTGGCAAAACAATATTAGATTTTTTGAAATCAGATGGTTTTG
CCAATCGCAATTTTATGCAGCTGATCCATGATGATAGTTTGACATTTAAAGAAGACA
TTCAAAAAGCACAAGTGTCTGGACAAGGCGATAGTTTACATGAACATATTGCAAATT
TAGCTGGTAGCCCTGCTATTAAAAAAGGTATTTTACAGACTGTAAAAGTTGTTGATG
AATTGGTCAAAGTAATGGGGCGGCATAAGCCAGAAAATATCGTTATTGAAATGGCA
CGTGAAAATCAGACAACTCAAAAGGGCCAGAAAAATTCGCGAGAGCGTATGAAAC
GAATCGAAGAAGGTATCAAAGAATTAGGAAGTCAGATTCTTAAAGAGCATCCTGTT
GAAAATACTCAATTGCAAAATGAAAAGCTCTATCTCTATTATCTCCAAAATGGAAGA
GACATGTATGTGGACCAAGAATTAGATATTAATCGTTTAAGTGATTATGATGTCGAT
CACATTGTTCCACAAAGTTTCCTTAAAGACGATTCAATAGACAATAAGGTCTTAACG
CGTTCTGATAAAAATCGTGGTAAATCGGATAACGTTCCAAGTGAAGAAGTAGTCAA
AAAGATGAAAAACTATTGGAGACAACTTCTAAACGCCAAGTTAATCACTCAACGTA
AGTTTGATAATTTAACGAAAGCTGAACGTGGAGGTTTGAGTGAACTTGATAAAGCTG
GTTTTATCAAACGCCAATTGGTTGAAACTCGCCAAATCACTAAGCATGTGGCACAAA
TTTTGGATAGTCGCATGAATACTAAATACGATGAAAATGATAAACTTATTCGAGAGG
TTAAAGTGATTACCTTAAAATCTAAATTAGTTTCTGACTTCCGAAAAGATTTCCAATT
CTATAAAGTACGTGAGATTAACAATTACCATCATGCCCATGATGCGTATCTAAATGC
CGTCGTTGGAACTGCTTTGATTAAGAAATATCCAAAACTTGAATCGGAGTTTGTCTA
TGGTGATTATAAAGTTTATGATGTTCGTAAAATGATTGCTAAGTCTGAGCAAGAAAT
AGGCAAAGCAACCGCAAAATATTTCTTTTACTCTAATATCATGAACTTCTTCAAAAC
AGAAATTACACTTGCAAATGGAGAGATTCGCAAACGCCCTCTAATCGAAACTAATG
GGGAAACTGGAGAAATTGTCTGGGATAAAGGGCGAGATTTTGCCACAGTGCGCAAA
GTATTGTCCATGCCCCAAGTCAATATTGTCAAGAAAACAGAAGTACAGACAGGCGG
ATTCTCCAAGGAGTCAATTTTACCAAAAAGAAATTCGGACAAGCTTATTGCTCGTAA
AAAAGACTGGGATCCAAAAAAATATGGTGGTTTTGATAGTCCAACGGTAGCTTATTC
AGTCCTAGTGGTTGCTAAGGTGGAAAAAGGGAAATCGAAGAAGTTAAAATCCGTTA
AAGAGTTACTAGGGATCACAATTATGGAAAGAAGTTCCTTTGAAAAAAATCCGATT
GACTTTTTAGAAGCTAAAGGATATAAGGAAGTTAAAAAAGACTTAATCATTAAACT
ACCTAAATATAGTCTTTTTGAGTTAGAAAACGGTCGTAAACGGATGCTGGCTAGTGC
CGGAGAATTACAAAAAGGAAATGAGCTGGCTCTGCCAAGCAAATATGTGAATTTTT
TATATTTAGCTAGTCATTATGAAAAGTTGAAGGGTAGTCCAGAAGATAACGAACAA
AAACAATTGTTTGTGGAGCAGCATAAGCATTATTTAGATGAGATTATTGAGCAAATC
AGTGAATTTTCTAAGCGTGTTATTTTAGCAGATGCCAATTTAGATAAAGTTCTTAGTG
CATATAACAAACATAGAGACAAACCAATACGTGAACAAGCAGAAAATATTATTCAT
TTATTTACGTTGACGAATCTTGGAGCTCCCGCTGCTTTTAAATATTTTGATACAACAA
TTGATCGTAAACGATATACGTCTACAAAAGAAGTTTTAGATGCCACTCTTATCCATC
AATCCATCACTGGTCTTTATGAAACACGCATTGATTTGAGTCAGCTAGGAGGTGACT
GATAATCAAATGTCAGACGAAAATGCCAATTATTGAAGCGGCTAACGCCGCTTTTTT
TGTTTCTGGTCTCCCTTGCTTGTACTTTACAGTATAGCTTCTAGAGATCTGCAGTGAC
TGGAGCTTCTAGAGATC
>pCas9g1-D15-cons
(SEQ ID No: 231)
GATTAACTAATAAGGAGGGCAAACTACTAGAGATGAGTAAATCCTGGGGCAAATTC
ATCGAGGAGGAAGAGGCGGAAATGGCTTCCCGTCGTAATCTAATGATTGTCGATGG
AACTAACTTAGGCTTTCGCTTCAAACATAACAATAGTAAAAAACCATTTGCCTCAAG
TTATGTTTCAACTATTCAATCTCTGGCAAAATCCTACTCTGCCAGAACTACGATTGTT
CTAGGTGATAAGGGCAAAAGCGTGTTCAGACTAGAACATCTACCAGAGTATAAAGG
TAATCGTGATGAAAAGTACGCACAACGTACGGAAGAGGAGAAAGCGCTAGATGAG
CAGTTCTTCGAATACCTCAAAGATGCTTTCGAGTTGTGTAAAACTACATTCCCAACTT
TTACCATTCGTGGTGTAGAAGCAGATGACATGGCGGCGTATATTGTTAAGCTCATCG
GGCATCTTTATGATCACGTTTGGCTAATATCTACAGATGGTGACTGGGATACTTTATT
AACGGATAAAGTTTCTCGTTTTTCTTTCACAACACGTCGTGAGTATCATCTTCGTGAT
ATGTATGAGCACCATAACGTGGACGACGTAGAACAGTTTATCTCCCTGAAAGCAATT
ATGGGAGATCTAGGAGATAATATTCGTGGTGTTGAAGGAATAGGAGCAAAACGCGG
ATATAATATTATTCGTGAGTTTGGTAACGTACTGGATATTATTGATCAGCTTCCACTG
CCTGGAAAGCAGAAATATATACAGAACCTGAATGCATCGGAAGAACTGCTTTTCCG
AAACTTGATTCTGGTTGATTTACCTACCTACTGTGTGGATGCTATTGCTGCTGTAGGT
CAAGATGTGTTAGATAAGTTCACGAAAGACATCCTTGAGATCGCGGAACAATGATA
AGCTCTTAAAGGGGGTTTTAGATACCAGAGATGGATAAGAAATACTCAATAGGCTT
AGATATCGGCACAAATAGCGTCGGATGGGCGGTGATCACTGATGAATATAAGGTTC
CGTCTAAAAAGTTCAAGGTTCTGGGAAATACAGACCGCCACAGTATCAAAAAAAAT
CTTATAGGGGCTCTTTTATTTGACAGTGGAGAGACAGCGGAAGCGACTCGTCTCAAA
CGGACAGCTCGTAGAAGGTATACACGTCGGAAGAATCGTATTTGTTATCTACAGGA
GATTTTTTCAAATGAGATGGCGAAAGTAGATGATAGTTTCTTTCATCGACTTGAAGA
GTCTTTTTTGGTGGAAGAAGACAAGAAGCATGAACGTCATCCTATTTTTGGAAATAT
AGTAGATGAAGTTGCTTATCATGAGAAATATCCAACTATCTATCATCTGCGAAAAAA
ATTGGTAGATTCTACTGATAAAGCGGATTTGCGCTTAATCTATTTGGCCTTAGCGCAT
ATGATTAAGTTTCGTGGTCATTTTTTGATTGAGGGAGATTTAAATCCTGATAATAGTG
ATGTGGACAAACTATTTATCCAGTTGGTACAAACCTACAATCAATTATTTGAAGAAA
ACCCTATTAACGCAAGTGGAGTAGATGCTAAAGCGATTCTTTCTGCACGATTGAGTA
AATCAAGACGATTAGAAAATCTCATTGCTCAGCTCCCCGGTGAGAAGAAAAATGGC
TTATTTGGGAATCTCATTGCTTTGTCATTGGGTTTGACCCCTAATTTTAAATCAAATT
TTGATTTGGCAGAAGATGCTAAATTACAGCTTTCAAAAGATACTTACGATGATGATT
TAGATAATTTATTGGCGCAAATTGGAGATCAATATGCTGATTTGTTTTTGGCAGCTA
AGAATTTATCAGATGCTATTTTACTTTCAGATATCCTAAGAGTAAATACTGAAATAA
CTAAGGCTCCCCTATCAGCTTCAATGATTAAACGCTACGATGAACATCATCAAGACT
TGACTCTTTTAAAAGCTTTAGTTCGACAACAACTTCCAGAAAAGTATAAAGAAATCT
TTTTTGATCAATCAAAAAACGGATATGCAGGTTATATTGATGGGGGAGCTAGCCAAG
AAGAATTTTATAAATTTATCAAACCAATTTTAGAAAAAATGGATGGTACTGAGGAAT
TATTGGTGAAACTAAATCGTGAAGATTTGCTGCGCAAGCAACGGACCTTTGACAACG
GCTCTATTCCCCATCAAATTCACTTGGGTGAGCTGCATGCTATTTTGAGAAGACAAG
AAGACTTTTATCCATTTTTAAAAGACAATCGTGAGAAGATTGAAAAAATCTTGACTT
TTCGAATTCCTTATTATGTTGGTCCATTGGCGCGTGGCAATAGTCGTTTTGCATGGAT
GACTCGGAAGTCTGAAGAAACAATTACCCCATGGAATTTTGAAGAAGTTGTCGATA
AAGGTGCTTCAGCTCAATCATTTATTGAACGCATGACAAACTTTGATAAAAATCTTC
CAAATGAAAAAGTACTACCAAAACATAGTTTGCTTTATGAGTATTTTACGGTTTATA
ACGAATTGACAAAGGTCAAATATGTTACTGAAGGAATGCGAAAACCAGCATTTCTTT
CAGGTGAACAGAAGAAAGCCATTGTTGATTTACTCTTCAAAACAAATCGAAAAGTA
ACCGTTAAGCAATTAAAAGAAGATTATTTCAAAAAAATAGAATGTTTTGATAGTGTT
GAAATTTCAGGAGTTGAAGATAGATTTAATGCTTCATTAGGTACCTACCATGATTTG
CTAAAAATTATTAAAGATAAAGATTTTTTGGATAATGAAGAAAATGAAGATATCTTA
GAGGATATTGTTTTAACATTGACCTTATTTGAAGATAGGGAGATGATTGAGGAAAGA
CTTAAAACATATGCTCACCTCTTTGATGATAAGGTGATGAAACAGCTTAAACGTCGC
CGTTATACTGGTTGGGGACGTTTGTCTCGAAAATTGATTAATGGTATTAGGGATAAG
CAATCTGGCAAAACAATATTAGATTTTTTGAAATCAGATGGTTTTGCCAATCGCAAT
TTTATGCAGCTGATCCATGATGATAGTTTGACATTTAAAGAAGACATTCAAAAAGCA
CAAGTGTCTGGACAAGGCGATAGTTTACATGAACATATTGCAAATTTAGCTGGTAGC
CCTGCTATTAAAAAAGGTATTTTACAGACTGTAAAAGTTGTTGATGAATTGGTCAAA
GTAATGGGGCGGCATAAGCCAGAAAATATCGTTATTGAAATGGCACGTGAAAATCA
GACAACTCAAAAGGGCCAGAAAAATTCGCGAGAGCGTATGAAACGAATCGAAGAA
GGTATCAAAGAATTAGGAAGTCAGATTCTTAAAGAGCATCCTGTTGAAAATACTCA
ATTGCAAAATGAAAAGCTCTATCTCTATTATCTCCAAAATGGAAGAGACATGTATGT
GGACCAAGAATTAGATATTAATCGTTTAAGTGATTATGATGTCGATCACATTGTTCC
ACAAAGTTTCCTTAAAGACGATTCAATAGACAATAAGGTCTTAACGCGTTCTGATAA
AAATCGTGGTAAATCGGATAACGTTCCAAGTGAAGAAGTAGTCAAAAAGATGAAAA
ACTATTGGAGACAACTTCTAAACGCCAAGTTAATCACTCAACGTAAGTTTGATAATT
TAACGAAAGCTGAACGTGGAGGTTTGAGTGAACTTGATAAAGCTGGTTTTATCAAAC
GCCAATTGGTTGAAACTCGCCAAATCACTAAGCATGTGGCACAAATTTTGGATAGTC
GCATGAATACTAAATACGATGAAAATGATAAACTTATTCGAGAGGTTAAAGTGATT
ACCTTAAAATCTAAATTAGTTTCTGACTTCCGAAAAGATTTCCAATTCTATAAAGTA
CGTGAGATTAACAATTACCATCATGCCCATGATGCGTATCTAAATGCCGTCGTTGGA
ACTGCTTTGATTAAGAAATATCCAAAACTTGAATCGGAGTTTGTCTATGGTGATTAT
AAAGTTTATGATGTTCGTAAAATGATTGCTAAGTCTGAGCAAGAAATAGGCAAAGC
AACCGCAAAATATTTCTTTTACTCTAATATCATGAACTTCTTCAAAACAGAAATTAC
ACTTGCAAATGGAGAGATTCGCAAACGCCCTCTAATCGAAACTAATGGGGAAACTG
GAGAAATTGTCTGGGATAAAGGGCGAGATTTTGCCACAGTGCGCAAAGTATTGTCC
ATGCCCCAAGTCAATATTGTCAAGAAAACAGAAGTACAGACAGGCGGATTCTCCAA
GGAGTCAATTTTACCAAAAAGAAATTCGGACAAGCTTATTGCTCGTAAAAAAGACT
GGGATCCAAAAAAATATGGTGGTTTTGATAGTCCAACGGTAGCTTATTCAGTCCTAG
TGGTTGCTAAGGTGGAAAAAGGGAAATCGAAGAAGTTAAAATCCGTTAAAGAGTTA
CTAGGGATCACAATTATGGAAAGAAGTTCCTTTGAAAAAAATCCGATTGACTTTTTA
GAAGCTAAAGGATATAAGGAAGTTAAAAAAGACTTAATCATTAAACTACCTAAATA
TAGTCTTTTTGAGTTAGAAAACGGTCGTAAACGGATGCTGGCTAGTGCCGGAGAATT
ACAAAAAGGAAATGAGCTGGCTCTGCCAAGCAAATATGTGAATTTTTTATATTTAGC
TAGTCATTATGAAAAGTTGAAGGGTAGTCCAGAAGATAACGAACAAAAACAATTGT
TTGTGGAGCAGCATAAGCATTATTTAGATGAGATTATTGAGCAAATCAGTGAATTTT
CTAAGCGTGTTATTTTAGCAGATGCCAATTTAGATAAAGTTCTTAGTGCATATAACA
AACATAGAGACAAACCAATACGTGAACAAGCAGAAAATATTATTCATTTATTTACGT
TGACGAATCTTGGAGCTCCCGCTGCTTTTAAATATTTTGATACAACAATTGATCGTA
AACGATATACGTCTACAAAAGAAGTTTTAGATGCCACTCTTATCCATCAATCCATCA
CTGGTCTTTATGAAACACGCATTGATTTGAGTCAGCTAGGAGGTGACTGATAATCAA
ATGTCAGACGAAAATGCCAATTATTGAAGCGGCTAACGCCGCTTTTTTTGTTTCTGG
TCTCCCTTGCTTGTACTTTACAGTATAGCTTCTAGAGATCTGCAGTGACTGGAGCTTC
TAGAGATCTGCAGTGACTGGGAAAACCCTGGCGACTAGTCTTGGACTCCTGTTGATA
GATCCAGTAATGACCTCAGAACTCCATCTGGATTTGTTCAGAACGCTCGGTTGCCGC
CGGGCGTTTTTTATTGGTGAGAATCCAGGGGTCCCCAATAATTACGATTTAAATCCT
TCAAACTTCCCAAAGGCGAGCCCTAGTGACATTAGAAAACCGACTGTAAAAAGTAC
AGTCGGCATTATCTCATATTATAAAAGCCAGTCATTAGGCCTATCTGACAATTCCTG
AATAGAGTTCATAAACAATCCTGCATGATAACCATCACAAACAGAATGATGTACCT
GTAAAGATAGCGGTAAATATATTGAATTACCTTTATTAATGAATTTTCCTGCTGTAAT
AATGGGTAGAAGGTAATTACTATTATTATTGATATTTAAGTTAAACCCAGTAAATGA
AGTCCATGGAATAATAGAAAGAGAAAAAGCATTTTCAGGTATAGGTGTTTTGGGAA
ACAATTTCCCCGAACCATTATATTTCTCTACATCAGAAAGGTATAAATCATAAAACT
CTTTGAAGTCATTCTTTACAGGAGTCCAAATACCAGAGAATGTTTTAGATACACCAT
CAAAAATTGTATAAAGTGGCTCTAACTTATCCCAATAACCTAACTCTCCGTCGCTAT
TGTAACCAGTTCTAAAAGCTGTATTTGAGTTTATCACCCTTGTCACTAAGAAAATAA
ATGCAGGGTAAAATTTATATCCTTCTTGTTTTATGTTTCGGTATAAAACACTAATATC
AATTTCTGTGGTTATACTAAAAGTCGTTTGTTGGTTCAAATAATGATTAAATATCTCT
TTTCTCTTCCAATTGTCTAAATCAATTTTATTAAAGTTCATTTGATATGCCTCCTAAAT
TTTTATCTAAAGTGAATTTAGGAGGCTTACTTGTCTGCTTTCTTCATTAGAATCAATC
CTTTTTTAAAAGTCAATCCCGTTTGTTGAAGACTTTTGTCCTTTTCCGCTGCATAAAC
AATATGGCCCGTTTGTTGAACTACTCTTTAATAAAATAATTTTTCCGTTCCCAATTCC
ACATTGCAATAATAGAAAATCCATCTTCATCGGCTTTTTCGTCATCATCTGTATGAAT
CAAATCGCCTTCTTCTGTGTCATCAAGGTTTAATTTTTTATGTATTTCTTTTAACAAAC
CACCATAGGAGATTAACCTTTTACGGTGTAAACCTTCCTCCAAATCAGACAAACGTT
TCAAATTCTTTTCTTCATCATCGGTCATAAAATCCGTATCCTTTACAGGATATTTTGC
AGTTTCGTCAATTGCCGATTGTATATCCGATTTATATTTATTTTTCGGTCGAATCATTT
GAACTTTTACATTTGGATCATAGTCTAATTTCATTGCCTTTTTCCAAAATTGAATCCA
TTGTTTTTGATTCACGTAGTTTTCTGTATTCTTAAAATAAGTTGGTTCCACACATACC
AATACATGCATGTGCTGATTATAAGAATTATCTTTATTATTTATTGTCACTTCCGTTG
CACGCATAAAACCAACAAGATTTTTATTAATTTTTTTATATTGCATCATTCGGCGAAA
TCCTTGAGCCATATCTGACAAACTCTTATTTAATTCTTCGCCATCATAAACATTTTTA
ACTGTTAATGTGAGAAACAACCAACGAACTGTTGGCTTTTGTTTAATAACTTCAGCA
ACAACCTTTTGTGACTGAATGCCATGTTTCATTGCTCTCCTCCAGTTGCACATTGGAC
AAAGCCTGGATTTACAAAACCACACTCGATACAACTTTCTTTCGCCTGTTTCACGATT
TTGTTTATACTCTAATATTTCAGCACAATCTTTTACTCTTTCAGCCTTTTTAAATTCAA
GAATATGCAGAAGTTCAAAGTAATCAACATTAGCGATTTTCTTTTCTCTCCATGGTCT
CACTTTTCCACTTTTTGTCTTGTCCACTAAAACCCTTGATTTTTCATCTGAATAAATGC
TACTATTAGGACACATAATATTAAAAGAAACCCCCATCTATTTAGTTATTTGTTTGGT
CACTTATAACTTTAACAGATGGGGTTTTTCTGTGCAACCAATTTTAAGGGTTTTCCAA
TACTTTAAAACACATACATACCAACACTTCAACGCACCTTTCAGCAACTAAAATAAA
AATGACGTTATTTCTATATGTATCAAGATAAGAAAGAACAAGTTCAAAACCATCAA
AAAAAGACACCTTTTCAGGTGCTTTTTTTATTTTATAAACTCATTCCCTGATCTCGAC
TTCGTTCTTTTTTTACCTCTCGGTTATGAGTTAGTTCAAATTCGTTCTTTTTAGGTTCT
AAATCGTGTTTTTCTTGGAATTGTGCTGTTTTATCCTTTACCTTGTCTACAAACCCCTT
AAAAACGTTTTTAAAGGCTTTTAAGCGTCTGTACGTTCCTTAAGGAATTATTCCTTAG
TGCTTTCTAGGTTAATGTCATGATAATAATGGTTTCTTAGACGTCAGGTGGCACTTTT
CGGGGAAATGTCCGCGGAACCCCTATTTGTTTATTTTTCTAAATACATTCAAATATGT
ATCCGCTCATGAGACAATAACCCTGATAAATGCTTCAATAATATTGAAAAAGGAAG
AGTATGAGTATTCAACATTTCCGTGTCGCCCTTATTCCCTTTTTTGCGGCATTTTGCCT
TCCTGTTTTTGCTCACCCAGAAACGCTGGTGAAAGTAAAAGATGCTGAAGATCAGTT
GGGTGCACGAGTGGGTTACATCGAACTGGATCTCAACAGCGGTAAGATCCTTGAGA
GTTTTCGCCCCGAAGAACGTTTTCCAATGATGAGCACTTTTAAAGTTCTGCTATGTGG
CGCGGTATTATCCCGTGTTGACGCCGGGCAAGAGCAACTCGGTCGCCGCATACACTA
TTCTCAGAATGACTTGGTTGAGTACTCACCAGTCACAGAAAAGCATCTTACGGATGG
CATGACAGTAAGAGAATTATGCAGTGCTGCCATAACCATGAGTGATAACACTGCGG
CCAACTTACTTCTGACAACGATCGGAGGACCGAAGGAGCTAACCGCTTTTTTGCACA
ACATGGGGGATCATGTAACTCGCCTTGATCGTTGGGAACCGGAGCTGAATGAAGCC
ATACCAAACGACGAGCGTGACACCACGATGCCTGCAGCAATGGCAACAACGTTGCG
CAAACTATTAACTGGCGAACTACTTACTCTAGCTTCCCGGCAACAATTAATAGACTG
GATGGAGGCGGATAAAGTTGCAGGACCACTTCTGCGCTCGGCCCTTCCGGCTGGCTG
GTTTATTGCTGATAAATCTGGAGCCGGTGAGCGTGGGTCTCGCGGTATCATTGCAGC
ACTGGGGCCAGATGGTAAGCCCTCCCGTATCGTAGTTATCTACACGACGGGGAGTCA
GGCAACTATGGATGAACGAAATAGACAGATCGCTGAGATAGGTGCCTCACTGATTA
AGCATTGGTAACTGTCAGACCAAGTTTACTCATATATACTTTAGATTGATTTAAAAC
TTCATTTTTAATTTAAAAGGATCTAGGTGAAGATCCTTTTTGATAATCTCATGACCAA
AATCCCTTAACGTGAGTTTTCGTTCCACTGAGCGTCAGACCCCTTAATAAGATGATC
TTCTTGAGATCGTTTTGGTCTGCGCGTAATCTCTTGCTCTGAAAACGAAAAAACCGC
CTTGCAGGGAGGTTTTTCGAAGGTTCTCTGAGCTACCAACTCTTTGAACCGAGGTAA
CTGGCTTGCAGGAGCGCAGTCACCAAAACTTGTCCTTTCAGTTTAGCCTTAACCGGC
GCATGACTTCAAGACTAACTCCTCTAAATCAATTACCAGTGGCTGCTGCCAGTGGTG
CTTTTGCATGTCTTTCCGGGTTGGACTCAAGACGATAGTTACCGGATAAGGCGCAGC
GGTCGGACTGAACGGGGGGTTCGTGCATACAGTCCAGCTTGGAGCGAACTGCCTAC
CCGGAACTGAGTGTCAGGCGTGGAATGAGACAAACGCGGCCATAACAGCGGAATGA
CACCGGTAAACCGAAAGGCAGGAACAGGAGAGCGCACGAGGGAGCCGCCAGGGGG
AAACGCCTGGTATCTTTATAGTCCTGTCGGGTTTCGCCACCACTGATTTGAGCGTCA
GATTTCGTGATGCTTGTCAGGGGGCGGAGCCTATGGAAAAACGCTTTGCCCGAGCCT
ATGGAAAAACGCTTTGCCCCACACAGGCTCTTGAACAAGGGATAACAATTTGTCATA
GCTGTATTCGTTACAAGGAGGATTTTCGAAAAAACACCCTAACGGGTGTTTTTTTAT
AGCTGGTCTCCCCAAGTGCTGTAAAAAACGCCGAATGAAGCGGATGAAATTGCTCA
TCGTCTCGTGAAAAAGCCGAAGAAGGTTAAGCCTGGGTATAAAAAGAAAATGAGCT
ATGAGATGGAGAAAGCCATAACCATGAGTGATAACACTGGTTTTAGAGCTAGAAAT
AGCAAGTTAAAATAAGGCTAGTCCGTTATCAACTTGAAAAAGTGGCACCGAGTCGG
TGCTTTTTTTCTCGGTACCAAATTCCAGAAAAGAGGCCTCCCGAAAGGGGGGCCTTT
TTTCGTTTTGGTCCCTCAATGATCTCCTTATCCGGTTAAGATGGCAAGCTTGACAAGT
ATTTCTTCAGAATTTACAATGAAGTTTACATGAC
>pCas9g2-D15-cons
(SEQ ID No: 232)
GATTAACTAATAAGGAGGGCAAACTACTAGAGATGAGTAAATCCTGGGGCAAATTC
ATCGAGGAGGAAGAGGCGGAAATGGCTTCCCGTCGTAATCTAATGATTGTCGATGG
AACTAACTTAGGCTTTCGCTTCAAACATAACAATAGTAAAAAACCATTTGCCTCAAG
TTATGTTTCAACTATTCAATCTCTGGCAAAATCCTACTCTGCCAGAACTACGATTGTT
CTAGGTGATAAGGGCAAAAGCGTGTTCAGACTAGAACATCTACCAGAGTATAAAGG
TAATCGTGATGAAAAGTACGCACAACGTACGGAAGAGGAGAAAGCGCTAGATGAG
CAGTTCTTCGAATACCTCAAAGATGCTTTCGAGTTGTGTAAAACTACATTCCCAACTT
TTACCATTCGTGGTGTAGAAGCAGATGACATGGCGGCGTATATTGTTAAGCTCATCG
GGCATCTTTATGATCACGTTTGGCTAATATCTACAGATGGTGACTGGGATACTTTATT
AACGGATAAAGTTTCTCGTTTTTCTTTCACAACACGTCGTGAGTATCATCTTCGTGAT
ATGTATGAGCACCATAACGTGGACGACGTAGAACAGTTTATCTCCCTGAAAGCAATT
ATGGGAGATCTAGGAGATAATATTCGTGGTGTTGAAGGAATAGGAGCAAAACGCGG
ATATAATATTATTCGTGAGTTTGGTAACGTACTGGATATTATTGATCAGCTTCCACTG
CCTGGAAAGCAGAAATATATACAGAACCTGAATGCATCGGAAGAACTGCTTTTCCG
AAACTTGATTCTGGTTGATTTACCTACCTACTGTGTGGATGCTATTGCTGCTGTAGGT
CAAGATGTGTTAGATAAGTTCACGAAAGACATCCTTGAGATCGCGGAACAATGATA
AGCTCTTAAAGGGGGTTTTAGATACCAGAGATGGATAAGAAATACTCAATAGGCTT
AGATATCGGCACAAATAGCGTCGGATGGGCGGTGATCACTGATGAATATAAGGTTC
CGTCTAAAAAGTTCAAGGTTCTGGGAAATACAGACCGCCACAGTATCAAAAAAAAT
CTTATAGGGGCTCTTTTATTTGACAGTGGAGAGACAGCGGAAGCGACTCGTCTCAAA
CGGACAGCTCGTAGAAGGTATACACGTCGGAAGAATCGTATTTGTTATCTACAGGA
GATTTTTTCAAATGAGATGGCGAAAGTAGATGATAGTTTCTTTCATCGACTTGAAGA
GTCTTTTTTGGTGGAAGAAGACAAGAAGCATGAACGTCATCCTATTTTTGGAAATAT
AGTAGATGAAGTTGCTTATCATGAGAAATATCCAACTATCTATCATCTGCGAAAAAA
ATTGGTAGATTCTACTGATAAAGCGGATTTGCGCTTAATCTATTTGGCCTTAGCGCAT
ATGATTAAGTTTCGTGGTCATTTTTTGATTGAGGGAGATTTAAATCCTGATAATAGTG
ATGTGGACAAACTATTTATCCAGTTGGTACAAACCTACAATCAATTATTTGAAGAAA
ACCCTATTAACGCAAGTGGAGTAGATGCTAAAGCGATTCTTTCTGCACGATTGAGTA
AATCAAGACGATTAGAAAATCTCATTGCTCAGCTCCCCGGTGAGAAGAAAAATGGC
TTATTTGGGAATCTCATTGCTTTGTCATTGGGTTTGACCCCTAATTTTAAATCAAATT
TTGATTTGGCAGAAGATGCTAAATTACAGCTTTCAAAAGATACTTACGATGATGATT
TAGATAATTTATTGGCGCAAATTGGAGATCAATATGCTGATTTGTTTTTGGCAGCTA
AGAATTTATCAGATGCTATTTTACTTTCAGATATCCTAAGAGTAAATACTGAAATAA
CTAAGGCTCCCCTATCAGCTTCAATGATTAAACGCTACGATGAACATCATCAAGACT
TGACTCTTTTAAAAGCTTTAGTTCGACAACAACTTCCAGAAAAGTATAAAGAAATCT
TTTTTGATCAATCAAAAAACGGATATGCAGGTTATATTGATGGGGGAGCTAGCCAAG
AAGAATTTTATAAATTTATCAAACCAATTTTAGAAAAAATGGATGGTACTGAGGAAT
TATTGGTGAAACTAAATCGTGAAGATTTGCTGCGCAAGCAACGGACCTTTGACAACG
GCTCTATTCCCCATCAAATTCACTTGGGTGAGCTGCATGCTATTTTGAGAAGACAAG
AAGACTTTTATCCATTTTTAAAAGACAATCGTGAGAAGATTGAAAAAATCTTGACTT
TTCGAATTCCTTATTATGTTGGTCCATTGGCGCGTGGCAATAGTCGTTTTGCATGGAT
GACTCGGAAGTCTGAAGAAACAATTACCCCATGGAATTTTGAAGAAGTTGTCGATA
AAGGTGCTTCAGCTCAATCATTTATTGAACGCATGACAAACTTTGATAAAAATCTTC
CAAATGAAAAAGTACTACCAAAACATAGTTTGCTTTATGAGTATTTTACGGTTTATA
ACGAATTGACAAAGGTCAAATATGTTACTGAAGGAATGCGAAAACCAGCATTTCTTT
CAGGTGAACAGAAGAAAGCCATTGTTGATTTACTCTTCAAAACAAATCGAAAAGTA
ACCGTTAAGCAATTAAAAGAAGATTATTTCAAAAAAATAGAATGTTTTGATAGTGTT
GAAATTTCAGGAGTTGAAGATAGATTTAATGCTTCATTAGGTACCTACCATGATTTG
CTAAAAATTATTAAAGATAAAGATTTTTTGGATAATGAAGAAAATGAAGATATCTTA
GAGGATATTGTTTTAACATTGACCTTATTTGAAGATAGGGAGATGATTGAGGAAAGA
CTTAAAACATATGCTCACCTCTTTGATGATAAGGTGATGAAACAGCTTAAACGTCGC
CGTTATACTGGTTGGGGACGTTTGTCTCGAAAATTGATTAATGGTATTAGGGATAAG
CAATCTGGCAAAACAATATTAGATTTTTTGAAATCAGATGGTTTTGCCAATCGCAAT
TTTATGCAGCTGATCCATGATGATAGTTTGACATTTAAAGAAGACATTCAAAAAGCA
CAAGTGTCTGGACAAGGCGATAGTTTACATGAACATATTGCAAATTTAGCTGGTAGC
CCTGCTATTAAAAAAGGTATTTTACAGACTGTAAAAGTTGTTGATGAATTGGTCAAA
GTAATGGGGCGGCATAAGCCAGAAAATATCGTTATTGAAATGGCACGTGAAAATCA
GACAACTCAAAAGGGCCAGAAAAATTCGCGAGAGCGTATGAAACGAATCGAAGAA
GGTATCAAAGAATTAGGAAGTCAGATTCTTAAAGAGCATCCTGTTGAAAATACTCA
ATTGCAAAATGAAAAGCTCTATCTCTATTATCTCCAAAATGGAAGAGACATGTATGT
GGACCAAGAATTAGATATTAATCGTTTAAGTGATTATGATGTCGATCACATTGTTCC
ACAAAGTTTCCTTAAAGACGATTCAATAGACAATAAGGTCTTAACGCGTTCTGATAA
AAATCGTGGTAAATCGGATAACGTTCCAAGTGAAGAAGTAGTCAAAAAGATGAAAA
ACTATTGGAGACAACTTCTAAACGCCAAGTTAATCACTCAACGTAAGTTTGATAATT
TAACGAAAGCTGAACGTGGAGGTTTGAGTGAACTTGATAAAGCTGGTTTTATCAAAC
GCCAATTGGTTGAAACTCGCCAAATCACTAAGCATGTGGCACAAATTTTGGATAGTC
GCATGAATACTAAATACGATGAAAATGATAAACTTATTCGAGAGGTTAAAGTGATT
ACCTTAAAATCTAAATTAGTTTCTGACTTCCGAAAAGATTTCCAATTCTATAAAGTA
CGTGAGATTAACAATTACCATCATGCCCATGATGCGTATCTAAATGCCGTCGTTGGA
ACTGCTTTGATTAAGAAATATCCAAAACTTGAATCGGAGTTTGTCTATGGTGATTAT
AAAGTTTATGATGTTCGTAAAATGATTGCTAAGTCTGAGCAAGAAATAGGCAAAGC
AACCGCAAAATATTTCTTTTACTCTAATATCATGAACTTCTTCAAAACAGAAATTAC
ACTTGCAAATGGAGAGATTCGCAAACGCCCTCTAATCGAAACTAATGGGGAAACTG
GAGAAATTGTCTGGGATAAAGGGCGAGATTTTGCCACAGTGCGCAAAGTATTGTCC
ATGCCCCAAGTCAATATTGTCAAGAAAACAGAAGTACAGACAGGCGGATTCTCCAA
GGAGTCAATTTTACCAAAAAGAAATTCGGACAAGCTTATTGCTCGTAAAAAAGACT
GGGATCCAAAAAAATATGGTGGTTTTGATAGTCCAACGGTAGCTTATTCAGTCCTAG
TGGTTGCTAAGGTGGAAAAAGGGAAATCGAAGAAGTTAAAATCCGTTAAAGAGTTA
CTAGGGATCACAATTATGGAAAGAAGTTCCTTTGAAAAAAATCCGATTGACTTTTTA
GAAGCTAAAGGATATAAGGAAGTTAAAAAAGACTTAATCATTAAACTACCTAAATA
TAGTCTTTTTGAGTTAGAAAACGGTCGTAAACGGATGCTGGCTAGTGCCGGAGAATT
ACAAAAAGGAAATGAGCTGGCTCTGCCAAGCAAATATGTGAATTTTTTATATTTAGC
TAGTCATTATGAAAAGTTGAAGGGTAGTCCAGAAGATAACGAACAAAAACAATTGT
TTGTGGAGCAGCATAAGCATTATTTAGATGAGATTATTGAGCAAATCAGTGAATTTT
CTAAGCGTGTTATTTTAGCAGATGCCAATTTAGATAAAGTTCTTAGTGCATATAACA
AACATAGAGACAAACCAATACGTGAACAAGCAGAAAATATTATTCATTTATTTACGT
TGACGAATCTTGGAGCTCCCGCTGCTTTTAAATATTTTGATACAACAATTGATCGTA
AACGATATACGTCTACAAAAGAAGTTTTAGATGCCACTCTTATCCATCAATCCATCA
CTGGTCTTTATGAAACACGCATTGATTTGAGTCAGCTAGGAGGTGACTGATAATCAA
ATGTCAGACGAAAATGCCAATTATTGAAGCGGCTAACGCCGCTTTTTTTGTTTCTGG
TCTCCCTTGCTTGTACTTTACAGTATAGCTTCTAGAGATCTGCAGTGACTGGAGCTTC
TAGAGATCAGGTCAGACGCGATTTCCTGGGTGCTCGAGCCATGGGAAAGCGGTGGT
GGAAAAAACGCCGAATGAAGCGGATGAAATTGCTCATCGTCTCGTGAAAAAGCCGA
AGAAGGTTAAGCCTGGGTATAAAAAGAAAATGAGCTATGAGATGGAGAAATTGAA
GTCATGCGCCGGTTAGTTTTAGAGCTAGAAATAGCAAGTTAAAATAAGGCTAGTCCG
TTATCAACTTGAAAAAGTGGCACCGAGTCGGTGCTTTTTTTCTCGGTACCAAATTCC
AGAAAAGAGGCCTCCCGAAAGGGGGGCCTTTTTTCGTTTTGGTCCCTCAATGATCCG
ACAGGAAGTGGTATTAGCGACTCCTACAAGGAATAGGTGTCTGGGTCATGCAGTGA
CTGGGAAAACCCTGGCGACTAGTCTTGGACTCCTGTTGATAGATCCAGTAATGACCT
CAGAACTCCATCTGGATTTGTTCAGAACGCTCGGTTGCCGCCGGGCGTTTTTTATTGG
TGAGAATCCAGGGGTCCCCAATAATTACGATTTAAATCCTTCAAACTTCCCAAAGGC
GAGCCCTAGTGACATTAGAAAACCGACTGTAAAAAGTACAGTCGGCATTATCTCAT
ATTATAAAAGCCAGTCATTAGGCCTATCTGACAATTCCTGAATAGAGTTCATAAACA
ATCCTGCATGATAACCATCACAAACAGAATGATGTACCTGTAAAGATAGCGGTAAA
TATATTGAATTACCTTTATTAATGAATTTTCCTGCTGTAATAATGGGTAGAAGGTAAT
TACTATTATTATTGATATTTAAGTTAAACCCAGTAAATGAAGTCCATGGAATAATAG
AAAGAGAAAAAGCATTTTCAGGTATAGGTGTTTTGGGAAACAATTTCCCCGAACCAT
TATATTTCTCTACATCAGAAAGGTATAAATCATAAAACTCTTTGAAGTCATTCTTTAC
AGGAGTCCAAATACCAGAGAATGTTTTAGATACACCATCAAAAATTGTATAAAGTG
GCTCTAACTTATCCCAATAACCTAACTCTCCGTCGCTATTGTAACCAGTTCTAAAAGC
TGTATTTGAGTTTATCACCCTTGTCACTAAGAAAATAAATGCAGGGTAAAATTTATA
TCCTTCTTGTTTTATGTTTCGGTATAAAACACTAATATCAATTTCTGTGGTTATACTA
AAAGTCGTTTGTTGGTTCAAATAATGATTAAATATCTCTTTTCTCTTCCAATTGTCTA
AATCAATTTTATTAAAGTTCATTTGATATGCCTCCTAAATTTTTATCTAAAGTGAATT
TAGGAGGCTTACTTGTCTGCTTTCTTCATTAGAATCAATCCTTTTTTAAAAGTCAATC
CCGTTTGTTGAAGACTTTTGTCCTTTTCCGCTGCATAAACAATATGGCCCGTTTGTTG
AACTACTCTTTAATAAAATAATTTTTCCGTTCCCAATTCCACATTGCAATAATAGAAA
ATCCATCTTCATCGGCTTTTTCGTCATCATCTGTATGAATCAAATCGCCTTCTTCTGT
GTCATCAAGGTTTAATTTTTTATGTATTTCTTTTAACAAACCACCATAGGAGATTAAC
CTTTTACGGTGTAAACCTTCCTCCAAATCAGACAAACGTTTCAAATTCTTTTCTTCAT
CATCGGTCATAAAATCCGTATCCTTTACAGGATATTTTGCAGTTTCGTCAATTGCCGA
TTGTATATCCGATTTATATTTATTTTTCGGTCGAATCATTTGAACTTTTACATTTGGAT
CATAGTCTAATTTCATTGCCTTTTTCCAAAATTGAATCCATTGTTTTTGATTCACGTA
GTTTTCTGTATTCTTAAAATAAGTTGGTTCCACACATACCAATACATGCATGTGCTGA
TTATAAGAATTATCTTTATTATTTATTGTCACTTCCGTTGCACGCATAAAACCAACAA
GATTTTTATTAATTTTTTTATATTGCATCATTCGGCGAAATCCTTGAGCCATATCTGA
CAAACTCTTATTTAATTCTTCGCCATCATAAACATTTTTAACTGTTAATGTGAGAAAC
AACCAACGAACTGTTGGCTTTTGTTTAATAACTTCAGCAACAACCTTTTGTGACTGA
ATGCCATGTTTCATTGCTCTCCTCCAGTTGCACATTGGACAAAGCCTGGATTTACAA
AACCACACTCGATACAACTTTCTTTCGCCTGTTTCACGATTTTGTTTATACTCTAATA
TTTCAGCACAATCTTTTACTCTTTCAGCCTTTTTAAATTCAAGAATATGCAGAAGTTC
AAAGTAATCAACATTAGCGATTTTCTTTTCTCTCCATGGTCTCACTTTTCCACTTTTTG
TCTTGTCCACTAAAACCCTTGATTTTTCATCTGAATAAATGCTACTATTAGGACACAT
AATATTAAAAGAAACCCCCATCTATTTAGTTATTTGTTTGGTCACTTATAACTTTAAC
AGATGGGGTTTTTCTGTGCAACCAATTTTAAGGGTTTTCCAATACTTTAAAACACAT
ACATACCAACACTTCAACGCACCTTTCAGCAACTAAAATAAAAATGACGTTATTTCT
ATATGTATCAAGATAAGAAAGAACAAGTTCAAAACCATCAAAAAAAGACACCTTTT
CAGGTGCTTTTTTTATTTTATAAACTCATTCCCTGATCTCGACTTCGTTCTTTTTTTAC
CTCTCGGTTATGAGTTAGTTCAAATTCGTTCTTTTTAGGTTCTAAATCGTGTTTTTCTT
GGAATTGTGCTGTTTTATCCTTTACCTTGTCTACAAACCCCTTAAAAACGTTTTTAAA
GGCTTTTAAGCGTCTGTACGTTCCTTAAGGAATTATTCCTTAGTGCTTTCTAGGTTAA
TGTCATGATAATAATGGTTTCTTAGACGTCAGGTGGCACTTTTCGGGGAAATGTCCG
CGGAACCCCTATTTGTTTATTTTTCTAAATACATTCAAATATGTATCCGCTCATGAGA
CAATAACCCTGATAAATGCTTCAATAATATTGAAAAAGGAAGAGTATGAGTATTCA
ACATTTCCGTGTCGCCCTTATTCCCTTTTTTGCGGCATTTTGCCTTCCTGTTTTTGCTC
ACCCAGAAACGCTGGTGAAAGTAAAAGATGCTGAAGATCAGTTGGGTGCACGAGTG
GGTTACATCGAACTGGATCTCAACAGCGGTAAGATCCTTGAGAGTTTTCGCCCCGAA
GAACGTTTTCCAATGATGAGCACTTTTAAAGTTCTGCTATGTGGCGCGGTATTATCCC
GTGTTGACGCCGGGCAAGAGCAACTCGGTCGCCGCATACACTATTCTCAGAATGACT
TGGTTGAGTACTCACCAGTCACAGAAAAGCATCTTACGGATGGCATGACAGTAAGA
GAATTATGCAGTGCTGCCATAACCATGAGTGATAACACTGCGGCCAACTTACTTCTG
ACAACGATCGGAGGACCGAAGGAGCTAACCGCTTTTTTGCACAACATGGGGGATCA
TGTAACTCGCCTTGATCGTTGGGAACCGGAGCTGAATGAAGCCATACCAAACGACG
AGCGTGACACCACGATGCCTGCAGCAATGGCAACAACGTTGCGCAAACTATTAACT
GGCGAACTACTTACTCTAGCTTCCCGGCAACAATTAATAGACTGGATGGAGGCGGAT
AAAGTTGCAGGACCACTTCTGCGCTCGGCCCTTCCGGCTGGCTGGTTTATTGCTGAT
AAATCTGGAGCCGGTGAGCGTGGGTCTCGCGGTATCATTGCAGCACTGGGGCCAGA
TGGTAAGCCCTCCCGTATCGTAGTTATCTACACGACGGGGAGTCAGGCAACTATGGA
TGAACGAAATAGACAGATCGCTGAGATAGGTGCCTCACTGATTAAGCATTGGTAAC
TGTCAGACCAAGTTTACTCATATATACTTTAGATTGATTTAAAACTTCATTTTTAATT
TAAAAGGATCTAGGTGAAGATCCTTTTTGATAATCTCATGACCAAAATCCCTTAACG
TGAGTTTTCGTTCCACTGAGCGTCAGACCCCTTAATAAGATGATCTTCTTGAGATCGT
TTTGGTCTGCGCGTAATCTCTTGCTCTGAAAACGAAAAAACCGCCTTGCAGGGAGGT
TTTTCGAAGGTTCTCTGAGCTACCAACTCTTTGAACCGAGGTAACTGGCTTGCAGGA
GCGCAGTCACCAAAACTTGTCCTTTCAGTTTAGCCTTAACCGGCGCATGACTTCAAG
ACTAACTCCTCTAAATCAATTACCAGTGGCTGCTGCCAGTGGTGCTTTTGCATGTCTT
TCCGGGTTGGACTCAAGACGATAGTTACCGGATAAGGCGCAGCGGTCGGACTGAAC
GGGGGGTTCGTGCATACAGTCCAGCTTGGAGCGAACTGCCTACCCGGAACTGAGTG
TCAGGCGTGGAATGAGACAAACGCGGCCATAACAGCGGAATGACACCGGTAAACC
GAAAGGCAGGAACAGGAGAGCGCACGAGGGAGCCGCCAGGGGGAAACGCCTGGTA
TCTTTATAGTCCTGTCGGGTTTCGCCACCACTGATTTGAGCGTCAGATTTCGTGATGC
TTGTCAGGGGGCGGAGCCTATGGAAAAACGCTTTGCCCGAGCCTATGGAAAAACGC
TTTGCCCCACACAGGCTCTTGAACAAGGGATAACAATTTGTCATAGCTGTATTCGTT
ACAAGGAGGATTTTCGAAAAAACACCCTAACGGGTGTTTTTTTATAGCTGGTCTCCC
CAAGTGCTGTAAAAAACGCCGAATGAAGCGGATGAAATTGCTCATCGTCTCGTGAA
AAAGCCGAAGAAGGTTAAGCCTGGGTATAAAAAGAAAATGAGCTATGAGATGGAG
AAAGCCATAACCATGAGTGATAACACTGGTTTTAGAGCTAGAAATAGCAAGTTAAA
ATAAGGCTAGTCCGTTATCAACTTGAAAAAGTGGCACCGAGTCGGTGCTTTTTTTCT
CGGTACCAAATTCCAGAAAAGAGGCCTCCCGAAAGGGGGGCCTTTTTTCGTTTTGGT
CCCTCAATGATCTCCTTATCCGGTTAAGATGGCAAGCTTGACAAGTATTTCTTCAGA
ATTTACAATGAAGTTTACATGAC
>pCas9g1
(SEQ ID No: 233)
tccttatccggttaagatggCTTTTAAATCAATTTTCAGCTCCTGTATACAATTACCAAAG
TTTTTCTGAATGAAGCCATGTGTTTTGACACATTCTATACTCACAAGGAGGTGAGACACat
ggataagaaatactcaataggctTAGATATCGGCACAAATAGCGTCGGATGGGCGGTGATC
ACTGATGAATATAAGGTTCCGTCTAAAAAGTTCAAGGTTCTGGGAAATACAGACCGCCACA
GTATCAAAAAAAATCTTATAGGGGCTCTTTTATTTGACAGTGGAGAGACAGCGGAAG
CGACTCGTCTCAAACGGACAGCTCGTAGAAGGTATACACGTCGGAAGAATCGTATTT
GTTATCTACAGGAGATTTTTTCAAATGAGATGGCGAAAGTAGATGATAGTTTCTTTC
ATCGACTTGAAGAGTCTTTTTTGGTGGAAGAAGACAAGAAGCATGAACGTCATCCTA
TTTTTGGAAATATAGTAGATGAAGTTGCTTATCATGAGAAATATCCAACTATCTATC
ATCTGCGAAAAAAATTGGTAGATTCTACTGATAAAGCGGATTTGCGCTTAATCTATT
TGGCCTTAGCGCATATGATTAAGTTTCGTGGTCATTTTTTGATTGAGGGAGATTTAAA
TCCTGATAATAGTGATGTGGACAAACTATTTATCCAGTTGGTACAAACCTACAATCA
ATTATTTGAAGAAAACCCTATTAACGCAAGTGGAGTAGATGCTAAAGCGATTCTTTC
TGCACGATTGAGTAAATCAAGACGATTAGAAAATCTCATTGCTCAGCTCCCCGGTGA
GAAGAAAAATGGCTTATTTGGGAATCTCATTGCTTTGTCATTGGGTTTGACCCCTAA
TTTTAAATCAAATTTTGATTTGGCAGAAGATGCTAAATTACAGCTTTCAAAAGATAC
TTACGATGATGATTTAGATAATTTATTGGCGCAAATTGGAGATCAATATGCTGATTT
GTTTTTGGCAGCTAAGAATTTATCAGATGCTATTTTACTTTCAGATATCCTAAGAGTA
AATACTGAAATAACTAAGGCTCCCCTATCAGCTTCAATGATTAAACGCTACGATGAA
CATCATCAAGACTTGACTCTTTTAAAAGCTTTAGTTCGACAACAACTTCCAGAAAAG
TATAAAGAAATCTTTTTTGATCAATCAAAAAACGGATATGCAGGTTATATTGATGGG
GGAGCTAGCCAAGAAGAATTTTATAAATTTATCAAACCAATTTTAGAAAAAATGGA
TGGTACTGAGGAATTATTGGTGAAACTAAATCGTGAAGATTTGCTGCGCAAGCAAC
GGACCTTTGACAACGGCTCTATTCCCCATCAAATTCACTTGGGTGAGCTGCATGCTA
TTTTGAGAAGACAAGAAGACTTTTATCCATTTTTAAAAGACAATCGTGAGAAGATTG
AAAAAATCTTGACTTTTCGAATTCCTTATTATGTTGGTCCATTGGCGCGTGGCAATAG
TCGTTTTGCATGGATGACTCGGAAGTCTGAAGAAACAATTACCCCATGGAATTTTGA
AGAAGTTGTCGATAAAGGTGCTTCAGCTCAATCATTTATTGAACGCATGACAAACTT
TGATAAAAATCTTCCAAATGAAAAAGTACTACCAAAACATAGTTTGCTTTATGAGTA
TTTTACGGTTTATAACGAATTGACAAAGGTCAAATATGTTACTGAAGGAATGCGAAA
ACCAGCATTTCTTTCAGGTGAACAGAAGAAAGCCATTGTTGATTTACTCTTCAAAAC
AAATCGAAAAGTAACCGTTAAGCAATTAAAAGAAGATTATTTCAAAAAAATAGAAT
GTTTTGATAGTGTTGAAATTTCAGGAGTTGAAGATAGATTTAATGCTTCATTAGGTA
CCTACCATGATTTGCTAAAAATTATTAAAGATAAAGATTTTTTGGATAATGAAGAAA
ATGAAGATATCTTAGAGGATATTGTTTTAACATTGACCTTATTTGAAGATAGGGAGA
TGATTGAGGAAAGACTTAAAACATATGCTCACCTCTTTGATGATAAGGTGATGAAAC
AGCTTAAACGTCGCCGTTATACTGGTTGGGGACGTTTGTCTCGAAAATTGATTAATG
GTATTAGGGATAAGCAATCTGGCAAAACAATATTAGATTTTTTGAAATCAGATGGTT
TTGCCAATCGCAATTTTATGCAGCTGATCCATGATGATAGTTTGACATTTAAAGAAG
ACATTCAAAAAGCACAAGTGTCTGGACAAGGCGATAGTTTACATGAACATATTGCA
AATTTAGCTGGTAGCCCTGCTATTAAAAAAGGTATTTTACAGACTGTAAAAGTTGTT
GATGAATTGGTCAAAGTAATGGGGCGGCATAAGCCAGAAAATATCGTTATTGAAAT
GGCACGTGAAAATCAGACAACTCAAAAGGGCCAGAAAAATTCGCGAGAGCGTATG
AAACGAATCGAAGAAGGTATCAAAGAATTAGGAAGTCAGATTCTTAAAGAGCATCC
TGTTGAAAATACTCAATTGCAAAATGAAAAGCTCTATCTCTATTATCTCCAAAATGG
AAGAGACATGTATGTGGACCAAGAATTAGATATTAATCGTTTAAGTGATTATGATGT
CGATCACATTGTTCCACAAAGTTTCCTTAAAGACGATTCAATAGACAATAAGGTCTT
AACGCGTTCTGATAAAAATCGTGGTAAATCGGATAACGTTCCAAGTGAAGAAGTAG
TCAAAAAGATGAAAAACTATTGGAGACAACTTCTAAACGCCAAGTTAATCACTCAA
CGTAAGTTTGATAATTTAACGAAAGCTGAACGTGGAGGTTTGAGTGAACTTGATAAA
GCTGGTTTTATCAAACGCCAATTGGTTGAAACTCGCCAAATCACTAAGCATGTGGCA
CAAATTTTGGATAGTCGCATGAATACTAAATACGATGAAAATGATAAACTTATTCGA
GAGGTTAAAGTGATTACCTTAAAATCTAAATTAGTTTCTGACTTCCGAAAAGATTTC
CAATTCTATAAAGTACGTGAGATTAACAATTACCATCATGCCCATGATGCGTATCTA
AATGCCGTCGTTGGAACTGCTTTGATTAAGAAATATCCAAAACTTGAATCGGAGTTT
GTCTATGGTGATTATAAAGTTTATGATGTTCGTAAAATGATTGCTAAGTCTGAGCAA
GAAATAGGCAAAGCAACCGCAAAATATTTCTTTTACTCTAATATCATGAACTTCTTC
AAAACAGAAATTACACTTGCAAATGGAGAGATTCGCAAACGCCCTCTAATCGAAAC
TAATGGGGAAACTGGAGAAATTGTCTGGGATAAAGGGCGAGATTTTGCCACAGTGC
GCAAAGTATTGTCCATGCCCCAAGTCAATATTGTCAAGAAAACAGAAGTACAGACA
GGCGGATTCTCCAAGGAGTCAATTTTACCAAAAAGAAATTCGGACAAGCTTATTGCT
CGTAAAAAAGACTGGGATCCAAAAAAATATGGTGGTTTTGATAGTCCAACGGTAGC
TTATTCAGTCCTAGTGGTTGCTAAGGTGGAAAAAGGGAAATCGAAGAAGTTAAAAT
CCGTTAAAGAGTTACTAGGGATCACAATTATGGAAAGAAGTTCCTTTGAAAAAAAT
CCGATTGACTTTTTAGAAGCTAAAGGATATAAGGAAGTTAAAAAAGACTTAATCATT
AAACTACCTAAATATAGTCTTTTTGAGTTAGAAAACGGTCGTAAACGGATGCTGGCT
AGTGCCGGAGAATTACAAAAAGGAAATGAGCTGGCTCTGCCAAGCAAATATGTGAA
TTTTTTATATTTAGCTAGTCATTATGAAAAGTTGAAGGGTAGTCCAGAAGATAACGA
ACAAAAACAATTGTTTGTGGAGCAGCATAAGCATTATTTAGATGAGATTATTGAGCA
AATCAGTGAATTTTCTAAGCGTGTTATTTTAGCAGATGCCAATTTAGATAAAGTTCTT
AGTGCATATAACAAACATAGAGACAAACCAATACGTGAACAAGCAGAAAATATTAT
TCATTTATTTACGTTGACGAATCTTGGAGCTCCCGCTGCTTTTAAATATTTTGATACA
ACAATTGATCGTAAACGATATACGTCTACAAAAGAAGTTTTAGATGCCACTCTTATC
CATCAATCCATCACTGGTCTTTATGAAACACGCATTGATTTGAGTCAGCTAGGAGGT
GACTGATAATCAAATGTCAGACGAAAATGCCAATTATTGAAGCGGCTAACGCCGCT
TTTTTTGTTTCTGGTCTCCCTTGCTTGTACTTTACAGTATAGCTTCTAGAGATCTGCAG
TGACTGGAGCTTCTAGAGATCTGCAGTGACTGGGAAAACCCTGGCGACTAGTCTTGG
ACTCCTGTTGATAGATCCAGTAATGACCTCAGAACTCCATCTGGATTTGTTCAGAAC
GCTCGGTTGCCGCCGGGCGTTTTTTATTGGTGAGAATCCAGGGGTCCCCAATAATTA
CGATTTAAATCCTTCAAACTTCCCAAAGGCGAGCCCTAGTGACATTAGAAAACCGAC
TGTAAAAAGTACAGTCGGCATTATCTCATATTATAAAAGCCAGTCATTAGGCCTATC
TGACAATTCCTGAATAGAGTTCATAAACAATCCTGCATGATAACCATCACAAACAGA
ATGATGTACCTGTAAAGATAGCGGTAAATATATTGAATTACCTTTATTAATGAATTT
TCCTGCTGTAATAATGGGTAGAAGGTAATTACTATTATTATTGATATTTAAGTTAAA
CCCAGTAAATGAAGTCCATGGAATAATAGAAAGAGAAAAAGCATTTTCAGGTATAG
GTGTTTTGGGAAACAATTTCCCCGAACCATTATATTTCTCTACATCAGAAAGGTATA
AATCATAAAACTCTTTGAAGTCATTCTTTACAGGAGTCCAAATACCAGAGAATGTTT
TAGATACACCATCAAAAATTGTATAAAGTGGCTCTAACTTATCCCAATAACCTAACT
CTCCGTCGCTATTGTAACCAGTTCTAAAAGCTGTATTTGAGTTTATCACCCTTGTCAC
TAAGAAAATAAATGCAGGGTAAAATTTATATCCTTCTTGTTTTATGTTTCGGTATAA
AACACTAATATCAATTTCTGTGGTTATACTAAAAGTCGTTTGTTGGTTCAAATAATG
ATTAAATATCTCTTTTCTCTTCCAATTGTCTAAATCAATTTTATTAAAGTTCATTTGAT
ATGCCTCCTAAATTTTTATCTAAAGTGAATTTAGGAGGCTTACTTGTCTGCTTTCTTC
ATTAGAATCAATCCTTTTTTAAAAGTCAATCCCGTTTGTTGAAGACTTTTGTCCTTTT
CCGCTGCATAAACAATATGGCCCGTTTGTTGAACTACTCTTTAATAAAATAATTTTTC
CGTTCCCAATTCCACATTGCAATAATAGAAAATCCATCTTCATCGGCTTTTTCGTCAT
CATCTGTATGAATCAAATCGCCTTCTTCTGTGTCATCAAGGTTTAATTTTTTATGTAT
TTCTTTTAACAAACCACCATAGGAGATTAACCTTTTACGGTGTAAACCTTCCTCCAA
ATCAGACAAACGTTTCAAATTCTTTTCTTCATCATCGGTCATAAAATCCGTATCCTTT
ACAGGATATTTTGCAGTTTCGTCAATTGCCGATTGTATATCCGATTTATATTTATTTT
TCGGTCGAATCATTTGAACTTTTACATTTGGATCATAGTCTAATTTCATTGCCTTTTTC
CAAAATTGAATCCATTGTTTTTGATTCACGTAGTTTTCTGTATTCTTAAAATAAGTTG
GTTCCACACATACCAATACATGCATGTGCTGATTATAAGAATTATCTTTATTATTTAT
TGTCACTTCCGTTGCACGCATAAAACCAACAAGATTTTTATTAATTTTTTTATATTGC
ATCATTCGGCGAAATCCTTGAGCCATATCTGACAAACTCTTATTTAATTCTTCGCCAT
CATAAACATTTTTAACTGTTAATGTGAGAAACAACCAACGAACTGTTGGCTTTTGTT
TAATAACTTCAGCAACAACCTTTTGTGACTGAATGCCATGTTTCATTGCTCTCCTCCA
GTTGCACATTGGACAAAGCCTGGATTTACAAAACCACACTCGATACAACTTTCTTTC
GCCTGTTTCACGATTTTGTTTATACTCTAATATTTCAGCACAATCTTTTACTCTTTCAG
CCTTTTTAAATTCAAGAATATGCAGAAGTTCAAAGTAATCAACATTAGCGATTTTCT
TTTCTCTCCATGGTCTCACTTTTCCACTTTTTGTCTTGTCCACTAAAACCCTTGATTTT
TCATCTGAATAAATGCTACTATTAGGACACATAATATTAAAAGAAACCCCCATCTAT
TTAGTTATTTGTTTGGTCACTTATAACTTTAACAGATGGGGTTTTTCTGTGCAACCAA
TTTTAAGGGTTTTCCAATACTTTAAAACACATACATACCAACACTTCAACGCACCTTT
CAGCAACTAAAATAAAAATGACGTTATTTCTATATGTATCAAGATAAGAAAGAACA
AGTTCAAAACCATCAAAAAAAGACACCTTTTCAGGTGCTTTTTTTATTTTATAAACTC
ATTCCCTGATCTCGACTTCGTTCTTTTTTTACCTCTCGGTTATGAGTTAGTTCAAATTC
GTTCTTTTTAGGTTCTAAATCGTGTTTTTCTTGGAATTGTGCTGTTTTATCCTTTACCT
TGTCTACAAACCCCTTAAAAACGTTTTTAAAGGCTTTTAAGCGTCTGTACGTTCCTTA
AGGAATTATTCCTTAGTGCTTTCTAGGTTAATGTCATGATAATAATGGTTTCTTAGAC
GTCAGGTGGCACTTTTCGGGGAAATGTCCGCGGAACCCCTATTTGTTTATTTTTCTAA
ATACATTCAAATATGTATCCGCTCATGAGACAATAACCCTGATAAATGCTTCAATAA
TATTGAAAAAGGAAGAGTATGAGTATTCAACATTTCCGTGTCGCCCTTATTCCCTTTT
TTGCGGCATTTTGCCTTCCTGTTTTTGCTCACCCAGAAACGCTGGTGAAAGTAAAAG
ATGCTGAAGATCAGTTGGGTGCACGAGTGGGTTACATCGAACTGGATCTCAACAGC
GGTAAGATCCTTGAGAGTTTTCGCCCCGAAGAACGTTTTCCAATGATGAGCACTTTT
AAAGTTCTGCTATGTGGCGCGGTATTATCCCGTGTTGACGCCGGGCAAGAGCAACTC
GGTCGCCGCATACACTATTCTCAGAATGACTTGGTTGAGTACTCACCAGTCACAGAA
AAGCATCTTACGGATGGCATGACAGTAAGAGAATTATGCAGTGCTGCCATAACCAT
GAGTGATAACACTGCGGCCAACTTACTTCTGACAACGATCGGAGGACCGAAGGAGC
TAACCGCTTTTTTGCACAACATGGGGGATCATGTAACTCGCCTTGATCGTTGGGAAC
CGGAGCTGAATGAAGCCATACCAAACGACGAGCGTGACACCACGATGCCTGCAGCA
ATGGCAACAACGTTGCGCAAACTATTAACTGGCGAACTACTTACTCTAGCTTCCCGG
CAACAATTAATAGACTGGATGGAGGCGGATAAAGTTGCAGGACCACTTCTGCGCTC
GGCCCTTCCGGCTGGCTGGTTTATTGCTGATAAATCTGGAGCCGGTGAGCGTGGGTC
TCGCGGTATCATTGCAGCACTGGGGCCAGATGGTAAGCCCTCCCGTATCGTAGTTAT
CTACACGACGGGGAGTCAGGCAACTATGGATGAACGAAATAGACAGATCGCTGAGA
TAGGTGCCTCACTGATTAAGCATTGGTAACTGTCAGACCAAGTTTACTCATATATAC
TTTAGATTGATTTAAAACTTCATTTTTAATTTAAAAGGATCTAGGTGAAGATCCTTTT
TGATAATCTCATGACCAAAATCCCTTAACGTGAGTTTTCGTTCCACTGAGCGTCAGA
CCCCTTAATAAGATGATCTTCTTGAGATCGTTTTGGTCTGCGCGTAATCTCTTGCTCT
GAAAACGAAAAAACCGCCTTGCAGGGAGGTTTTTCGAAGGTTCTCTGAGCTACCAA
CTCTTTGAACCGAGGTAACTGGCTTGCAGGAGCGCAGTCACCAAAACTTGTCCTTTC
AGTTTAGCCTTAACCGGCGCATGACTTCAAGACTAACTCCTCTAAATCAATTACCAG
TGGCTGCTGCCAGTGGTGCTTTTGCATGTCTTTCCGGGTTGGACTCAAGACGATAGTT
ACCGGATAAGGCGCAGCGGTCGGACTGAACGGGGGGTTCGTGCATACAGTCCAGCT
TGGAGCGAACTGCCTACCCGGAACTGAGTGTCAGGCGTGGAATGAGACAAACGCGG
CCATAACAGCGGAATGACACCGGTAAACCGAAAGGCAGGAACAGGAGAGCGCACG
AGGGAGCCGCCAGGGGGAAACGCCTGGTATCTTTATAGTCCTGTCGGGTTTCGCCAC
CACTGATTTGAGCGTCAGATTTCGTGATGCTTGTCAGGGGGCGGAGCCTATGGAAAA
ACGCTTTGCCCGAGCCTATGGAAAAACGCTTTGCCCCACACAGGCTCTTGAACAAGG
GATAACAATTTGTCATAGCTGTATTCGTTACAAGGAGGATTTTCGAAAAAACACCCT
AACGGGTGTTTTTTTATAGCTGGTCTCCCCAAGTGCTGTAAAAAACGCCGAATGAAG
CGGATGAAATTGCTCATCGTCTCGTGAAAAAGCCGAAGAAGGTTAAGCCTGGGTAT
AAAAAGAAAATGAGCTATGAGATGGAGAAAGCCATAACCATGAGTGATAACACTG
GTTTTAGAGCTAGAAATAGCAAGTTAAAATAAGGCTAGTCCGTTATCAACTTGAAAA
AGTGGCACCGAGTCGGTGCTTTTTTTCTCGGTACCAAATTCCAGAAAAGAGGCCTCC
CGAAAGGGGGGCCTTTTTTCGTTTTGGTCCCTCAATGATC
>pCas9g2
(SEQ ID No: 234)
tccttatccggttaagatggCTTTTAAATCAATTTTCAGCTCCTGTATACAATTACCAAAG
TTTTTCTGAATGAAGCCATGTGTTTTGACACATTCTATACTCACAAGGAGGTGAGACACat
ggataagaaatactcaataggctTAGATATCGGCACAAATAGCGTCGGATGGGCGGTGATC
ACTGATGAATATAAGGTTCCGTCTAAAAAGTTCAAGGTTCTGGGAAATACAGACCGCCACA
GTATCAAAAAAAATCTTATAGGGGCTCTTTTATTTGACAGTGGAGAGACAGCGGAAG
CGACTCGTCTCAAACGGACAGCTCGTAGAAGGTATACACGTCGGAAGAATCGTATTT
GTTATCTACAGGAGATTTTTTCAAATGAGATGGCGAAAGTAGATGATAGTTTCTTTC
ATCGACTTGAAGAGTCTTTTTTGGTGGAAGAAGACAAGAAGCATGAACGTCATCCTA
TTTTTGGAAATATAGTAGATGAAGTTGCTTATCATGAGAAATATCCAACTATCTATC
ATCTGCGAAAAAAATTGGTAGATTCTACTGATAAAGCGGATTTGCGCTTAATCTATT
TGGCCTTAGCGCATATGATTAAGTTTCGTGGTCATTTTTTGATTGAGGGAGATTTAAA
TCCTGATAATAGTGATGTGGACAAACTATTTATCCAGTTGGTACAAACCTACAATCA
ATTATTTGAAGAAAACCCTATTAACGCAAGTGGAGTAGATGCTAAAGCGATTCTTTC
TGCACGATTGAGTAAATCAAGACGATTAGAAAATCTCATTGCTCAGCTCCCCGGTGA
GAAGAAAAATGGCTTATTTGGGAATCTCATTGCTTTGTCATTGGGTTTGACCCCTAA
TTTTAAATCAAATTTTGATTTGGCAGAAGATGCTAAATTACAGCTTTCAAAAGATAC
TTACGATGATGATTTAGATAATTTATTGGCGCAAATTGGAGATCAATATGCTGATTT
GTTTTTGGCAGCTAAGAATTTATCAGATGCTATTTTACTTTCAGATATCCTAAGAGTA
AATACTGAAATAACTAAGGCTCCCCTATCAGCTTCAATGATTAAACGCTACGATGAA
CATCATCAAGACTTGACTCTTTTAAAAGCTTTAGTTCGACAACAACTTCCAGAAAAG
TATAAAGAAATCTTTTTTGATCAATCAAAAAACGGATATGCAGGTTATATTGATGGG
GGAGCTAGCCAAGAAGAATTTTATAAATTTATCAAACCAATTTTAGAAAAAATGGA
TGGTACTGAGGAATTATTGGTGAAACTAAATCGTGAAGATTTGCTGCGCAAGCAAC
GGACCTTTGACAACGGCTCTATTCCCCATCAAATTCACTTGGGTGAGCTGCATGCTA
TTTTGAGAAGACAAGAAGACTTTTATCCATTTTTAAAAGACAATCGTGAGAAGATTG
AAAAAATCTTGACTTTTCGAATTCCTTATTATGTTGGTCCATTGGCGCGTGGCAATAG
TCGTTTTGCATGGATGACTCGGAAGTCTGAAGAAACAATTACCCCATGGAATTTTGA
AGAAGTTGTCGATAAAGGTGCTTCAGCTCAATCATTTATTGAACGCATGACAAACTT
TGATAAAAATCTTCCAAATGAAAAAGTACTACCAAAACATAGTTTGCTTTATGAGTA
TTTTACGGTTTATAACGAATTGACAAAGGTCAAATATGTTACTGAAGGAATGCGAAA
ACCAGCATTTCTTTCAGGTGAACAGAAGAAAGCCATTGTTGATTTACTCTTCAAAAC
AAATCGAAAAGTAACCGTTAAGCAATTAAAAGAAGATTATTTCAAAAAAATAGAAT
GTTTTGATAGTGTTGAAATTTCAGGAGTTGAAGATAGATTTAATGCTTCATTAGGTA
CCTACCATGATTTGCTAAAAATTATTAAAGATAAAGATTTTTTGGATAATGAAGAAA
ATGAAGATATCTTAGAGGATATTGTTTTAACATTGACCTTATTTGAAGATAGGGAGA
TGATTGAGGAAAGACTTAAAACATATGCTCACCTCTTTGATGATAAGGTGATGAAAC
AGCTTAAACGTCGCCGTTATACTGGTTGGGGACGTTTGTCTCGAAAATTGATTAATG
GTATTAGGGATAAGCAATCTGGCAAAACAATATTAGATTTTTTGAAATCAGATGGTT
TTGCCAATCGCAATTTTATGCAGCTGATCCATGATGATAGTTTGACATTTAAAGAAG
ACATTCAAAAAGCACAAGTGTCTGGACAAGGCGATAGTTTACATGAACATATTGCA
AATTTAGCTGGTAGCCCTGCTATTAAAAAAGGTATTTTACAGACTGTAAAAGTTGTT
GATGAATTGGTCAAAGTAATGGGGCGGCATAAGCCAGAAAATATCGTTATTGAAAT
GGCACGTGAAAATCAGACAACTCAAAAGGGCCAGAAAAATTCGCGAGAGCGTATG
AAACGAATCGAAGAAGGTATCAAAGAATTAGGAAGTCAGATTCTTAAAGAGCATCC
TGTTGAAAATACTCAATTGCAAAATGAAAAGCTCTATCTCTATTATCTCCAAAATGG
AAGAGACATGTATGTGGACCAAGAATTAGATATTAATCGTTTAAGTGATTATGATGT
CGATCACATTGTTCCACAAAGTTTCCTTAAAGACGATTCAATAGACAATAAGGTCTT
AACGCGTTCTGATAAAAATCGTGGTAAATCGGATAACGTTCCAAGTGAAGAAGTAG
TCAAAAAGATGAAAAACTATTGGAGACAACTTCTAAACGCCAAGTTAATCACTCAA
CGTAAGTTTGATAATTTAACGAAAGCTGAACGTGGAGGTTTGAGTGAACTTGATAAA
GCTGGTTTTATCAAACGCCAATTGGTTGAAACTCGCCAAATCACTAAGCATGTGGCA
CAAATTTTGGATAGTCGCATGAATACTAAATACGATGAAAATGATAAACTTATTCGA
GAGGTTAAAGTGATTACCTTAAAATCTAAATTAGTTTCTGACTTCCGAAAAGATTTC
CAATTCTATAAAGTACGTGAGATTAACAATTACCATCATGCCCATGATGCGTATCTA
AATGCCGTCGTTGGAACTGCTTTGATTAAGAAATATCCAAAACTTGAATCGGAGTTT
GTCTATGGTGATTATAAAGTTTATGATGTTCGTAAAATGATTGCTAAGTCTGAGCAA
GAAATAGGCAAAGCAACCGCAAAATATTTCTTTTACTCTAATATCATGAACTTCTTC
AAAACAGAAATTACACTTGCAAATGGAGAGATTCGCAAACGCCCTCTAATCGAAAC
TAATGGGGAAACTGGAGAAATTGTCTGGGATAAAGGGCGAGATTTTGCCACAGTGC
GCAAAGTATTGTCCATGCCCCAAGTCAATATTGTCAAGAAAACAGAAGTACAGACA
GGCGGATTCTCCAAGGAGTCAATTTTACCAAAAAGAAATTCGGACAAGCTTATTGCT
CGTAAAAAAGACTGGGATCCAAAAAAATATGGTGGTTTTGATAGTCCAACGGTAGC
TTATTCAGTCCTAGTGGTTGCTAAGGTGGAAAAAGGGAAATCGAAGAAGTTAAAAT
CCGTTAAAGAGTTACTAGGGATCACAATTATGGAAAGAAGTTCCTTTGAAAAAAAT
CCGATTGACTTTTTAGAAGCTAAAGGATATAAGGAAGTTAAAAAAGACTTAATCATT
AAACTACCTAAATATAGTCTTTTTGAGTTAGAAAACGGTCGTAAACGGATGCTGGCT
AGTGCCGGAGAATTACAAAAAGGAAATGAGCTGGCTCTGCCAAGCAAATATGTGAA
TTTTTTATATTTAGCTAGTCATTATGAAAAGTTGAAGGGTAGTCCAGAAGATAACGA
ACAAAAACAATTGTTTGTGGAGCAGCATAAGCATTATTTAGATGAGATTATTGAGCA
AATCAGTGAATTTTCTAAGCGTGTTATTTTAGCAGATGCCAATTTAGATAAAGTTCTT
AGTGCATATAACAAACATAGAGACAAACCAATACGTGAACAAGCAGAAAATATTAT
TCATTTATTTACGTTGACGAATCTTGGAGCTCCCGCTGCTTTTAAATATTTTGATACA
ACAATTGATCGTAAACGATATACGTCTACAAAAGAAGTTTTAGATGCCACTCTTATC
CATCAATCCATCACTGGTCTTTATGAAACACGCATTGATTTGAGTCAGCTAGGAGGT
GACTGATAATCAAATGTCAGACGAAAATGCCAATTATTGAAGCGGCTAACGCCGCT
TTTTTTGTTTCTGGTCTCCCTTGCTTGTACTTTACAGTATAGCTTCTAGAGATCTGCAG
TGACTGGAGCTTCTAGAGATCAGGTCAGACGCGATTTCCTGGGTGCTCGAGCCATGG
GAAAGCGGTGGTGGAAAAAACGCCGAATGAAGCGGATGAAATTGCTCATCGTCTCG
TGAAAAAGCCGAAGAAGGTTAAGCCTGGGTATAAAAAGAAAATGAGCTATGAGAT
GGAGAAATTGAAGTCATGCGCCGGTTAGTTTTAGAGCTAGAAATAGCAAGTTAAAA
TAAGGCTAGTCCGTTATCAACTTGAAAAAGTGGCACCGAGTCGGTGCTTTTTTTCTC
GGTACCAAATTCCAGAAAAGAGGCCTCCCGAAAGGGGGGCCTTTTTTCGTTTTGGTC
CCTCAATGATCCGACAGGAAGTGGTATTAGCGACTCCTACAAGGAATAGGTGTCTG
GGTCATGCAGTGACTGGGAAAACCCTGGCGACTAGTCTTGGACTCCTGTTGATAGAT
CCAGTAATGACCTCAGAACTCCATCTGGATTTGTTCAGAACGCTCGGTTGCCGCCGG
GCGTTTTTTATTGGTGAGAATCCAGGGGTCCCCAATAATTACGATTTAAATCCTTCA
AACTTCCCAAAGGCGAGCCCTAGTGACATTAGAAAACCGACTGTAAAAAGTACAGT
CGGCATTATCTCATATTATAAAAGCCAGTCATTAGGCCTATCTGACAATTCCTGAAT
AGAGTTCATAAACAATCCTGCATGATAACCATCACAAACAGAATGATGTACCTGTA
AAGATAGCGGTAAATATATTGAATTACCTTTATTAATGAATTTTCCTGCTGTAATAAT
GGGTAGAAGGTAATTACTATTATTATTGATATTTAAGTTAAACCCAGTAAATGAAGT
CCATGGAATAATAGAAAGAGAAAAAGCATTTTCAGGTATAGGTGTTTTGGGAAACA
ATTTCCCCGAACCATTATATTTCTCTACATCAGAAAGGTATAAATCATAAAACTCTTT
GAAGTCATTCTTTACAGGAGTCCAAATACCAGAGAATGTTTTAGATACACCATCAAA
AATTGTATAAAGTGGCTCTAACTTATCCCAATAACCTAACTCTCCGTCGCTATTGTAA
CCAGTTCTAAAAGCTGTATTTGAGTTTATCACCCTTGTCACTAAGAAAATAAATGCA
GGGTAAAATTTATATCCTTCTTGTTTTATGTTTCGGTATAAAACACTAATATCAATTT
CTGTGGTTATACTAAAAGTCGTTTGTTGGTTCAAATAATGATTAAATATCTCTTTTCT
CTTCCAATTGTCTAAATCAATTTTATTAAAGTTCATTTGATATGCCTCCTAAATTTTT
ATCTAAAGTGAATTTAGGAGGCTTACTTGTCTGCTTTCTTCATTAGAATCAATCCTTT
TTTAAAAGTCAATCCCGTTTGTTGAAGACTTTTGTCCTTTTCCGCTGCATAAACAATA
TGGCCCGTTTGTTGAACTACTCTTTAATAAAATAATTTTTCCGTTCCCAATTCCACAT
TGCAATAATAGAAAATCCATCTTCATCGGCTTTTTCGTCATCATCTGTATGAATCAAA
TCGCCTTCTTCTGTGTCATCAAGGTTTAATTTTTTATGTATTTCTTTTAACAAACCACC
ATAGGAGATTAACCTTTTACGGTGTAAACCTTCCTCCAAATCAGACAAACGTTTCAA
ATTCTTTTCTTCATCATCGGTCATAAAATCCGTATCCTTTACAGGATATTTTGCAGTT
TCGTCAATTGCCGATTGTATATCCGATTTATATTTATTTTTCGGTCGAATCATTTGAA
CTTTTACATTTGGATCATAGTCTAATTTCATTGCCTTTTTCCAAAATTGAATCCATTGT
TTTTGATTCACGTAGTTTTCTGTATTCTTAAAATAAGTTGGTTCCACACATACCAATA
CATGCATGTGCTGATTATAAGAATTATCTTTATTATTTATTGTCACTTCCGTTGCACG
CATAAAACCAACAAGATTTTTATTAATTTTTTTATATTGCATCATTCGGCGAAATCCT
TGAGCCATATCTGACAAACTCTTATTTAATTCTTCGCCATCATAAACATTTTTAACTG
TTAATGTGAGAAACAACCAACGAACTGTTGGCTTTTGTTTAATAACTTCAGCAACAA
CCTTTTGTGACTGAATGCCATGTTTCATTGCTCTCCTCCAGTTGCACATTGGACAAAG
CCTGGATTTACAAAACCACACTCGATACAACTTTCTTTCGCCTGTTTCACGATTTTGT
TTATACTCTAATATTTCAGCACAATCTTTTACTCTTTCAGCCTTTTTAAATTCAAGAA
TATGCAGAAGTTCAAAGTAATCAACATTAGCGATTTTCTTTTCTCTCCATGGTCTCAC
TTTTCCACTTTTTGTCTTGTCCACTAAAACCCTTGATTTTTCATCTGAATAAATGCTAC
TATTAGGACACATAATATTAAAAGAAACCCCCATCTATTTAGTTATTTGTTTGGTCAC
TTATAACTTTAACAGATGGGGTTTTTCTGTGCAACCAATTTTAAGGGTTTTCCAATAC
TTTAAAACACATACATACCAACACTTCAACGCACCTTTCAGCAACTAAAATAAAAAT
GACGTTATTTCTATATGTATCAAGATAAGAAAGAACAAGTTCAAAACCATCAAAAA
AAGACACCTTTTCAGGTGCTTTTTTTATTTTATAAACTCATTCCCTGATCTCGACTTC
GTTCTTTTTTTACCTCTCGGTTATGAGTTAGTTCAAATTCGTTCTTTTTAGGTTCTAAA
TCGTGTTTTTCTTGGAATTGTGCTGTTTTATCCTTTACCTTGTCTACAAACCCCTTAAA
AACGTTTTTAAAGGCTTTTAAGCGTCTGTACGTTCCTTAAGGAATTATTCCTTAGTGC
TTTCTAGGTTAATGTCATGATAATAATGGTTTCTTAGACGTCAGGTGGCACTTTTCGG
GGAAATGTCCGCGGAACCCCTATTTGTTTATTTTTCTAAATACATTCAAATATGTATC
CGCTCATGAGACAATAACCCTGATAAATGCTTCAATAATATTGAAAAAGGAAGAGT
ATGAGTATTCAACATTTCCGTGTCGCCCTTATTCCCTTTTTTGCGGCATTTTGCCTTCC
TGTTTTTGCTCACCCAGAAACGCTGGTGAAAGTAAAAGATGCTGAAGATCAGTTGGG
TGCACGAGTGGGTTACATCGAACTGGATCTCAACAGCGGTAAGATCCTTGAGAGTTT
TCGCCCCGAAGAACGTTTTCCAATGATGAGCACTTTTAAAGTTCTGCTATGTGGCGC
GGTATTATCCCGTGTTGACGCCGGGCAAGAGCAACTCGGTCGCCGCATACACTATTC
TCAGAATGACTTGGTTGAGTACTCACCAGTCACAGAAAAGCATCTTACGGATGGCAT
GACAGTAAGAGAATTATGCAGTGCTGCCATAACCATGAGTGATAACACTGCGGCCA
ACTTACTTCTGACAACGATCGGAGGACCGAAGGAGCTAACCGCTTTTTTGCACAACA
TGGGGGATCATGTAACTCGCCTTGATCGTTGGGAACCGGAGCTGAATGAAGCCATA
CCAAACGACGAGCGTGACACCACGATGCCTGCAGCAATGGCAACAACGTTGCGCAA
ACTATTAACTGGCGAACTACTTACTCTAGCTTCCCGGCAACAATTAATAGACTGGAT
GGAGGCGGATAAAGTTGCAGGACCACTTCTGCGCTCGGCCCTTCCGGCTGGCTGGTT
TATTGCTGATAAATCTGGAGCCGGTGAGCGTGGGTCTCGCGGTATCATTGCAGCACT
GGGGCCAGATGGTAAGCCCTCCCGTATCGTAGTTATCTACACGACGGGGAGTCAGG
CAACTATGGATGAACGAAATAGACAGATCGCTGAGATAGGTGCCTCACTGATTAAG
CATTGGTAACTGTCAGACCAAGTTTACTCATATATACTTTAGATTGATTTAAAACTTC
ATTTTTAATTTAAAAGGATCTAGGTGAAGATCCTTTTTGATAATCTCATGACCAAAA
TCCCTTAACGTGAGTTTTCGTTCCACTGAGCGTCAGACCCCTTAATAAGATGATCTTC
TTGAGATCGTTTTGGTCTGCGCGTAATCTCTTGCTCTGAAAACGAAAAAACCGCCTT
GCAGGGAGGTTTTTCGAAGGTTCTCTGAGCTACCAACTCTTTGAACCGAGGTAACTG
GCTTGCAGGAGCGCAGTCACCAAAACTTGTCCTTTCAGTTTAGCCTTAACCGGCGCA
TGACTTCAAGACTAACTCCTCTAAATCAATTACCAGTGGCTGCTGCCAGTGGTGCTT
TTGCATGTCTTTCCGGGTTGGACTCAAGACGATAGTTACCGGATAAGGCGCAGCGGT
CGGACTGAACGGGGGGTTCGTGCATACAGTCCAGCTTGGAGCGAACTGCCTACCCG
GAACTGAGTGTCAGGCGTGGAATGAGACAAACGCGGCCATAACAGCGGAATGACAC
CGGTAAACCGAAAGGCAGGAACAGGAGAGCGCACGAGGGAGCCGCCAGGGGGAAA
CGCCTGGTATCTTTATAGTCCTGTCGGGTTTCGCCACCACTGATTTGAGCGTCAGATT
TCGTGATGCTTGTCAGGGGGCGGAGCCTATGGAAAAACGCTTTGCCCGAGCCTATGG
AAAAACGCTTTGCCCCACACAGGCTCTTGAACAAGGGATAACAATTTGTCATAGCTG
TATTCGTTACAAGGAGGATTTTCGAAAAAACACCCTAACGGGTGTTTTTTTATAGCT
GGTCTCCCCAAGTGCTGTAAAAAACGCCGAATGAAGCGGATGAAATTGCTCATCGT
CTCGTGAAAAAGCCGAAGAAGGTTAAGCCTGGGTATAAAAAGAAAATGAGCTATGA
GATGGAGAAAGCCATAACCATGAGTGATAACACTGGTTTTAGAGCTAGAAATAGCA
AGTTAAAATAAGGCTAGTCCGTTATCAACTTGAAAAAGTGGCACCGAGTCGGTGCTT
TTTTTCTCGGTACCAAATTCCAGAAAAGAGGCCTCCCGAAAGGGGGGCCTTTTTTCG
TTTTGGTCCCTCAATGATC
>pCas9g1-D15
(SEQ ID No: 235)
tccttatccggttaagatggCTTTTAAATCAATTTTCAGCTCCTGTATACAATTACCAAA
GTTTTTCTGAATGAAGCCATGTGTTTTGACACATTCTATACTCACAAGGAGGTGAGACAC
atgagtaaatcctggggcaaATTCATCGAGGAGGAAGAGGCGGAAATGGCTTCCCGTCGT
AATCTAATGATTGTCGATGGAACTAACTTAGGCTTTCGCTTCAAACATAACAATAGTAAA
AAACCATTTGCCTCAAGTTATGTTTCAACTATTCAATCTCTGGCAAAATCCTACTCTGCC
AGAACTACGATTGTTCTAGGTGATAAGGGCAAAAGCGTGTTCAGACTAGAACATCTACC
AGAGTATAAAGGTAATCGTGATGAAAAGTACGCACAACGTACGGAAGAGGAGAAA
GCGCTAGATGAGCAGTTCTTCGAATACCTCAAAGATGCTTTCGAGTTGTGTAAAACT
ACATTCCCAACTTTTACCATTCGTGGTGTAGAAGCAGATGACATGGCGGCGTATATT
GTTAAGCTCATCGGGCATCTTTATGATCACGTTTGGCTAATATCTACAGATGGTGAC
TGGGATACTTTATTAACGGATAAAGTTTCTCGTTTTTCTTTCACAACACGTCGTGAGT
ATCATCTTCGTGATATGTATGAGCACCATAACGTGGACGACGTAGAACAGTTTATCT
CCCTGAAAGCAATTATGGGAGATCTAGGAGATAATATTCGTGGTGTTGAAGGAATA
GGAGCAAAACGCGGATATAATATTATTCGTGAGTTTGGTAACGTACTGGATATTATT
GATCAGCTTCCACTGCCTGGAAAGCAGAAATATATACAGAACCTGAATGCATCGGA
AGAACTGCTTTTCCGAAACTTGATTCTGGTTGATTTACCTACCTACTGTGTGGATGCT
ATTGCTGCTGTAGGTCAAGATGTGTTAGATAAGTTCACGAAAGACATCCTTGAGATC
GCGGAACAATGATAAGCTCTTAAAGGGGGTTTTAGATACCAGAGATGGATAAGAAA
TACTCAATAGGCTTAGATATCGGCACAAATAGCGTCGGATGGGCGGTGATCACTGAT
GAATATAAGGTTCCGTCTAAAAAGTTCAAGGTTCTGGGAAATACAGACCGCCACAG
TATCAAAAAAAATCTTATAGGGGCTCTTTTATTTGACAGTGGAGAGACAGCGGAAG
CGACTCGTCTCAAACGGACAGCTCGTAGAAGGTATACACGTCGGAAGAATCGTATTT
GTTATCTACAGGAGATTTTTTCAAATGAGATGGCGAAAGTAGATGATAGTTTCTTTC
ATCGACTTGAAGAGTCTTTTTTGGTGGAAGAAGACAAGAAGCATGAACGTCATCCTA
TTTTTGGAAATATAGTAGATGAAGTTGCTTATCATGAGAAATATCCAACTATCTATC
ATCTGCGAAAAAAATTGGTAGATTCTACTGATAAAGCGGATTTGCGCTTAATCTATT
TGGCCTTAGCGCATATGATTAAGTTTCGTGGTCATTTTTTGATTGAGGGAGATTTAAA
TCCTGATAATAGTGATGTGGACAAACTATTTATCCAGTTGGTACAAACCTACAATCA
ATTATTTGAAGAAAACCCTATTAACGCAAGTGGAGTAGATGCTAAAGCGATTCTTTC
TGCACGATTGAGTAAATCAAGACGATTAGAAAATCTCATTGCTCAGCTCCCCGGTGA
GAAGAAAAATGGCTTATTTGGGAATCTCATTGCTTTGTCATTGGGTTTGACCCCTAA
TTTTAAATCAAATTTTGATTTGGCAGAAGATGCTAAATTACAGCTTTCAAAAGATAC
TTACGATGATGATTTAGATAATTTATTGGCGCAAATTGGAGATCAATATGCTGATTT
GTTTTTGGCAGCTAAGAATTTATCAGATGCTATTTTACTTTCAGATATCCTAAGAGTA
AATACTGAAATAACTAAGGCTCCCCTATCAGCTTCAATGATTAAACGCTACGATGAA
CATCATCAAGACTTGACTCTTTTAAAAGCTTTAGTTCGACAACAACTTCCAGAAAAG
TATAAAGAAATCTTTTTTGATCAATCAAAAAACGGATATGCAGGTTATATTGATGGG
GGAGCTAGCCAAGAAGAATTTTATAAATTTATCAAACCAATTTTAGAAAAAATGGA
TGGTACTGAGGAATTATTGGTGAAACTAAATCGTGAAGATTTGCTGCGCAAGCAAC
GGACCTTTGACAACGGCTCTATTCCCCATCAAATTCACTTGGGTGAGCTGCATGCTA
TTTTGAGAAGACAAGAAGACTTTTATCCATTTTTAAAAGACAATCGTGAGAAGATTG
AAAAAATCTTGACTTTTCGAATTCCTTATTATGTTGGTCCATTGGCGCGTGGCAATAG
TCGTTTTGCATGGATGACTCGGAAGTCTGAAGAAACAATTACCCCATGGAATTTTGA
AGAAGTTGTCGATAAAGGTGCTTCAGCTCAATCATTTATTGAACGCATGACAAACTT
TGATAAAAATCTTCCAAATGAAAAAGTACTACCAAAACATAGTTTGCTTTATGAGTA
TTTTACGGTTTATAACGAATTGACAAAGGTCAAATATGTTACTGAAGGAATGCGAAA
ACCAGCATTTCTTTCAGGTGAACAGAAGAAAGCCATTGTTGATTTACTCTTCAAAAC
AAATCGAAAAGTAACCGTTAAGCAATTAAAAGAAGATTATTTCAAAAAAATAGAAT
GTTTTGATAGTGTTGAAATTTCAGGAGTTGAAGATAGATTTAATGCTTCATTAGGTA
CCTACCATGATTTGCTAAAAATTATTAAAGATAAAGATTTTTTGGATAATGAAGAAA
ATGAAGATATCTTAGAGGATATTGTTTTAACATTGACCTTATTTGAAGATAGGGAGA
TGATTGAGGAAAGACTTAAAACATATGCTCACCTCTTTGATGATAAGGTGATGAAAC
AGCTTAAACGTCGCCGTTATACTGGTTGGGGACGTTTGTCTCGAAAATTGATTAATG
GTATTAGGGATAAGCAATCTGGCAAAACAATATTAGATTTTTTGAAATCAGATGGTT
TTGCCAATCGCAATTTTATGCAGCTGATCCATGATGATAGTTTGACATTTAAAGAAG
ACATTCAAAAAGCACAAGTGTCTGGACAAGGCGATAGTTTACATGAACATATTGCA
AATTTAGCTGGTAGCCCTGCTATTAAAAAAGGTATTTTACAGACTGTAAAAGTTGTT
GATGAATTGGTCAAAGTAATGGGGCGGCATAAGCCAGAAAATATCGTTATTGAAAT
GGCACGTGAAAATCAGACAACTCAAAAGGGCCAGAAAAATTCGCGAGAGCGTATG
AAACGAATCGAAGAAGGTATCAAAGAATTAGGAAGTCAGATTCTTAAAGAGCATCC
TGTTGAAAATACTCAATTGCAAAATGAAAAGCTCTATCTCTATTATCTCCAAAATGG
AAGAGACATGTATGTGGACCAAGAATTAGATATTAATCGTTTAAGTGATTATGATGT
CGATCACATTGTTCCACAAAGTTTCCTTAAAGACGATTCAATAGACAATAAGGTCTT
AACGCGTTCTGATAAAAATCGTGGTAAATCGGATAACGTTCCAAGTGAAGAAGTAG
TCAAAAAGATGAAAAACTATTGGAGACAACTTCTAAACGCCAAGTTAATCACTCAA
CGTAAGTTTGATAATTTAACGAAAGCTGAACGTGGAGGTTTGAGTGAACTTGATAAA
GCTGGTTTTATCAAACGCCAATTGGTTGAAACTCGCCAAATCACTAAGCATGTGGCA
CAAATTTTGGATAGTCGCATGAATACTAAATACGATGAAAATGATAAACTTATTCGA
GAGGTTAAAGTGATTACCTTAAAATCTAAATTAGTTTCTGACTTCCGAAAAGATTTC
CAATTCTATAAAGTACGTGAGATTAACAATTACCATCATGCCCATGATGCGTATCTA
AATGCCGTCGTTGGAACTGCTTTGATTAAGAAATATCCAAAACTTGAATCGGAGTTT
GTCTATGGTGATTATAAAGTTTATGATGTTCGTAAAATGATTGCTAAGTCTGAGCAA
GAAATAGGCAAAGCAACCGCAAAATATTTCTTTTACTCTAATATCATGAACTTCTTC
AAAACAGAAATTACACTTGCAAATGGAGAGATTCGCAAACGCCCTCTAATCGAAAC
TAATGGGGAAACTGGAGAAATTGTCTGGGATAAAGGGCGAGATTTTGCCACAGTGC
GCAAAGTATTGTCCATGCCCCAAGTCAATATTGTCAAGAAAACAGAAGTACAGACA
GGCGGATTCTCCAAGGAGTCAATTTTACCAAAAAGAAATTCGGACAAGCTTATTGCT
CGTAAAAAAGACTGGGATCCAAAAAAATATGGTGGTTTTGATAGTCCAACGGTAGC
TTATTCAGTCCTAGTGGTTGCTAAGGTGGAAAAAGGGAAATCGAAGAAGTTAAAAT
CCGTTAAAGAGTTACTAGGGATCACAATTATGGAAAGAAGTTCCTTTGAAAAAAAT
CCGATTGACTTTTTAGAAGCTAAAGGATATAAGGAAGTTAAAAAAGACTTAATCATT
AAACTACCTAAATATAGTCTTTTTGAGTTAGAAAACGGTCGTAAACGGATGCTGGCT
AGTGCCGGAGAATTACAAAAAGGAAATGAGCTGGCTCTGCCAAGCAAATATGTGAA
TTTTTTATATTTAGCTAGTCATTATGAAAAGTTGAAGGGTAGTCCAGAAGATAACGA
ACAAAAACAATTGTTTGTGGAGCAGCATAAGCATTATTTAGATGAGATTATTGAGCA
AATCAGTGAATTTTCTAAGCGTGTTATTTTAGCAGATGCCAATTTAGATAAAGTTCTT
AGTGCATATAACAAACATAGAGACAAACCAATACGTGAACAAGCAGAAAATATTAT
TCATTTATTTACGTTGACGAATCTTGGAGCTCCCGCTGCTTTTAAATATTTTGATACA
ACAATTGATCGTAAACGATATACGTCTACAAAAGAAGTTTTAGATGCCACTCTTATC
CATCAATCCATCACTGGTCTTTATGAAACACGCATTGATTTGAGTCAGCTAGGAGGT
GACTGATAATCAAATGTCAGACGAAAATGCCAATTATTGAAGCGGCTAACGCCGCT
TTTTTTGTTTCTGGTCTCCCTTGCTTGTACTTTACAGTATAGCTTCTAGAGATCTGCAG
TGACTGGAGCTTCTAGAGATCTGCAGTGACTGGGAAAACCCTGGCGACTAGTCTTGG
ACTCCTGTTGATAGATCCAGTAATGACCTCAGAACTCCATCTGGATTTGTTCAGAAC
GCTCGGTTGCCGCCGGGCGTTTTTTATTGGTGAGAATCCAGGGGTCCCCAATAATTA
CGATTTAAATCCTTCAAACTTCCCAAAGGCGAGCCCTAGTGACATTAGAAAACCGAC
TGTAAAAAGTACAGTCGGCATTATCTCATATTATAAAAGCCAGTCATTAGGCCTATC
TGACAATTCCTGAATAGAGTTCATAAACAATCCTGCATGATAACCATCACAAACAGA
ATGATGTACCTGTAAAGATAGCGGTAAATATATTGAATTACCTTTATTAATGAATTT
TCCTGCTGTAATAATGGGTAGAAGGTAATTACTATTATTATTGATATTTAAGTTAAA
CCCAGTAAATGAAGTCCATGGAATAATAGAAAGAGAAAAAGCATTTTCAGGTATAG
GTGTTTTGGGAAACAATTTCCCCGAACCATTATATTTCTCTACATCAGAAAGGTATA
AATCATAAAACTCTTTGAAGTCATTCTTTACAGGAGTCCAAATACCAGAGAATGTTT
TAGATACACCATCAAAAATTGTATAAAGTGGCTCTAACTTATCCCAATAACCTAACT
CTCCGTCGCTATTGTAACCAGTTCTAAAAGCTGTATTTGAGTTTATCACCCTTGTCAC
TAAGAAAATAAATGCAGGGTAAAATTTATATCCTTCTTGTTTTATGTTTCGGTATAA
AACACTAATATCAATTTCTGTGGTTATACTAAAAGTCGTTTGTTGGTTCAAATAATG
ATTAAATATCTCTTTTCTCTTCCAATTGTCTAAATCAATTTTATTAAAGTTCATTTGAT
ATGCCTCCTAAATTTTTATCTAAAGTGAATTTAGGAGGCTTACTTGTCTGCTTTCTTC
ATTAGAATCAATCCTTTTTTAAAAGTCAATCCCGTTTGTTGAAGACTTTTGTCCTTTT
CCGCTGCATAAACAATATGGCCCGTTTGTTGAACTACTCTTTAATAAAATAATTTTTC
CGTTCCCAATTCCACATTGCAATAATAGAAAATCCATCTTCATCGGCTTTTTCGTCAT
CATCTGTATGAATCAAATCGCCTTCTTCTGTGTCATCAAGGTTTAATTTTTTATGTAT
TTCTTTTAACAAACCACCATAGGAGATTAACCTTTTACGGTGTAAACCTTCCTCCAA
ATCAGACAAACGTTTCAAATTCTTTTCTTCATCATCGGTCATAAAATCCGTATCCTTT
ACAGGATATTTTGCAGTTTCGTCAATTGCCGATTGTATATCCGATTTATATTTATTTT
TCGGTCGAATCATTTGAACTTTTACATTTGGATCATAGTCTAATTTCATTGCCTTTTTC
CAAAATTGAATCCATTGTTTTTGATTCACGTAGTTTTCTGTATTCTTAAAATAAGTTG
GTTCCACACATACCAATACATGCATGTGCTGATTATAAGAATTATCTTTATTATTTAT
TGTCACTTCCGTTGCACGCATAAAACCAACAAGATTTTTATTAATTTTTTTATATTGC
ATCATTCGGCGAAATCCTTGAGCCATATCTGACAAACTCTTATTTAATTCTTCGCCAT
CATAAACATTTTTAACTGTTAATGTGAGAAACAACCAACGAACTGTTGGCTTTTGTT
TAATAACTTCAGCAACAACCTTTTGTGACTGAATGCCATGTTTCATTGCTCTCCTCCA
GTTGCACATTGGACAAAGCCTGGATTTACAAAACCACACTCGATACAACTTTCTTTC
GCCTGTTTCACGATTTTGTTTATACTCTAATATTTCAGCACAATCTTTTACTCTTTCAG
CCTTTTTAAATTCAAGAATATGCAGAAGTTCAAAGTAATCAACATTAGCGATTTTCT
TTTCTCTCCATGGTCTCACTTTTCCACTTTTTGTCTTGTCCACTAAAACCCTTGATTTT
TCATCTGAATAAATGCTACTATTAGGACACATAATATTAAAAGAAACCCCCATCTAT
TTAGTTATTTGTTTGGTCACTTATAACTTTAACAGATGGGGTTTTTCTGTGCAACCAA
TTTTAAGGGTTTTCCAATACTTTAAAACACATACATACCAACACTTCAACGCACCTTT
CAGCAACTAAAATAAAAATGACGTTATTTCTATATGTATCAAGATAAGAAAGAACA
AGTTCAAAACCATCAAAAAAAGACACCTTTTCAGGTGCTTTTTTTATTTTATAAACTC
ATTCCCTGATCTCGACTTCGTTCTTTTTTTACCTCTCGGTTATGAGTTAGTTCAAATTC
GTTCTTTTTAGGTTCTAAATCGTGTTTTTCTTGGAATTGTGCTGTTTTATCCTTTACCT
TGTCTACAAACCCCTTAAAAACGTTTTTAAAGGCTTTTAAGCGTCTGTACGTTCCTTA
AGGAATTATTCCTTAGTGCTTTCTAGGTTAATGTCATGATAATAATGGTTTCTTAGAC
GTCAGGTGGCACTTTTCGGGGAAATGTCCGCGGAACCCCTATTTGTTTATTTTTCTAA
ATACATTCAAATATGTATCCGCTCATGAGACAATAACCCTGATAAATGCTTCAATAA
TATTGAAAAAGGAAGAGTATGAGTATTCAACATTTCCGTGTCGCCCTTATTCCCTTTT
TTGCGGCATTTTGCCTTCCTGTTTTTGCTCACCCAGAAACGCTGGTGAAAGTAAAAG
ATGCTGAAGATCAGTTGGGTGCACGAGTGGGTTACATCGAACTGGATCTCAACAGC
GGTAAGATCCTTGAGAGTTTTCGCCCCGAAGAACGTTTTCCAATGATGAGCACTTTT
AAAGTTCTGCTATGTGGCGCGGTATTATCCCGTGTTGACGCCGGGCAAGAGCAACTC
GGTCGCCGCATACACTATTCTCAGAATGACTTGGTTGAGTACTCACCAGTCACAGAA
AAGCATCTTACGGATGGCATGACAGTAAGAGAATTATGCAGTGCTGCCATAACCAT
GAGTGATAACACTGCGGCCAACTTACTTCTGACAACGATCGGAGGACCGAAGGAGC
TAACCGCTTTTTTGCACAACATGGGGGATCATGTAACTCGCCTTGATCGTTGGGAAC
CGGAGCTGAATGAAGCCATACCAAACGACGAGCGTGACACCACGATGCCTGCAGCA
ATGGCAACAACGTTGCGCAAACTATTAACTGGCGAACTACTTACTCTAGCTTCCCGG
CAACAATTAATAGACTGGATGGAGGCGGATAAAGTTGCAGGACCACTTCTGCGCTC
GGCCCTTCCGGCTGGCTGGTTTATTGCTGATAAATCTGGAGCCGGTGAGCGTGGGTC
TCGCGGTATCATTGCAGCACTGGGGCCAGATGGTAAGCCCTCCCGTATCGTAGTTAT
CTACACGACGGGGAGTCAGGCAACTATGGATGAACGAAATAGACAGATCGCTGAGA
TAGGTGCCTCACTGATTAAGCATTGGTAACTGTCAGACCAAGTTTACTCATATATAC
TTTAGATTGATTTAAAACTTCATTTTTAATTTAAAAGGATCTAGGTGAAGATCCTTTT
TGATAATCTCATGACCAAAATCCCTTAACGTGAGTTTTCGTTCCACTGAGCGTCAGA
CCCCTTAATAAGATGATCTTCTTGAGATCGTTTTGGTCTGCGCGTAATCTCTTGCTCT
GAAAACGAAAAAACCGCCTTGCAGGGAGGTTTTTCGAAGGTTCTCTGAGCTACCAA
CTCTTTGAACCGAGGTAACTGGCTTGCAGGAGCGCAGTCACCAAAACTTGTCCTTTC
AGTTTAGCCTTAACCGGCGCATGACTTCAAGACTAACTCCTCTAAATCAATTACCAG
TGGCTGCTGCCAGTGGTGCTTTTGCATGTCTTTCCGGGTTGGACTCAAGACGATAGTT
ACCGGATAAGGCGCAGCGGTCGGACTGAACGGGGGGTTCGTGCATACAGTCCAGCT
TGGAGCGAACTGCCTACCCGGAACTGAGTGTCAGGCGTGGAATGAGACAAACGCGG
CCATAACAGCGGAATGACACCGGTAAACCGAAAGGCAGGAACAGGAGAGCGCACG
AGGGAGCCGCCAGGGGGAAACGCCTGGTATCTTTATAGTCCTGTCGGGTTTCGCCAC
CACTGATTTGAGCGTCAGATTTCGTGATGCTTGTCAGGGGGCGGAGCCTATGGAAAA
ACGCTTTGCCCGAGCCTATGGAAAAACGCTTTGCCCCACACAGGCTCTTGAACAAGG
GATAACAATTTGTCATAGCTGTATTCGTTACAAGGAGGATTTTCGAAAAAACACCCT
AACGGGTGTTTTTTTATAGCTGGTCTCCCCAAGTGCTGTAAAAAACGCCGAATGAAG
CGGATGAAATTGCTCATCGTCTCGTGAAAAAGCCGAAGAAGGTTAAGCCTGGGTAT
AAAAAGAAAATGAGCTATGAGATGGAGAAAGCCATAACCATGAGTGATAACACTG
GTTTTAGAGCTAGAAATAGCAAGTTAAAATAAGGCTAGTCCGTTATCAACTTGAAAA
AGTGGCACCGAGTCGGTGCTTTTTTTCTCGGTACCAAATTCCAGAAAAGAGGCCTCC
CGAAAGGGGGGCCTTTTTTCGTTTTGGTCCCTCAATGATC
>pCas9g2-D15
(SEQ ID No: 236)
tccttatccggttaagatggCTTTTAAATCAATTTTCAGCTCCTGTATACAATTACCAAAG
TTTTTCTGAATGAAGCCATGTGTTTTGACACATTCTATACTCACAAGGAGGTGAGACACat
gagtaaatcctggggcaaATTCATCGAGGAGGAAGAGGCGGAAATGGCTTCCCGTCGTAAT
CTAATGATTGTCGATGGAACTAACTTAGGCTTTCGCTTCAAACATAACAATAGTAAAAAAC
CATTTGCCTCAAGTTATGTTTCAACTATTCAATCTCTGGCAAAATCCTACTCTGCCAG
AACTACGATTGTTCTAGGTGATAAGGGCAAAAGCGTGTTCAGACTAGAACATCTACC
AGAGTATAAAGGTAATCGTGATGAAAAGTACGCACAACGTACGGAAGAGGAGAAA
GCGCTAGATGAGCAGTTCTTCGAATACCTCAAAGATGCTTTCGAGTTGTGTAAAACT
ACATTCCCAACTTTTACCATTCGTGGTGTAGAAGCAGATGACATGGCGGCGTATATT
GTTAAGCTCATCGGGCATCTTTATGATCACGTTTGGCTAATATCTACAGATGGTGAC
TGGGATACTTTATTAACGGATAAAGTTTCTCGTTTTTCTTTCACAACACGTCGTGAGT
ATCATCTTCGTGATATGTATGAGCACCATAACGTGGACGACGTAGAACAGTTTATCT
CCCTGAAAGCAATTATGGGAGATCTAGGAGATAATATTCGTGGTGTTGAAGGAATA
GGAGCAAAACGCGGATATAATATTATTCGTGAGTTTGGTAACGTACTGGATATTATT
GATCAGCTTCCACTGCCTGGAAAGCAGAAATATATACAGAACCTGAATGCATCGGA
AGAACTGCTTTTCCGAAACTTGATTCTGGTTGATTTACCTACCTACTGTGTGGATGCT
ATTGCTGCTGTAGGTCAAGATGTGTTAGATAAGTTCACGAAAGACATCCTTGAGATC
GCGGAACAATGATAAGCTCTTAAAGGGGGTTTTAGATACCAGAGATGGATAAGAAA
TACTCAATAGGCTTAGATATCGGCACAAATAGCGTCGGATGGGCGGTGATCACTGAT
GAATATAAGGTTCCGTCTAAAAAGTTCAAGGTTCTGGGAAATACAGACCGCCACAG
TATCAAAAAAAATCTTATAGGGGCTCTTTTATTTGACAGTGGAGAGACAGCGGAAG
CGACTCGTCTCAAACGGACAGCTCGTAGAAGGTATACACGTCGGAAGAATCGTATTT
GTTATCTACAGGAGATTTTTTCAAATGAGATGGCGAAAGTAGATGATAGTTTCTTTC
ATCGACTTGAAGAGTCTTTTTTGGTGGAAGAAGACAAGAAGCATGAACGTCATCCTA
TTTTTGGAAATATAGTAGATGAAGTTGCTTATCATGAGAAATATCCAACTATCTATC
ATCTGCGAAAAAAATTGGTAGATTCTACTGATAAAGCGGATTTGCGCTTAATCTATT
TGGCCTTAGCGCATATGATTAAGTTTCGTGGTCATTTTTTGATTGAGGGAGATTTAAA
TCCTGATAATAGTGATGTGGACAAACTATTTATCCAGTTGGTACAAACCTACAATCA
ATTATTTGAAGAAAACCCTATTAACGCAAGTGGAGTAGATGCTAAAGCGATTCTTTC
TGCACGATTGAGTAAATCAAGACGATTAGAAAATCTCATTGCTCAGCTCCCCGGTGA
GAAGAAAAATGGCTTATTTGGGAATCTCATTGCTTTGTCATTGGGTTTGACCCCTAA
TTTTAAATCAAATTTTGATTTGGCAGAAGATGCTAAATTACAGCTTTCAAAAGATAC
TTACGATGATGATTTAGATAATTTATTGGCGCAAATTGGAGATCAATATGCTGATTT
GTTTTTGGCAGCTAAGAATTTATCAGATGCTATTTTACTTTCAGATATCCTAAGAGTA
AATACTGAAATAACTAAGGCTCCCCTATCAGCTTCAATGATTAAACGCTACGATGAA
CATCATCAAGACTTGACTCTTTTAAAAGCTTTAGTTCGACAACAACTTCCAGAAAAG
TATAAAGAAATCTTTTTTGATCAATCAAAAAACGGATATGCAGGTTATATTGATGGG
GGAGCTAGCCAAGAAGAATTTTATAAATTTATCAAACCAATTTTAGAAAAAATGGA
TGGTACTGAGGAATTATTGGTGAAACTAAATCGTGAAGATTTGCTGCGCAAGCAAC
GGACCTTTGACAACGGCTCTATTCCCCATCAAATTCACTTGGGTGAGCTGCATGCTA
TTTTGAGAAGACAAGAAGACTTTTATCCATTTTTAAAAGACAATCGTGAGAAGATTG
AAAAAATCTTGACTTTTCGAATTCCTTATTATGTTGGTCCATTGGCGCGTGGCAATAG
TCGTTTTGCATGGATGACTCGGAAGTCTGAAGAAACAATTACCCCATGGAATTTTGA
AGAAGTTGTCGATAAAGGTGCTTCAGCTCAATCATTTATTGAACGCATGACAAACTT
TGATAAAAATCTTCCAAATGAAAAAGTACTACCAAAACATAGTTTGCTTTATGAGTA
TTTTACGGTTTATAACGAATTGACAAAGGTCAAATATGTTACTGAAGGAATGCGAAA
ACCAGCATTTCTTTCAGGTGAACAGAAGAAAGCCATTGTTGATTTACTCTTCAAAAC
AAATCGAAAAGTAACCGTTAAGCAATTAAAAGAAGATTATTTCAAAAAAATAGAAT
GTTTTGATAGTGTTGAAATTTCAGGAGTTGAAGATAGATTTAATGCTTCATTAGGTA
CCTACCATGATTTGCTAAAAATTATTAAAGATAAAGATTTTTTGGATAATGAAGAAA
ATGAAGATATCTTAGAGGATATTGTTTTAACATTGACCTTATTTGAAGATAGGGAGA
TGATTGAGGAAAGACTTAAAACATATGCTCACCTCTTTGATGATAAGGTGATGAAAC
AGCTTAAACGTCGCCGTTATACTGGTTGGGGACGTTTGTCTCGAAAATTGATTAATG
GTATTAGGGATAAGCAATCTGGCAAAACAATATTAGATTTTTTGAAATCAGATGGTT
TTGCCAATCGCAATTTTATGCAGCTGATCCATGATGATAGTTTGACATTTAAAGAAG
ACATTCAAAAAGCACAAGTGTCTGGACAAGGCGATAGTTTACATGAACATATTGCA
AATTTAGCTGGTAGCCCTGCTATTAAAAAAGGTATTTTACAGACTGTAAAAGTTGTT
GATGAATTGGTCAAAGTAATGGGGCGGCATAAGCCAGAAAATATCGTTATTGAAAT
GGCACGTGAAAATCAGACAACTCAAAAGGGCCAGAAAAATTCGCGAGAGCGTATG
AAACGAATCGAAGAAGGTATCAAAGAATTAGGAAGTCAGATTCTTAAAGAGCATCC
TGTTGAAAATACTCAATTGCAAAATGAAAAGCTCTATCTCTATTATCTCCAAAATGG
AAGAGACATGTATGTGGACCAAGAATTAGATATTAATCGTTTAAGTGATTATGATGT
CGATCACATTGTTCCACAAAGTTTCCTTAAAGACGATTCAATAGACAATAAGGTCTT
AACGCGTTCTGATAAAAATCGTGGTAAATCGGATAACGTTCCAAGTGAAGAAGTAG
TCAAAAAGATGAAAAACTATTGGAGACAACTTCTAAACGCCAAGTTAATCACTCAA
CGTAAGTTTGATAATTTAACGAAAGCTGAACGTGGAGGTTTGAGTGAACTTGATAAA
GCTGGTTTTATCAAACGCCAATTGGTTGAAACTCGCCAAATCACTAAGCATGTGGCA
CAAATTTTGGATAGTCGCATGAATACTAAATACGATGAAAATGATAAACTTATTCGA
GAGGTTAAAGTGATTACCTTAAAATCTAAATTAGTTTCTGACTTCCGAAAAGATTTC
CAATTCTATAAAGTACGTGAGATTAACAATTACCATCATGCCCATGATGCGTATCTA
AATGCCGTCGTTGGAACTGCTTTGATTAAGAAATATCCAAAACTTGAATCGGAGTTT
GTCTATGGTGATTATAAAGTTTATGATGTTCGTAAAATGATTGCTAAGTCTGAGCAA
GAAATAGGCAAAGCAACCGCAAAATATTTCTTTTACTCTAATATCATGAACTTCTTC
AAAACAGAAATTACACTTGCAAATGGAGAGATTCGCAAACGCCCTCTAATCGAAAC
TAATGGGGAAACTGGAGAAATTGTCTGGGATAAAGGGCGAGATTTTGCCACAGTGC
GCAAAGTATTGTCCATGCCCCAAGTCAATATTGTCAAGAAAACAGAAGTACAGACA
GGCGGATTCTCCAAGGAGTCAATTTTACCAAAAAGAAATTCGGACAAGCTTATTGCT
CGTAAAAAAGACTGGGATCCAAAAAAATATGGTGGTTTTGATAGTCCAACGGTAGC
TTATTCAGTCCTAGTGGTTGCTAAGGTGGAAAAAGGGAAATCGAAGAAGTTAAAAT
CCGTTAAAGAGTTACTAGGGATCACAATTATGGAAAGAAGTTCCTTTGAAAAAAAT
CCGATTGACTTTTTAGAAGCTAAAGGATATAAGGAAGTTAAAAAAGACTTAATCATT
AAACTACCTAAATATAGTCTTTTTGAGTTAGAAAACGGTCGTAAACGGATGCTGGCT
AGTGCCGGAGAATTACAAAAAGGAAATGAGCTGGCTCTGCCAAGCAAATATGTGAA
TTTTTTATATTTAGCTAGTCATTATGAAAAGTTGAAGGGTAGTCCAGAAGATAACGA
ACAAAAACAATTGTTTGTGGAGCAGCATAAGCATTATTTAGATGAGATTATTGAGCA
AATCAGTGAATTTTCTAAGCGTGTTATTTTAGCAGATGCCAATTTAGATAAAGTTCTT
AGTGCATATAACAAACATAGAGACAAACCAATACGTGAACAAGCAGAAAATATTAT
TCATTTATTTACGTTGACGAATCTTGGAGCTCCCGCTGCTTTTAAATATTTTGATACA
ACAATTGATCGTAAACGATATACGTCTACAAAAGAAGTTTTAGATGCCACTCTTATC
CATCAATCCATCACTGGTCTTTATGAAACACGCATTGATTTGAGTCAGCTAGGAGGT
GACTGATAATCAAATGTCAGACGAAAATGCCAATTATTGAAGCGGCTAACGCCGCT
TTTTTTGTTTCTGGTCTCCCTTGCTTGTACTTTACAGTATAGCTTCTAGAGATCTGCAG
TGACTGGAGCTTCTAGAGATCAGGTCAGACGCGATTTCCTGGGTGCTCGAGCCATGG
GAAAGCGGTGGTGGAAAAAACGCCGAATGAAGCGGATGAAATTGCTCATCGTCTCG
TGAAAAAGCCGAAGAAGGTTAAGCCTGGGTATAAAAAGAAAATGAGCTATGAGAT
GGAGAAATTGAAGTCATGCGCCGGTTAGTTTTAGAGCTAGAAATAGCAAGTTAAAA
TAAGGCTAGTCCGTTATCAACTTGAAAAAGTGGCACCGAGTCGGTGCTTTTTTTCTC
GGTACCAAATTCCAGAAAAGAGGCCTCCCGAAAGGGGGGCCTTTTTTCGTTTTGGTC
CCTCAATGATCCGACAGGAAGTGGTATTAGCGACTCCTACAAGGAATAGGTGTCTG
GGTCATGCAGTGACTGGGAAAACCCTGGCGACTAGTCTTGGACTCCTGTTGATAGAT
CCAGTAATGACCTCAGAACTCCATCTGGATTTGTTCAGAACGCTCGGTTGCCGCCGG
GCGTTTTTTATTGGTGAGAATCCAGGGGTCCCCAATAATTACGATTTAAATCCTTCA
AACTTCCCAAAGGCGAGCCCTAGTGACATTAGAAAACCGACTGTAAAAAGTACAGT
CGGCATTATCTCATATTATAAAAGCCAGTCATTAGGCCTATCTGACAATTCCTGAAT
AGAGTTCATAAACAATCCTGCATGATAACCATCACAAACAGAATGATGTACCTGTA
AAGATAGCGGTAAATATATTGAATTACCTTTATTAATGAATTTTCCTGCTGTAATAAT
GGGTAGAAGGTAATTACTATTATTATTGATATTTAAGTTAAACCCAGTAAATGAAGT
CCATGGAATAATAGAAAGAGAAAAAGCATTTTCAGGTATAGGTGTTTTGGGAAACA
ATTTCCCCGAACCATTATATTTCTCTACATCAGAAAGGTATAAATCATAAAACTCTTT
GAAGTCATTCTTTACAGGAGTCCAAATACCAGAGAATGTTTTAGATACACCATCAAA
AATTGTATAAAGTGGCTCTAACTTATCCCAATAACCTAACTCTCCGTCGCTATTGTAA
CCAGTTCTAAAAGCTGTATTTGAGTTTATCACCCTTGTCACTAAGAAAATAAATGCA
GGGTAAAATTTATATCCTTCTTGTTTTATGTTTCGGTATAAAACACTAATATCAATTT
CTGTGGTTATACTAAAAGTCGTTTGTTGGTTCAAATAATGATTAAATATCTCTTTTCT
CTTCCAATTGTCTAAATCAATTTTATTAAAGTTCATTTGATATGCCTCCTAAATTTTT
ATCTAAAGTGAATTTAGGAGGCTTACTTGTCTGCTTTCTTCATTAGAATCAATCCTTT
TTTAAAAGTCAATCCCGTTTGTTGAAGACTTTTGTCCTTTTCCGCTGCATAAACAATA
TGGCCCGTTTGTTGAACTACTCTTTAATAAAATAATTTTTCCGTTCCCAATTCCACAT
TGCAATAATAGAAAATCCATCTTCATCGGCTTTTTCGTCATCATCTGTATGAATCAAA
TCGCCTTCTTCTGTGTCATCAAGGTTTAATTTTTTATGTATTTCTTTTAACAAACCACC
ATAGGAGATTAACCTTTTACGGTGTAAACCTTCCTCCAAATCAGACAAACGTTTCAA
ATTCTTTTCTTCATCATCGGTCATAAAATCCGTATCCTTTACAGGATATTTTGCAGTT
TCGTCAATTGCCGATTGTATATCCGATTTATATTTATTTTTCGGTCGAATCATTTGAA
CTTTTACATTTGGATCATAGTCTAATTTCATTGCCTTTTTCCAAAATTGAATCCATTGT
TTTTGATTCACGTAGTTTTCTGTATTCTTAAAATAAGTTGGTTCCACACATACCAATA
CATGCATGTGCTGATTATAAGAATTATCTTTATTATTTATTGTCACTTCCGTTGCACG
CATAAAACCAACAAGATTTTTATTAATTTTTTTATATTGCATCATTCGGCGAAATCCT
TGAGCCATATCTGACAAACTCTTATTTAATTCTTCGCCATCATAAACATTTTTAACTG
TTAATGTGAGAAACAACCAACGAACTGTTGGCTTTTGTTTAATAACTTCAGCAACAA
CCTTTTGTGACTGAATGCCATGTTTCATTGCTCTCCTCCAGTTGCACATTGGACAAAG
CCTGGATTTACAAAACCACACTCGATACAACTTTCTTTCGCCTGTTTCACGATTTTGT
TTATACTCTAATATTTCAGCACAATCTTTTACTCTTTCAGCCTTTTTAAATTCAAGAA
TATGCAGAAGTTCAAAGTAATCAACATTAGCGATTTTCTTTTCTCTCCATGGTCTCAC
TTTTCCACTTTTTGTCTTGTCCACTAAAACCCTTGATTTTTCATCTGAATAAATGCTAC
TATTAGGACACATAATATTAAAAGAAACCCCCATCTATTTAGTTATTTGTTTGGTCAC
TTATAACTTTAACAGATGGGGTTTTTCTGTGCAACCAATTTTAAGGGTTTTCCAATAC
TTTAAAACACATACATACCAACACTTCAACGCACCTTTCAGCAACTAAAATAAAAAT
GACGTTATTTCTATATGTATCAAGATAAGAAAGAACAAGTTCAAAACCATCAAAAA
AAGACACCTTTTCAGGTGCTTTTTTTATTTTATAAACTCATTCCCTGATCTCGACTTC
GTTCTTTTTTTACCTCTCGGTTATGAGTTAGTTCAAATTCGTTCTTTTTAGGTTCTAAA
TCGTGTTTTTCTTGGAATTGTGCTGTTTTATCCTTTACCTTGTCTACAAACCCCTTAAA
AACGTTTTTAAAGGCTTTTAAGCGTCTGTACGTTCCTTAAGGAATTATTCCTTAGTGC
TTTCTAGGTTAATGTCATGATAATAATGGTTTCTTAGACGTCAGGTGGCACTTTTCGG
GGAAATGTCCGCGGAACCCCTATTTGTTTATTTTTCTAAATACATTCAAATATGTATC
CGCTCATGAGACAATAACCCTGATAAATGCTTCAATAATATTGAAAAAGGAAGAGT
ATGAGTATTCAACATTTCCGTGTCGCCCTTATTCCCTTTTTTGCGGCATTTTGCCTTCC
TGTTTTTGCTCACCCAGAAACGCTGGTGAAAGTAAAAGATGCTGAAGATCAGTTGGG
TGCACGAGTGGGTTACATCGAACTGGATCTCAACAGCGGTAAGATCCTTGAGAGTTT
TCGCCCCGAAGAACGTTTTCCAATGATGAGCACTTTTAAAGTTCTGCTATGTGGCGC
GGTATTATCCCGTGTTGACGCCGGGCAAGAGCAACTCGGTCGCCGCATACACTATTC
TCAGAATGACTTGGTTGAGTACTCACCAGTCACAGAAAAGCATCTTACGGATGGCAT
GACAGTAAGAGAATTATGCAGTGCTGCCATAACCATGAGTGATAACACTGCGGCCA
ACTTACTTCTGACAACGATCGGAGGACCGAAGGAGCTAACCGCTTTTTTGCACAACA
TGGGGGATCATGTAACTCGCCTTGATCGTTGGGAACCGGAGCTGAATGAAGCCATA
CCAAACGACGAGCGTGACACCACGATGCCTGCAGCAATGGCAACAACGTTGCGCAA
ACTATTAACTGGCGAACTACTTACTCTAGCTTCCCGGCAACAATTAATAGACTGGAT
GGAGGCGGATAAAGTTGCAGGACCACTTCTGCGCTCGGCCCTTCCGGCTGGCTGGTT
TATTGCTGATAAATCTGGAGCCGGTGAGCGTGGGTCTCGCGGTATCATTGCAGCACT
GGGGCCAGATGGTAAGCCCTCCCGTATCGTAGTTATCTACACGACGGGGAGTCAGG
CAACTATGGATGAACGAAATAGACAGATCGCTGAGATAGGTGCCTCACTGATTAAG
CATTGGTAACTGTCAGACCAAGTTTACTCATATATACTTTAGATTGATTTAAAACTTC
ATTTTTAATTTAAAAGGATCTAGGTGAAGATCCTTTTTGATAATCTCATGACCAAAA
TCCCTTAACGTGAGTTTTCGTTCCACTGAGCGTCAGACCCCTTAATAAGATGATCTTC
TTGAGATCGTTTTGGTCTGCGCGTAATCTCTTGCTCTGAAAACGAAAAAACCGCCTT
GCAGGGAGGTTTTTCGAAGGTTCTCTGAGCTACCAACTCTTTGAACCGAGGTAACTG
GCTTGCAGGAGCGCAGTCACCAAAACTTGTCCTTTCAGTTTAGCCTTAACCGGCGCA
TGACTTCAAGACTAACTCCTCTAAATCAATTACCAGTGGCTGCTGCCAGTGGTGCTT
TTGCATGTCTTTCCGGGTTGGACTCAAGACGATAGTTACCGGATAAGGCGCAGCGGT
CGGACTGAACGGGGGGTTCGTGCATACAGTCCAGCTTGGAGCGAACTGCCTACCCG
GAACTGAGTGTCAGGCGTGGAATGAGACAAACGCGGCCATAACAGCGGAATGACAC
CGGTAAACCGAAAGGCAGGAACAGGAGAGCGCACGAGGGAGCCGCCAGGGGGAAA
CGCCTGGTATCTTTATAGTCCTGTCGGGTTTCGCCACCACTGATTTGAGCGTCAGATT
TCGTGATGCTTGTCAGGGGGCGGAGCCTATGGAAAAACGCTTTGCCCGAGCCTATGG
AAAAACGCTTTGCCCCACACAGGCTCTTGAACAAGGGATAACAATTTGTCATAGCTG
TATTCGTTACAAGGAGGATTTTCGAAAAAACACCCTAACGGGTGTTTTTTTATAGCT
GGTCTCCCCAAGTGCTGTAAAAAACGCCGAATGAAGCGGATGAAATTGCTCATCGT
CTCGTGAAAAAGCCGAAGAAGGTTAAGCCTGGGTATAAAAAGAAAATGAGCTATGA
GATGGAGAAAGCCATAACCATGAGTGATAACACTGGTTTTAGAGCTAGAAATAGCA
AGTTAAAATAAGGCTAGTCCGTTATCAACTTGAAAAAGTGGCACCGAGTCGGTGCTT
TTTTTCTCGGTACCAAATTCCAGAAAAGAGGCCTCCCGAAAGGGGGGCCTTTTTTCG
TTTTGGTCCCTCAATGATC
>pCas9g1-D15-mut
(SEQ ID No: 237)
GAGTAAATCCTGGGGCAAATTCATCGAGGAGGAAGAGGCGGAAATGGCTTCCCGTC
GTAATCTAATGATTGTCGATGGAACTAACTTAGGCTTTCGCTTCAAACATAACAATA
GTAAAAAACCATTTGCCTCAAGTTATGTTTCAACTATTCAATCTCTGGCAAAATCCT
ACTCTGCCAGAACTACGATTGTTCTAGGTGATAAGGGCAAAAGCGTGTTCAGACTAG
AACATCTACCAGAGTATAAAGGTAATCGTGATGAAAAGTACGCACAACGTACGGAA
GAGGAGAAAGCGCTAGATGAGCAGTTCTTCGAATACCTCAAAGATGCTTTCGAGTT
GTGTAAAACTACATTCCCAACTTTTACCATTCGTGGTGTAGAAGCAGATGACATGGC
GGCGTATATTGTTAAGCTCATCGGGCATCTTTATGATCACGTTTGGCTAATATCTACA
GATGGTGACTGGGATACTTTATTAACGGATAAAGTTTCTCGTTTTTCTTTCACAACAC
GTCGTGAGTATCATCTTCGTGATATGTATGAGCACCATAACGTGGACGACGTAGAAC
AGTTTATCTCCCTGAAAGCAATTATGGGAGATCTAGGAGATAATATTCGTGGTGTTG
AAGGAATAGGAGCAAAACGCGGATATAATATTATTCGTGAGTTTGGTAACGTACTG
GATATTATTGATCAGCTTCCACTGCCTGGAAAGCAGAAATATATACAGAACCTGAAT
GCATCGGAAGAACTGCTTTTCCGAAACTTGATTCTGGTTGATTTACCTACCTACTGTG
TGGATGCTATTGCTGCTGTAGGTCAAGATGTGTTAGATAAGTTCACGAAAGACATCC
TTGAGATCGCGGAACAATGATAAGCTCTTAAAGGGGGTTTTAGATACCAGAGATGG
ATAAGAAATACTCAATAGGCTTAGATATCGGCACAAATAGCGTCGGATGGGCGGTG
ATCACTGATGAATATAAGGTTCCGTCTAAAAAGTTCAAGGTTCTGGGAAATACAGAC
CGCCACAGTATCAAAAAAAATCTTATAGGGGCTCTTTTATTTGACAGTGGAGAGACA
GCGGAAGCGACTCGTCTCAAACGGACAGCTCGTAGAAGGTATACACGTCGGAAGAA
TCGTATTTGTTATCTACAGGAGATTTTTTCAAATGAGATGGCGAAAGTAGATGATAG
TTTCTTTCATCGACTTGAAGAGTCTTTTTTGGTGGAAGAAGACAAGAAGCATGAACG
TCATCCTATTTTTGGAAATATAGTAGATGAAGTTGCTTATCATGAGAAATATCCAAC
TATCTATCATCTGCGAAAAAAATTGGTAGATTCTACTGATAAAGCGGATTTGCGCTT
AATCTATTTGGCCTTAGCGCATATGATTAAGTTTCGTGGTCATTTTTTGATTGAGGGA
GATTTAAATCCTGATAATAGTGATGTGGACAAACTATTTATCCAGTTGGTACAAACC
TACAATCAATTATTTGAAGAAAACCCTATTAACGCAAGTGGAGTAGATGCTAAAGC
GATTCTTTCTGCACGATTGAGTAAATCAAGACGATTAGAAAATCTCATTGCTCAGCT
CCCCGGTGAGAAGAAAAATGGCTTATTTGGGAATCTCATTGCTTTGTCATTGGGTTT
GACCCCTAATTTTAAATCAAATTTTGATTTGGCAGAAGATGCTAAATTACAGCTTTC
AAAAGATACTTACGATGATGATTTAGATAATTTATTGGCGCAAATTGGAGATCAATA
TGCTGATTTGTTTTTGGCAGCTAAGAATTTATCAGATGCTATTTTACTTTCAGATATC
CTAAGAGTAAATACTGAAATAACTAAGGCTCCCCTATCAGCTTCAATGATTAAACGC
TACGATGAACATCATCAAGACTTGACTCTTTTAAAAGCTTTAGTTCGACAACAACTT
CCAGAAAAGTATAAAGAAATCTTTTTTGATCAATCAAAAAACGGATATGCAGGTTAT
ATTGATGGGGGAGCTAGCCAAGAAGAATTTTATAAATTTATCAAACCAATTTTAGAA
AAAATGGATGGTACTGAGGAATTATTGGTGAAACTAAATCGTGAAGATTTGCTGCG
CAAGCAACGGACCTTTGACAACGGCTCTATTCCCCATCAAATTCACTTGGGTGAGCT
GCATGCTATTTTGAGAAGACAAGAAGACTTTTATCCATTTTTAAAAGACAATCGTGA
GAAGATTGAAAAAATCTTGACTTTTCGAATTCCTTATTATGTTGGTCCATTGGCGCGT
GGCAATAGTCGTTTTGCATGGATGACTCGGAAGTCTGAAGAAACAATTACCCCATGG
AATTTTGAAGAAGTTGTCGATAAAGGTGCTTCAGCTCAATCATTTATTGAACGCATG
ACAAACTTTGATAAAAATCTTCCAAATGAAAAAGTACTACCAAAACATAGTTTGCTT
TATGAGTATTTTACGGTTTATAACGAATTGACAAAGGTCAAATATGTTACTGAAGGA
ATGCGAAAACCAGCATTTCTTTCAGGTGAACAGAAGAAAGCCATTGTTGATTTACTC
TTCAAAACAAATCGAAAAGTAACCGTTAAGCAATTAAAAGAAGATTATTTCAAAAA
AATAGAATGTTTTGATAGTGTTGAAATTTCAGGAGTTGAAGATAGATTTAATGCTTC
ATTAGGTACCTACCATGATTTGCTAAAAATTATTAAAGATAAAGATTTTTTGGATAA
TGAAGAAAATGAAGATATCTTAGAGGATATTGTTTTAACATTGACCTTATTTGAAGA
TAGGGAGATGATTGAGGAAAGACTTAAAACATATGCTCACCTCTTTGATGATAAGGT
GATGAAACAGCTTAAACGTCGCCGTTATACTGGTTGGGGACGTTTGTCTCGAAAATT
GATTAATGGTATTAGGGATAAGCAATCTGGCAAAACAATATTAGATTTTTTGAAATC
AGATGGTTTTGCCAATCGCAATTTTATGCAGCTGATCCATGATGATAGTTTGACATTT
AAAGAAGACATTCAAAAAGCACAAGTGTCTGGACAAGGCGATAGTTTACATGAACA
TATTGCAAATTTAGCTGGTAGCCCTGCTATTAAAAAAGGTATTTTACAGACTGTAAA
AGTTGTTGATGAATTGGTCAAAGTAATGGGGCGGCATAAGCCAGAAAATATCGTTA
TTGAAATGGCACGTGAAAATCAGACAACTCAAAAGGGCCAGAAAAATTCGCGAGAG
CGTATGAAACGAATCGAAGAAGGTATCAAAGAATTAGGAAGTCAGATTCTTAAAGA
GCATCCTGTTGAAAATACTCAATTGCAAAATGAAAAGCTCTATCTCTATTATCTCCA
AAATGGAAGAGACATGTATGTGGACCAAGAATTAGATATTAATCGTTTAAGTGATT
ATGATGTCGATCACATTGTTCCACAAAGTTTCCTTAAAGACGATTCAATAGACAATA
AGGTCTTAACGCGTTCTGATAAAAATCGTGGTAAATCGGATAACGTTCCAAGTGAAG
AAGTAGTCAAAAAGATGAAAAACTATTGGAGACAACTTCTAAACGCCAAGTTAATC
ACTCAACGTAAGTTTGATAATTTAACGAAAGCTGAACGTGGAGGTTTGAGTGAACTT
GATAAAGCTGGTTTTATCAAACGCCAATTGGTTGAAACTCGCCAAATCACTAAGCAT
GTGGCACAAATTTTGGATAGTCGCATGAATACTAAATACGATGAAAATGATAAACTT
ATTCGAGAGGTTAAAGTGATTACCTTAAAATCTAAATTAGTTTCTGACTTCCGAAAA
GATTTCCAATTCTATAAAGTACGTGAGATTAACAATTACCATCATGCCCATGATGCG
TATCTAAATGCCGTCGTTGGAACTGCTTTGATTAAGAAATATCCAAAACTTGAATCG
GAGTTTGTCTATGGTGATTATAAAGTTTATGATGTTCGTAAAATGATTGCTAAGTCTG
AGCAAGAAATAGGCAAAGCAACCGCAAAATATTTCTTTTACTCTAATATCATGAACT
TCTTCAAAACAGAAATTACACTTGCAAATGGAGAGATTCGCAAACGCCCTCTAATCG
AAACTAATGGGGAAACTGGAGAAATTGTCTGGGATAAAGGGCGAGATTTTGCCACA
GTGCGCAAAGTATTGTCCATGCCCCAAGTCAATATTGTCAAGAAAACAGAAGTACA
GACAGGCGGATTCTCCAAGGAGTCAATTTTACCAAAAAGAAATTCGGACAAGCTTA
TTGCTCGTAAAAAAGACTGGGATCCAAAAAAATATGGTGGTTTTGATAGTCCAACG
GTAGCTTATTCAGTCCTAGTGGTTGCTAAGGTGGAAAAAGGGAAATCGAAGAAGTT
AAAATCCGTTAAAGAGTTACTAGGGATCACAATTATGGAAAGAAGTTCCTTTGAAA
AAAATCCGATTGACTTTTTAGAAGCTAAAGGATATAAGGAAGTTAAAAAAGACTTA
ATCATTAAACTACCTAAATATAGTCTTTTTGAGTTAGAAAACGGTCGTAAACGGATG
CTGGCTAGTGCCGGAGAATTACAAAAAGGAAATGAGCTGGCTCTGCCAAGCAAATA
TGTGAATTTTTTATATTTAGCTAGTCATTATGAAAAGTTGAAGGGTAGTCCAGAAGA
TAACGAACAAAAACAATTGTTTGTGGAGCAGCATAAGCATTATTTAGATGAGATTAT
TGAGCAAATCAGTGAATTTTCTAAGCGTGTTATTTTAGCAGATGCCAATTTAGATAA
AGTTCTTAGTGCATATAACAAACATAGAGACAAACCAATACGTGAACAAGCAGAAA
ATATTATTCATTTATTTACGTTGACGAATCTTGGAGCTCCCGCTGCTTTTAAATATTT
TGATACAACAATTGATCGTAAACGATATACGTCTACAAAAGAAGTTTTAGATGCCAC
TCTTATCCATCAATCCATCACTGGTCTTTATGAAACACGCATTGATTTGAGTCAGCTA
GGAGGTGACTGATAATCAAATGTCAGACGAAAATGCCAATTATTGAAGCGGCTAAC
GCCGCTTTTTTTGTTTCTGGTCTCCCTTGCTTGTACTTTACAGTATAGCTTCTAGAGAT
CTGCAGTGACTGGAGCTTCTAGAGATCTGCAGTGACTGGGAAAACCCTGGCGACTA
GTCTTGGACTCCTGTTGATAGATCCAGTAATGACCTCAGAACTCCATCTGGATTTGTT
CAGAACGCTCGGTTGCCGCCGGGCGTTTTTTATTGGTGAGAATCCAGGGGTCCCCAA
TAATTACGATTTAAATCCTTCAAACTTCCCAAAGGCGAGCCCTAGTGACATTAGAAA
ACCGACTGTAAAAAGTACAGTCGGCATTATCTCATATTATAAAAGCCAGTCATTAGG
CCTATCTGACAATTCCTGAATAGAGTTCATAAACAATCCTGCATGATAACCATCACA
AACAGAATGATGTACCTGTAAAGATAGCGGTAAATATATTGAATTACCTTTATTAAT
GAATTTTCCTGCTGTAATAATGGGTAGAAGGTAATTACTATTATTATTGATATTTAAG
TTAAACCCAGTAAATGAAGTCCATGGAATAATAGAAAGAGAAAAAGCATTTTCAGG
TATAGGTGTTTTGGGAAACAATTTCCCCGAACCATTATATTTCTCTACATCAGAAAG
GTATAAATCATAAAACTCTTTGAAGTCATTCTTTACAGGAGTCCAAATACCAGAGAA
TGTTTTAGATACACCATCAAAAATTGTATAAAGTGGCTCTAACTTATCCCAATAACC
TAACTCTCCGTCGCTATTGTAACCAGTTCTAAAAGCTGTATTTGAGTTTATCACCCTT
GTCACTAAGAAAATAAATGCAGGGTAAAATTTATATCCTTCTTGTTTTATGTTTCGGT
ATAAAACACTAATATCAATTTCTGTGGTTATACTAAAAGTCGTTTGTTGGTTCAAAT
AATGATTAAATATCTCTTTTCTCTTCCAATTGTCTAAATCAATTTTATTAAAGTTCATT
TGATATGCCTCCTAAATTTTTATCTAAAGTGAATTTAGGAGGCTTACTTGTCTGCTTT
CTTCATTAGAATCAATCCTTTTTTAAAAGTCAATCCCGTTTGTTGAAGACTTTTGTCC
TTTTCCGCTGCATAAACAATATGGCCCGTTTGTTGAACTACTCTTTAATAAAATAATT
TTTCCGTTCCCAATTCCACATTGCAATAATAGAAAATCCATCTTCATCGGCTTTTTCG
TCATCATCTGTATGAATCAAATCGCCTTCTTCTGTGTCATCAAGGTTTAATTTTTTAT
GTATTTCTTTTAACAAACCACCATAGGAGATTAACCTTTTACGGTGTAAACCTTCCTC
CAAATCAGACAAACGTTTCAAATTCTTTTCTTCATCATCGGTCATAAAATCCGTATCC
TTTACAGGATATTTTGCAGTTTCGTCAATTGCCGATTGTATATCCGATTTATATTTAT
TTTTCGGTCGAATCATTTGAACTTTTACATTTGGATCATAGTCTAATTTCATTGCCTTT
TTCCAAAATTGAATCCATTGTTTTTGATTCACGTAGTTTTCTGTATTCTTAAAATAAG
TTGGTTCCACACATACCAATACATGCATGTGCTGATTATAAGAATTATCTTTATTATT
TATTGTCACTTCCGTTGCACGCATAAAACCAACAAGATTTTTATTAATTTTTTTATAT
TGCATCATTCGGCGAAATCCTTGAGCCATATCTGACAAACTCTTATTTAATTCTTCGC
CATCATAAACATTTTTAACTGTTAATGTGAGAAACAACCAACGAACTGTTGGCTTTT
GTTTAATAACTTCAGCAACAACCTTTTGTGACTGAATGCCATGTTTCATTGCTCTCCT
CCAGTTGCACATTGGACAAAGCCTGGATTTACAAAACCACACTCGATACAACTTTCT
TTCGCCTGTTTCACGATTTTGTTTATACTCTAATATTTCAGCACAATCTTTTACTCTTT
CAGCCTTTTTAAATTCAAGAATATGCAGAAGTTCAAAGTAATCAACATTAGCGATTT
TCTTTTCTCTCCATGGTCTCACTTTTCCACTTTTTGTCTTGTCCACTAAAACCCTTGAT
TTTTCATCTGAATAAATGCTACTATTAGGACACATAATATTAAAAGAAACCCCCATC
TATTTAGTTATTTGTTTGGTCACTTATAACTTTAACAGATGGGGTTTTTCTGTGCAAC
CAATTTTAAGGGTTTTCCAATACTTTAAAACACATACATACCAACACTTCAACGCAC
CTTTCAGCAACTAAAATAAAAATGACGTTATTTCTATATGTATCAAGATAAGAAAGA
ACAAGTTCAAAACCATCAAAAAAAGACACCTTTTCAGGTGCTTTTTTTATTTTATAA
ACTCATTCCCTGATCTCGACTTCGTTCTTTTTTTACCTCTCGGTTATGAGTTAGTTCAA
ATTCGTTCTTTTTAGGTTCTAAATCGTGTTTTTCTTGGAATTGTGCTGTTTTATCCTTT
ACCTTGTCTACAAACCCCTTAAAAACGTTTTTAAAGGCTTTTAAGCGTCTGTACGTTC
CTTAAGGAATTATTCCTTAGTGCTTTCTAGGTTAATGTCATGATAATAATGGTTTCTT
AGACGTCAGGTGGCACTTTTCGGGGAAATGTCCGCGGAACCCCTATTTGTTTATTTTT
CTAAATACATTCAAATATGTATCCGCTCATGAGACAATAACCCTGATAAATGCTTCA
ATAATATTGAAAAAGGAAGAGTATGAGTATTCAACATTTCCGTGTCGCCCTTATTCC
CTTTTTTGCGGCATTTTGCCTTCCTGTTTTTGCTCACCCAGAAACGCTGGTGAAAGTA
AAAGATGCTGAAGATCAGTTGGGTGCACGAGTGGGTTACATCGAACTGGATCTCAA
CAGCGGTAAGATCCTTGAGAGTTTTCGCCCCGAAGAACGTTTTCCAATGATGAGCAC
TTTTAAAGTTCTGCTATGTGGCGCGGTATTATCCCGTGTTGACGCCGGGCAAGAGCA
ACTCGGTCGCCGCATACACTATTCTCAGAATGACTTGGTTGAGTACTCACCAGTCAC
AGAAAAGCATCTTACGGATGGCATGACAGTAAGAGAATTATGCAGTGCTGCCATAA
CCATGAGTGATAACACTGCGGCCAACTTACTTCTGACAACGATCGGAGGACCGAAG
GAGCTAACCGCTTTTTTGCACAACATGGGGGATCATGTAACTCGCCTTGATCGTTGG
GAACCGGAGCTGAATGAAGCCATACCAAACGACGAGCGTGACACCACGATGCCTGC
AGCAATGGCAACAACGTTGCGCAAACTATTAACTGGCGAACTACTTACTCTAGCTTC
CCGGCAACAATTAATAGACTGGATGGAGGCGGATAAAGTTGCAGGACCACTTCTGC
GCTCGGCCCTTCCGGCTGGCTGGTTTATTGCTGATAAATCTGGAGCCGGTGAGCGTG
GGTCTCGCGGTATCATTGCAGCACTGGGGCCAGATGGTAAGCCCTCCCGTATCGTAG
TTATCTACACGACGGGGAGTCAGGCAACTATGGATGAACGAAATAGACAGATCGCT
GAGATAGGTGCCTCACTGATTAAGCATTGGTAACTGTCAGACCAAGTTTACTCATAT
ATACTTTAGATTGATTTAAAACTTCATTTTTAATTTAAAAGGATCTAGGTGAAGATCC
TTTTTGATAATCTCATGACCAAAATCCCTTAACGTGAGTTTTCGTTCCACTGAGCGTC
AGACCCCTTAATAAGATGATCTTCTTGAGATCGTTTTGGTCTGCGCGTAATCTCTTGC
TCTGAAAACGAAAAAACCGCCTTGCAGGGAGGTTTTTCGAAGGTTCTCTGAGCTACC
AACTCTTTGAACCGAGGTAACTGGCTTGCAGGAGCGCAGTCACCAAAACTTGTCCTT
TCAGTTTAGCCTTAACCGGCGCATGACTTCAAGACTAACTCCTCTAAATCAATTACC
AGTGGCTGCTGCCAGTGGTGCTTTTGCATGTCTTTCCGGGTTGGACTCAAGACGATA
GTTACCGGATAAGGCGCAGCGGTCGGACTGAACGGGGGGTTCGTGCATACAGTCCA
GCTTGGAGCGAACTGCCTACCCGGAACTGAGTGTCAGGCGTGGAATGAGACAAACG
CGGCCATAACAGCGGAATGACACCGGTAAACCGAAAGGCAGGAACAGGAGAGCGC
ACGAGGGAGCCGCCAGGGGGAAACGCCTGGTATCTTTATAGTCCTGTCGGGTTTCGC
CACCACTGATTTGAGCGTCAGATTTCGTGATGCTTGTCAGGGGGCGGAGCCTATGGA
AAAACGCTTTGCCCGAGCCTATGGAAAAACGCTTTGCCCCACACAGGCTCTTGAACA
AGGGATAACAATTTGTCATAGCTGTATTCGTTACAAGGAGGATTTTCGAAAAAACAC
CCTAACGGGTGTTTTTTTATAGCTGGTCTCCCCAAGTGCTGTAAAAAACGCCGAATG
AAGCGGATGAAATTGCTCATCGTCTCGTGAAAAAGCCGAAGAAGGTTAAGCCTGGG
TATAAAAAGAAAATGAGCTATGAGATGGAGAAAGCCATAACCATGAGTGATAACAC
TGGTTTTAGAGCTAGAAATAGCAAGTTAAAATAAGGCTAGTCCGTTATCAACTTGAA
AAAGTGGCACCGAGTCGGTGCTTTTTTTCTCGGTACCAAATTCCAGAAAAGAGGCCT
CCCGAAAGGGGGGCCTTTTTTCGTTTTGGTCCCTCAATGATCTCCTTATCCGGTTAAG
ATGGC
>pCas9g2-D15-mut
(SEQ ID No: 238)
GAGTAAATCCTGGGGCAAATTCATCGAGGAGGAAGAGGCGGAAATGGCTTCCCGTC
GTAATCTAATGATTGTCGATGGAACTAACTTAGGCTTTCGCTTCAAACATAACAATA
GTAAAAAACCATTTGCCTCAAGTTATGTTTCAACTATTCAATCTCTGGCAAAATCCT
ACTCTGCCAGAACTACGATTGTTCTAGGTGATAAGGGCAAAAGCGTGTTCAGACTAG
AACATCTACCAGAGTATAAAGGTAATCGTGATGAAAAGTACGCACAACGTACGGAA
GAGGAGAAAGCGCTAGATGAGCAGTTCTTCGAATACCTCAAAGATGCTTTCGAGTT
GTGTAAAACTACATTCCCAACTTTTACCATTCGTGGTGTAGAAGCAGATGACATGGC
GGCGTATATTGTTAAGCTCATCGGGCATCTTTATGATCACGTTTGGCTAATATCTACA
GATGGTGACTGGGATACTTTATTAACGGATAAAGTTTCTCGTTTTTCTTTCACAACAC
GTCGTGAGTATCATCTTCGTGATATGTATGAGCACCATAACGTGGACGACGTAGAAC
AGTTTATCTCCCTGAAAGCAATTATGGGAGATCTAGGAGATAATATTCGTGGTGTTG
AAGGAATAGGAGCAAAACGCGGATATAATATTATTCGTGAGTTTGGTAACGTACTG
GATATTATTGATCAGCTTCCACTGCCTGGAAAGCAGAAATATATACAGAACCTGAAT
GCATCGGAAGAACTGCTTTTCCGAAACTTGATTCTGGTTGATTTACCTACCTACTGTG
TGGATGCTATTGCTGCTGTAGGTCAAGATGTGTTAGATAAGTTCACGAAAGACATCC
TTGAGATCGCGGAACAATGATAAGCTCTTAAAGGGGGTTTTAGATACCAGAGATGG
ATAAGAAATACTCAATAGGCTTAGATATCGGCACAAATAGCGTCGGATGGGCGGTG
ATCACTGATGAATATAAGGTTCCGTCTAAAAAGTTCAAGGTTCTGGGAAATACAGAC
CGCCACAGTATCAAAAAAAATCTTATAGGGGCTCTTTTATTTGACAGTGGAGAGACA
GCGGAAGCGACTCGTCTCAAACGGACAGCTCGTAGAAGGTATACACGTCGGAAGAA
TCGTATTTGTTATCTACAGGAGATTTTTTCAAATGAGATGGCGAAAGTAGATGATAG
TTTCTTTCATCGACTTGAAGAGTCTTTTTTGGTGGAAGAAGACAAGAAGCATGAACG
TCATCCTATTTTTGGAAATATAGTAGATGAAGTTGCTTATCATGAGAAATATCCAAC
TATCTATCATCTGCGAAAAAAATTGGTAGATTCTACTGATAAAGCGGATTTGCGCTT
AATCTATTTGGCCTTAGCGCATATGATTAAGTTTCGTGGTCATTTTTTGATTGAGGGA
GATTTAAATCCTGATAATAGTGATGTGGACAAACTATTTATCCAGTTGGTACAAACC
TACAATCAATTATTTGAAGAAAACCCTATTAACGCAAGTGGAGTAGATGCTAAAGC
GATTCTTTCTGCACGATTGAGTAAATCAAGACGATTAGAAAATCTCATTGCTCAGCT
CCCCGGTGAGAAGAAAAATGGCTTATTTGGGAATCTCATTGCTTTGTCATTGGGTTT
GACCCCTAATTTTAAATCAAATTTTGATTTGGCAGAAGATGCTAAATTACAGCTTTC
AAAAGATACTTACGATGATGATTTAGATAATTTATTGGCGCAAATTGGAGATCAATA
TGCTGATTTGTTTTTGGCAGCTAAGAATTTATCAGATGCTATTTTACTTTCAGATATC
CTAAGAGTAAATACTGAAATAACTAAGGCTCCCCTATCAGCTTCAATGATTAAACGC
TACGATGAACATCATCAAGACTTGACTCTTTTAAAAGCTTTAGTTCGACAACAACTT
CCAGAAAAGTATAAAGAAATCTTTTTTGATCAATCAAAAAACGGATATGCAGGTTAT
ATTGATGGGGGAGCTAGCCAAGAAGAATTTTATAAATTTATCAAACCAATTTTAGAA
AAAATGGATGGTACTGAGGAATTATTGGTGAAACTAAATCGTGAAGATTTGCTGCG
CAAGCAACGGACCTTTGACAACGGCTCTATTCCCCATCAAATTCACTTGGGTGAGCT
GCATGCTATTTTGAGAAGACAAGAAGACTTTTATCCATTTTTAAAAGACAATCGTGA
GAAGATTGAAAAAATCTTGACTTTTCGAATTCCTTATTATGTTGGTCCATTGGCGCGT
GGCAATAGTCGTTTTGCATGGATGACTCGGAAGTCTGAAGAAACAATTACCCCATGG
AATTTTGAAGAAGTTGTCGATAAAGGTGCTTCAGCTCAATCATTTATTGAACGCATG
ACAAACTTTGATAAAAATCTTCCAAATGAAAAAGTACTACCAAAACATAGTTTGCTT
TATGAGTATTTTACGGTTTATAACGAATTGACAAAGGTCAAATATGTTACTGAAGGA
ATGCGAAAACCAGCATTTCTTTCAGGTGAACAGAAGAAAGCCATTGTTGATTTACTC
TTCAAAACAAATCGAAAAGTAACCGTTAAGCAATTAAAAGAAGATTATTTCAAAAA
AATAGAATGTTTTGATAGTGTTGAAATTTCAGGAGTTGAAGATAGATTTAATGCTTC
ATTAGGTACCTACCATGATTTGCTAAAAATTATTAAAGATAAAGATTTTTTGGATAA
TGAAGAAAATGAAGATATCTTAGAGGATATTGTTTTAACATTGACCTTATTTGAAGA
TAGGGAGATGATTGAGGAAAGACTTAAAACATATGCTCACCTCTTTGATGATAAGGT
GATGAAACAGCTTAAACGTCGCCGTTATACTGGTTGGGGACGTTTGTCTCGAAAATT
GATTAATGGTATTAGGGATAAGCAATCTGGCAAAACAATATTAGATTTTTTGAAATC
AGATGGTTTTGCCAATCGCAATTTTATGCAGCTGATCCATGATGATAGTTTGACATTT
AAAGAAGACATTCAAAAAGCACAAGTGTCTGGACAAGGCGATAGTTTACATGAACA
TATTGCAAATTTAGCTGGTAGCCCTGCTATTAAAAAAGGTATTTTACAGACTGTAAA
AGTTGTTGATGAATTGGTCAAAGTAATGGGGCGGCATAAGCCAGAAAATATCGTTA
TTGAAATGGCACGTGAAAATCAGACAACTCAAAAGGGCCAGAAAAATTCGCGAGAG
CGTATGAAACGAATCGAAGAAGGTATCAAAGAATTAGGAAGTCAGATTCTTAAAGA
GCATCCTGTTGAAAATACTCAATTGCAAAATGAAAAGCTCTATCTCTATTATCTCCA
AAATGGAAGAGACATGTATGTGGACCAAGAATTAGATATTAATCGTTTAAGTGATT
ATGATGTCGATCACATTGTTCCACAAAGTTTCCTTAAAGACGATTCAATAGACAATA
AGGTCTTAACGCGTTCTGATAAAAATCGTGGTAAATCGGATAACGTTCCAAGTGAAG
AAGTAGTCAAAAAGATGAAAAACTATTGGAGACAACTTCTAAACGCCAAGTTAATC
ACTCAACGTAAGTTTGATAATTTAACGAAAGCTGAACGTGGAGGTTTGAGTGAACTT
GATAAAGCTGGTTTTATCAAACGCCAATTGGTTGAAACTCGCCAAATCACTAAGCAT
GTGGCACAAATTTTGGATAGTCGCATGAATACTAAATACGATGAAAATGATAAACTT
ATTCGAGAGGTTAAAGTGATTACCTTAAAATCTAAATTAGTTTCTGACTTCCGAAAA
GATTTCCAATTCTATAAAGTACGTGAGATTAACAATTACCATCATGCCCATGATGCG
TATCTAAATGCCGTCGTTGGAACTGCTTTGATTAAGAAATATCCAAAACTTGAATCG
GAGTTTGTCTATGGTGATTATAAAGTTTATGATGTTCGTAAAATGATTGCTAAGTCTG
AGCAAGAAATAGGCAAAGCAACCGCAAAATATTTCTTTTACTCTAATATCATGAACT
TCTTCAAAACAGAAATTACACTTGCAAATGGAGAGATTCGCAAACGCCCTCTAATCG
AAACTAATGGGGAAACTGGAGAAATTGTCTGGGATAAAGGGCGAGATTTTGCCACA
GTGCGCAAAGTATTGTCCATGCCCCAAGTCAATATTGTCAAGAAAACAGAAGTACA
GACAGGCGGATTCTCCAAGGAGTCAATTTTACCAAAAAGAAATTCGGACAAGCTTA
TTGCTCGTAAAAAAGACTGGGATCCAAAAAAATATGGTGGTTTTGATAGTCCAACG
GTAGCTTATTCAGTCCTAGTGGTTGCTAAGGTGGAAAAAGGGAAATCGAAGAAGTT
AAAATCCGTTAAAGAGTTACTAGGGATCACAATTATGGAAAGAAGTTCCTTTGAAA
AAAATCCGATTGACTTTTTAGAAGCTAAAGGATATAAGGAAGTTAAAAAAGACTTA
ATCATTAAACTACCTAAATATAGTCTTTTTGAGTTAGAAAACGGTCGTAAACGGATG
CTGGCTAGTGCCGGAGAATTACAAAAAGGAAATGAGCTGGCTCTGCCAAGCAAATA
TGTGAATTTTTTATATTTAGCTAGTCATTATGAAAAGTTGAAGGGTAGTCCAGAAGA
TAACGAACAAAAACAATTGTTTGTGGAGCAGCATAAGCATTATTTAGATGAGATTAT
TGAGCAAATCAGTGAATTTTCTAAGCGTGTTATTTTAGCAGATGCCAATTTAGATAA
AGTTCTTAGTGCATATAACAAACATAGAGACAAACCAATACGTGAACAAGCAGAAA
ATATTATTCATTTATTTACGTTGACGAATCTTGGAGCTCCCGCTGCTTTTAAATATTT
TGATACAACAATTGATCGTAAACGATATACGTCTACAAAAGAAGTTTTAGATGCCAC
TCTTATCCATCAATCCATCACTGGTCTTTATGAAACACGCATTGATTTGAGTCAGCTA
GGAGGTGACTGATAATCAAATGTCAGACGAAAATGCCAATTATTGAAGCGGCTAAC
GCCGCTTTTTTTGTTTCTGGTCTCCCTTGCTTGTACTTTACAGTATAGCTTCTAGAGAT
CTGCAGTGACTGGAGCTTCTAGAGATCAGGTCAGACGCGATTTCCTGGGTGCTCGAG
CCATGGGAAAGCGGTGGTGGAAAAAACGCCGAATGAAGCGGATGAAATTGCTCATC
GTCTCGTGAAAAAGCCGAAGAAGGTTAAGCCTGGGTATAAAAAGAAAATGAGCTAT
GAGATGGAGAAATTGAAGTCATGCGCCGGTTAGTTTTAGAGCTAGAAATAGCAAGT
TAAAATAAGGCTAGTCCGTTATCAACTTGAAAAAGTGGCACCGAGTCGGTGCTTTTT
TTCTCGGTACCAAATTCCAGAAAAGAGGCCTCCCGAAAGGGGGGCCTTTTTTCGTTT
TGGTCCCTCAATGATCCGACAGGAAGTGGTATTAGCGACTCCTACAAGGAATAGGT
GTCTGGGTCATGCAGTGACTGGGAAAACCCTGGCGACTAGTCTTGGACTCCTGTTGA
TAGATCCAGTAATGACCTCAGAACTCCATCTGGATTTGTTCAGAACGCTCGGTTGCC
GCCGGGCGTTTTTTATTGGTGAGAATCCAGGGGTCCCCAATAATTACGATTTAAATC
CTTCAAACTTCCCAAAGGCGAGCCCTAGTGACATTAGAAAACCGACTGTAAAAAGT
ACAGTCGGCATTATCTCATATTATAAAAGCCAGTCATTAGGCCTATCTGACAATTCC
TGAATAGAGTTCATAAACAATCCTGCATGATAACCATCACAAACAGAATGATGTAC
CTGTAAAGATAGCGGTAAATATATTGAATTACCTTTATTAATGAATTTTCCTGCTGTA
ATAATGGGTAGAAGGTAATTACTATTATTATTGATATTTAAGTTAAACCCAGTAAAT
GAAGTCCATGGAATAATAGAAAGAGAAAAAGCATTTTCAGGTATAGGTGTTTTGGG
AAACAATTTCCCCGAACCATTATATTTCTCTACATCAGAAAGGTATAAATCATAAAA
CTCTTTGAAGTCATTCTTTACAGGAGTCCAAATACCAGAGAATGTTTTAGATACACC
ATCAAAAATTGTATAAAGTGGCTCTAACTTATCCCAATAACCTAACTCTCCGTCGCT
ATTGTAACCAGTTCTAAAAGCTGTATTTGAGTTTATCACCCTTGTCACTAAGAAAAT
AAATGCAGGGTAAAATTTATATCCTTCTTGTTTTATGTTTCGGTATAAAACACTAATA
TCAATTTCTGTGGTTATACTAAAAGTCGTTTGTTGGTTCAAATAATGATTAAATATCT
CTTTTCTCTTCCAATTGTCTAAATCAATTTTATTAAAGTTCATTTGATATGCCTCCTAA
ATTTTTATCTAAAGTGAATTTAGGAGGCTTACTTGTCTGCTTTCTTCATTAGAATCAA
TCCTTTTTTAAAAGTCAATCCCGTTTGTTGAAGACTTTTGTCCTTTTCCGCTGCATAA
ACAATATGGCCCGTTTGTTGAACTACTCTTTAATAAAATAATTTTTCCGTTCCCAATT
CCACATTGCAATAATAGAAAATCCATCTTCATCGGCTTTTTCGTCATCATCTGTATGA
ATCAAATCGCCTTCTTCTGTGTCATCAAGGTTTAATTTTTTATGTATTTCTTTTAACAA
ACCACCATAGGAGATTAACCTTTTACGGTGTAAACCTTCCTCCAAATCAGACAAACG
TTTCAAATTCTTTTCTTCATCATCGGTCATAAAATCCGTATCCTTTACAGGATATTTT
GCAGTTTCGTCAATTGCCGATTGTATATCCGATTTATATTTATTTTTCGGTCGAATCA
TTTGAACTTTTACATTTGGATCATAGTCTAATTTCATTGCCTTTTTCCAAAATTGAAT
CCATTGTTTTTGATTCACGTAGTTTTCTGTATTCTTAAAATAAGTTGGTTCCACACAT
ACCAATACATGCATGTGCTGATTATAAGAATTATCTTTATTATTTATTGTCACTTCCG
TTGCACGCATAAAACCAACAAGATTTTTATTAATTTTTTTATATTGCATCATTCGGCG
AAATCCTTGAGCCATATCTGACAAACTCTTATTTAATTCTTCGCCATCATAAACATTT
TTAACTGTTAATGTGAGAAACAACCAACGAACTGTTGGCTTTTGTTTAATAACTTCA
GCAACAACCTTTTGTGACTGAATGCCATGTTTCATTGCTCTCCTCCAGTTGCACATTG
GACAAAGCCTGGATTTACAAAACCACACTCGATACAACTTTCTTTCGCCTGTTTCAC
GATTTTGTTTATACTCTAATATTTCAGCACAATCTTTTACTCTTTCAGCCTTTTTAAAT
TCAAGAATATGCAGAAGTTCAAAGTAATCAACATTAGCGATTTTCTTTTCTCTCCAT
GGTCTCACTTTTCCACTTTTTGTCTTGTCCACTAAAACCCTTGATTTTTCATCTGAATA
AATGCTACTATTAGGACACATAATATTAAAAGAAACCCCCATCTATTTAGTTATTTG
TTTGGTCACTTATAACTTTAACAGATGGGGTTTTTCTGTGCAACCAATTTTAAGGGTT
TTCCAATACTTTAAAACACATACATACCAACACTTCAACGCACCTTTCAGCAACTAA
AATAAAAATGACGTTATTTCTATATGTATCAAGATAAGAAAGAACAAGTTCAAAAC
CATCAAAAAAAGACACCTTTTCAGGTGCTTTTTTTATTTTATAAACTCATTCCCTGAT
CTCGACTTCGTTCTTTTTTTACCTCTCGGTTATGAGTTAGTTCAAATTCGTTCTTTTTA
GGTTCTAAATCGTGTTTTTCTTGGAATTGTGCTGTTTTATCCTTTACCTTGTCTACAAA
CCCCTTAAAAACGTTTTTAAAGGCTTTTAAGCGTCTGTACGTTCCTTAAGGAATTATT
CCTTAGTGCTTTCTAGGTTAATGTCATGATAATAATGGTTTCTTAGACGTCAGGTGGC
ACTTTTCGGGGAAATGTCCGCGGAACCCCTATTTGTTTATTTTTCTAAATACATTCAA
ATATGTATCCGCTCATGAGACAATAACCCTGATAAATGCTTCAATAATATTGAAAAA
GGAAGAGTATGAGTATTCAACATTTCCGTGTCGCCCTTATTCCCTTTTTTGCGGCATT
TTGCCTTCCTGTTTTTGCTCACCCAGAAACGCTGGTGAAAGTAAAAGATGCTGAAGA
TCAGTTGGGTGCACGAGTGGGTTACATCGAACTGGATCTCAACAGCGGTAAGATCCT
TGAGAGTTTTCGCCCCGAAGAACGTTTTCCAATGATGAGCACTTTTAAAGTTCTGCT
ATGTGGCGCGGTATTATCCCGTGTTGACGCCGGGCAAGAGCAACTCGGTCGCCGCAT
ACACTATTCTCAGAATGACTTGGTTGAGTACTCACCAGTCACAGAAAAGCATCTTAC
GGATGGCATGACAGTAAGAGAATTATGCAGTGCTGCCATAACCATGAGTGATAACA
CTGCGGCCAACTTACTTCTGACAACGATCGGAGGACCGAAGGAGCTAACCGCTTTTT
TGCACAACATGGGGGATCATGTAACTCGCCTTGATCGTTGGGAACCGGAGCTGAAT
GAAGCCATACCAAACGACGAGCGTGACACCACGATGCCTGCAGCAATGGCAACAAC
GTTGCGCAAACTATTAACTGGCGAACTACTTACTCTAGCTTCCCGGCAACAATTAAT
AGACTGGATGGAGGCGGATAAAGTTGCAGGACCACTTCTGCGCTCGGCCCTTCCGG
CTGGCTGGTTTATTGCTGATAAATCTGGAGCCGGTGAGCGTGGGTCTCGCGGTATCA
TTGCAGCACTGGGGCCAGATGGTAAGCCCTCCCGTATCGTAGTTATCTACACGACGG
GGAGTCAGGCAACTATGGATGAACGAAATAGACAGATCGCTGAGATAGGTGCCTCA
CTGATTAAGCATTGGTAACTGTCAGACCAAGTTTACTCATATATACTTTAGATTGATT
TAAAACTTCATTTTTAATTTAAAAGGATCTAGGTGAAGATCCTTTTTGATAATCTCAT
GACCAAAATCCCTTAACGTGAGTTTTCGTTCCACTGAGCGTCAGACCCCTTAATAAG
ATGATCTTCTTGAGATCGTTTTGGTCTGCGCGTAATCTCTTGCTCTGAAAACGAAAA
AACCGCCTTGCAGGGAGGTTTTTCGAAGGTTCTCTGAGCTACCAACTCTTTGAACCG
AGGTAACTGGCTTGCAGGAGCGCAGTCACCAAAACTTGTCCTTTCAGTTTAGCCTTA
ACCGGCGCATGACTTCAAGACTAACTCCTCTAAATCAATTACCAGTGGCTGCTGCCA
GTGGTGCTTTTGCATGTCTTTCCGGGTTGGACTCAAGACGATAGTTACCGGATAAGG
CGCAGCGGTCGGACTGAACGGGGGGTTCGTGCATACAGTCCAGCTTGGAGCGAACT
GCCTACCCGGAACTGAGTGTCAGGCGTGGAATGAGACAAACGCGGCCATAACAGCG
GAATGACACCGGTAAACCGAAAGGCAGGAACAGGAGAGCGCACGAGGGAGCCGCC
AGGGGGAAACGCCTGGTATCTTTATAGTCCTGTCGGGTTTCGCCACCACTGATTTGA
GCGTCAGATTTCGTGATGCTTGTCAGGGGGCGGAGCCTATGGAAAAACGCTTTGCCC
GAGCCTATGGAAAAACGCTTTGCCCCACACAGGCTCTTGAACAAGGGATAACAATT
TGTCATAGCTGTATTCGTTACAAGGAGGATTTTCGAAAAAACACCCTAACGGGTGTT
TTTTTATAGCTGGTCTCCCCAAGTGCTGTAAAAAACGCCGAATGAAGCGGATGAAAT
TGCTCATCGTCTCGTGAAAAAGCCGAAGAAGGTTAAGCCTGGGTATAAAAAGAAAA
TGAGCTATGAGATGGAGAAAGCCATAACCATGAGTGATAACACTGGTTTTAGAGCT
AGAAATAGCAAGTTAAAATAAGGCTAGTCCGTTATCAACTTGAAAAAGTGGCACCG
AGTCGGTGCTTTTTTTCTCGGTACCAAATTCCAGAAAAGAGGCCTCCCGAAAGGGGG
GCCTTTTTTCGTTTTGGTCCCTCAATGATCTCCTTATCCGGTTAAGATGGC
>pCas9g1-D15-GFP
(SEQ ID No: 239)
GTGACTGGGAAAACCCTGGCGACTAGTCTTGGACTCCTGTTGATAGATCCAGTAATG
ACCTCAGAACTCCATCTGGATTTGTTCAGAACGCTCGGTTGCCGCCGGGCGTTTTTTA
TTGGTGAGAATCCAGGGGTCCCCAATAATTACGATTTAAATCCTTCAAACTTCCCAA
AGGCGAGCCCTAGTGACATTAGAAAACCGACTGTAAAAAGTACAGTCGGCATTATC
TCATATTATAAAAGCCAGTCATTAGGCCTATCTGACAATTCCTGAATAGAGTTCATA
AACAATCCTGCATGATAACCATCACAAACAGAATGATGTACCTGTAAAGATAGCGG
TAAATATATTGAATTACCTTTATTAATGAATTTTCCTGCTGTAATAATGGGTAGAAG
GTAATTACTATTATTATTGATATTTAAGTTAAACCCAGTAAATGAAGTCCATGGAAT
AATAGAAAGAGAAAAAGCATTTTCAGGTATAGGTGTTTTGGGAAACAATTTCCCCG
AACCATTATATTTCTCTACATCAGAAAGGTATAAATCATAAAACTCTTTGAAGTCAT
TCTTTACAGGAGTCCAAATACCAGAGAATGTTTTAGATACACCATCAAAAATTGTAT
AAAGTGGCTCTAACTTATCCCAATAACCTAACTCTCCGTCGCTATTGTAACCAGTTCT
AAAAGCTGTATTTGAGTTTATCACCCTTGTCACTAAGAAAATAAATGCAGGGTAAAA
TTTATATCCTTCTTGTTTTATGTTTCGGTATAAAACACTAATATCAATTTCTGTGGTTA
TACTAAAAGTCGTTTGTTGGTTCAAATAATGATTAAATATCTCTTTTCTCTTCCAATT
GTCTAAATCAATTTTATTAAAGTTCATTTGATATGCCTCCTAAATTTTTATCTAAAGT
GAATTTAGGAGGCTTACTTGTCTGCTTTCTTCATTAGAATCAATCCTTTTTTAAAAGT
CAATCCCGTTTGTTGAAGACTTTTGTCCTTTTCCGCTGCATAAACAATATGGCCCGTT
TGTTGAACTACTCTTTAATAAAATAATTTTTCCGTTCCCAATTCCACATTGCAATAAT
AGAAAATCCATCTTCATCGGCTTTTTCGTCATCATCTGTATGAATCAAATCGCCTTCT
TCTGTGTCATCAAGGTTTAATTTTTTATGTATTTCTTTTAACAAACCACCATAGGAGA
TTAACCTTTTACGGTGTAAACCTTCCTCCAAATCAGACAAACGTTTCAAATTCTTTTC
TTCATCATCGGTCATAAAATCCGTATCCTTTACAGGATATTTTGCAGTTTCGTCAATT
GCCGATTGTATATCCGATTTATATTTATTTTTCGGTCGAATCATTTGAACTTTTACATT
TGGATCATAGTCTAATTTCATTGCCTTTTTCCAAAATTGAATCCATTGTTTTTGATTC
ACGTAGTTTTCTGTATTCTTAAAATAAGTTGGTTCCACACATACCAATACATGCATGT
GCTGATTATAAGAATTATCTTTATTATTTATTGTCACTTCCGTTGCACGCATAAAACC
AACAAGATTTTTATTAATTTTTTTATATTGCATCATTCGGCGAAATCCTTGAGCCATA
TCTGACAAACTCTTATTTAATTCTTCGCCATCATAAACATTTTTAACTGTTAATGTGA
GAAACAACCAACGAACTGTTGGCTTTTGTTTAATAACTTCAGCAACAACCTTTTGTG
ACTGAATGCCATGTTTCATTGCTCTCCTCCAGTTGCACATTGGACAAAGCCTGGATTT
ACAAAACCACACTCGATACAACTTTCTTTCGCCTGTTTCACGATTTTGTTTATACTCT
AATATTTCAGCACAATCTTTTACTCTTTCAGCCTTTTTAAATTCAAGAATATGCAGAA
GTTCAAAGTAATCAACATTAGCGATTTTCTTTTCTCTCCATGGTCTCACTTTTCCACTT
TTTGTCTTGTCCACTAAAACCCTTGATTTTTCATCTGAATAAATGCTACTATTAGGAC
ACATAATATTAAAAGAAACCCCCATCTATTTAGTTATTTGTTTGGTCACTTATAACTT
TAACAGATGGGGTTTTTCTGTGCAACCAATTTTAAGGGTTTTCCAATACTTTAAAAC
ACATACATACCAACACTTCAACGCACCTTTCAGCAACTAAAATAAAAATGACGTTAT
TTCTATATGTATCAAGATAAGAAAGAACAAGTTCAAAACCATCAAAAAAAGACACC
TTTTCAGGTGCTTTTTTTATTTTATAAACTCATTCCCTGATCTCGACTTCGTTCTTTTTT
TACCTCTCGGTTATGAGTTAGTTCAAATTCGTTCTTTTTAGGTTCTAAATCGTGTTTTT
CTTGGAATTGTGCTGTTTTATCCTTTACCTTGTCTACAAACCCCTTAAAAACGTTTTT
AAAGGCTTTTAAGCGTCTGTACGTTCCTTAAGGAATTATTCCTTAGTGCTTTCTAGGT
TAATGTCATGATAATAATGGTTTCTTAGACGTCAGGTGGCACTTTTCGGGGAAATGT
CCGCGGAACCCCTATTTGTTTATTTTTCTAAATACATTCAAATATGTATCCGCTCATG
AGACAATAACCCTGATAAATGCTTCAATAATATTGAAAAAGGAAGAGTATGAGTAT
TCAACATTTCCGTGTCGCCCTTATTCCCTTTTTTGCGGCATTTTGCCTTCCTGTTTTTG
CTCACCCAGAAACGCTGGTGAAAGTAAAAGATGCTGAAGATCAGTTGGGTGCACGA
GTGGGTTACATCGAACTGGATCTCAACAGCGGTAAGATCCTTGAGAGTTTTCGCCCC
GAAGAACGTTTTCCAATGATGAGCACTTTTAAAGTTCTGCTATGTGGCGCGGTATTA
TCCCGTGTTGACGCCGGGCAAGAGCAACTCGGTCGCCGCATACACTATTCTCAGAAT
GACTTGGTTGAGTACTCACCAGTCACAGAAAAGCATCTTACGGATGGCATGACAGT
AAGAGAATTATGCAGTGCTGCCATAACCATGAGTGATAACACTGCGGCCAACTTACT
TCTGACAACGATCGGAGGACCGAAGGAGCTAACCGCTTTTTTGCACAACATGGGGG
ATCATGTAACTCGCCTTGATCGTTGGGAACCGGAGCTGAATGAAGCCATACCAAAC
GACGAGCGTGACACCACGATGCCTGCAGCAATGGCAACAACGTTGCGCAAACTATT
AACTGGCGAACTACTTACTCTAGCTTCCCGGCAACAATTAATAGACTGGATGGAGGC
GGATAAAGTTGCAGGACCACTTCTGCGCTCGGCCCTTCCGGCTGGCTGGTTTATTGC
TGATAAATCTGGAGCCGGTGAGCGTGGGTCTCGCGGTATCATTGCAGCACTGGGGCC
AGATGGTAAGCCCTCCCGTATCGTAGTTATCTACACGACGGGGAGTCAGGCAACTAT
GGATGAACGAAATAGACAGATCGCTGAGATAGGTGCCTCACTGATTAAGCATTGGT
AACTGTCAGACCAAGTTTACTCATATATACTTTAGATTGATTTAAAACTTCATTTTTA
ATTTAAAAGGATCTAGGTGAAGATCCTTTTTGATAATCTCATGACCAAAATCCCTTA
ACGTGAGTTTTCGTTCCACTGAGCGTCAGACCCCTTAATAAGATGATCTTCTTGAGA
TCGTTTTGGTCTGCGCGTAATCTCTTGCTCTGAAAACGAAAAAACCGCCTTGCAGGG
AGGTTTTTCGAAGGTTCTCTGAGCTACCAACTCTTTGAACCGAGGTAACTGGCTTGC
AGGAGCGCAGTCACCAAAACTTGTCCTTTCAGTTTAGCCTTAACCGGCGCATGACTT
CAAGACTAACTCCTCTAAATCAATTACCAGTGGCTGCTGCCAGTGGTGCTTTTGCAT
GTCTTTCCGGGTTGGACTCAAGACGATAGTTACCGGATAAGGCGCAGCGGTCGGACT
GAACGGGGGGTTCGTGCATACAGTCCAGCTTGGAGCGAACTGCCTACCCGGAACTG
AGTGTCAGGCGTGGAATGAGACAAACGCGGCCATAACAGCGGAATGACACCGGTAA
ACCGAAAGGCAGGAACAGGAGAGCGCACGAGGGAGCCGCCAGGGGGAAACGCCTG
GTATCTTTATAGTCCTGTCGGGTTTCGCCACCACTGATTTGAGCGTCAGATTTCGTGA
TGCTTGTCAGGGGGCGGAGCCTATGGAAAAACGCTTTGCCCGAGCCTATGGAAAAA
CGCTTTGCCCCACACAGGCTCTTGAACAAGGGATAACAATTTGTCATAGCTGTATTC
GTTACAAGGAGGATTTTCGAAAAAACACCCTAACGGGTGTTTTTTTATAGCTGGTCT
CCCCAAGTGCTGTAAAAAACGCCGAATGAAGCGGATGAAATTGCTCATCGTCTCGT
GAAAAAGCCGAAGAAGGTTAAGCCTGGGTATAAAAAGAAAATGAGCTATGAGATG
GAGAAAGCCATAACCATGAGTGATAACACTGGTTTTAGAGCTAGAAATAGCAAGTT
AAAATAAGGCTAGTCCGTTATCAACTTGAAAAAGTGGCACCGAGTCGGTGCTTTTTT
TCTCGGTACCAAATTCCAGAAAAGAGGCCTCCCGAAAGGGGGGCCTTTTTTCGTTTT
GGTCCCTCAATGATCTCCTTATCCGGTTAAGATGGCTTTTAAATCAATTTTCAGCTCC
TGTATACAATTACCAAAGTTTTTCTGAATGAAGCCATGTGTTTTGACACATTCTATAC
TCACAAGGAGGTGAGACACATGAGTAAATCCTGGGGCAAATTCATCGAGGAGGAAG
AGGCGGAAATGGCTTCCCGTCGTAATCTAATGATTGTCGATGGAACTAACTTAGGCT
TTCGCTTCAAACATAACAATAGTAAAAAACCATTTGCCTCAAGTTATGTTTCAACTA
TTCAATCTCTGGCAAAATCCTACTCTGCCAGAACTACGATTGTTCTAGGTGATAAGG
GCAAAAGCGTGTTCAGACTAGAACATCTACCAGAGTATAAAGGTAATCGTGATGAA
AAGTACGCACAACGTACGGAAGAGGAGAAAGCGCTAGATGAGCAGTTCTTCGAATA
CCTCAAAGATGCTTTCGAGTTGTGTAAAACTACATTCCCAACTTTTACCATTCGTGGT
GTAGAAGCAGATGACATGGCGGCGTATATTGTTAAGCTCATCGGGCATCTTTATGAT
CACGTTTGGCTAATATCTACAGATGGTGACTGGGATACTTTATTAACGGATAAAGTT
TCTCGTTTTTCTTTCACAACACGTCGTGAGTATCATCTTCGTGATATGTATGAGCACC
ATAACGTGGACGACGTAGAACAGTTTATCTCCCTGAAAGCAATTATGGGAGATCTA
GGAGATAATATTCGTGGTGTTGAAGGAATAGGAGCAAAACGCGGATATAATATTAT
TCGTGAGTTTGGTAACGTACTGGATATTATTGATCAGCTTCCACTGCCTGGAAAGCA
GAAATATATACAGAACCTGAATGCATCGGAAGAACTGCTTTTCCGAAACTTGATTCT
GGTTGATTTACCTACCTACTGTGTGGATGCTATTGCTGCTGTAGGTCAAGATGTGTTA
GATAAGTTCACGAAAGACATCCTTGAGATCGCGGAACAATGATAAGCTCTTAAAGG
GGGTTTTAGATACCAGAGATGGATAAGAAATACTCAATAGGCTTAGATATCGGCAC
AAATAGCGTCGGATGGGCGGTGATCACTGATGAATATAAGGTTCCGTCTAAAAAGT
TCAAGGTTCTGGGAAATACAGACCGCCACAGTATCAAAAAAAATCTTATAGGGGCT
CTTTTATTTGACAGTGGAGAGACAGCGGAAGCGACTCGTCTCAAACGGACAGCTCGT
AGAAGGTATACACGTCGGAAGAATCGTATTTGTTATCTACAGGAGATTTTTTCAAAT
GAGATGGCGAAAGTAGATGATAGTTTCTTTCATCGACTTGAAGAGTCTTTTTTGGTG
GAAGAAGACAAGAAGCATGAACGTCATCCTATTTTTGGAAATATAGTAGATGAAGT
TGCTTATCATGAGAAATATCCAACTATCTATCATCTGCGAAAAAAATTGGTAGATTC
TACTGATAAAGCGGATTTGCGCTTAATCTATTTGGCCTTAGCGCATATGATTAAGTTT
CGTGGTCATTTTTTGATTGAGGGAGATTTAAATCCTGATAATAGTGATGTGGACAAA
CTATTTATCCAGTTGGTACAAACCTACAATCAATTATTTGAAGAAAACCCTATTAAC
GCAAGTGGAGTAGATGCTAAAGCGATTCTTTCTGCACGATTGAGTAAATCAAGACG
ATTAGAAAATCTCATTGCTCAGCTCCCCGGTGAGAAGAAAAATGGCTTATTTGGGAA
TCTCATTGCTTTGTCATTGGGTTTGACCCCTAATTTTAAATCAAATTTTGATTTGGCA
GAAGATGCTAAATTACAGCTTTCAAAAGATACTTACGATGATGATTTAGATAATTTA
TTGGCGCAAATTGGAGATCAATATGCTGATTTGTTTTTGGCAGCTAAGAATTTATCA
GATGCTATTTTACTTTCAGATATCCTAAGAGTAAATACTGAAATAACTAAGGCTCCC
CTATCAGCTTCAATGATTAAACGCTACGATGAACATCATCAAGACTTGACTCTTTTA
AAAGCTTTAGTTCGACAACAACTTCCAGAAAAGTATAAAGAAATCTTTTTTGATCAA
TCAAAAAACGGATATGCAGGTTATATTGATGGGGGAGCTAGCCAAGAAGAATTTTA
TAAATTTATCAAACCAATTTTAGAAAAAATGGATGGTACTGAGGAATTATTGGTGAA
ACTAAATCGTGAAGATTTGCTGCGCAAGCAACGGACCTTTGACAACGGCTCTATTCC
CCATCAAATTCACTTGGGTGAGCTGCATGCTATTTTGAGAAGACAAGAAGACTTTTA
TCCATTTTTAAAAGACAATCGTGAGAAGATTGAAAAAATCTTGACTTTTCGAATTCC
TTATTATGTTGGTCCATTGGCGCGTGGCAATAGTCGTTTTGCATGGATGACTCGGAA
GTCTGAAGAAACAATTACCCCATGGAATTTTGAAGAAGTTGTCGATAAAGGTGCTTC
AGCTCAATCATTTATTGAACGCATGACAAACTTTGATAAAAATCTTCCAAATGAAAA
AGTACTACCAAAACATAGTTTGCTTTATGAGTATTTTACGGTTTATAACGAATTGAC
AAAGGTCAAATATGTTACTGAAGGAATGCGAAAACCAGCATTTCTTTCAGGTGAAC
AGAAGAAAGCCATTGTTGATTTACTCTTCAAAACAAATCGAAAAGTAACCGTTAAG
CAATTAAAAGAAGATTATTTCAAAAAAATAGAATGTTTTGATAGTGTTGAAATTTCA
GGAGTTGAAGATAGATTTAATGCTTCATTAGGTACCTACCATGATTTGCTAAAAATT
ATTAAAGATAAAGATTTTTTGGATAATGAAGAAAATGAAGATATCTTAGAGGATATT
GTTTTAACATTGACCTTATTTGAAGATAGGGAGATGATTGAGGAAAGACTTAAAACA
TATGCTCACCTCTTTGATGATAAGGTGATGAAACAGCTTAAACGTCGCCGTTATACT
GGTTGGGGACGTTTGTCTCGAAAATTGATTAATGGTATTAGGGATAAGCAATCTGGC
AAAACAATATTAGATTTTTTGAAATCAGATGGTTTTGCCAATCGCAATTTTATGCAG
CTGATCCATGATGATAGTTTGACATTTAAAGAAGACATTCAAAAAGCACAAGTGTCT
GGACAAGGCGATAGTTTACATGAACATATTGCAAATTTAGCTGGTAGCCCTGCTATT
AAAAAAGGTATTTTACAGACTGTAAAAGTTGTTGATGAATTGGTCAAAGTAATGGG
GCGGCATAAGCCAGAAAATATCGTTATTGAAATGGCACGTGAAAATCAGACAACTC
AAAAGGGCCAGAAAAATTCGCGAGAGCGTATGAAACGAATCGAAGAAGGTATCAA
AGAATTAGGAAGTCAGATTCTTAAAGAGCATCCTGTTGAAAATACTCAATTGCAAA
ATGAAAAGCTCTATCTCTATTATCTCCAAAATGGAAGAGACATGTATGTGGACCAAG
AATTAGATATTAATCGTTTAAGTGATTATGATGTCGATCACATTGTTCCACAAAGTTT
CCTTAAAGACGATTCAATAGACAATAAGGTCTTAACGCGTTCTGATAAAAATCGTGG
TAAATCGGATAACGTTCCAAGTGAAGAAGTAGTCAAAAAGATGAAAAACTATTGGA
GACAACTTCTAAACGCCAAGTTAATCACTCAACGTAAGTTTGATAATTTAACGAAAG
CTGAACGTGGAGGTTTGAGTGAACTTGATAAAGCTGGTTTTATCAAACGCCAATTGG
TTGAAACTCGCCAAATCACTAAGCATGTGGCACAAATTTTGGATAGTCGCATGAATA
CTAAATACGATGAAAATGATAAACTTATTCGAGAGGTTAAAGTGATTACCTTAAAAT
CTAAATTAGTTTCTGACTTCCGAAAAGATTTCCAATTCTATAAAGTACGTGAGATTA
ACAATTACCATCATGCCCATGATGCGTATCTAAATGCCGTCGTTGGAACTGCTTTGA
TTAAGAAATATCCAAAACTTGAATCGGAGTTTGTCTATGGTGATTATAAAGTTTATG
ATGTTCGTAAAATGATTGCTAAGTCTGAGCAAGAAATAGGCAAAGCAACCGCAAAA
TATTTCTTTTACTCTAATATCATGAACTTCTTCAAAACAGAAATTACACTTGCAAATG
GAGAGATTCGCAAACGCCCTCTAATCGAAACTAATGGGGAAACTGGAGAAATTGTC
TGGGATAAAGGGCGAGATTTTGCCACAGTGCGCAAAGTATTGTCCATGCCCCAAGTC
AATATTGTCAAGAAAACAGAAGTACAGACAGGCGGATTCTCCAAGGAGTCAATTTT
ACCAAAAAGAAATTCGGACAAGCTTATTGCTCGTAAAAAAGACTGGGATCCAAAAA
AATATGGTGGTTTTGATAGTCCAACGGTAGCTTATTCAGTCCTAGTGGTTGCTAAGG
TGGAAAAAGGGAAATCGAAGAAGTTAAAATCCGTTAAAGAGTTACTAGGGATCACA
ATTATGGAAAGAAGTTCCTTTGAAAAAAATCCGATTGACTTTTTAGAAGCTAAAGGA
TATAAGGAAGTTAAAAAAGACTTAATCATTAAACTACCTAAATATAGTCTTTTTGAG
TTAGAAAACGGTCGTAAACGGATGCTGGCTAGTGCCGGAGAATTACAAAAAGGAAA
TGAGCTGGCTCTGCCAAGCAAATATGTGAATTTTTTATATTTAGCTAGTCATTATGAA
AAGTTGAAGGGTAGTCCAGAAGATAACGAACAAAAACAATTGTTTGTGGAGCAGCA
TAAGCATTATTTAGATGAGATTATTGAGCAAATCAGTGAATTTTCTAAGCGTGTTAT
TTTAGCAGATGCCAATTTAGATAAAGTTCTTAGTGCATATAACAAACATAGAGACAA
ACCAATACGTGAACAAGCAGAAAATATTATTCATTTATTTACGTTGACGAATCTTGG
AGCTCCCGCTGCTTTTAAATATTTTGATACAACAATTGATCGTAAACGATATACGTCT
ACAAAAGAAGTTTTAGATGCCACTCTTATCCATCAATCCATCACTGGTCTTTATGAA
ACACGCATTGATTTGAGTCAGCTAGGAGGTGACTGATAATCAAATGTCAGACGAAA
ATGCCAATTATTGAAGCGGCTAACGCCGCTTTTTTTGTTTCTGGTCTCCCTTGCTTGT
ACTTTACAGTATAGCTTCCTCAATGATCTCCTTATCCGGTTAAGATGGCAAGCTTGAC
AAGTATTTCTTCAGAATTTACAATGAAGTTTACTAGAGGATTAACTAATAAGGAGGG
CAAACTACTAGAGATGTCTAAACGTCGTATGAAGTACCACAGCAACAATGAGATCA
GCTATTACAATTTCCTACATAGCATGAAGGACAAAATCGTGACGGTGTATAGAGGC
GGCCCTGAGAGCAAGAAGGGCAAGCTGACGGCAGTGAAGAGCGACTACATCGCGC
TGCAGGCGGAGAAGAAGATCATCTACTATCAACTTGAACACGTTAAGTCAATCACG
GAAGACACAAACAACTCAACAACGACGATCGAAACGGAAGAGATGTTAGACGCGG
ACGACTTCCACTCACTGATCGGCCACCTGATCAATCAGAGCGTGCAGTTCAATCAGG
GAGGACCAGAAAGCAAGAAAGGCCGCCTTGTATGGTTAGGCGACGACTATGCGGCA
CTGAATACGAACGAAGACGGAGTGGTATACTTCAACATTCACCACATTAAGTCCATC
TCAAAGCACGAACCGGACCTTAAGATCGAGGAACAAACACCGGTGGGCGTGCTTGA
GGCGGACGACCTGTCAGAAGTGTTCAAATCATTAACGCACAAGTGGGTGAGCATCA
ACAGAGGCGGCCCTGAGGCAATCGAAGGCATTTTAGTGGACAACGCAGATGGACAC
TACACGATCGTTAAGAACCAGGAAGTATTGAGAATTTACCCGTTCCACATCAAATCA
ATTTCACTGGGCCCGAAGGGAAGTTATAAAAAGGAAGACCAGAAGAACGAGCAGA
ATCAAGAGGATAACAACGACAAAGATTCAAACAGCTTTATCAGCAGCAAAAGCTAT
TCAAGCAGTAAAAGCTCAAAGCGGAGCTTGAAGAGCTCTGACGACCAGAGCAGTAA
GTCAGGCAGAAGTTCCAGAAGCAAGAGTAGCTCCAAGAGTAGTAAGAGGAGTTTGA
AGTCCAGCGACTACCAGAGCAGTAAAAGTGGAAGAAGTTCCAGGAGCAAGTCCTCA
AGTAAGTCCAGTAAGAGAAGCCTGAAGAGTAGCGACTACCAGAGCAGCAAAAGCTC
AAAAAGGAGCCCGCGCAGTAGCGACTACCAGAGTAGTCGTAGCCCGGGATATAGCT
CCTCCATCAAGTCTAGTGGCAAGCAGAAAGAGGACTATTCATACGAGACAATCGTT
CGCACAATCGATTATCATTGGAAGAGGAAATTCGGTTTCAAACTCGGGGGGGACGG
AGGTGAAATGTCTAAGGGCGAAGAGTTATTCACGGGCGTAGTTCCGATTCTTGTGGA
GCTCGACGGTGATGTAAACGGGCACAAGTTTTCTGTTTCTGGGGAAGGTGAAGGCG
ACGCGACCTATGGGAAACTCACTCTTAAGTTCATCTGCACCACGGGGAAGTTACCAG
TCCCGTGGCCAACACTGGTGACTACCTTCGCATACGGACTGCAGTGTTTTGCGCGTT
ATCCTGATCACATGAAACAACACGATTTTTTCAAGAGCGCCATGCCAGAAGGGTAC
GTCCAAGAACGCACGATTTTTTTCAAAGATGATGGGAATTACAAAACGCGTGCAGA
AGTAAAATTTGAGGGCGACACCCTGGTAAACCGTATTGAACTCAAAGGAATCGACT
TTAAGGAGGACGGAAACATTCTGGGCCACAAACTGGAATATAACTACAACTCACAT
AACGTTTATATAATGGCTGATAAGCAAAAAAATGGTATCAAGGTGAACTTTAAGAT
AAGACATAACATTGAGGACGGCAGTGTCCAACTTGCAGACCATTACCAGCAGAACA
CACCAATTGGCGACGGTCCAGTACTTTTACCGGACAACCACTATCTCTCAACTCAGA
GTGCTCTGTCAAAGGACCCTAATGAAAAACGCGACCACATGGTACTGCTCGAGTTTG
TGACTGCGGCCGGAATCACCCACGGGATGGACGAGCTTTACAAGTAATCAAATGTC
AGACGAAAATGCCAATTATTGAAGCGGCTAACGCCGCTTTTTTTGTTTCTGGTCTCCC
AACTTTGCTAAAGAGCGTCTCAGCTTCTAGAG
>pCas9g2-D15-GFP
(SEQ ID No: 240)
TAGAGATCTGCAGTGACTGGAGCTTCTAGAGATCAGGTCAGACGCGATTTCCTGGGT
GCTCGAGCCATGGGAAAGCGGTGGTGGAAAAAACGCCGAATGAAGCGGATGAAAT
TGCTCATCGTCTCGTGAAAAAGCCGAAGAAGGTTAAGCCTGGGTATAAAAAGAAAA
TGAGCTATGAGATGGAGAAATTGAAGTCATGCGCCGGTTAGTTTTAGAGCTAGAAA
TAGCAAGTTAAAATAAGGCTAGTCCGTTATCAACTTGAAAAAGTGGCACCGAGTCG
GTGCTTTTTTTCTCGGTACCAAATTCCAGAAAAGAGGCCTCCCGAAAGGGGGGCCTT
TTTTCGTTTTGGTCCCTCAATGATCCGACAGGAAGTGGTATTAGCGACTCCTACAAG
GAATAGGTGTCTGGGTCATGCAGTGACTGGGAAAACCCTGGCGACTAGTCTTGGACT
CCTGTTGATAGATCCAGTAATGACCTCAGAACTCCATCTGGATTTGTTCAGAACGCT
CGGTTGCCGCCGGGCGTTTTTTATTGGTGAGAATCCAGGGGTCCCCAATAATTACGA
TTTAAATCCTTCAAACTTCCCAAAGGCGAGCCCTAGTGACATTAGAAAACCGACTGT
AAAAAGTACAGTCGGCATTATCTCATATTATAAAAGCCAGTCATTAGGCCTATCTGA
CAATTCCTGAATAGAGTTCATAAACAATCCTGCATGATAACCATCACAAACAGAATG
ATGTACCTGTAAAGATAGCGGTAAATATATTGAATTACCTTTATTAATGAATTTTCCT
GCTGTAATAATGGGTAGAAGGTAATTACTATTATTATTGATATTTAAGTTAAACCCA
GTAAATGAAGTCCATGGAATAATAGAAAGAGAAAAAGCATTTTCAGGTATAGGTGT
TTTGGGAAACAATTTCCCCGAACCATTATATTTCTCTACATCAGAAAGGTATAAATC
ATAAAACTCTTTGAAGTCATTCTTTACAGGAGTCCAAATACCAGAGAATGTTTTAGA
TACACCATCAAAAATTGTATAAAGTGGCTCTAACTTATCCCAATAACCTAACTCTCC
GTCGCTATTGTAACCAGTTCTAAAAGCTGTATTTGAGTTTATCACCCTTGTCACTAAG
AAAATAAATGCAGGGTAAAATTTATATCCTTCTTGTTTTATGTTTCGGTATAAAACA
CTAATATCAATTTCTGTGGTTATACTAAAAGTCGTTTGTTGGTTCAAATAATGATTAA
ATATCTCTTTTCTCTTCCAATTGTCTAAATCAATTTTATTAAAGTTCATTTGATATGCC
TCCTAAATTTTTATCTAAAGTGAATTTAGGAGGCTTACTTGTCTGCTTTCTTCATTAG
AATCAATCCTTTTTTAAAAGTCAATCCCGTTTGTTGAAGACTTTTGTCCTTTTCCGCT
GCATAAACAATATGGCCCGTTTGTTGAACTACTCTTTAATAAAATAATTTTTCCGTTC
CCAATTCCACATTGCAATAATAGAAAATCCATCTTCATCGGCTTTTTCGTCATCATCT
GTATGAATCAAATCGCCTTCTTCTGTGTCATCAAGGTTTAATTTTTTATGTATTTCTTT
TAACAAACCACCATAGGAGATTAACCTTTTACGGTGTAAACCTTCCTCCAAATCAGA
CAAACGTTTCAAATTCTTTTCTTCATCATCGGTCATAAAATCCGTATCCTTTACAGGA
TATTTTGCAGTTTCGTCAATTGCCGATTGTATATCCGATTTATATTTATTTTTCGGTCG
AATCATTTGAACTTTTACATTTGGATCATAGTCTAATTTCATTGCCTTTTTCCAAAAT
TGAATCCATTGTTTTTGATTCACGTAGTTTTCTGTATTCTTAAAATAAGTTGGTTCCA
CACATACCAATACATGCATGTGCTGATTATAAGAATTATCTTTATTATTTATTGTCAC
TTCCGTTGCACGCATAAAACCAACAAGATTTTTATTAATTTTTTTATATTGCATCATT
CGGCGAAATCCTTGAGCCATATCTGACAAACTCTTATTTAATTCTTCGCCATCATAA
ACATTTTTAACTGTTAATGTGAGAAACAACCAACGAACTGTTGGCTTTTGTTTAATA
ACTTCAGCAACAACCTTTTGTGACTGAATGCCATGTTTCATTGCTCTCCTCCAGTTGC
ACATTGGACAAAGCCTGGATTTACAAAACCACACTCGATACAACTTTCTTTCGCCTG
TTTCACGATTTTGTTTATACTCTAATATTTCAGCACAATCTTTTACTCTTTCAGCCTTT
TTAAATTCAAGAATATGCAGAAGTTCAAAGTAATCAACATTAGCGATTTTCTTTTCT
CTCCATGGTCTCACTTTTCCACTTTTTGTCTTGTCCACTAAAACCCTTGATTTTTCATC
TGAATAAATGCTACTATTAGGACACATAATATTAAAAGAAACCCCCATCTATTTAGT
TATTTGTTTGGTCACTTATAACTTTAACAGATGGGGTTTTTCTGTGCAACCAATTTTA
AGGGTTTTCCAATACTTTAAAACACATACATACCAACACTTCAACGCACCTTTCAGC
AACTAAAATAAAAATGACGTTATTTCTATATGTATCAAGATAAGAAAGAACAAGTT
CAAAACCATCAAAAAAAGACACCTTTTCAGGTGCTTTTTTTATTTTATAAACTCATTC
CCTGATCTCGACTTCGTTCTTTTTTTACCTCTCGGTTATGAGTTAGTTCAAATTCGTTC
TTTTTAGGTTCTAAATCGTGTTTTTCTTGGAATTGTGCTGTTTTATCCTTTACCTTGTC
TACAAACCCCTTAAAAACGTTTTTAAAGGCTTTTAAGCGTCTGTACGTTCCTTAAGG
AATTATTCCTTAGTGCTTTCTAGGTTAATGTCATGATAATAATGGTTTCTTAGACGTC
AGGTGGCACTTTTCGGGGAAATGTCCGCGGAACCCCTATTTGTTTATTTTTCTAAATA
CATTCAAATATGTATCCGCTCATGAGACAATAACCCTGATAAATGCTTCAATAATAT
TGAAAAAGGAAGAGTATGAGTATTCAACATTTCCGTGTCGCCCTTATTCCCTTTTTTG
CGGCATTTTGCCTTCCTGTTTTTGCTCACCCAGAAACGCTGGTGAAAGTAAAAGATG
CTGAAGATCAGTTGGGTGCACGAGTGGGTTACATCGAACTGGATCTCAACAGCGGT
AAGATCCTTGAGAGTTTTCGCCCCGAAGAACGTTTTCCAATGATGAGCACTTTTAAA
GTTCTGCTATGTGGCGCGGTATTATCCCGTGTTGACGCCGGGCAAGAGCAACTCGGT
CGCCGCATACACTATTCTCAGAATGACTTGGTTGAGTACTCACCAGTCACAGAAAAG
CATCTTACGGATGGCATGACAGTAAGAGAATTATGCAGTGCTGCCATAACCATGAGT
GATAACACTGCGGCCAACTTACTTCTGACAACGATCGGAGGACCGAAGGAGCTAAC
CGCTTTTTTGCACAACATGGGGGATCATGTAACTCGCCTTGATCGTTGGGAACCGGA
GCTGAATGAAGCCATACCAAACGACGAGCGTGACACCACGATGCCTGCAGCAATGG
CAACAACGTTGCGCAAACTATTAACTGGCGAACTACTTACTCTAGCTTCCCGGCAAC
AATTAATAGACTGGATGGAGGCGGATAAAGTTGCAGGACCACTTCTGCGCTCGGCC
CTTCCGGCTGGCTGGTTTATTGCTGATAAATCTGGAGCCGGTGAGCGTGGGTCTCGC
GGTATCATTGCAGCACTGGGGCCAGATGGTAAGCCCTCCCGTATCGTAGTTATCTAC
ACGACGGGGAGTCAGGCAACTATGGATGAACGAAATAGACAGATCGCTGAGATAG
GTGCCTCACTGATTAAGCATTGGTAACTGTCAGACCAAGTTTACTCATATATACTTTA
GATTGATTTAAAACTTCATTTTTAATTTAAAAGGATCTAGGTGAAGATCCTTTTTGAT
AATCTCATGACCAAAATCCCTTAACGTGAGTTTTCGTTCCACTGAGCGTCAGACCCC
TTAATAAGATGATCTTCTTGAGATCGTTTTGGTCTGCGCGTAATCTCTTGCTCTGAAA
ACGAAAAAACCGCCTTGCAGGGAGGTTTTTCGAAGGTTCTCTGAGCTACCAACTCTT
TGAACCGAGGTAACTGGCTTGCAGGAGCGCAGTCACCAAAACTTGTCCTTTCAGTTT
AGCCTTAACCGGCGCATGACTTCAAGACTAACTCCTCTAAATCAATTACCAGTGGCT
GCTGCCAGTGGTGCTTTTGCATGTCTTTCCGGGTTGGACTCAAGACGATAGTTACCG
GATAAGGCGCAGCGGTCGGACTGAACGGGGGGTTCGTGCATACAGTCCAGCTTGGA
GCGAACTGCCTACCCGGAACTGAGTGTCAGGCGTGGAATGAGACAAACGCGGCCAT
AACAGCGGAATGACACCGGTAAACCGAAAGGCAGGAACAGGAGAGCGCACGAGGG
AGCCGCCAGGGGGAAACGCCTGGTATCTTTATAGTCCTGTCGGGTTTCGCCACCACT
GATTTGAGCGTCAGATTTCGTGATGCTTGTCAGGGGGCGGAGCCTATGGAAAAACG
CTTTGCCCGAGCCTATGGAAAAACGCTTTGCCCCACACAGGCTCTTGAACAAGGGAT
AACAATTTGTCATAGCTGTATTCGTTACAAGGAGGATTTTCGAAAAAACACCCTAAC
GGGTGTTTTTTTATAGCTGGTCTCCCCAAGTGCTGTAAAAAACGCCGAATGAAGCGG
ATGAAATTGCTCATCGTCTCGTGAAAAAGCCGAAGAAGGTTAAGCCTGGGTATAAA
AAGAAAATGAGCTATGAGATGGAGAAAGCCATAACCATGAGTGATAACACTGGTTT
TAGAGCTAGAAATAGCAAGTTAAAATAAGGCTAGTCCGTTATCAACTTGAAAAAGT
GGCACCGAGTCGGTGCTTTTTTTCTCGGTACCAAATTCCAGAAAAGAGGCCTCCCGA
AAGGGGGGCCTTTTTTCGTTTTGGTCCCTCAATGATCTCCTTATCCGGTTAAGATGGC
TTTTAAATCAATTTTCAGCTCCTGTATACAATTACCAAAGTTTTTCTGAATGAAGCCA
TGTGTTTTGACACATTCTATACTCACAAGGAGGTGAGACACATGAGTAAATCCTGGG
GCAAATTCATCGAGGAGGAAGAGGCGGAAATGGCTTCCCGTCGTAATCTAATGATT
GTCGATGGAACTAACTTAGGCTTTCGCTTCAAACATAACAATAGTAAAAAACCATTT
GCCTCAAGTTATGTTTCAACTATTCAATCTCTGGCAAAATCCTACTCTGCCAGAACTA
CGATTGTTCTAGGTGATAAGGGCAAAAGCGTGTTCAGACTAGAACATCTACCAGAG
TATAAAGGTAATCGTGATGAAAAGTACGCACAACGTACGGAAGAGGAGAAAGCGCT
AGATGAGCAGTTCTTCGAATACCTCAAAGATGCTTTCGAGTTGTGTAAAACTACATT
CCCAACTTTTACCATTCGTGGTGTAGAAGCAGATGACATGGCGGCGTATATTGTTAA
GCTCATCGGGCATCTTTATGATCACGTTTGGCTAATATCTACAGATGGTGACTGGGA
TACTTTATTAACGGATAAAGTTTCTCGTTTTTCTTTCACAACACGTCGTGAGTATCAT
CTTCGTGATATGTATGAGCACCATAACGTGGACGACGTAGAACAGTTTATCTCCCTG
AAAGCAATTATGGGAGATCTAGGAGATAATATTCGTGGTGTTGAAGGAATAGGAGC
AAAACGCGGATATAATATTATTCGTGAGTTTGGTAACGTACTGGATATTATTGATCA
GCTTCCACTGCCTGGAAAGCAGAAATATATACAGAACCTGAATGCATCGGAAGAAC
TGCTTTTCCGAAACTTGATTCTGGTTGATTTACCTACCTACTGTGTGGATGCTATTGC
TGCTGTAGGTCAAGATGTGTTAGATAAGTTCACGAAAGACATCCTTGAGATCGCGGA
ACAATGATAAGCTCTTAAAGGGGGTTTTAGATACCAGAGATGGATAAGAAATACTC
AATAGGCTTAGATATCGGCACAAATAGCGTCGGATGGGCGGTGATCACTGATGAAT
ATAAGGTTCCGTCTAAAAAGTTCAAGGTTCTGGGAAATACAGACCGCCACAGTATC
AAAAAAAATCTTATAGGGGCTCTTTTATTTGACAGTGGAGAGACAGCGGAAGCGAC
TCGTCTCAAACGGACAGCTCGTAGAAGGTATACACGTCGGAAGAATCGTATTTGTTA
TCTACAGGAGATTTTTTCAAATGAGATGGCGAAAGTAGATGATAGTTTCTTTCATCG
ACTTGAAGAGTCTTTTTTGGTGGAAGAAGACAAGAAGCATGAACGTCATCCTATTTT
TGGAAATATAGTAGATGAAGTTGCTTATCATGAGAAATATCCAACTATCTATCATCT
GCGAAAAAAATTGGTAGATTCTACTGATAAAGCGGATTTGCGCTTAATCTATTTGGC
CTTAGCGCATATGATTAAGTTTCGTGGTCATTTTTTGATTGAGGGAGATTTAAATCCT
GATAATAGTGATGTGGACAAACTATTTATCCAGTTGGTACAAACCTACAATCAATTA
TTTGAAGAAAACCCTATTAACGCAAGTGGAGTAGATGCTAAAGCGATTCTTTCTGCA
CGATTGAGTAAATCAAGACGATTAGAAAATCTCATTGCTCAGCTCCCCGGTGAGAA
GAAAAATGGCTTATTTGGGAATCTCATTGCTTTGTCATTGGGTTTGACCCCTAATTTT
AAATCAAATTTTGATTTGGCAGAAGATGCTAAATTACAGCTTTCAAAAGATACTTAC
GATGATGATTTAGATAATTTATTGGCGCAAATTGGAGATCAATATGCTGATTTGTTTT
TGGCAGCTAAGAATTTATCAGATGCTATTTTACTTTCAGATATCCTAAGAGTAAATA
CTGAAATAACTAAGGCTCCCCTATCAGCTTCAATGATTAAACGCTACGATGAACATC
ATCAAGACTTGACTCTTTTAAAAGCTTTAGTTCGACAACAACTTCCAGAAAAGTATA
AAGAAATCTTTTTTGATCAATCAAAAAACGGATATGCAGGTTATATTGATGGGGGAG
CTAGCCAAGAAGAATTTTATAAATTTATCAAACCAATTTTAGAAAAAATGGATGGTA
CTGAGGAATTATTGGTGAAACTAAATCGTGAAGATTTGCTGCGCAAGCAACGGACC
TTTGACAACGGCTCTATTCCCCATCAAATTCACTTGGGTGAGCTGCATGCTATTTTGA
GAAGACAAGAAGACTTTTATCCATTTTTAAAAGACAATCGTGAGAAGATTGAAAAA
ATCTTGACTTTTCGAATTCCTTATTATGTTGGTCCATTGGCGCGTGGCAATAGTCGTT
TTGCATGGATGACTCGGAAGTCTGAAGAAACAATTACCCCATGGAATTTTGAAGAA
GTTGTCGATAAAGGTGCTTCAGCTCAATCATTTATTGAACGCATGACAAACTTTGAT
AAAAATCTTCCAAATGAAAAAGTACTACCAAAACATAGTTTGCTTTATGAGTATTTT
ACGGTTTATAACGAATTGACAAAGGTCAAATATGTTACTGAAGGAATGCGAAAACC
AGCATTTCTTTCAGGTGAACAGAAGAAAGCCATTGTTGATTTACTCTTCAAAACAAA
TCGAAAAGTAACCGTTAAGCAATTAAAAGAAGATTATTTCAAAAAAATAGAATGTT
TTGATAGTGTTGAAATTTCAGGAGTTGAAGATAGATTTAATGCTTCATTAGGTACCT
ACCATGATTTGCTAAAAATTATTAAAGATAAAGATTTTTTGGATAATGAAGAAAATG
AAGATATCTTAGAGGATATTGTTTTAACATTGACCTTATTTGAAGATAGGGAGATGA
TTGAGGAAAGACTTAAAACATATGCTCACCTCTTTGATGATAAGGTGATGAAACAGC
TTAAACGTCGCCGTTATACTGGTTGGGGACGTTTGTCTCGAAAATTGATTAATGGTA
TTAGGGATAAGCAATCTGGCAAAACAATATTAGATTTTTTGAAATCAGATGGTTTTG
CCAATCGCAATTTTATGCAGCTGATCCATGATGATAGTTTGACATTTAAAGAAGACA
TTCAAAAAGCACAAGTGTCTGGACAAGGCGATAGTTTACATGAACATATTGCAAATT
TAGCTGGTAGCCCTGCTATTAAAAAAGGTATTTTACAGACTGTAAAAGTTGTTGATG
AATTGGTCAAAGTAATGGGGCGGCATAAGCCAGAAAATATCGTTATTGAAATGGCA
CGTGAAAATCAGACAACTCAAAAGGGCCAGAAAAATTCGCGAGAGCGTATGAAAC
GAATCGAAGAAGGTATCAAAGAATTAGGAAGTCAGATTCTTAAAGAGCATCCTGTT
GAAAATACTCAATTGCAAAATGAAAAGCTCTATCTCTATTATCTCCAAAATGGAAGA
GACATGTATGTGGACCAAGAATTAGATATTAATCGTTTAAGTGATTATGATGTCGAT
CACATTGTTCCACAAAGTTTCCTTAAAGACGATTCAATAGACAATAAGGTCTTAACG
CGTTCTGATAAAAATCGTGGTAAATCGGATAACGTTCCAAGTGAAGAAGTAGTCAA
AAAGATGAAAAACTATTGGAGACAACTTCTAAACGCCAAGTTAATCACTCAACGTA
AGTTTGATAATTTAACGAAAGCTGAACGTGGAGGTTTGAGTGAACTTGATAAAGCTG
GTTTTATCAAACGCCAATTGGTTGAAACTCGCCAAATCACTAAGCATGTGGCACAAA
TTTTGGATAGTCGCATGAATACTAAATACGATGAAAATGATAAACTTATTCGAGAGG
TTAAAGTGATTACCTTAAAATCTAAATTAGTTTCTGACTTCCGAAAAGATTTCCAATT
CTATAAAGTACGTGAGATTAACAATTACCATCATGCCCATGATGCGTATCTAAATGC
CGTCGTTGGAACTGCTTTGATTAAGAAATATCCAAAACTTGAATCGGAGTTTGTCTA
TGGTGATTATAAAGTTTATGATGTTCGTAAAATGATTGCTAAGTCTGAGCAAGAAAT
AGGCAAAGCAACCGCAAAATATTTCTTTTACTCTAATATCATGAACTTCTTCAAAAC
AGAAATTACACTTGCAAATGGAGAGATTCGCAAACGCCCTCTAATCGAAACTAATG
GGGAAACTGGAGAAATTGTCTGGGATAAAGGGCGAGATTTTGCCACAGTGCGCAAA
GTATTGTCCATGCCCCAAGTCAATATTGTCAAGAAAACAGAAGTACAGACAGGCGG
ATTCTCCAAGGAGTCAATTTTACCAAAAAGAAATTCGGACAAGCTTATTGCTCGTAA
AAAAGACTGGGATCCAAAAAAATATGGTGGTTTTGATAGTCCAACGGTAGCTTATTC
AGTCCTAGTGGTTGCTAAGGTGGAAAAAGGGAAATCGAAGAAGTTAAAATCCGTTA
AAGAGTTACTAGGGATCACAATTATGGAAAGAAGTTCCTTTGAAAAAAATCCGATT
GACTTTTTAGAAGCTAAAGGATATAAGGAAGTTAAAAAAGACTTAATCATTAAACT
ACCTAAATATAGTCTTTTTGAGTTAGAAAACGGTCGTAAACGGATGCTGGCTAGTGC
CGGAGAATTACAAAAAGGAAATGAGCTGGCTCTGCCAAGCAAATATGTGAATTTTT
TATATTTAGCTAGTCATTATGAAAAGTTGAAGGGTAGTCCAGAAGATAACGAACAA
AAACAATTGTTTGTGGAGCAGCATAAGCATTATTTAGATGAGATTATTGAGCAAATC
AGTGAATTTTCTAAGCGTGTTATTTTAGCAGATGCCAATTTAGATAAAGTTCTTAGTG
CATATAACAAACATAGAGACAAACCAATACGTGAACAAGCAGAAAATATTATTCAT
TTATTTACGTTGACGAATCTTGGAGCTCCCGCTGCTTTTAAATATTTTGATACAACAA
TTGATCGTAAACGATATACGTCTACAAAAGAAGTTTTAGATGCCACTCTTATCCATC
AATCCATCACTGGTCTTTATGAAACACGCATTGATTTGAGTCAGCTAGGAGGTGACT
GATAATCAAATGTCAGACGAAAATGCCAATTATTGAAGCGGCTAACGCCGCTTTTTT
TGTTTCTGGTCTCCCTTGCTTGTACTTTACAGTATAGCTTCCTCAATGATCTCCTTATC
CGGTTAAGATGGCAAGCTTGACAAGTATTTCTTCAGAATTTACAATGAAGTTTACTA
GAGGATTAACTAATAAGGAGGGCAAACTACTAGAGATGTCTAAACGTCGTATGAAG
TACCACAGCAACAATGAGATCAGCTATTACAATTTCCTACATAGCATGAAGGACAA
AATCGTGACGGTGTATAGAGGCGGCCCTGAGAGCAAGAAGGGCAAGCTGACGGCA
GTGAAGAGCGACTACATCGCGCTGCAGGCGGAGAAGAAGATCATCTACTATCAACT
TGAACACGTTAAGTCAATCACGGAAGACACAAACAACTCAACAACGACGATCGAAA
CGGAAGAGATGTTAGACGCGGACGACTTCCACTCACTGATCGGCCACCTGATCAAT
CAGAGCGTGCAGTTCAATCAGGGAGGACCAGAAAGCAAGAAAGGCCGCCTTGTATG
GTTAGGCGACGACTATGCGGCACTGAATACGAACGAAGACGGAGTGGTATACTTCA
ACATTCACCACATTAAGTCCATCTCAAAGCACGAACCGGACCTTAAGATCGAGGAA
CAAACACCGGTGGGCGTGCTTGAGGCGGACGACCTGTCAGAAGTGTTCAAATCATT
AACGCACAAGTGGGTGAGCATCAACAGAGGCGGCCCTGAGGCAATCGAAGGCATTT
TAGTGGACAACGCAGATGGACACTACACGATCGTTAAGAACCAGGAAGTATTGAGA
ATTTACCCGTTCCACATCAAATCAATTTCACTGGGCCCGAAGGGAAGTTATAAAAAG
GAAGACCAGAAGAACGAGCAGAATCAAGAGGATAACAACGACAAAGATTCAAACA
GCTTTATCAGCAGCAAAAGCTATTCAAGCAGTAAAAGCTCAAAGCGGAGCTTGAAG
AGCTCTGACGACCAGAGCAGTAAGTCAGGCAGAAGTTCCAGAAGCAAGAGTAGCTC
CAAGAGTAGTAAGAGGAGTTTGAAGTCCAGCGACTACCAGAGCAGTAAAAGTGGAA
GAAGTTCCAGGAGCAAGTCCTCAAGTAAGTCCAGTAAGAGAAGCCTGAAGAGTAGC
GACTACCAGAGCAGCAAAAGCTCAAAAAGGAGCCCGCGCAGTAGCGACTACCAGA
GTAGTCGTAGCCCGGGATATAGCTCCTCCATCAAGTCTAGTGGCAAGCAGAAAGAG
GACTATTCATACGAGACAATCGTTCGCACAATCGATTATCATTGGAAGAGGAAATTC
GGTTTCAAACTCGGGGGGGACGGAGGTGAAATGTCTAAGGGCGAAGAGTTATTCAC
GGGCGTAGTTCCGATTCTTGTGGAGCTCGACGGTGATGTAAACGGGCACAAGTTTTC
TGTTTCTGGGGAAGGTGAAGGCGACGCGACCTATGGGAAACTCACTCTTAAGTTCAT
CTGCACCACGGGGAAGTTACCAGTCCCGTGGCCAACACTGGTGACTACCTTCGCATA
CGGACTGCAGTGTTTTGCGCGTTATCCTGATCACATGAAACAACACGATTTTTTCAA
GAGCGCCATGCCAGAAGGGTACGTCCAAGAACGCACGATTTTTTTCAAAGATGATG
GGAATTACAAAACGCGTGCAGAAGTAAAATTTGAGGGCGACACCCTGGTAAACCGT
ATTGAACTCAAAGGAATCGACTTTAAGGAGGACGGAAACATTCTGGGCCACAAACT
GGAATATAACTACAACTCACATAACGTTTATATAATGGCTGATAAGCAAAAAAATG
GTATCAAGGTGAACTTTAAGATAAGACATAACATTGAGGACGGCAGTGTCCAACTT
GCAGACCATTACCAGCAGAACACACCAATTGGCGACGGTCCAGTACTTTTACCGGA
CAACCACTATCTCTCAACTCAGAGTGCTCTGTCAAAGGACCCTAATGAAAAACGCG
ACCACATGGTACTGCTCGAGTTTGTGACTGCGGCCGGAATCACCCACGGGATGGAC
GAGCTTTACAAGTAATCAAATGTCAGACGAAAATGCCAATTATTGAAGCGGCTAAC
GCCGCTTTTTTTGTTTCTGGTCTCCCAACTTTGCTAAAGAGCGTCTCAGCTTCTAGAG
>pCas9g1-D15-KinA
(SEQ ID No: 241)
ttaagatggcaagcttgacagccgaattcggattcccgcgTATATCTTGAATTAAAATTA
AAATCTGCCAAGATCGAAAAATAAAAAGGATTTTTTGTGTCATTGGCGAATTATGATCTA
TTGAAGCAACCGTTAAGCTTACATAAGGAGGAACTACTATGGAACAGGATACGCAGCATG
TTAAACCACTTCAAACAAAAACCGATATTCATGCAGTCTTGGCCTCTAATGGACGCATCA
TTTATATATCTGCCAACTCCAAACTGCATTTGGGCTATCTCCAAGGAGAGATGATCGGA
TCATTCCTCAAAACGTTTCTGCATGAGGAAGACCAATTTTTGGTTGAAAGCTATTTTT
ATAATGAACATCATCTGATGCCGTGCACCTTTCGTTTTATTAAAAAAGATCATACGA
TTGTGTGGGTGGAGGCTGCGGTAGAAATTGTTACGACAAGAGCTGAGCGGACAGAA
CGGGAAATCATTTTGAAAATGAAGGTTCTTGAAGAAGAAACAGGCCATCAATCCCT
AAACTGCGAAAAACATGAAATCGAACCTGCAAGCCCGGAATCGACTACATATATAA
CGGATGATTATGAACGGTTGGTTGAAAATCTCCCGAGTCCGCTATGCATCAGTGTCA
AAGGCAAGATCGTCTATGTAAACAGCGCGATGCTTTCAATGCTGGGAGCCAAAAGC
AAGGATGCTATTATTGGTAAATCGTCCTATGAATTTATTGAAGAAGAATATCATGAT
ATCGTGAAAAACAGGATTATACGAATGCAAAAAGGAATGGAAGTCGGAATGATTGA
ACAGACGTGGAAAAGGCTTGATGGCACACCTGTTCATTTAGAAGTGAAAGCATCCC
CGACCGTCTACAAAAACCAGCAGGCTGAGCTGCTGCTGCTGATCGATATCTCTTCAA
GGAAAAAATTCCAAACCATCCTGCAAAAAAGCCGTGAACGATATCAGCTGCTGATT
CAAAATTCCATTGATACCATTGCGGTGATTCACAATGGAAAATGGGTATTTATGAAT
GAATCGGGAATTTCCCTGTTTGAAGCGGCTACATATGAGGATTTAATTGGCAAAAAC
ATATACGATCAGCTGCATCCTTGCGATCACGAGGATGTAAAAGAGAGAATCCAAAA
CATTGCCGAGCAAAAAACAGAATCTGAAATTGTCAAGCAATCCTGGTTCACCTTTCA
GAACAGGGTCATCTATACGGAGATGGTCTGCATTCCGACGACCTTTTTTGGTGAAGC
GGCCGTCCAGGTCATTCTTCGGGACATCTCAGAGAGAAAACAAACAGAAGAATTGA
TGCTGAAATCGGAAAAATTATCAATCGCAGGGCAGCTCGCGGCGGGAATCGCCCAT
GAGATCCGCAACCCTCTTACAGCGATCAAAGGATTTTTACAGCTGATGAAACCGACA
ATGGAAGGCAACGAACATTACTTTGATATTGTGTTTTCTGAACTCAGCCGTATCGAA
TTAATACTCAGTGAACTGCTCATGCTGGCGAAACCTCAGCAAAATGCTGTCAAAGAA
TATTTGAACTTGAAAAAATTAATTGGTGAGGTTTCAGCCCTGTTAGAAACGCAGGCG
AATTTAAATGGCATTTTTATCAGAACAAGTTATGAAAAAGACAGCATTTATATAAAC
GGGGATCAAAACCAATTAAAGCAGGTATTCATTAATTTAATCAAAAATGCAGTTGA
ATCAATGCCTGATGGGGGAACAGTAGACATTATCATAACCGAAGATGAGCATTCTG
TTCATGTTACTGTCAAAGACGAAGGGGAAGGTATACCTGAAAAGGTACTAAACCGG
ATTGGAGAGCCATTTTTAACAACAAAAGAAAAAGGTACGGGGCTTGGATTAATGGT
GACATTTAATATCATTGAAAACCATCAGGGAGTTATACATGTGGACAGCCATCCTGA
AAAAGGCACAGCGTTTAAAATTTCATTTCcaaaaaaataagcatgcccggggatggacg
agctttacaaGTAATCAAATGTCAGACGAAAATGCCAATTATTGAAGCGGCTAACGCCG
CTTTTTTTGTTTCTGGTCTCCCAACTTTGCTAAAGAGCGTCTCAGCTTCTAGAGGTGAC
TGGGAAAACCCTGGCGACTAGTCTTGGACTCCTGTTGATAGATCCAGTAATGACCTCAG
AACTCCATCTGGATTTGTTCAGAACGCTCGGTTGCCGCCGGGCGTTTTTTATTGGTGAG
AATCCAGGGGTCCCCAATAATTACGATTTAAATCCTTCAAACTTCCCAAAGGCGAGCCC
TAGTGACATTAGAAAACCGACTGTAAAAAGTACAGTCGGCATTATCTCATATTATAAAAG
CCAGTCATTAGGCCTATCTGACAATTCCTGAATAGAGTTCATAAACAATCCTGCATG
ATAACCATCACAAACAGAATGATGTACCTGTAAAGATAGCGGTAAATATATTGAAT
TACCTTTATTAATGAATTTTCCTGCTGTAATAATGGGTAGAAGGTAATTACTATTATT
ATTGATATTTAAGTTAAACCCAGTAAATGAAGTCCATGGAATAATAGAAAGAGAAA
AAGCATTTTCAGGTATAGGTGTTTTGGGAAACAATTTCCCCGAACCATTATATTTCTC
TACATCAGAAAGGTATAAATCATAAAACTCTTTGAAGTCATTCTTTACAGGAGTCCA
AATACCAGAGAATGTTTTAGATACACCATCAAAAATTGTATAAAGTGGCTCTAACTT
ATCCCAATAACCTAACTCTCCGTCGCTATTGTAACCAGTTCTAAAAGCTGTATTTGA
GTTTATCACCCTTGTCACTAAGAAAATAAATGCAGGGTAAAATTTATATCCTTCTTGT
TTTATGTTTCGGTATAAAACACTAATATCAATTTCTGTGGTTATACTAAAAGTCGTTT
GTTGGTTCAAATAATGATTAAATATCTCTTTTCTCTTCCAATTGTCTAAATCAATTTT
ATTAAAGTTCATTTGATATGCCTCCTAAATTTTTATCTAAAGTGAATTTAGGAGGCTT
ACTTGTCTGCTTTCTTCATTAGAATCAATCCTTTTTTAAAAGTCAATCCCGTTTGTTG
AAGACTTTTGTCCTTTTCCGCTGCATAAACAATATGGCCCGTTTGTTGAACTACTCTT
TAATAAAATAATTTTTCCGTTCCCAATTCCACATTGCAATAATAGAAAATCCATCTTC
ATCGGCTTTTTCGTCATCATCTGTATGAATCAAATCGCCTTCTTCTGTGTCATCAAGG
TTTAATTTTTTATGTATTTCTTTTAACAAACCACCATAGGAGATTAACCTTTTACGGT
GTAAACCTTCCTCCAAATCAGACAAACGTTTCAAATTCTTTTCTTCATCATCGGTCAT
AAAATCCGTATCCTTTACAGGATATTTTGCAGTTTCGTCAATTGCCGATTGTATATCC
GATTTATATTTATTTTTCGGTCGAATCATTTGAACTTTTACATTTGGATCATAGTCTA
ATTTCATTGCCTTTTTCCAAAATTGAATCCATTGTTTTTGATTCACGTAGTTTTCTGTA
TTCTTAAAATAAGTTGGTTCCACACATACCAATACATGCATGTGCTGATTATAAGAA
TTATCTTTATTATTTATTGTCACTTCCGTTGCACGCATAAAACCAACAAGATTTTTAT
TAATTTTTTTATATTGCATCATTCGGCGAAATCCTTGAGCCATATCTGACAAACTCTT
ATTTAATTCTTCGCCATCATAAACATTTTTAACTGTTAATGTGAGAAACAACCAACG
AACTGTTGGCTTTTGTTTAATAACTTCAGCAACAACCTTTTGTGACTGAATGCCATGT
TTCATTGCTCTCCTCCAGTTGCACATTGGACAAAGCCTGGATTTACAAAACCACACT
CGATACAACTTTCTTTCGCCTGTTTCACGATTTTGTTTATACTCTAATATTTCAGCAC
AATCTTTTACTCTTTCAGCCTTTTTAAATTCAAGAATATGCAGAAGTTCAAAGTAATC
AACATTAGCGATTTTCTTTTCTCTCCATGGTCTCACTTTTCCACTTTTTGTCTTGTCCA
CTAAAACCCTTGATTTTTCATCTGAATAAATGCTACTATTAGGACACATAATATTAA
AAGAAACCCCCATCTATTTAGTTATTTGTTTGGTCACTTATAACTTTAACAGATGGGG
TTTTTCTGTGCAACCAATTTTAAGGGTTTTCCAATACTTTAAAACACATACATACCAA
CACTTCAACGCACCTTTCAGCAACTAAAATAAAAATGACGTTATTTCTATATGTATC
AAGATAAGAAAGAACAAGTTCAAAACCATCAAAAAAAGACACCTTTTCAGGTGCTT
TTTTTATTTTATAAACTCATTCCCTGATCTCGACTTCGTTCTTTTTTTACCTCTCGGTT
ATGAGTTAGTTCAAATTCGTTCTTTTTAGGTTCTAAATCGTGTTTTTCTTGGAATTGT
GCTGTTTTATCCTTTACCTTGTCTACAAACCCCTTAAAAACGTTTTTAAAGGCTTTTA
AGCGTCTGTACGTTCCTTAAGGAATTATTCCTTAGTGCTTTCTAGGTTAATGTCATGA
TAATAATGGTTTCTTAGACGTCAGGTGGCACTTTTCGGGGAAATGTCCGCGGAACCC
CTATTTGTTTATTTTTCTAAATACATTCAAATATGTATCCGCTCATGAGACAATAACC
CTGATAAATGCTTCAATAATATTGAAAAAGGAAGAGTATGAGTATTCAACATTTCCG
TGTCGCCCTTATTCCCTTTTTTGCGGCATTTTGCCTTCCTGTTTTTGCTCACCCAGAAA
CGCTGGTGAAAGTAAAAGATGCTGAAGATCAGTTGGGTGCACGAGTGGGTTACATC
GAACTGGATCTCAACAGCGGTAAGATCCTTGAGAGTTTTCGCCCCGAAGAACGTTTT
CCAATGATGAGCACTTTTAAAGTTCTGCTATGTGGCGCGGTATTATCCCGTGTTGAC
GCCGGGCAAGAGCAACTCGGTCGCCGCATACACTATTCTCAGAATGACTTGGTTGAG
TACTCACCAGTCACAGAAAAGCATCTTACGGATGGCATGACAGTAAGAGAATTATG
CAGTGCTGCCATAACCATGAGTGATAACACTGCGGCCAACTTACTTCTGACAACGAT
CGGAGGACCGAAGGAGCTAACCGCTTTTTTGCACAACATGGGGGATCATGTAACTC
GCCTTGATCGTTGGGAACCGGAGCTGAATGAAGCCATACCAAACGACGAGCGTGAC
ACCACGATGCCTGCAGCAATGGCAACAACGTTGCGCAAACTATTAACTGGCGAACT
ACTTACTCTAGCTTCCCGGCAACAATTAATAGACTGGATGGAGGCGGATAAAGTTGC
AGGACCACTTCTGCGCTCGGCCCTTCCGGCTGGCTGGTTTATTGCTGATAAATCTGG
AGCCGGTGAGCGTGGGTCTCGCGGTATCATTGCAGCACTGGGGCCAGATGGTAAGC
CCTCCCGTATCGTAGTTATCTACACGACGGGGAGTCAGGCAACTATGGATGAACGA
AATAGACAGATCGCTGAGATAGGTGCCTCACTGATTAAGCATTGGTAACTGTCAGAC
CAAGTTTACTCATATATACTTTAGATTGATTTAAAACTTCATTTTTAATTTAAAAGGA
TCTAGGTGAAGATCCTTTTTGATAATCTCATGACCAAAATCCCTTAACGTGAGTTTTC
GTTCCACTGAGCGTCAGACCCCTTAATAAGATGATCTTCTTGAGATCGTTTTGGTCTG
CGCGTAATCTCTTGCTCTGAAAACGAAAAAACCGCCTTGCAGGGAGGTTTTTCGAAG
GTTCTCTGAGCTACCAACTCTTTGAACCGAGGTAACTGGCTTGCAGGAGCGCAGTCA
CCAAAACTTGTCCTTTCAGTTTAGCCTTAACCGGCGCATGACTTCAAGACTAACTCCT
CTAAATCAATTACCAGTGGCTGCTGCCAGTGGTGCTTTTGCATGTCTTTCCGGGTTGG
ACTCAAGACGATAGTTACCGGATAAGGCGCAGCGGTCGGACTGAACGGGGGGTTCG
TGCATACAGTCCAGCTTGGAGCGAACTGCCTACCCGGAACTGAGTGTCAGGCGTGG
AATGAGACAAACGCGGCCATAACAGCGGAATGACACCGGTAAACCGAAAGGCAGG
AACAGGAGAGCGCACGAGGGAGCCGCCAGGGGGAAACGCCTGGTATCTTTATAGTC
CTGTCGGGTTTCGCCACCACTGATTTGAGCGTCAGATTTCGTGATGCTTGTCAGGGG
GCGGAGCCTATGGAAAAACGCTTTGCCCGAGCCTATGGAAAAACGCTTTGCCCCAC
ACAGGCTCTTGAACAAGGGATAACAATTTGTCATAGCTGTATTCGTTACAAGGAGGA
TTTTCGAAAAAACACCCTAACGGGTGTTTTTTTATAGCTGGTCTCCCCAAGTGCTGTA
AAAAACGCCGAATGAAGCGGATGAAATTGCTCATCGTCTCGTGAAAAAGCCGAAGA
AGGTTAAGCCTGGGTATAAAAAGAAAATGAGCTATGAGATGGAGAAAGCCATAACC
ATGAGTGATAACACTGGTTTTAGAGCTAGAAATAGCAAGTTAAAATAAGGCTAGTC
CGTTATCAACTTGAAAAAGTGGCACCGAGTCGGTGCTTTTTTTCTCGGTACCAAATT
CCAGAAAAGAGGCCTCCCGAAAGGGGGGCCTTTTTTCGTTTTGGTCCCTCAATGATC
TCCTTATCCGGTTAAGATGGCTTTTAAATCAATTTTCAGCTCCTGTATACAATTACCA
AAGTTTTTCTGAATGAAGCCATGTGTTTTGACACATTCTATACTCACAAGGAGGTGA
GACACATGAGTAAATCCTGGGGCAAATTCATCGAGGAGGAAGAGGCGGAAATGGCT
TCCCGTCGTAATCTAATGATTGTCGATGGAACTAACTTAGGCTTTCGCTTCAAACAT
AACAATAGTAAAAAACCATTTGCCTCAAGTTATGTTTCAACTATTCAATCTCTGGCA
AAATCCTACTCTGCCAGAACTACGATTGTTCTAGGTGATAAGGGCAAAAGCGTGTTC
AGACTAGAACATCTACCAGAGTATAAAGGTAATCGTGATGAAAAGTACGCACAACG
TACGGAAGAGGAGAAAGCGCTAGATGAGCAGTTCTTCGAATACCTCAAAGATGCTT
TCGAGTTGTGTAAAACTACATTCCCAACTTTTACCATTCGTGGTGTAGAAGCAGATG
ACATGGCGGCGTATATTGTTAAGCTCATCGGGCATCTTTATGATCACGTTTGGCTAA
TATCTACAGATGGTGACTGGGATACTTTATTAACGGATAAAGTTTCTCGTTTTTCTTT
CACAACACGTCGTGAGTATCATCTTCGTGATATGTATGAGCACCATAACGTGGACGA
CGTAGAACAGTTTATCTCCCTGAAAGCAATTATGGGAGATCTAGGAGATAATATTCG
TGGTGTTGAAGGAATAGGAGCAAAACGCGGATATAATATTATTCGTGAGTTTGGTA
ACGTACTGGATATTATTGATCAGCTTCCACTGCCTGGAAAGCAGAAATATATACAGA
ACCTGAATGCATCGGAAGAACTGCTTTTCCGAAACTTGATTCTGGTTGATTTACCTA
CCTACTGTGTGGATGCTATTGCTGCTGTAGGTCAAGATGTGTTAGATAAGTTCACGA
AAGACATCCTTGAGATCGCGGAACAATGATAAGCTCTTAAAGGGGGTTTTAGATAC
CAGAGATGGATAAGAAATACTCAATAGGCTTAGATATCGGCACAAATAGCGTCGGA
TGGGCGGTGATCACTGATGAATATAAGGTTCCGTCTAAAAAGTTCAAGGTTCTGGGA
AATACAGACCGCCACAGTATCAAAAAAAATCTTATAGGGGCTCTTTTATTTGACAGT
GGAGAGACAGCGGAAGCGACTCGTCTCAAACGGACAGCTCGTAGAAGGTATACACG
TCGGAAGAATCGTATTTGTTATCTACAGGAGATTTTTTCAAATGAGATGGCGAAAGT
AGATGATAGTTTCTTTCATCGACTTGAAGAGTCTTTTTTGGTGGAAGAAGACAAGAA
GCATGAACGTCATCCTATTTTTGGAAATATAGTAGATGAAGTTGCTTATCATGAGAA
ATATCCAACTATCTATCATCTGCGAAAAAAATTGGTAGATTCTACTGATAAAGCGGA
TTTGCGCTTAATCTATTTGGCCTTAGCGCATATGATTAAGTTTCGTGGTCATTTTTTG
ATTGAGGGAGATTTAAATCCTGATAATAGTGATGTGGACAAACTATTTATCCAGTTG
GTACAAACCTACAATCAATTATTTGAAGAAAACCCTATTAACGCAAGTGGAGTAGA
TGCTAAAGCGATTCTTTCTGCACGATTGAGTAAATCAAGACGATTAGAAAATCTCAT
TGCTCAGCTCCCCGGTGAGAAGAAAAATGGCTTATTTGGGAATCTCATTGCTTTGTC
ATTGGGTTTGACCCCTAATTTTAAATCAAATTTTGATTTGGCAGAAGATGCTAAATT
ACAGCTTTCAAAAGATACTTACGATGATGATTTAGATAATTTATTGGCGCAAATTGG
AGATCAATATGCTGATTTGTTTTTGGCAGCTAAGAATTTATCAGATGCTATTTTACTT
TCAGATATCCTAAGAGTAAATACTGAAATAACTAAGGCTCCCCTATCAGCTTCAATG
ATTAAACGCTACGATGAACATCATCAAGACTTGACTCTTTTAAAAGCTTTAGTTCGA
CAACAACTTCCAGAAAAGTATAAAGAAATCTTTTTTGATCAATCAAAAAACGGATAT
GCAGGTTATATTGATGGGGGAGCTAGCCAAGAAGAATTTTATAAATTTATCAAACCA
ATTTTAGAAAAAATGGATGGTACTGAGGAATTATTGGTGAAACTAAATCGTGAAGA
TTTGCTGCGCAAGCAACGGACCTTTGACAACGGCTCTATTCCCCATCAAATTCACTT
GGGTGAGCTGCATGCTATTTTGAGAAGACAAGAAGACTTTTATCCATTTTTAAAAGA
CAATCGTGAGAAGATTGAAAAAATCTTGACTTTTCGAATTCCTTATTATGTTGGTCC
ATTGGCGCGTGGCAATAGTCGTTTTGCATGGATGACTCGGAAGTCTGAAGAAACAAT
TACCCCATGGAATTTTGAAGAAGTTGTCGATAAAGGTGCTTCAGCTCAATCATTTAT
TGAACGCATGACAAACTTTGATAAAAATCTTCCAAATGAAAAAGTACTACCAAAAC
ATAGTTTGCTTTATGAGTATTTTACGGTTTATAACGAATTGACAAAGGTCAAATATG
TTACTGAAGGAATGCGAAAACCAGCATTTCTTTCAGGTGAACAGAAGAAAGCCATT
GTTGATTTACTCTTCAAAACAAATCGAAAAGTAACCGTTAAGCAATTAAAAGAAGA
TTATTTCAAAAAAATAGAATGTTTTGATAGTGTTGAAATTTCAGGAGTTGAAGATAG
ATTTAATGCTTCATTAGGTACCTACCATGATTTGCTAAAAATTATTAAAGATAAAGA
TTTTTTGGATAATGAAGAAAATGAAGATATCTTAGAGGATATTGTTTTAACATTGAC
CTTATTTGAAGATAGGGAGATGATTGAGGAAAGACTTAAAACATATGCTCACCTCTT
TGATGATAAGGTGATGAAACAGCTTAAACGTCGCCGTTATACTGGTTGGGGACGTTT
GTCTCGAAAATTGATTAATGGTATTAGGGATAAGCAATCTGGCAAAACAATATTAG
ATTTTTTGAAATCAGATGGTTTTGCCAATCGCAATTTTATGCAGCTGATCCATGATGA
TAGTTTGACATTTAAAGAAGACATTCAAAAAGCACAAGTGTCTGGACAAGGCGATA
GTTTACATGAACATATTGCAAATTTAGCTGGTAGCCCTGCTATTAAAAAAGGTATTT
TACAGACTGTAAAAGTTGTTGATGAATTGGTCAAAGTAATGGGGCGGCATAAGCCA
GAAAATATCGTTATTGAAATGGCACGTGAAAATCAGACAACTCAAAAGGGCCAGAA
AAATTCGCGAGAGCGTATGAAACGAATCGAAGAAGGTATCAAAGAATTAGGAAGTC
AGATTCTTAAAGAGCATCCTGTTGAAAATACTCAATTGCAAAATGAAAAGCTCTATC
TCTATTATCTCCAAAATGGAAGAGACATGTATGTGGACCAAGAATTAGATATTAATC
GTTTAAGTGATTATGATGTCGATCACATTGTTCCACAAAGTTTCCTTAAAGACGATTC
AATAGACAATAAGGTCTTAACGCGTTCTGATAAAAATCGTGGTAAATCGGATAACG
TTCCAAGTGAAGAAGTAGTCAAAAAGATGAAAAACTATTGGAGACAACTTCTAAAC
GCCAAGTTAATCACTCAACGTAAGTTTGATAATTTAACGAAAGCTGAACGTGGAGGT
TTGAGTGAACTTGATAAAGCTGGTTTTATCAAACGCCAATTGGTTGAAACTCGCCAA
ATCACTAAGCATGTGGCACAAATTTTGGATAGTCGCATGAATACTAAATACGATGAA
AATGATAAACTTATTCGAGAGGTTAAAGTGATTACCTTAAAATCTAAATTAGTTTCT
GACTTCCGAAAAGATTTCCAATTCTATAAAGTACGTGAGATTAACAATTACCATCAT
GCCCATGATGCGTATCTAAATGCCGTCGTTGGAACTGCTTTGATTAAGAAATATCCA
AAACTTGAATCGGAGTTTGTCTATGGTGATTATAAAGTTTATGATGTTCGTAAAATG
ATTGCTAAGTCTGAGCAAGAAATAGGCAAAGCAACCGCAAAATATTTCTTTTACTCT
AATATCATGAACTTCTTCAAAACAGAAATTACACTTGCAAATGGAGAGATTCGCAA
ACGCCCTCTAATCGAAACTAATGGGGAAACTGGAGAAATTGTCTGGGATAAAGGGC
GAGATTTTGCCACAGTGCGCAAAGTATTGTCCATGCCCCAAGTCAATATTGTCAAGA
AAACAGAAGTACAGACAGGCGGATTCTCCAAGGAGTCAATTTTACCAAAAAGAAAT
TCGGACAAGCTTATTGCTCGTAAAAAAGACTGGGATCCAAAAAAATATGGTGGTTTT
GATAGTCCAACGGTAGCTTATTCAGTCCTAGTGGTTGCTAAGGTGGAAAAAGGGAA
ATCGAAGAAGTTAAAATCCGTTAAAGAGTTACTAGGGATCACAATTATGGAAAGAA
GTTCCTTTGAAAAAAATCCGATTGACTTTTTAGAAGCTAAAGGATATAAGGAAGTTA
AAAAAGACTTAATCATTAAACTACCTAAATATAGTCTTTTTGAGTTAGAAAACGGTC
GTAAACGGATGCTGGCTAGTGCCGGAGAATTACAAAAAGGAAATGAGCTGGCTCTG
CCAAGCAAATATGTGAATTTTTTATATTTAGCTAGTCATTATGAAAAGTTGAAGGGT
AGTCCAGAAGATAACGAACAAAAACAATTGTTTGTGGAGCAGCATAAGCATTATTT
AGATGAGATTATTGAGCAAATCAGTGAATTTTCTAAGCGTGTTATTTTAGCAGATGC
CAATTTAGATAAAGTTCTTAGTGCATATAACAAACATAGAGACAAACCAATACGTG
AACAAGCAGAAAATATTATTCATTTATTTACGTTGACGAATCTTGGAGCTCCCGCTG
CTTTTAAATATTTTGATACAACAATTGATCGTAAACGATATACGTCTACAAAAGAAG
TTTTAGATGCCACTCTTATCCATCAATCCATCACTGGTCTTTATGAAACACGCATTGA
TTTGAGTCAGCTAGGAGGTGACTGATAATCAAATGTCAGACGAAAATGCCAATTATT
GAAGCGGCTAACGCCGCTTTTTTTGTTTCTGGTCTCCCTTGCTTGTACTTTACAGTAT
AGCTTCCTCAATGATCTCCTTATCCGG
>pCas9g2-D15-KinA
(SEQ ID No: 242)
ttaagatggcaagcttgacagccgaattcggattcccgcg
TATATCTTGAATTAAAATTAAAATCTGCCAAG
ATCGAAAAATAAAAAGGATTTTTTGTGTCATTGGCGAATTATGATCTATTGAAGCAA
CCGTTAAGCTTACATAAGGAGGAACTACTATGGAACAGGATACGCAGCATGTTAAA
CCACTTCAAACAAAAACCGATATTCATGCAGTCTTGGCCTCTAATGGACGCATCATT
TATATATCTGCCAACTCCAAACTGCATTTGGGCTATCTCCAAGGAGAGATGATCGGA
TCATTCCTCAAAACGTTTCTGCATGAGGAAGACCAATTTTTGGTTGAAAGCTATTTTT
ATAATGAACATCATCTGATGCCGTGCACCTTTCGTTTTATTAAAAAAGATCATACGA
TTGTGTGGGTGGAGGCTGCGGTAGAAATTGTTACGACAAGAGCTGAGCGGACAGAA
CGGGAAATCATTTTGAAAATGAAGGTTCTTGAAGAAGAAACAGGCCATCAATCCCT
AAACTGCGAAAAACATGAAATCGAACCTGCAAGCCCGGAATCGACTACATATATAA
CGGATGATTATGAACGGTTGGTTGAAAATCTCCCGAGTCCGCTATGCATCAGTGTCA
AAGGCAAGATCGTCTATGTAAACAGCGCGATGCTTTCAATGCTGGGAGCCAAAAGC
AAGGATGCTATTATTGGTAAATCGTCCTATGAATTTATTGAAGAAGAATATCATGAT
ATCGTGAAAAACAGGATTATACGAATGCAAAAAGGAATGGAAGTCGGAATGATTGA
ACAGACGTGGAAAAGGCTTGATGGCACACCTGTTCATTTAGAAGTGAAAGCATCCC
CGACCGTCTACAAAAACCAGCAGGCTGAGCTGCTGCTGCTGATCGATATCTCTTCAA
GGAAAAAATTCCAAACCATCCTGCAAAAAAGCCGTGAACGATATCAGCTGCTGATT
CAAAATTCCATTGATACCATTGCGGTGATTCACAATGGAAAATGGGTATTTATGAAT
GAATCGGGAATTTCCCTGTTTGAAGCGGCTACATATGAGGATTTAATTGGCAAAAAC
ATATACGATCAGCTGCATCCTTGCGATCACGAGGATGTAAAAGAGAGAATCCAAAA
CATTGCCGAGCAAAAAACAGAATCTGAAATTGTCAAGCAATCCTGGTTCACCTTTCA
GAACAGGGTCATCTATACGGAGATGGTCTGCATTCCGACGACCTTTTTTGGTGAAGC
GGCCGTCCAGGTCATTCTTCGGGACATCTCAGAGAGAAAACAAACAGAAGAATTGA
TGCTGAAATCGGAAAAATTATCAATCGCAGGGCAGCTCGCGGCGGGAATCGCCCAT
GAGATCCGCAACCCTCTTACAGCGATCAAAGGATTTTTACAGCTGATGAAACCGACA
ATGGAAGGCAACGAACATTACTTTGATATTGTGTTTTCTGAACTCAGCCGTATCGAA
TTAATACTCAGTGAACTGCTCATGCTGGCGAAACCTCAGCAAAATGCTGTCAAAGAA
TATTTGAACTTGAAAAAATTAATTGGTGAGGTTTCAGCCCTGTTAGAAACGCAGGCG
AATTTAAATGGCATTTTTATCAGAACAAGTTATGAAAAAGACAGCATTTATATAAAC
GGGGATCAAAACCAATTAAAGCAGGTATTCATTAATTTAATCAAAAATGCAGTTGA
ATCAATGCCTGATGGGGGAACAGTAGACATTATCATAACCGAAGATGAGCATTCTG
TTCATGTTACTGTCAAAGACGAAGGGGAAGGTATACCTGAAAAGGTACTAAACCGG
ATTGGAGAGCCATTTTTAACAACAAAAGAAAAAGGTACGGGGCTTGGATTAATGGT
GACATTTAATATCATTGAAAACCATCAGGGAGTTATACATGTGGACAGCCATCCTGA
AAAAGGCACAGCGTTTAAAATTTCATTTCcaaaaaaataagcatgcccggggatggacga
gctttacaaGTAATCAAATGTCAGACGAAAATGCCAATTATTGAAGCGGCTAACGCCGCT
TTTTTTGTTTCTGGTCTCCCAACTTTGCTAAAGAGCGTCTCAGCTTCTAGAGTAGAGATC
TGCAGTGACTGGAGCTTCTAGAGATCAGGTCAGACGCGATTTCCTGGGTGCTCGAGCCAT
GGGAAAGCGGTGGTGGAAAAAACGCCGAATGAAGCGGATGAAATTGCTCATCGTCTCG
TGAAAAAGCCGAAGAAGGTTAAGCCTGGGTATAAAAAGAAAATGAGCTATGAGAT
GGAGAAATTGAAGTCATGCGCCGGTTAGTTTTAGAGCTAGAAATAGCAAGTTAAAA
TAAGGCTAGTCCGTTATCAACTTGAAAAAGTGGCACCGAGTCGGTGCTTTTTTTCTC
GGTACCAAATTCCAGAAAAGAGGCCTCCCGAAAGGGGGGCCTTTTTTCGTTTTGGTC
CCTCAATGATCCGACAGGAAGTGGTATTAGCGACTCCTACAAGGAATAGGTGTCTG
GGTCATGCAGTGACTGGGAAAACCCTGGCGACTAGTCTTGGACTCCTGTTGATAGAT
CCAGTAATGACCTCAGAACTCCATCTGGATTTGTTCAGAACGCTCGGTTGCCGCCGG
GCGTTTTTTATTGGTGAGAATCCAGGGGTCCCCAATAATTACGATTTAAATCCTTCA
AACTTCCCAAAGGCGAGCCCTAGTGACATTAGAAAACCGACTGTAAAAAGTACAGT
CGGCATTATCTCATATTATAAAAGCCAGTCATTAGGCCTATCTGACAATTCCTGAAT
AGAGTTCATAAACAATCCTGCATGATAACCATCACAAACAGAATGATGTACCTGTA
AAGATAGCGGTAAATATATTGAATTACCTTTATTAATGAATTTTCCTGCTGTAATAAT
GGGTAGAAGGTAATTACTATTATTATTGATATTTAAGTTAAACCCAGTAAATGAAGT
CCATGGAATAATAGAAAGAGAAAAAGCATTTTCAGGTATAGGTGTTTTGGGAAACA
ATTTCCCCGAACCATTATATTTCTCTACATCAGAAAGGTATAAATCATAAAACTCTTT
GAAGTCATTCTTTACAGGAGTCCAAATACCAGAGAATGTTTTAGATACACCATCAAA
AATTGTATAAAGTGGCTCTAACTTATCCCAATAACCTAACTCTCCGTCGCTATTGTAA
CCAGTTCTAAAAGCTGTATTTGAGTTTATCACCCTTGTCACTAAGAAAATAAATGCA
GGGTAAAATTTATATCCTTCTTGTTTTATGTTTCGGTATAAAACACTAATATCAATTT
CTGTGGTTATACTAAAAGTCGTTTGTTGGTTCAAATAATGATTAAATATCTCTTTTCT
CTTCCAATTGTCTAAATCAATTTTATTAAAGTTCATTTGATATGCCTCCTAAATTTTT
ATCTAAAGTGAATTTAGGAGGCTTACTTGTCTGCTTTCTTCATTAGAATCAATCCTTT
TTTAAAAGTCAATCCCGTTTGTTGAAGACTTTTGTCCTTTTCCGCTGCATAAACAATA
TGGCCCGTTTGTTGAACTACTCTTTAATAAAATAATTTTTCCGTTCCCAATTCCACAT
TGCAATAATAGAAAATCCATCTTCATCGGCTTTTTCGTCATCATCTGTATGAATCAAA
TCGCCTTCTTCTGTGTCATCAAGGTTTAATTTTTTATGTATTTCTTTTAACAAACCACC
ATAGGAGATTAACCTTTTACGGTGTAAACCTTCCTCCAAATCAGACAAACGTTTCAA
ATTCTTTTCTTCATCATCGGTCATAAAATCCGTATCCTTTACAGGATATTTTGCAGTT
TCGTCAATTGCCGATTGTATATCCGATTTATATTTATTTTTCGGTCGAATCATTTGAA
CTTTTACATTTGGATCATAGTCTAATTTCATTGCCTTTTTCCAAAATTGAATCCATTGT
TTTTGATTCACGTAGTTTTCTGTATTCTTAAAATAAGTTGGTTCCACACATACCAATA
CATGCATGTGCTGATTATAAGAATTATCTTTATTATTTATTGTCACTTCCGTTGCACG
CATAAAACCAACAAGATTTTTATTAATTTTTTTATATTGCATCATTCGGCGAAATCCT
TGAGCCATATCTGACAAACTCTTATTTAATTCTTCGCCATCATAAACATTTTTAACTG
TTAATGTGAGAAACAACCAACGAACTGTTGGCTTTTGTTTAATAACTTCAGCAACAA
CCTTTTGTGACTGAATGCCATGTTTCATTGCTCTCCTCCAGTTGCACATTGGACAAAG
CCTGGATTTACAAAACCACACTCGATACAACTTTCTTTCGCCTGTTTCACGATTTTGT
TTATACTCTAATATTTCAGCACAATCTTTTACTCTTTCAGCCTTTTTAAATTCAAGAA
TATGCAGAAGTTCAAAGTAATCAACATTAGCGATTTTCTTTTCTCTCCATGGTCTCAC
TTTTCCACTTTTTGTCTTGTCCACTAAAACCCTTGATTTTTCATCTGAATAAATGCTAC
TATTAGGACACATAATATTAAAAGAAACCCCCATCTATTTAGTTATTTGTTTGGTCAC
TTATAACTTTAACAGATGGGGTTTTTCTGTGCAACCAATTTTAAGGGTTTTCCAATAC
TTTAAAACACATACATACCAACACTTCAACGCACCTTTCAGCAACTAAAATAAAAAT
GACGTTATTTCTATATGTATCAAGATAAGAAAGAACAAGTTCAAAACCATCAAAAA
AAGACACCTTTTCAGGTGCTTTTTTTATTTTATAAACTCATTCCCTGATCTCGACTTC
GTTCTTTTTTTACCTCTCGGTTATGAGTTAGTTCAAATTCGTTCTTTTTAGGTTCTAAA
TCGTGTTTTTCTTGGAATTGTGCTGTTTTATCCTTTACCTTGTCTACAAACCCCTTAAA
AACGTTTTTAAAGGCTTTTAAGCGTCTGTACGTTCCTTAAGGAATTATTCCTTAGTGC
TTTCTAGGTTAATGTCATGATAATAATGGTTTCTTAGACGTCAGGTGGCACTTTTCGG
GGAAATGTCCGCGGAACCCCTATTTGTTTATTTTTCTAAATACATTCAAATATGTATC
CGCTCATGAGACAATAACCCTGATAAATGCTTCAATAATATTGAAAAAGGAAGAGT
ATGAGTATTCAACATTTCCGTGTCGCCCTTATTCCCTTTTTTGCGGCATTTTGCCTTCC
TGTTTTTGCTCACCCAGAAACGCTGGTGAAAGTAAAAGATGCTGAAGATCAGTTGGG
TGCACGAGTGGGTTACATCGAACTGGATCTCAACAGCGGTAAGATCCTTGAGAGTTT
TCGCCCCGAAGAACGTTTTCCAATGATGAGCACTTTTAAAGTTCTGCTATGTGGCGC
GGTATTATCCCGTGTTGACGCCGGGCAAGAGCAACTCGGTCGCCGCATACACTATTC
TCAGAATGACTTGGTTGAGTACTCACCAGTCACAGAAAAGCATCTTACGGATGGCAT
GACAGTAAGAGAATTATGCAGTGCTGCCATAACCATGAGTGATAACACTGCGGCCA
ACTTACTTCTGACAACGATCGGAGGACCGAAGGAGCTAACCGCTTTTTTGCACAACA
TGGGGGATCATGTAACTCGCCTTGATCGTTGGGAACCGGAGCTGAATGAAGCCATA
CCAAACGACGAGCGTGACACCACGATGCCTGCAGCAATGGCAACAACGTTGCGCAA
ACTATTAACTGGCGAACTACTTACTCTAGCTTCCCGGCAACAATTAATAGACTGGAT
GGAGGCGGATAAAGTTGCAGGACCACTTCTGCGCTCGGCCCTTCCGGCTGGCTGGTT
TATTGCTGATAAATCTGGAGCCGGTGAGCGTGGGTCTCGCGGTATCATTGCAGCACT
GGGGCCAGATGGTAAGCCCTCCCGTATCGTAGTTATCTACACGACGGGGAGTCAGG
CAACTATGGATGAACGAAATAGACAGATCGCTGAGATAGGTGCCTCACTGATTAAG
CATTGGTAACTGTCAGACCAAGTTTACTCATATATACTTTAGATTGATTTAAAACTTC
ATTTTTAATTTAAAAGGATCTAGGTGAAGATCCTTTTTGATAATCTCATGACCAAAA
TCCCTTAACGTGAGTTTTCGTTCCACTGAGCGTCAGACCCCTTAATAAGATGATCTTC
TTGAGATCGTTTTGGTCTGCGCGTAATCTCTTGCTCTGAAAACGAAAAAACCGCCTT
GCAGGGAGGTTTTTCGAAGGTTCTCTGAGCTACCAACTCTTTGAACCGAGGTAACTG
GCTTGCAGGAGCGCAGTCACCAAAACTTGTCCTTTCAGTTTAGCCTTAACCGGCGCA
TGACTTCAAGACTAACTCCTCTAAATCAATTACCAGTGGCTGCTGCCAGTGGTGCTT
TTGCATGTCTTTCCGGGTTGGACTCAAGACGATAGTTACCGGATAAGGCGCAGCGGT
CGGACTGAACGGGGGGTTCGTGCATACAGTCCAGCTTGGAGCGAACTGCCTACCCG
GAACTGAGTGTCAGGCGTGGAATGAGACAAACGCGGCCATAACAGCGGAATGACAC
CGGTAAACCGAAAGGCAGGAACAGGAGAGCGCACGAGGGAGCCGCCAGGGGGAAA
CGCCTGGTATCTTTATAGTCCTGTCGGGTTTCGCCACCACTGATTTGAGCGTCAGATT
TCGTGATGCTTGTCAGGGGGCGGAGCCTATGGAAAAACGCTTTGCCCGAGCCTATGG
AAAAACGCTTTGCCCCACACAGGCTCTTGAACAAGGGATAACAATTTGTCATAGCTG
TATTCGTTACAAGGAGGATTTTCGAAAAAACACCCTAACGGGTGTTTTTTTATAGCT
GGTCTCCCCAAGTGCTGTAAAAAACGCCGAATGAAGCGGATGAAATTGCTCATCGT
CTCGTGAAAAAGCCGAAGAAGGTTAAGCCTGGGTATAAAAAGAAAATGAGCTATGA
GATGGAGAAAGCCATAACCATGAGTGATAACACTGGTTTTAGAGCTAGAAATAGCA
AGTTAAAATAAGGCTAGTCCGTTATCAACTTGAAAAAGTGGCACCGAGTCGGTGCTT
TTTTTCTCGGTACCAAATTCCAGAAAAGAGGCCTCCCGAAAGGGGGGCCTTTTTTCG
TTTTGGTCCCTCAATGATCTCCTTATCCGGTTAAGATGGCTTTTAAATCAATTTTCAG
CTCCTGTATACAATTACCAAAGTTTTTCTGAATGAAGCCATGTGTTTTGACACATTCT
ATACTCACAAGGAGGTGAGACACATGAGTAAATCCTGGGGCAAATTCATCGAGGAG
GAAGAGGCGGAAATGGCTTCCCGTCGTAATCTAATGATTGTCGATGGAACTAACTTA
GGCTTTCGCTTCAAACATAACAATAGTAAAAAACCATTTGCCTCAAGTTATGTTTCA
ACTATTCAATCTCTGGCAAAATCCTACTCTGCCAGAACTACGATTGTTCTAGGTGAT
AAGGGCAAAAGCGTGTTCAGACTAGAACATCTACCAGAGTATAAAGGTAATCGTGA
TGAAAAGTACGCACAACGTACGGAAGAGGAGAAAGCGCTAGATGAGCAGTTCTTCG
AATACCTCAAAGATGCTTTCGAGTTGTGTAAAACTACATTCCCAACTTTTACCATTCG
TGGTGTAGAAGCAGATGACATGGCGGCGTATATTGTTAAGCTCATCGGGCATCTTTA
TGATCACGTTTGGCTAATATCTACAGATGGTGACTGGGATACTTTATTAACGGATAA
AGTTTCTCGTTTTTCTTTCACAACACGTCGTGAGTATCATCTTCGTGATATGTATGAG
CACCATAACGTGGACGACGTAGAACAGTTTATCTCCCTGAAAGCAATTATGGGAGA
TCTAGGAGATAATATTCGTGGTGTTGAAGGAATAGGAGCAAAACGCGGATATAATA
TTATTCGTGAGTTTGGTAACGTACTGGATATTATTGATCAGCTTCCACTGCCTGGAAA
GCAGAAATATATACAGAACCTGAATGCATCGGAAGAACTGCTTTTCCGAAACTTGAT
TCTGGTTGATTTACCTACCTACTGTGTGGATGCTATTGCTGCTGTAGGTCAAGATGTG
TTAGATAAGTTCACGAAAGACATCCTTGAGATCGCGGAACAATGATAAGCTCTTAA
AGGGGGTTTTAGATACCAGAGATGGATAAGAAATACTCAATAGGCTTAGATATCGG
CACAAATAGCGTCGGATGGGCGGTGATCACTGATGAATATAAGGTTCCGTCTAAAA
AGTTCAAGGTTCTGGGAAATACAGACCGCCACAGTATCAAAAAAAATCTTATAGGG
GCTCTTTTATTTGACAGTGGAGAGACAGCGGAAGCGACTCGTCTCAAACGGACAGCT
CGTAGAAGGTATACACGTCGGAAGAATCGTATTTGTTATCTACAGGAGATTTTTTCA
AATGAGATGGCGAAAGTAGATGATAGTTTCTTTCATCGACTTGAAGAGTCTTTTTTG
GTGGAAGAAGACAAGAAGCATGAACGTCATCCTATTTTTGGAAATATAGTAGATGA
AGTTGCTTATCATGAGAAATATCCAACTATCTATCATCTGCGAAAAAAATTGGTAGA
TTCTACTGATAAAGCGGATTTGCGCTTAATCTATTTGGCCTTAGCGCATATGATTAAG
TTTCGTGGTCATTTTTTGATTGAGGGAGATTTAAATCCTGATAATAGTGATGTGGAC
AAACTATTTATCCAGTTGGTACAAACCTACAATCAATTATTTGAAGAAAACCCTATT
AACGCAAGTGGAGTAGATGCTAAAGCGATTCTTTCTGCACGATTGAGTAAATCAAG
ACGATTAGAAAATCTCATTGCTCAGCTCCCCGGTGAGAAGAAAAATGGCTTATTTGG
GAATCTCATTGCTTTGTCATTGGGTTTGACCCCTAATTTTAAATCAAATTTTGATTTG
GCAGAAGATGCTAAATTACAGCTTTCAAAAGATACTTACGATGATGATTTAGATAAT
TTATTGGCGCAAATTGGAGATCAATATGCTGATTTGTTTTTGGCAGCTAAGAATTTAT
CAGATGCTATTTTACTTTCAGATATCCTAAGAGTAAATACTGAAATAACTAAGGCTC
CCCTATCAGCTTCAATGATTAAACGCTACGATGAACATCATCAAGACTTGACTCTTT
TAAAAGCTTTAGTTCGACAACAACTTCCAGAAAAGTATAAAGAAATCTTTTTTGATC
AATCAAAAAACGGATATGCAGGTTATATTGATGGGGGAGCTAGCCAAGAAGAATTT
TATAAATTTATCAAACCAATTTTAGAAAAAATGGATGGTACTGAGGAATTATTGGTG
AAACTAAATCGTGAAGATTTGCTGCGCAAGCAACGGACCTTTGACAACGGCTCTATT
CCCCATCAAATTCACTTGGGTGAGCTGCATGCTATTTTGAGAAGACAAGAAGACTTT
TATCCATTTTTAAAAGACAATCGTGAGAAGATTGAAAAAATCTTGACTTTTCGAATT
CCTTATTATGTTGGTCCATTGGCGCGTGGCAATAGTCGTTTTGCATGGATGACTCGG
AAGTCTGAAGAAACAATTACCCCATGGAATTTTGAAGAAGTTGTCGATAAAGGTGC
TTCAGCTCAATCATTTATTGAACGCATGACAAACTTTGATAAAAATCTTCCAAATGA
AAAAGTACTACCAAAACATAGTTTGCTTTATGAGTATTTTACGGTTTATAACGAATT
GACAAAGGTCAAATATGTTACTGAAGGAATGCGAAAACCAGCATTTCTTTCAGGTG
AACAGAAGAAAGCCATTGTTGATTTACTCTTCAAAACAAATCGAAAAGTAACCGTT
AAGCAATTAAAAGAAGATTATTTCAAAAAAATAGAATGTTTTGATAGTGTTGAAATT
TCAGGAGTTGAAGATAGATTTAATGCTTCATTAGGTACCTACCATGATTTGCTAAAA
ATTATTAAAGATAAAGATTTTTTGGATAATGAAGAAAATGAAGATATCTTAGAGGAT
ATTGTTTTAACATTGACCTTATTTGAAGATAGGGAGATGATTGAGGAAAGACTTAAA
ACATATGCTCACCTCTTTGATGATAAGGTGATGAAACAGCTTAAACGTCGCCGTTAT
ACTGGTTGGGGACGTTTGTCTCGAAAATTGATTAATGGTATTAGGGATAAGCAATCT
GGCAAAACAATATTAGATTTTTTGAAATCAGATGGTTTTGCCAATCGCAATTTTATG
CAGCTGATCCATGATGATAGTTTGACATTTAAAGAAGACATTCAAAAAGCACAAGT
GTCTGGACAAGGCGATAGTTTACATGAACATATTGCAAATTTAGCTGGTAGCCCTGC
TATTAAAAAAGGTATTTTACAGACTGTAAAAGTTGTTGATGAATTGGTCAAAGTAAT
GGGGCGGCATAAGCCAGAAAATATCGTTATTGAAATGGCACGTGAAAATCAGACAA
CTCAAAAGGGCCAGAAAAATTCGCGAGAGCGTATGAAACGAATCGAAGAAGGTATC
AAAGAATTAGGAAGTCAGATTCTTAAAGAGCATCCTGTTGAAAATACTCAATTGCA
AAATGAAAAGCTCTATCTCTATTATCTCCAAAATGGAAGAGACATGTATGTGGACCA
AGAATTAGATATTAATCGTTTAAGTGATTATGATGTCGATCACATTGTTCCACAAAG
TTTCCTTAAAGACGATTCAATAGACAATAAGGTCTTAACGCGTTCTGATAAAAATCG
TGGTAAATCGGATAACGTTCCAAGTGAAGAAGTAGTCAAAAAGATGAAAAACTATT
GGAGACAACTTCTAAACGCCAAGTTAATCACTCAACGTAAGTTTGATAATTTAACGA
AAGCTGAACGTGGAGGTTTGAGTGAACTTGATAAAGCTGGTTTTATCAAACGCCAAT
TGGTTGAAACTCGCCAAATCACTAAGCATGTGGCACAAATTTTGGATAGTCGCATGA
ATACTAAATACGATGAAAATGATAAACTTATTCGAGAGGTTAAAGTGATTACCTTAA
AATCTAAATTAGTTTCTGACTTCCGAAAAGATTTCCAATTCTATAAAGTACGTGAGA
TTAACAATTACCATCATGCCCATGATGCGTATCTAAATGCCGTCGTTGGAACTGCTTT
GATTAAGAAATATCCAAAACTTGAATCGGAGTTTGTCTATGGTGATTATAAAGTTTA
TGATGTTCGTAAAATGATTGCTAAGTCTGAGCAAGAAATAGGCAAAGCAACCGCAA
AATATTTCTTTTACTCTAATATCATGAACTTCTTCAAAACAGAAATTACACTTGCAAA
TGGAGAGATTCGCAAACGCCCTCTAATCGAAACTAATGGGGAAACTGGAGAAATTG
TCTGGGATAAAGGGCGAGATTTTGCCACAGTGCGCAAAGTATTGTCCATGCCCCAAG
TCAATATTGTCAAGAAAACAGAAGTACAGACAGGCGGATTCTCCAAGGAGTCAATT
TTACCAAAAAGAAATTCGGACAAGCTTATTGCTCGTAAAAAAGACTGGGATCCAAA
AAAATATGGTGGTTTTGATAGTCCAACGGTAGCTTATTCAGTCCTAGTGGTTGCTAA
GGTGGAAAAAGGGAAATCGAAGAAGTTAAAATCCGTTAAAGAGTTACTAGGGATCA
CAATTATGGAAAGAAGTTCCTTTGAAAAAAATCCGATTGACTTTTTAGAAGCTAAAG
GATATAAGGAAGTTAAAAAAGACTTAATCATTAAACTACCTAAATATAGTCTTTTTG
AGTTAGAAAACGGTCGTAAACGGATGCTGGCTAGTGCCGGAGAATTACAAAAAGGA
AATGAGCTGGCTCTGCCAAGCAAATATGTGAATTTTTTATATTTAGCTAGTCATTATG
AAAAGTTGAAGGGTAGTCCAGAAGATAACGAACAAAAACAATTGTTTGTGGAGCAG
CATAAGCATTATTTAGATGAGATTATTGAGCAAATCAGTGAATTTTCTAAGCGTGTT
ATTTTAGCAGATGCCAATTTAGATAAAGTTCTTAGTGCATATAACAAACATAGAGAC
AAACCAATACGTGAACAAGCAGAAAATATTATTCATTTATTTACGTTGACGAATCTT
GGAGCTCCCGCTGCTTTTAAATATTTTGATACAACAATTGATCGTAAACGATATACG
TCTACAAAAGAAGTTTTAGATGCCACTCTTATCCATCAATCCATCACTGGTCTTTATG
AAACACGCATTGATTTGAGTCAGCTAGGAGGTGACTGATAATCAAATGTCAGACGA
AAATGCCAATTATTGAAGCGGCTAACGCCGCTTTTTTTGTTTCTGGTCTCCCTTGCTT
GTACTTTACAGTATAGCTTCCTCAATGATCTCCTTATCCGG

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description is offered by way of illustration only and not as a limitation.

1. A method of producing a modified microbial spore, the method comprising:
introducing at least one vector into a spore-forming microorganism, wherein the at least one vector encodes (i) a sequence-specific endonuclease system which targets the at least one vector, and (ii) an exogenous gene product, wherein the endonuclease system is operably linked to a spore-specific promoter, and
culturing the spore-forming microorganism to generate a modified microbial spore.

2. The method of claim 1, wherein the at least one vector further encodes an exonuclease.

3. The method of claim 1, wherein the at least one vector comprises a vector encoding the sequence-specific endonuclease system and the exogenous gene product.

4. The method of claim 1, wherein the spore-forming microorganism is a spore-forming bacterium or a spore-forming fungus.

5. The method of claim 4, wherein the spore-forming bacterium is Bacillus subtilis, Bacillus firmus, Bacillus megaterium, Bacillus amyloliquefaciens, Bacillus licheniformis, or Bacillus pumilus.

6. The method of claim 1, wherein the sequence-specific endonuclease system comprises an RNA-guided endonuclease.

7. The method of claim 6, wherein the RNA-guided endonuclease comprises a CRISPR/Cas system.

8. The method of claim 6, wherein the RNA-guided DNA endonuclease is a Cas enzyme or a variant thereof.

9. The method of claim 8, wherein the Cas enzyme is Cas9.

10. The method of claim 1, wherein the at least one vector encodes a Cas enzyme and a guide RNA (gRNA).

11. The method of claim 1, wherein the at least one vector encodes a Cas enzyme and two gRNAs.

12. The method of claim 2, wherein the exonuclease is D15.

13. The method of claim 1, wherein the spore-specific promoter is PsspA.

14. The method of claim 1, wherein the at least one vector is a plasmid.

15. The method of claim 1, wherein the exogenous gene product is a protein.

16. The method of claim 1, wherein the exogenous gene product is an enzyme.

17. A phenotypically modified microbial spore, comprising an exogenous gene product, wherein the microbial spore is substantially free of exogenous genes.

18. The spore of claim 17, wherein the microbial spore is a bacterial spore or a fungal spore.

19. The spore of claim 17, wherein the exogenous gene product is a protein.

20. A composition comprising the microbial spore of claim 17.


SEQUENCE LISTING (246 sequences)

SEQ ID NO: 1 - DNA, 38 bp, Artificial Sequence
  Description of Artificial Sequence Synthetic primer
ttgtccttttccgctgcataaacaatatggcccgtttg

SEQ ID NO: 2 - DNA, 38 bp, Artificial Sequence
  Description of Artificial Sequence Synthetic primer
gccagggttttcccagtcactgcagatctctagaagct

SEQ ID NO: 3 - DNA, 37 bp, Artificial Sequence
  Description of Artificial Sequence Synthetic primer
caagcttctagagatctgcagtgactgggaaaaccct

SEQ ID NO: 4 - DNA, 38 bp, Artificial Sequence
  Description of Artificial Sequence Synthetic primer
aacaaacgggccatattgtttatgcagcggaaaaggac

SEQ ID NO: 5 - DNA, 40 bp, Artificial Sequence
  Description of Artificial Sequence Synthetic primer
tatggaaaaacgctttgccccacacaggctcttgaacaag

SEQ ID NO: 6 - DNA, 21 bp, Artificial Sequence
  Description of Artificial Sequence Synthetic primer
aacaccattgactccttcatc

SEQ ID NO: 7 - DNA, 20 bp, Artificial Sequence
  Description of Artificial Sequence Synthetic primer
gcagccaagtgaatacactg

SEQ ID NO: 8 - DNA, 42 bp, Artificial Sequence
  Description of Artificial Sequence Synthetic primer
actgcagatctctagaagctatgctgaatgacttcttacagc

SEQ ID NO: 9 - DNA, 38 bp, Artificial Sequence
  Description of Artificial Sequence Synthetic primer
tgtaagaagtcattcagcatagcttctagagatctgca

SEQ ID NO: 10 - DNA, 37 bp, Artificial Sequence
  Description of Artificial Sequence Synthetic primer
cttgttcaagagcctgtgtggggcaaagcgtttttcc

SEQ ID NO: 11 - DNA, 39 bp, Artificial Sequence
  Description of Artificial Sequence Synthetic primer
tacggttatccacagaatcagagggcaaactactagaga

SEQ ID NO: 12 - DNA, 40 bp, Artificial Sequence
  Description of Artificial Sequence Synthetic primer
ccaagtaatcgtgaatgtcgaccggataaggagatcattg

SEQ ID NO: 13 - DNA, 40 bp, Artificial Sequence
  Description of Artificial Sequence Synthetic primer
caatgatctccttatccggtcgacattcacgattacttgg

SEQ ID NO: 14 - DNA, 40 bp, Artificial Sequence
  Description of Artificial Sequence Synthetic primer
atctctagtagtttgccctctgattctgtggataaccgta

SEQ ID NO: 15 - DNA, 37 bp, Artificial Sequence
  Description of Artificial Sequence Synthetic primer
gcttgtcagggggcggagcctatggaaaaacgctttg

SEQ ID NO: 16 - DNA, 56 bp, Artificial Sequence
  Description of Artificial Sequence Synthetic primer
ccctttaagagcgtcatgtaaacttcattgtaaattctgaagaaatacttgtcaag

SEQ ID NO: 17 - DNA, 41 bp, Artificial Sequence
  Description of Artificial Sequence Synthetic primer
tcagaatttacaatgaagtttacatgacgctcttaaagggg

SEQ ID NO: 18 - DNA, 33 bp, Artificial Sequence
  Description of Artificial Sequence Synthetic primer
tcgccagggttttcccagtcactgcagatctct

SEQ ID NO: 19 - DNA, 18 bp, Artificial Sequence
  Description of Artificial Sequence Synthetic primer
agcttctagagatctgca

SEQ ID NO: 20 - DNA, 17 bp, Artificial Sequence
  Description of Artificial Sequence Synthetic primer
gggcaaagcgtttttcc

SEQ ID NO: 21 - DNA, 22 bp, Artificial Sequence
  Description of Artificial Sequence Synthetic primer
aataggcttagatatcggcaca

SEQ ID NO: 22 - DNA, 18 bp, Artificial Sequence
  Description of Artificial Sequence Synthetic primer
tcagtcacctcctagctg

SEQ ID NO: 23 - DNA, 21 bp, Artificial Sequence
  Description of Artificial Sequence Synthetic primer
gaaacacgcattgatttgagt

SEQ ID NO: 24 - DNA, 19 bp, Artificial Sequence
  Description of Artificial Sequence Synthetic primer
cccatccgacgctatttgt

SEQ ID NO: 25 - DNA, 38 bp, Artificial Sequence
  Description of Artificial Sequence Synthetic primer
gactggagcttctagagatcaggtcagacgcgatttcc

SEQ ID NO: 26 - DNA, 39 bp, Artificial Sequence
  Description of Artificial Sequence Synthetic primer
gggttttcccagtcactgcatgacccagacacctattcc

SEQ ID NO: 27 - DNA, 39 bp, Artificial Sequence
  Description of Artificial Sequence Synthetic primer
aggaataggtgtctgggtcatgcagtgactgggaaaacc

SEQ ID NO: 28 - DNA, 42 bp, Artificial Sequence
  Description of Artificial Sequence Synthetic primer
caggaaatcgcgtctgacctgatctctagaagctccagtcac

SEQ ID NO: 29 - DNA, 44 bp, Artificial Sequence
  Description of Artificial Sequence Synthetic primer
tacaatgaagtttacatgacgattaactaataaggagggcaaac

SEQ ID NO: 30 - DNA, 41 bp, Artificial Sequence
  Description of Artificial Sequence Synthetic primer
ctaaaaccccctttaagagcttatcattgttccgcgatctc

SEQ ID NO: 31 - DNA, 41 bp, Artificial Sequence
  Description of Artificial Sequence Synthetic primer
agatcgcggaacaatgataagctcttaaagggggttttaga

SEQ ID NO: 32 - DNA, 50 bp, Artificial Sequence
  Description of Artificial Sequence Synthetic primer
gccctccttattagttaatcgtcatgtaaacttcattgtaaattctgaag

SEQ ID NO: 33 - DNA, 44 bp, Artificial Sequence; identical to SEQ ID NO: 29
  Description of Artificial Sequence Synthetic primer

SEQ ID NO: 34 - DNA, 41 bp, Artificial Sequence; identical to SEQ ID NO: 30
  Description of Artificial Sequence Synthetic primer

SEQ ID NO: 35 - DNA, 41 bp, Artificial Sequence; identical to SEQ ID NO: 31
  Description of Artificial Sequence Synthetic primer

SEQ ID NO: 36 - DNA, 50 bp, Artificial Sequence; identical to SEQ ID NO: 32
  Description of Artificial Sequence Synthetic primer

SEQ ID NO: 37 - DNA, 40 bp, Artificial Sequence
  Description of Artificial Sequence Synthetic primer
gttttggtccctcaatgatctccttatccggttaagatgg

SEQ ID NO: 38 - DNA, 40 bp, Artificial Sequence
  Description of Artificial Sequence Synthetic primer
ttgtgccgatatctaagcctattgagtatttcttatccat

SEQ ID NO: 39 - DNA, 25 bp, Artificial Sequence
  Description of Artificial Sequence Synthetic primer
atggataagaaatactcaataggct

SEQ ID NO: 40 - DNA, 20 bp, Artificial Sequence
  Description of Artificial Sequence Synthetic primer
ccatcttaaccggataagga

SEQ ID NO: 41 - DNA, 40 bp, Artificial Sequence; identical to SEQ ID NO: 37
  Description of Artificial Sequence Synthetic primer

SEQ ID NO: 42 - DNA, 40 bp, Artificial Sequence; identical to SEQ ID NO: 38
  Description of Artificial Sequence Synthetic primer

SEQ ID NO: 43 - DNA, 25 bp, Artificial Sequence
  Description of Artificial Sequence Synthetic primer
atggataagaaatactcaataggct

SEQ ID NO: 44 - DNA, 20 bp, Artificial Sequence
  Description of Artificial Sequence Synthetic primer
ccatcttaaccggataagga

SEQ ID NO: 45 - DNA, 40 bp, Artificial Sequence; identical to SEQ ID NO: 37
  Description of Artificial Sequence Synthetic primer

SEQ ID NO: 46 - DNA, 40 bp, Artificial Sequence
  Description of Artificial Sequence Synthetic primer
cctcttcctcctcgatgaatttgccccaggatttactcat

SEQ ID NO: 47 - DNA, 20 bp, Artificial Sequence
  Description of Artificial Sequence Synthetic primer
atgagtaaatcctggggcaa

SEQ ID NO: 48 - DNA, 20 bp, Artificial Sequence
  Description of Artificial Sequence Synthetic primer
ccatcttaaccggataagga

SEQ ID NO: 49 - DNA, 40 bp, Artificial Sequence; identical to SEQ ID NO: 37
  Description of Artificial Sequence Synthetic primer

SEQ ID NO: 50 - DNA, 40 bp, Artificial Sequence; identical to SEQ ID NO: 46
  Description of Artificial Sequence Synthetic primer

SEQ ID NO: 51 - DNA, 20 bp, Artificial Sequence
  Description of Artificial Sequence Synthetic primer
atgagtaaatcctggggcaa

SEQ ID NO: 52 - DNA, 20 bp, Artificial Sequence
  Description of Artificial Sequence Synthetic primer
ccatcttaaccggataagga

SEQ ID NO: 53 - DNA, 39 bp, Artificial Sequence
  Description of Artificial Sequence Synthetic primer
ccttatccggttaagatggcgagtaaatcctggggcaaa

SEQ ID NO: 54 - DNA, 40 bp, Artificial Sequence
  Description of Artificial Sequence Synthetic primer
atttgccccaggatttactcgccatcttaaccggataagg

SEQ ID NO: 55 - DNA, 39 bp, Artificial Sequence; identical to SEQ ID NO: 53
  Description of Artificial Sequence Synthetic primer

SEQ ID NO: 56 - DNA, 40 bp, Artificial Sequence; identical to SEQ ID NO: 54
  Description of Artificial Sequence Synthetic primer

SEQ ID NO: 57 - DNA, 38 bp, Artificial Sequence
  Description of Artificial Sequence Synthetic primer
gagcgtctcagcttctagaggtgactgggaaaaccctg

SEQ ID NO: 58 - DNA, 45 bp, Artificial Sequence
  Description of Artificial Sequence Synthetic primer
cggataaggagatcattgaggaagctatactgtaaagtacaagca

SEQ ID NO: 59 - DNA, 41 bp, Artificial Sequence
  Description of Artificial Sequence Synthetic primer
gtactttacagtatagcttcctcaatgatctccttatccgg

SEQ ID NO: 60 - DNA, 39 bp, Artificial Sequence
  Description of Artificial Sequence Synthetic primer
gccagggttttcccagtcacctctagaagctgagacgct

SEQ ID NO: 61 - DNA, 40 bp, Artificial Sequence
  Description of Artificial Sequence Synthetic primer
gagcgtctcagcttctagagtagagatctgcagtgactgg

SEQ ID NO: 62 - DNA, 45 bp, Artificial Sequence; identical to SEQ ID NO: 58
  Description of Artificial Sequence Synthetic primer

SEQ ID NO: 63 - DNA, 41 bp, Artificial Sequence; identical to SEQ ID NO: 59
  Description of Artificial Sequence Synthetic primer

SEQ ID NO: 64 - DNA, 39 bp, Artificial Sequence
  Description of Artificial Sequence Synthetic primer
ccagtcactgcagatctctactctagaagctgagacgct

SEQ ID NO: 65 - DNA, 38 bp, Artificial Sequence
  Description of Artificial Sequence Synthetic primer
ttaagatggcaagcttgacagccgaattcggattcccg

SEQ ID NO: 66 - DNA, 42 bp, Artificial Sequence
  Description of Artificial Sequence Synthetic primer
ttgtaaagctcgtccatccccgggcatgcttatttttttgga

SEQ ID NO: 67 - DNA, 38 bp, Artificial Sequence
  Description of Artificial Sequence Synthetic primer
caaaaaaataagcatgcccggggatggacgagctttac

SEQ ID NO: 68 - DNA, 41 bp, Artificial Sequence
  Description of Artificial Sequence Synthetic primer
cgcgggaatccgaattcggctgtcaagcttgccatcttaac

SEQ ID NO: 69 - DNA, 38 bp, Artificial Sequence; identical to SEQ ID NO: 65
  Description of Artificial Sequence Synthetic primer

SEQ ID NO: 70 - DNA, 42 bp, Artificial Sequence; identical to SEQ ID NO: 66
  Description of Artificial Sequence Synthetic primer

SEQ ID NO: 71 - DNA, 38 bp, Artificial Sequence; identical to SEQ ID NO: 67
  Description of Artificial Sequence Synthetic primer

SEQ ID NO: 72 - DNA, 41 bp, Artificial Sequence; identical to SEQ ID NO: 68
  Description of Artificial Sequence Synthetic primer

SEQ ID NO: 73 - DNA, 25 bp, Artificial Sequence
  Description of Artificial Sequence Synthetic primer
cctgaatagagttcataaacaatcc

SEQ ID NO: 74 - DNA, 22 bp, Artificial Sequence
  Description of Artificial Sequence Synthetic primer
ttcaggaattgtcagataggcc

SEQ ID NO: 75 - DNA, 23 bp, Artificial Sequence
  Description of Artificial Sequence Synthetic primer
aacaatcctgcatgataaccatc

SEQ ID NO: 76 - DNA, 21 bp, Artificial Sequence
  Description of Artificial Sequence Synthetic primer
gtcagataggcctaatgactg

SEQ ID NO: 77 - DNA, 25 bp, Artificial Sequence
  Description of Artificial Sequence Synthetic primer
tgcagggtaaaatttatatccttct

SEQ ID NO: 78 - DNA, 23 bp, Artificial Sequence
  Description of Artificial Sequence Synthetic primer
ttagtgacaagggtgataaactc

SEQ ID NO: 79 - DNA, 25 bp, Artificial Sequence
  Description of Artificial Sequence Synthetic primer
atatccttcttgttttatgtttcgg

SEQ ID NO: 80 - DNA, 23 bp, Artificial Sequence
  Description of Artificial Sequence Synthetic primer
agggtgataaactcaaatacagc

SEQ ID NO: 81 - DNA, 21 bp, Artificial Sequence
  Description of Artificial Sequence Synthetic primer
tacggtgtaaaccttcctcca

SEQ ID NO: 82 - DNA, 23 bp, Artificial Sequence
  Description of Artificial Sequence Synthetic primer
tacaccgtaaaaggttaatctcc

SEQ ID NO: 83 - DNA, 23 bp, Artificial Sequence
  Description of Artificial Sequence Synthetic primer
cctccaaatcagacaaacgtttc

SEQ ID NO: 84 - DNA, 23 bp, Artificial Sequence
  Description of Artificial Sequence Synthetic primer
ggttaatctcctatggtggtttg

SEQ ID NO: 85 - DNA, 20 bp, Artificial Sequence
  Description of Artificial Sequence Synthetic primer
cattgctctcctccagttgc

SEQ ID NO: 86 - DNA, 23 bp, Artificial Sequence
  Description of Artificial Sequence Synthetic primer
aatgaaacatggcattcagtcac

SEQ ID NO: 87 - DNA, 21 bp, Artificial Sequence
  Description of Artificial Sequence Synthetic primer
cagttgcacattggacaaagc

SEQ ID NO: 88 - DNA, 23 bp, Artificial Sequence
  Description of Artificial Sequence Synthetic primer
gtcacaaaaggttgttgctgaag

SEQ ID NO: 89 - DNA, 22 bp, Artificial Sequence
  Description of Artificial Sequence Synthetic primer
cctctcggttatgagttagttc

SEQ ID NO: 90 - DNA, 21 bp, Artificial Sequence
  Description of Artificial Sequence Synthetic primer
aagaacgaagtcgagatcagg

SEQ ID NO: 91 - DNA, 25 bp, Artificial Sequence
  Description of Artificial Sequence Synthetic primer
gttatgagttagttcaaattcgttc

SEQ ID NO: 92 - DNA, 21 bp, Artificial Sequence
  Description of Artificial Sequence Synthetic primer
gtcgagatcagggaatgagtt

SEQ ID NO: 93 - DNA, 19 bp, Artificial Sequence
  Description of Artificial Sequence Synthetic primer
aactcggtcgccgcataca

SEQ ID NO: 94 - DNA, 18 bp, Artificial Sequence
  Description of Artificial Sequence Synthetic primer
ttgcccggcgtcaacacg

SEQ ID NO: 95 - DNA, 23 bp, Artificial Sequence
  Description of Artificial Sequence Synthetic primer
ccgcatacactattctcagaatg

SEQ ID NO: 96 - DNA, 19 bp, Artificial Sequence
  Description of Artificial Sequence Synthetic primer
acacgggataataccgcgc

SEQ ID NO: 97 - DNA, 21 bp, Artificial Sequence
  Description of Artificial Sequence Synthetic primer
tagttatctacacgacgggga

SEQ ID NO: 98 - DNA, 19 bp, Artificial Sequence
  Description of Artificial Sequence Synthetic primer
acgatacgggagggcttac

SEQ ID NO: 99 - DNA, 22 bp, Artificial Sequence
  Description of Artificial Sequence Synthetic primer
agtcaggcaactatggatgaac

SEQ ID NO: 100 - DNA, 18 bp, Artificial Sequence
  Description of Artificial Sequence Synthetic primer
ccatctggccccagtgct

SEQ ID NO: 101 - DNA, 18 bp, Artificial Sequence
  Description of Artificial Sequence Synthetic primer
ggataaggcgcagcggtc

SEQ ID NO: 102 - DNA, 21 bp, Artificial Sequence
  Description of Artificial Sequence Synthetic primer
ggtaactatcgtcttgagtcc

SEQ ID NO: 103 - DNA, 18 bp, Artificial Sequence
  Description of Artificial Sequence Synthetic primer
agcggtcggactgaacgg

SEQ ID NO: 104 - DNA, 20 bp, Artificial Sequence
  Description of Artificial Sequence Synthetic primer
gtccaacccggaaagacatg

SEQ ID NO: 105 - DNA, 21 bp, Artificial Sequence
  Description of Artificial Sequence Synthetic primer
agctatgagatggagaaagcc

SEQ ID NO: 106 - DNA, 21 bp, Artificial Sequence
  Description of Artificial Sequence Synthetic primer
tacccaggcttaaccttcttc

SEQ ID NO: 107 - DNA, 23 bp, Artificial Sequence
  Description of Artificial Sequence Synthetic primer
gccataaccatgagtgataacac

SEQ ID NO: 108 - DNA, 21 bp, Artificial Sequence
  Description of Artificial Sequence Synthetic primer
cttcttcggctttttcacgag

SEQ ID NO: 109 - DNA, 21 bp, Artificial Sequence
  Description of Artificial Sequence Synthetic primer
gcgtgttcagactagaacatc

SEQ ID NO: 110 - DNA, 23 bp, Artificial Sequence
  Description of Artificial Sequence Synthetic primer
cccttatcacctagaacaatcgt

SEQ ID NO: 111 - DNA, 23 bp, Artificial Sequence
  Description of Artificial Sequence Synthetic primer
accagagtataaaggtaatcgtg

SEQ ID NO: 112 - DNA, 21 bp, Artificial Sequence
  Description of Artificial Sequence Synthetic primer
atcgtagttctggcagagtag

SEQ ID NO: 113 - DNA, 21 bp, Artificial Sequence
  Description of Artificial Sequence Synthetic primer
tgctattgctgctgtaggtca

SEQ ID NO: 114 - DNA, 22 bp, Artificial Sequence
  Description of Artificial Sequence Synthetic primer
ccacacagtaggtaggtaaatc

SEQ ID NO: 115 - DNA, 24 bp, Artificial Sequence
  Description of Artificial Sequence Synthetic primer
caagatgtgttagataagttcacg

SEQ ID NO: 116 - DNA, 23 bp, Artificial Sequence
  Description of Artificial Sequence Synthetic primer
atcaaccagaatcaagtttcgga

SEQ ID NO: 117 - DNA, 23 bp, Artificial Sequence
  Description of Artificial Sequence Synthetic primer
gattgagggagatttaaatcctg

SEQ ID NO: 118 - DNA, 23 bp, Artificial Sequence
  Description of Artificial Sequence Synthetic primer
ccaaatagattaagcgcaaatcc

SEQ ID NO: 119 - DNA, 23 bp, Artificial Sequence
  Description of Artificial Sequence Synthetic primer
tcctgataatagtgatgtggaca

SEQ ID NO: 120 - DNA, 23 bp, Artificial Sequence
  Description of Artificial Sequence Synthetic primer
ccgctttatcagtagaatctacc

SEQ ID NO: 121 - DNA, 22 bp, Artificial Sequence
  Description of Artificial Sequence Synthetic primer
ttatattgatgggggagctagc

SEQ ID NO: 122 - DNA, 23 bp, Artificial Sequence
  Description of Artificial Sequence Synthetic primer
catcaatataacctgcatatccg

SEQ ID NO: 123 - DNA, 19 bp, Artificial Sequence
  Description of Artificial Sequence Synthetic primer
ggggagctagccaagaaga

SEQ ID NO: 124 - DNA, 23 bp, Artificial Sequence
  Description of Artificial Sequence Synthetic primer
cctgcatatccgttttttgattg

SEQ ID NO: 125 - DNA, 22 bp, Artificial Sequence
  Description of Artificial Sequence Synthetic primer
ccagcatttctttcaggtgaac

SEQ ID NO: 126 - DNA, 23 bp, Artificial Sequence
  Description of Artificial Sequence Synthetic primer
ggttttcgcattccttcagtaac

SEQ ID NO: 127 - DNA, 23 bp, Artificial Sequence
  Description of Artificial Sequence Synthetic primer
gaacagaagaaagccattgttga

SEQ ID NO: 128 - DNA, 25 bp, Artificial Sequence
  Description of Artificial Sequence Synthetic primer
aacatatttgacctttgtcaattcg

SEQ ID NO: 129 - DNA, 20 bp, Artificial Sequence
  Description of Artificial Sequence Synthetic primer
gtcaaagtaatggggcggca

SEQ ID NO: 130 - DNA, 23 bp, Artificial Sequence
  Description of Artificial Sequence Synthetic primer
tgttcatgtaaactatcgccttg

SEQ ID NO: 131 - DNA, 22 bp, Artificial Sequence
  Description of Artificial Sequence Synthetic primer
cggcataagccagaaaatatcg

SEQ ID NO: 132 - DNA, 20 bp, Artificial Sequence
  Description of Artificial Sequence Synthetic primer
ccttgtccagacacttgtgc

SEQ ID NO: 133 - DNA, 21 bp, Artificial Sequence
  Description of Artificial Sequence Synthetic primer
ggttgaaactcgccaaatcac

SEQ ID NO: 134 - DNA, 23 bp, Artificial Sequence
  Description of Artificial Sequence Synthetic primer
aattggcgtttgataaaaccagc

SEQ ID NO: 135 - DNA, 23 bp, Artificial Sequence
  Description of Artificial Sequence Synthetic primer
aaatcactaagcatgtggcacaa

SEQ ID NO: 136 - DNA, 23 bp, Artificial Sequence
  Description of Artificial Sequence Synthetic primer
gctttatcaagttcactcaaacc

SEQ ID NO: 137 - DNA, 21 bp, Artificial Sequence
  Description of Artificial Sequence Synthetic primer
cacagtgcgcaaagtattgtc

SEQ ID NO: 138 - DNA, 19 bp, Artificial Sequence
  Description of Artificial Sequence Synthetic primer
ctgtggcaaaatctcgccc

SEQ ID NO: 139 - DNA, 22 bp, Artificial Sequence
  Description of Artificial Sequence Synthetic primer
tccatgccccaagtcaatattg

SEQ ID NO: 140 - DNA, 20 bp, Artificial Sequence
  Description of Artificial Sequence Synthetic primer
ctcgccctttatcccagaca

SEQ ID NO: 141 - DNA, 24 bp, Artificial Sequence
  Description of Artificial Sequence Synthetic primer
gctagtcattatgaaaagttgaag

SEQ ID NO: 142 - DNA, 21 bp, Artificial Sequence
  Description of Artificial Sequence Synthetic primer
cacatatttgcttggcagagc

SEQ ID NO: 143 - DNA, 21 bp, Artificial Sequence
  Description of Artificial Sequence Synthetic primer
gttgaagggtagtccagaaga

SEQ ID NO: 144 - DNA, 19 bp, Artificial Sequence
  Description of Artificial Sequence Synthetic primer
gcagagccagctcatttcc

SEQ ID NO: 145 - DNA, 20 bp, Artificial Sequence
  Description of Artificial Sequence Synthetic primer
gagatctgcagtgactggga

SEQ ID NO: 146 - DNA, 21 bp, Artificial Sequence
  Description of Artificial Sequence Synthetic primer
atctctagaagctccagtcac

SEQ ID NO: 147 - DNA, 19 bp, Artificial Sequence
  Description of Artificial Sequence Synthetic primer
cctggcgactagtcttgga

SEQ ID NO: 148 - DNA, 21 bp, Artificial Sequence
  Description of Artificial Sequence Synthetic primer
tcactgcagatctctagaagc

SEQ ID NO: 149 - DNA, 20 bp, Artificial Sequence
  Description of Artificial Sequence Synthetic primer
gagatcaggtcagacgcgat

SEQ ID NO: 150 - DNA, 21 bp, Artificial Sequence
  Description of Artificial Sequence Synthetic primer
atctctagaagctccagtcac

SEQ ID NO: 151 - DNA, 19 bp, Artificial Sequence
  Description of Artificial Sequence Synthetic primer
gatttcctgggtgctcgag

SEQ ID NO: 152 - DNA, 21 bp, Artificial Sequence
  Description of Artificial Sequence Synthetic primer
tcactgcagatctctagaagc

SEQ ID NO: 153 - DNA, 23 bp, Artificial Sequence
  Description of Artificial Sequence Synthetic primer
ctccatctggatttgttcagaac

SEQ ID NO: 154 - DNA, 23 bp, Artificial Sequence
  Description of Artificial Sequence Synthetic primer
agttctgaggtcattactggatc

SEQ ID NO: 155 - DNA, 18 bp, Artificial Sequence
  Description of Artificial Sequence Synthetic primer
agaacgctcggttgccgc

SEQ ID NO: 156 - DNA, 21 bp, Artificial Sequence
  Description of Artificial Sequence Synthetic primer
tggatctatcaacaggagtcc

SEQ ID NO: 157 - DNA, 23 bp, Artificial Sequence
  Description of Artificial Sequence Synthetic primer
gcaagcttgacaagtatttcttc

SEQ ID NO: 158 - DNA, 21 bp, Artificial Sequence
  Description of Artificial Sequence Synthetic primer
tgccatcttaaccggataagg

SEQ ID NO: 159 - DNA, 22 bp, Artificial Sequence
  Description of Artificial Sequence Synthetic primer
ggagggcaaactactagagatg

SEQ ID NO: 160 - DNA, 23 bp, Artificial Sequence
  Description of Artificial Sequence Synthetic primer
gataaggagatcattgaggaagc

SEQ ID NO: 161 - DNA, 18 bp, Artificial Sequence
  Description of Artificial Sequence Synthetic primer
cgtgcttgaggcggacga

SEQ ID NO: 162 - DNA, 20 bp, Artificial Sequence
  Description of Artificial Sequence Synthetic primer
caccggtgtttgttcctcga

SEQ ID NO: 163 - DNA, 21 bp, Artificial Sequence
  Description of Artificial Sequence Synthetic primer
acgacctgtcagaagtgttca

SEQ ID NO: 164 - DNA, 20 bp, Artificial Sequence
  Description of Artificial Sequence Synthetic primer
cgatcttaaggtccggttcg

SEQ ID NO: 165 - DNA, 19 bp, Artificial Sequence
  Description of Artificial Sequence Synthetic primer
gcagtagcgactaccagag

SEQ ID NO: 166 - DNA, 21 bp, Artificial Sequence
  Description of Artificial Sequence Synthetic primer
tgagcttttgctgctctggta

SEQ ID NO: 167 - DNA, 19 bp, Artificial Sequence
  Description of Artificial Sequence Synthetic primer
accagagtagtcgtagccc

SEQ ID NO: 168 - DNA, 20 bp, Artificial Sequence
  Description of Artificial Sequence Synthetic primer
ggtagtcgctactcttcagg

SEQ ID NO: 169 - DNA, 20 bp, Artificial Sequence
  Description of Artificial Sequence Synthetic primer
ggaggacggaaacattctgg

SEQ ID NO: 170 - DNA, 23 bp, Artificial Sequence
  Description of Artificial Sequence Synthetic primer
tcgattcctttgagttcaatacg

SEQ ID NO: 171 - DNA, 21 bp, Artificial Sequence
  Description of Artificial Sequence Synthetic primer
ttctgggccacaaactggaat

SEQ ID NO: 172 - DNA, 20 bp, Artificial Sequence
  Description of Artificial Sequence Synthetic primer
tacggtttaccagggtgtcg

SEQ ID NO: 173 - DNA, 20 bp, Artificial Sequence
  Description of Artificial Sequence Synthetic primer
ttggtgagaatccaggggtc

SEQ ID NO: 174 - DNA, 23 bp, Artificial Sequence
  Description of Artificial Sequence Synthetic primer
gttctgaacaaatccagatggag

SEQ ID NO: 175 - DNA, 21 bp, Artificial Sequence
  Description of Artificial Sequence Synthetic primer
ggggtccccaataattacgat

SEQ ID NO: 176 - DNA, 21 bp, Artificial Sequence
  Description of Artificial Sequence Synthetic primer
ggagttctgaggtcattactg

SEQ ID NO: 177 - DNA, 19 bp, Artificial Sequence
  Description of Artificial Sequence Synthetic primer
cgccgaatgaagcggatga

SEQ ID NO: 178 - DNA, 19 bp, Artificial Sequence
  Description of Artificial Sequence Synthetic primer
tccaccaccgctttcccat

SEQ ID NO: 179 - DNA, 23 bp, Artificial Sequence
  Description of Artificial Sequence Synthetic primer
atgaaattgctcatcgtctcgtg

SEQ ID NO: 180 - DNA, 18 bp, Artificial Sequence
  Description of Artificial Sequence Synthetic primer
atggctcgagcacccagg

SEQ ID NO: 181 - DNA, 20 bp, Artificial Sequence
  Description of Artificial Sequence Synthetic primer
gacagccgaattcggattcc

SEQ ID NO: 182 - DNA, 21 bp, Artificial Sequence
  Description of Artificial Sequence Synthetic primer
aagcttgccatcttaaccgga

SEQ ID NO: 183 - DNA, 21 bp, Artificial Sequence
  Description of Artificial Sequence Synthetic primer
ggattcccgcgtatatcttga

SEQ ID NO: 184 - DNA, 23 bp, Artificial Sequence
  Description of Artificial Sequence Synthetic primer
gataaggagatcattgaggaagc

SEQ ID NO: 185 - DNA, 19 bp, Artificial Sequence
  Description of Artificial Sequence Synthetic primer
gaacctgcaagcccggaat

SEQ ID NO: 186 - DNA, 21 bp, Artificial Sequence
  Description of Artificial Sequence Synthetic primer
tcgcagtttagggattgatgg

SEQ ID NO: 187 - DNA, 23 bp, Artificial Sequence
  Description of Artificial Sequence Synthetic primer
ggaatcgactacatatataacgg

SEQ ID NO: 188 - DNA, 22 bp, Artificial Sequence
  Description of Artificial Sequence Synthetic primer
gatggcctgtttcttcttcaag

SEQ ID NO: 189 - DNA, 22 bp, Artificial Sequence
  Description of Artificial Sequence Synthetic primer
ttgtcaagcaatcctggttcac

SEQ ID NO: 190 - DNA, 23 bp, Artificial Sequence
  Description of Artificial Sequence Synthetic primer
tctcttttacatcctcgtgatcg

SEQ ID NO: 191 - DNA, 22 bp, Artificial Sequence
  Description of Artificial Sequence Synthetic primer
cctttcagaacagggtcatcta

SEQ ID NO: 192 - DNA, 20 bp, Artificial Sequence
  Description of Artificial Sequence Synthetic primer
tcgcaaggatgcagctgatc

SEQ ID NO: 193 - DNA, 22 bp, Artificial Sequence
  Description of Artificial Sequence Synthetic primer
accgaagatgagcattctgttc

SEQ ID NO: 194 - DNA, 23 bp, Artificial Sequence
  Description of Artificial Sequence Synthetic primer
tatgataatgtctactgttcccc

SEQ ID NO: 195 - DNA, 23 bp, Artificial Sequence
  Description of Artificial Sequence Synthetic primer
gttcatgttactgtcaaagacga

SEQ ID NO: 196 - DNA, 21 bp, Artificial Sequence
  Description of Artificial Sequence Synthetic primer
cccatcaggcattgattcaac

SEQ ID NO: 197 - DNA, 20 bp, Artificial Sequence
  Description of Artificial Sequence Synthetic primer
acatgtggacagccatcctg

SEQ ID NO: 198 - DNA, 24 bp, Artificial Sequence
  Description of Artificial Sequence Synthetic primer
atgtataactccctgatggttttc

SEQ ID NO: 199 - DNA, 21 bp, Artificial Sequence
  Description of Artificial Sequence Synthetic primer
atcctgaaaaaggcacagcgt

SEQ ID NO: 200 - DNA, 21 bp, Artificial Sequence
  Description of Artificial Sequence Synthetic primer
tgtcaccattaatccaagccc

SEQ ID NO: 201 - DNA, 21 bp, Artificial Sequence
  Description of Artificial Sequence Synthetic primer
gtcatagctgtttcctgtgtg

SEQ ID NO: 202 - DNA, 21 bp, Artificial Sequence
  Description of Artificial Sequence Synthetic primer
caagcttggcgtaatcatggt

SEQ ID NO: 203 - DNA, 23 bp, Artificial Sequence
  Description of Artificial Sequence Synthetic primer
tgtgtgaaattgttatccgctca

SEQ ID NO: 204 - DNA, 19 bp, Artificial Sequence
  Description of Artificial Sequence Synthetic primer
tgcagaggcctgcatgcaa

SEQ ID NO: 205 - DNA, 19 bp, Artificial Sequence
  Description of Artificial Sequence Synthetic primer
ccgcttaccggatacctgt

SEQ ID NO: 206 - DNA, 18 bp, Artificial Sequence
  Description of Artificial Sequence Synthetic primer
gaagctccctcgtgcgct

SEQ ID NO: 207 - DNA, 19 bp, Artificial Sequence
  Description of Artificial Sequence Synthetic primer
tgtccgcctttctcccttc

SEQ ID NO: 208 - DNA, 18 bp, Artificial Sequence
  Description of Artificial Sequence Synthetic primer
caggcgtttccccctgga

SEQ ID NO: 209 - DNA, 22 bp, Artificial Sequence
  Description of Artificial Sequence Synthetic primer
gaacgaaaactcacgttaaggg

SEQ ID NO: 210 - DNA, 18 bp, Artificial Sequence
  Description of Artificial Sequence Synthetic primer
acggggtctgacgctcag

SEQ ID NO: 211 - DNA, 23 bp, Artificial Sequence
  Description of Artificial Sequence Synthetic primer
gggattttggtcatgagattatc

SEQ ID NO: 212 - DNA, 23 bp, Artificial Sequence
  Description of Artificial Sequence Synthetic primer
ggatctcaagaagatcctttgat

SEQ ID NO: 213 - DNA, 19 bp, Artificial Sequence
  Description of Artificial Sequence Synthetic primer
agcggttagctccttcggt

SEQ ID NO: 214 - DNA, 20 bp, Artificial Sequence
  Description of Artificial Sequence Synthetic primer
cccaacgatcaaggcgagtt

SEQ ID NO: 215 - DNA, 19 bp, Artificial Sequence
  Description of Artificial Sequence Synthetic primer
ggtcctccgatcgttgtca

SEQ ID NO: 216 - DNA, 20 bp, Artificial Sequence
  Description of Artificial Sequence Synthetic primer
ggcttcattcagctccggtt

SEQ ID NO: 217 - DNA, 22 bp, Artificial Sequence
  Description of Artificial Sequence Synthetic primer
cacctgacgtctaagaaaccat

SEQ ID NO: 218 - DNA, 21 bp, Artificial Sequence
  Description of Artificial Sequence Synthetic primer
acatttccccgaaaagtgcca

SEQ ID NO: 219 - DNA, 18 bp, Artificial Sequence
  Description of Artificial Sequence Synthetic primer
aggcgtatcacgaggccc

SEQ ID NO: 220 - DNA, 20 bp, Artificial Sequence
  Description of Artificial Sequence Synthetic primer
taggggttccgcgcacattt

SEQ ID NO: 221 - DNA, 20 bp, Artificial Sequence
  Description of Artificial Sequence Synthetic primer
gaaagggggatgtgctgcaa

SEQ ID NO: 222 - DNA, 20 bp, Artificial Sequence
  Description of Artificial Sequence Synthetic primer
cttcgctattacgccagctg

SEQ ID NO: 223 - DNA, 17 bp, Artificial Sequence
  Description of Artificial Sequence Synthetic primer
tcggtgcgggcctcttc

SEQ ID NO: 224 - DNA, 21 bp, Artificial Sequence
  Description of Artificial Sequence Synthetic primer
gcaaggcgattaagttgggta

SEQ ID NO: 225 - DNA, 4245 bp, Artificial Sequence
  Description of Artificial Sequence Synthetic polynucleotide
ttgtccttttccgctgcataaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttccaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagcgtctgtacgttccttaaggaattattccttagtgctttctaggttaatgtcatgataataatggtttcttagacgtcaggtggcacttttcggggaaatgtccgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggaggtttttcgaaggttctctgagctaccaactctttgaaccgaggtaactggcttgcaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcagggggcggagcctatggaaaaacgctttgcccaagcttctagagatctgcagtgactgggaaaaccctggcgactagtcttggactcctgttgatagatccagtaatgacctcagaactccatctggatttgttcagaacgctcggttgccgccgggcgttttttattggtgagaatccaggggtccccaataattacgatttaaatccttcaaacttcccaaaggcgagccctagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatcccgtttgttgaagactt

SEQ ID NO: 226 - DNA, 6578 bp, Artificial Sequence
  Description of Artificial Sequence Synthetic polynucleotide
tatggaaaaacgctttgccccacacaggctcttgaacaagggataacaatttgtcatagctgtattcgttacaaggaggattttcgaaaaaacaccctaacgggtgtttttttatagctggtctcccttgcttgtactttacagtatgccatacgaaactgcgtcaatacacgttgacactcttttgtagttatgttaaattatcagatcgccatcattagtttgtttaaacaacaaactaatactagaggctcttaaagggggttttagataccagagatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaatagctgccacctttctgaactttggtaatgcgtctcggtaccaaattccagaaaagaggcctcccgaaaggggggccttttttcgttttggtccctcaatgatctccttatccggttaagatggcaagcttgacaagtatttcttcagaatttacaatgaagtttactagaggattaactaataaggagggcaaactactagagatgacgggcctgaacaagagcaccgtaagcagccaagtgaatacactgatgaaggagtcaatggtgttcgagatcggccagggccagagctcaggtggccgccgcccggtaatgttagtcttcaacaagaaagcgggctatagtgtgggcatcgacgtcggcgtagactacatcaacggaatccttacggatttagagggcacgattgtgttggaccagtatagacaccttgagagcaacagcccggagatcactaaggatatccttatcgacatgatccaccatttcatcacacagatgcctcagagcccttatggattcatcggcatcggcatctgtgtaccgggtttaatcgacaaggaccagaagatcgtgtttacgcctaatagcaattggcgtgacatcgatctgaagagcagcatccaggagaaatataacgtaagcgtgttcatcgagaacgaagcgaacgcaggagcgtacggcgagaagttgttcggtgcagcgaagaaccatgacaatatcatctatgtgtcaatttcaacgggcatcggaattggcgtgatcattaataaccacctgtaccgtggcgtgtcaggatttagcggtgagatgggccacatgacgatcgatttcaacggaccgaagtgttcatgtggcaatagaggctgttgggagctttacgcgagcgagaaagcgctgctgaagagcttgcaaacgaaggagaagaaactcagctaccaggacatcatcaatcttgcacaccttaacgacattggcacactgaacgcgctgcagaacttcggcttctacctgggcatcggattgacaaacatcttgaacacgttcaatccgcaggcagtgatcctgcgcaactcaatcatcgagagccacccgatggtgctgaacagcatgcgctcagaggtgagcagccgtgtgtacagccagctcggaaactcatacgagctgcttccgagcagtcttggccagaacgctcctgcgcttggcatgagcagcatcgtaatcgaccacttcttagatatgatcacgatgtaatagtcaaatgtcagacgaaaatgccaattattgaagcggctaacgccgctttttttgtttctggtctccccatagtttaacaagtgctgtaagaagtcattcagcatagcttctagagatctgcagtgactgggaaaaccctggcgactagtcttggactcctgttgatagatccagtaatgacctcagaactccatctggatttgttcagaacgctcggttgccgccgggcgttttttattggtgagaatccaggggtccccaataattacgatttaaatccttcaaacttcccaaaggcgagccctagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatcccgtttgttgaagacttttgtccttttccgctgcataaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttccaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagcgtctgtacgttccttaaggaattattccttagtgctttctaggttaatgtcatgataataatggtttcttagacgtcaggtggcacttttcggggaaatgtccgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggaggtttttcgaaggttctctgagctaccaactctttgaaccgaggtaactggcttgcaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcagggggcggagcc

SEQ ID NO: 227 - DNA, 12703 bp, Artificial Sequence
  Description of Artificial Sequence Synthetic polynucleotide
tacggttatccacagaatcagagggcaaactactagagatgacgggcctgaacaagagcaccgtaagcagccaagtgaatacactgatgaaggagtcaatggtgttcgagatcggccagggccagagctcaggtggccgccgcccggtaatgttagtcttcaacaagaaagcgggctatagtgtgggcatcgacgtcggcgtagactacatcaacggaatccttacggatttagagggcacgattgtgttggaccagtatagacaccttgagagcaacagcccggagatcactaaggatatccttatcgacatgatccaccatttcatcacacagatgcctcagagcccttatggattcatcggcatcggcatctgtgtaccgggtttaatcgacaaggaccagaagatcgtgtttacgcctaatagcaattggcgtgacatcgatctgaagagcagcatccaggagaaatataacgtaagcgtgttcatcgagaacgaagcgaacgcaggagcgtacggcgagaagttgttcggtgcagcgaagaaccatgacaatatcatctatgtgtcaatttcaacgggcatcggaattggcgtgatcattaataaccacctgtaccgtggcgtgtcaggatttagcggtgagatgggccacatgacgatcgatttcaacggaccgaagtgttcatgtggcaatagaggctgttgggagctttacgcgagcgagaaagcgctgctgaagagcttgcaaacgaaggagaagaaactcagctaccaggacatcatcaatcttgcacaccttaacgacattggcacactgaacgcgctgcagaacttcggcttctacctgggcatcggattgacaaacatcttgaacacgttcaatccgcaggcagtgatcctgcgcaactcaatcatcgagagccacccgatggtgctgaacagcatgcgctcagaggtgagcagccgtgtgtacagccagctcggaaactcatacgagctgcttccgagcagtcttggccagaacgctcctgcgcttggcatgagcagcatcgtaatcgaccacttcttagatatgatcacgatgtaatagtcaaatgtcagacgaaaatgccaattattgaagcggctaacgccgctttttttgtttctggtctccccatagtttaacaagtgctgtaagaagtcattcagcatagcttctagagatctgcagtgactgggaaaaccctggcgactagtcttggactcctgttgatagatccagtaatgacctcagaactccatctggatttgttcagaacgctcggttgccgccgggcgttttttattggtgagaatccaggggtccccaataattacgatttaaatccttcaaacttcccaaaggcgagccctagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatcccgtttgttgaagacttttgtccttttccgctgcataaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttccaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagcgtctgtacgttccttaaggaattattccttagtgctttctaggttaatgtcatgataataatggtttcttagacgtcaggtggcacttttcggggaaatgtccgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggaggtttttcgaaggttctctgagctaccaactctttgaaccgaggtaactggcttgcaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcagggggcggagcctatggaaaaacgctttgccccacacaggctcttgaacaagggataacaatttgtcatagctgtattcgttacaaggaggattttcgaaaaaacaccctaacgggtgtttttttatagctggtctcccttgcttgtactttacagtatgccatacgaaactgcgtcaatacacgttgacactcttttgtagttatgttaaattatcagatcgccatcattagtttgtttaaacaacaaactaatactagaggctcttaaagggggttttagataccagagatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaatagctgccacctttctgaactttggtaatgcgtctcggtaccaaattccagaaaagaggcctcccgaaaggggggccttttttcgttttggtccctccaatgatctccttatccggtcgacattcacgattacttggctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctatcgatgcatgcagccggtaccatcacaagcactttgggacgttctcccttagtgcttttttgatttctcataggccggcctgttagtcatatggacacttaagctctagacggtgatcaacacgctagccaggcatcaaataaaacgaaaggctcagtcgaaagactgggcctttcgttttatctgttgtttgtcggtgaacgctctctactagagtcacactggctcaccttcgggtgggcctttctgcgtttatagaattcactagtaattgtgaggcggataacaattctcactcattctacagtttattcttgacattgcactgtccccctggtataataactaattgtgagcgctcacaattaagatccccggggcggccgcgaattcaaaagatctaaagaggagaaaggatctatggataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatgccattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactaagtcgacctcgagactagtcaaggtcggcaattctgcagtactaggacgccgccaagccagcttaaacccagctcaatgagctgggttttttgtttgttaaaaatgaagaagaaactgtgaagcgtatttatagcaaagcactcaaaagtttaccttatgggtgcttttttcgtgcttttttgaaaagacaaaaaaaagaaccttgccaagcaagattcttttgatagcgctatcgctgagcgccggtcgctaccattaccagttggtctggtgtcaaaaataataataaccgggcaggccatgtctgcccgtatttcgcgtaaggaaatccattatgtactatttctggtgatgaaatcaacgtaacatttaaagctgtcaaagccaaagtcatgagatggcgtatggagcgtaaagctgacaagagcggtgttgcgatgattgagatgaccttccttgcaccaagtgaattgcctcaagaaagcactcaatcaaagattcttgtagatggaaaagaacttgctgatttcgctgaaaatcgtcaagactatcaaattacctataaaggtcaacggccaaaagtctcagttgaagaaaacaatcaagtagcttcaactgtggtagatagtggagaagatagccttccagtacttgttcgcctcgtttcagaaagtggaaaacaagtcaaggaataccgtatccagttgactaaggaaaaaccagtttctgctgtacaagaagatcttccaaaactcgaatttgttgaaaaagatttggcctacaagacagttgagaaaaaagattcaacactgtatctaggtgaaactcgtgtagaacaagaaggaaaagttggaaaagaacgtatctttacagtgattaatcctgatggaagtaaggaagaaaaactccgtgaagtggtagaagttccgacagaccgcatcgtcttggttggaaccaaaccagtagctcaagaagctaaaaaaccacaagtgtcagaaaaagcagatacaaaaccaattgattcaagtgaagctgatcaaactaataaagcccagttaccaaatacaggtagtgcggcaagccaagcagcagtagcagcaggtttagcctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaa

SEQ ID NO: 228 - DNA, 4980 bp, Artificial Sequence
  Description of Artificial Sequence Synthetic polynucleotide
gagcctatggaaaaacgctttgccccacacaggctcttgaacaagggataacaatttgtcatagctgtattcgttacaaggaggattttcgaaaaaacaccctaacgggtgtttttttatagctggtctccccaagtgctgtaaaaaacgccgaatgaagcggatgaaattgctcatcgtctcgtgaaaaagccgaagaaggttaagcctgggtataaaaagaaaatgagctatgagatggagaaagccataaccatgagtgataacactggttttagagctagaaatagcaagttaaaataaggctagtccgttatcaacttgaaaaagtggcaccgagtcggtgctttttttctcggtaccaaattccagaaaagaggcctcccgaaaggggggccttttttcgttttggtccctcaatgatctccttatccggttaagatggcaagcttgacaagtatttcttcagaatttacaatgaagtttacatgacgctcttaaagggggttttagataccagagatggataagaaatactcaataggcttagatatcggcacaaatagcgtcggatgggcggtgaaacacgcattgatttgagtcagctaggaggtgactgataatcaaatgtcagacgaaaatgccaattattgaagcggctaacgccgctttttttgtttctggtctcccttgcttgtactttacagtatagcttctagagatctgcagtgactggagcttctagagatctgcagtgactgggaaaaccctggcgactagtcttggactcctgttgatagatccagtaatgacctcagaactccatctggatttgttcagaacgctcggttgccgccgggcgttttttattggtgagaatccaggggtccccaataattacgatttaaatccttcaaacttcccaaaggcgagccctagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatcccgtttgttgaagacttttgtccttttccgctgcataaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttccaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagcgtctgtacgttccttaaggaattattccttagtgctttctaggttaatgtcatgataataatggtttcttagacgtcaggtggcacttttcggggaaatgtccgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggaggtttttcgaaggttctctgagctaccaactctttgaaccgaggtaactggcttgcaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcagggggcggagcctatggaaaaacgctttgccc

SEQ ID NO: 229 - DNA, 8989 bp, Artificial Sequence
  Description of Artificial Sequence Synthetic polynucleotide
aataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactgataatcaaatgtcagacgaaaatgccaattattgaagcggctaacgccgctttttttgtttctggtctcccttgcttgtactttacagtatagcttctagagatctgcagtgactggagcttctagagatctgcagtgactgggaaaaccctggcgactagtcttggactcctgttgatagatccagtaatgacctcagaactccatctggatttgttcagaacgctcggttgccgccgggcgttttttattggtgagaatccaggggtccccaataattacgatttaaatccttcaaacttcccaaaggcgagccctagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatcccgtttgttgaagacttttgtccttttccgctgcataaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttccaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagcgtctgtacgttccttaaggaattattccttagtgctttctaggttaatgtcatgataataatggtttcttagacgtcaggtggcacttttcggggaaatgtccgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggaggtttttcgaaggttctctgagctaccaactctttgaaccgaggtaactggcttgcaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcagggggcggagcctatggaaaaacgctttgcccgagcctatggaaaaacgctttgccccacacaggctcttgaacaagggataacaatttgtcatagctgtattcgttacaaggaggattttcgaaaaaacaccctaacgggtgtttttttatagctggtctccccaagtgctgtaaaaaacgccgaatgaagcggatgaaattgctcatcgtctcgtgaaaaagccgaagaaggttaagcctgggtataaaaagaaaatgagctatgagatggagaaagccataaccatgagtgataacactggttttagagctagaaatagcaagttaaaataaggctagtccgttatcaacttgaaaaagtggcaccgagtcggtgctttttttctcggtaccaaattccagaaaagaggcctcccgaaaggggggccttttttcgttttggtccctcaatgatctccttatccggttaagatggcaagcttgacaagtatttcttcagaatttacaatgaagtttacatgacgctcttaaagggggttttagataccagagatggataagaaatactc

SEQ ID NO: 230 - DNA, 9367 bp, Artificial Sequence
  Description of Artificial Sequence Synthetic polynucleotide
aggtcagacgcgatttcctgggtgctcgagccatgggaaagcggtggtggaaaaaacgccgaatgaagcggatgaaattgctcatcgtctcgtgaaaaagccgaagaaggttaagcctgggtataaaaagaaaatgagctatgagatggagaaattgaagtcatgcgccggttagttttagagctagaaatagcaagttaaaataaggctagtccgttatcaacttgaaaaagtggcaccgagtcggtgctttttttctcggtaccaaattccagaaaagaggcctcccgaaaggggggccttttttcgttttggtccctcaatgatccgacaggaagtggtattagcgactcctacaaggaataggtgtctgggtcatgcagtgactgggaaaaccctggcgactagtcttggactcctgttgatagatccagtaatgacctcagaactccatctggatttgttcagaacgctcggttgccgccgggcgttttttattggtgagaatccaggggtccccaataattacgatttaaatccttcaaacttcccaaaggcgagccctagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatcccgtttgttgaagacttttgtccttttccgctgcataaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttccaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagcgtctgtacgttccttaaggaattattccttagtgctttctaggttaatgtcatgataataatggtttcttagacgtcaggtggcacttttcggggaaatgtccgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggaggtttttcgaaggttctctgagctaccaactctttgaaccgaggtaactggcttgcaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcagggggcggagcctatggaaaaacgctttgcccgagcctatggaaaaacgctttgccccacacaggctcttgaacaagggataacaatttgtcatagctgtattcgttacaaggaggattttcgaaaaaacaccctaacgggtgtttttttatagctggtctccccaagtgctgtaaaaaacgccgaatgaagcggatgaaattgctcatcgtctcgtgaaaaagccgaagaaggttaagcctgggtataaaaagaaaatgagctatgagatggagaaagccataaccatgagtgataacactggttttagagctagaaatagcaagttaaaataaggctagtccgttatcaacttgaaaaagtggcaccgagtcggtgctttttttctcggtaccaaattccagaaaagaggcctcccgaaaggggggccttttttcgttttggtccctcaatgatctccttatccggttaagatggcaagcttgacaagtatttcttcagaatttacaatgaagtttacatgacgctcttaaagggggttttagataccagagatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactgataatcaaatgtcagacgaaaatgccaattattgaagcggctaacgccgctttttttgtttctggtctcccttgcttgtactttacagtatagcttctagagatctgcagtgactggagcttctagagatc

SEQ ID NO: 231 - DNA, 9900 bp, Artificial Sequence
  Description of Artificial Sequence Synthetic polynucleotide
gattaactaataaggagggcaaactactagagatgagtaaatcctggggcaaattcatcgaggaggaagaggcggaaatggcttcccgtcgtaatctaatgattgtcgatggaactaacttaggctttcgcttcaaacataacaatagtaaaaaaccatttgcctcaagttatgtttcaactattcaatctctggcaaaatcctactctgccagaactacgattgttctaggtgataagggcaaaagcgtgttcagactagaacatctaccagagtataaaggtaatcgtgatgaaaagtacgcacaacgtacggaagaggagaaagcgctagatgagcagttcttcgaatacctcaaagatgctttcgagttgtgtaaaactacattcccaacttttaccattcgtggtgtagaagcagatgacatggcggcgtatattgttaagctcatcgggcatctttatgatcacgtttggctaatatctacagatggtgactgggatactttattaacggataaagtttctcgtttttctttcacaacacgtcgtgagtatcatcttcgtgatatgtatgagcaccataacgtggacgacgtagaacagtttatctccctgaaagcaattatgggagatctaggagataatattcgtggtgttgaaggaataggagcaaaacgcggatataatattattcgtgagtttggtaacgtactggatattattgatcagcttccactgcctggaaagcagaaatatatacagaacctgaatgcatcggaagaactgcttttccgaaacttgattctggttgatttacctacctactgtgtggatgctattgctgctgtaggtcaagatgtgttagataagttcacgaaagacatccttgagatcgcggaacaatgataagctcttaaagggggttttagataccagagatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactgataatcaaatgtcagacgaaaatgccaattattgaagcggctaacgccgctttttttgtttctggtctcccttgcttgtactttacagtatagcttctagagatctgcagtgactggagcttctagagatctgcagtgactgggaaaaccctggcgactagtcttggactcctgttgatagatccagtaatgacctcagaactccatctggatttgttcagaacgctcggttgccgccgggcgttttttattggtgagaatccaggggtccccaataattacgatttaaatccttcaaacttcccaaaggcgagccctagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatcccgtttgttgaagacttttgtccttttccgctgcataaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttccaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagcgtctgtacgttccttaaggaattattccttagtgctttctaggttaatgtcatgataataatggtttcttagacgtcaggtggcacttttcggggaaatgtccgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggaggtttttcgaaggttctctgagctaccaactctttgaaccgaggtaactggcttgcaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcagggggcggagcctatggaaaaacgctttgcccgagcctatggaaaaacgctttgccccacacaggctcttgaacaagggataacaatttgtcatagctgtattcgttacaaggaggattttcgaaaaaacaccctaacgggtgtttttttatagctggtctccccaagtgctgtaaaaaacgccgaatgaagcggatgaaattgctcatcgtctcgtgaaaaagccgaagaaggttaagcctgggtataaaaagaaaatgagctatgagatggagaaagccataaccatgagtgataacactggttttagagctagaaatagcaagttaaaataaggctagtccgttatcaacttgaaaaagtggcaccgagtcggtgctttttttctcggtaccaaattccagaaaagaggcctcccgaaaggggggccttttttcgttttggtccctcaatgatctccttatccggttaagatggcaagcttgacaagtatttcttcagaatttacaatgaagtttacatgac

SEQ ID NO: 232 - DNA, 10278 bp, Artificial Sequence
  Description of Artificial Sequence Synthetic polynucleotide
gattaactaataaggagggcaaactactagagatgagtaaatcctggggcaaattcatcgaggaggaagaggcggaaatggcttcccgtcgtaatctaatgattgtcgatggaactaacttaggctttcgcttcaaacataacaatagtaaaaaaccatttgcctcaagttatgtttcaactattcaatctctggcaaaatcctactctgccagaactacgattgttctaggtgataagggcaaaagcgtgttcagactagaacatctaccagagtataaaggtaatcgtgatgaaaagtacgcacaacgtacggaagaggagaaagcgctagatgagcagttcttcgaatacctcaaagatgctttcgagttgtgtaaaactacattcccaacttttaccattcgtggtgtagaagcagatgacatggcggcgtatattgttaagctcatcgggcatctttatgatcacgtttggctaatatctacagatggtgactgggatactttattaacggataaagtttctcgtttttctttcacaacacgtcgtgagtatcatcttcgtgatatgtatgagcaccataacgtggacgacgtagaacagtttatctccctgaaagcaattatgggagatctaggagataatattcgtggtgttgaaggaataggagcaaaacgcggatataatattattcgtgagtttggtaacgtactggatattattgatcagcttccactgcctggaaagcagaaatatatacagaacctgaatgcatcggaagaactgcttttccgaaacttgattctggttgatttacctacctactgtgtggatgctattgctgctgtaggtcaagatgtgttagataagttcacgaaagacatccttgagatcgcggaacaatgataagctcttaaagggggttttagataccagagatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactgataatcaaatgtcagacgaaaatgccaattattgaagcggctaacgccgctttttttgtttctggtctcccttgcttgtactttacagtatagcttctagagatctgcagtgactggagcttctagagatcaggtcagacgcgatttcctgggtgctcgagccatgggaaagcggtggtggaaaaaacgccgaatgaagcggatgaaattgctcatcgtctcgtgaaaaagccgaagaaggttaagcctgggtataaaaagaaaatgagctatgagatggagaaattgaagtcatgcgccggttagttttagagctagaaatagcaagttaaaataaggctagtccgttatcaacttgaaaaagtggcaccgagtcggtgctttttttctcggtaccaaattccagaaaagaggcctcccgaaaggggggccttttttcgttttggtccctcaatgatccgacaggaagtggtattagcgactcctacaaggaataggtgtctgggtcatgcagtgactgggaaaaccctggcgactagtcttggactcctgttgatagatccagtaatgacctcagaactccatctggatttgttcagaacgctcggttgccgccgggcgttttttattggtgagaatccaggggtccccaataattacgatttaaatccttcaaacttcccaaaggcgagccctagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatcccgtttgttgaagacttttgtccttttccgctgcataaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttccaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagcgtctgtacgttccttaaggaattattccttagtgctttctaggttaatgtcatgataataatggtttcttagacgtcaggtggcacttttcggggaaatgtccgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggaggtttttcgaaggttctctgagctaccaactctttgaaccgaggtaactggcttgcaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcagggggcggagcctatggaaaaacgctttgcccgagcctatggaaaaacgctttgccccacacaggctcttgaacaagggataacaatttgtcatagctgtattcgttacaaggaggattttcgaaaaaacaccctaacgggtgtttttttatagctggtctccccaagtgctgtaaaaaacgccgaatgaagcggatgaaattgctcatcgtctcgtgaaaaagccgaagaaggttaagcctgggtataaaaagaaaatgagctatgagatggagaaagccataaccatgagtgataacactggttttagagctagaaatagcaagttaaaataaggctagtccgttatcaacttgaaaaagtggcaccgagtcggtgctttttttctcggtaccaaattccagaaaagaggcctcccgaaaggggggccttttttcgttttggtccctcaatgatctccttatccggttaagatggcaagcttgacaagtatttcttcagaatttacaatgaagtttacatgac

SEQ ID NO: 233 - DNA, 9012 bp, Artificial Sequence
  Description of Artificial Sequence Synthetic polynucleotide
tccttatccggttaagatggcttttaaatcaattttcagctcctgtatacaattaccaaagtttttctgaatgaagccatgtgttttgacacattctatactcacaaggaggtgagacacatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactgataatcaaatgtcagacgaaaatgccaattattgaagcggctaacgccgctttttttgtttctggtctcccttgcttgtactttacagtatagcttctagagatctgcagtgactggagcttctagagatctgcagtgactgggaaaaccctggcgactagtcttggactcctgttgatagatccagtaatgacctcagaactccatctggatttgttcagaacgctcggttgccgccgggcgttttttattggtgagaatccaggggtccccaataattacgatttaaatccttcaaacttcccaaaggcgagccctagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatcccgtttgttgaagacttttgtccttttccgctgcataaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttccaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagcgtctgtacgttccttaaggaattattccttagtgctttctaggttaatgtcatgataataatggtttcttagacgtcaggtggcacttttcggggaaatgtccgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggaggtttttcgaaggttctctgagctaccaactctttgaaccgaggtaactggcttgcaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcagggggcggagcctatggaaaaacgctttgcccgagcctatggaaaaacgctttgccccacacaggctcttgaacaagggataacaatttgtcatagctgtattcgttacaaggaggattttcgaaaaaacaccctaacgggtgtttttttatagctggtctccccaagtgctgtaaaaaacgccgaatgaagcggatgaaattgctcatcgtctcgtgaaaaagccgaagaaggttaagcctgggtataaaaagaaaatgagctatgagatggagaaagccataaccatgagtgataacactggttttagagctagaaatagcaagttaaaataaggctagtccgttatcaacttgaaaaagtggcaccgagtcggtgctttttttctcggtaccaaattccagaaaagaggcctcccgaaaggggggccttttttcgttttggtccctcaatgatc

SEQ ID NO: 234 - DNA, 9390 bp, Artificial Sequence
  Description of Artificial Sequence Synthetic polynucleotide
tccttatccggttaagatggcttttaaatcaattttcagctcctgtatacaattaccaaagtttttctgaatgaagccatgtgttttgacacattctatactcacaaggaggtgagacacatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactgataatcaaatgtcagacgaaaatgccaattattgaagcggctaacgccgctttttttgtttctggtctcccttgcttgtactttacagtatagcttctagagatctgcagtgactggagcttctagagatcaggtcagacgcgatttcctgggtgctcgagccatgggaaagcggtggtggaaaaaacgccgaatgaagcggatgaaattgctcatcgtctcgtgaaaaagccgaagaaggttaagcctgggtataaaaagaaaatgagctatgagatggagaaattgaagtcatgcgccggttagttttagagctagaaatagcaagttaaaataaggctagtccgttatcaacttgaaaaagtggcaccgagtcggtgctttttttctcggtaccaaattccagaaaagaggcctcccgaaaggggggccttttttcgttttggtccctcaatgatccgacaggaagtggtattagcgactcctacaaggaataggtgtctgggtcatgcagtgactgggaaaaccctggcgactagtcttggactcctgttgatagatccagtaatgacctcagaactccatctggatttgttcagaacgctcggttgccgccgggcgttttttattggtgagaatccaggggtccccaataattacgatttaaatccttcaaacttcccaaaggcgagccctagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatcccgtttgttgaagacttttgtccttttccgctgcataaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttccaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagcgtctgtacgttccttaaggaattattccttagtgctttctaggttaatgtcatgataataatggtttcttagacgtcaggtggcacttttcggggaaatgtccgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggaggtttttcgaaggttctctgagctaccaactctttgaaccgaggtaactggcttgcaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcagggggcggagcctatggaaaaacgctttgcccgagcctatggaaaaacgctttgccccacacaggctcttgaacaagggataacaatttgtcatagctgtattcgttacaaggaggattttcgaaaaaacaccctaacgggtgtttttttatagctggtctccccaagtgctgtaaaaaacgccgaatgaagcggatgaaattgctcatcgtctcgtgaaaaagccgaagaaggttaagcctgggtataaaaagaaaatgagctatgagatggagaaagccataaccatgagtgataacactggttttagagctagaaatagcaagttaaaataaggctagtccgttatcaacttgaaaaagtggcaccgagtcggtgctttttttctcggtaccaaattccagaaaagaggcctcccgaaaggggggccttttttcgttttggtccctcaatgatc

SEQ ID NO: 235 - DNA, 9920 bp, Artificial Sequence
  Description of Artificial Sequence Synthetic polynucleotide
tccttatccggttaagatggcttttaaatcaattttcagctcctgtatacaattaccaaagtttttctgaatgaagccatgtgttttgacacattctatactcacaaggaggtgagacacatgagtaaatcctggggcaaattcatcgaggaggaagaggcggaaatggcttcccgtcgtaatctaatgattgtcgatggaactaacttaggctttcgcttcaaacataacaatagtaaaaaaccatttgcctcaagttatgtttcaactattcaatctctggcaaaatcctactctgccagaactacgattgttctaggtgataagggcaaaagcgtgttcagactagaacatctaccagagtataaaggtaatcgtgatgaaaagtacgcacaacgtacggaagaggagaaagcgctagatgagcagttcttcgaatacctcaaagatgctttcgagttgtgtaaaactacattcccaacttttaccattcgtggtgtagaagcagatgacatggcggcgtatattgttaagctcatcgggcatctttatgatcacgtttggctaatatctacagatggtgactgggatactttattaacggataaagtttctcgtttttctttcacaacacgtcgtgagtatcatcttcgtgatatgtatgagcaccataacgtggacgacgtagaacagtttatctccctgaaagcaattatgggagatctaggagataatattcgtggtgttgaaggaataggagcaaaacgcggatataatattattcgtgagtttggtaacgtactggatattattgatcagcttccactgcctggaaagcagaaatatatacagaacctgaatgcatcggaagaactgcttttccgaaacttgattctggttgatttacctacctactgtgtggatgctattgctgctgtaggtcaagatgtgttagataagttcacgaaagacatccttgagatcgcggaacaatgataagctcttaaagggggttttagataccagagatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactgataatcaaatgtcagacgaaaatgccaattattgaagcggctaacgccgctttttttgtttctggtctcccttgcttgtactttacagtatagcttctagagatctgcagtgactggagcttctagagatctgcagtgactgggaaaaccctggcgactagtcttggactcctgttgatagatccagtaatgacctcagaactccatctggatttgttcagaacgctcggttgccgccgggcgttttttattggtgagaatccaggggtccccaataattacgatttaaatccttcaaacttcccaaaggcgagccctagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatcccgtttgttgaagacttttgtccttttccgctgcataaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttccaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagcgtctgtacgttccttaaggaattattccttagtgctttctaggttaatgtcatgataataatggtttcttagacgtcaggtggcacttttcggggaaatgtccgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggaggtttttcgaaggttctctgagctaccaactctttgaaccgaggtaactggcttgcaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcagggggcggagcctatggaaaaacgctttgcccgagcctatggaaaaacgctttgccccacacaggctcttgaacaagggataacaatttgtcatagctgtattcgttacaaggaggattttcgaaaaaacaccctaacgggtgtttttttatagctggtctccccaagtgctgtaaaaaacgccgaatgaagcggatgaaattgctcatcgtctcgtgaaaaagccgaagaaggttaagcctgggtataaaaagaaaatgagctatgagatggagaaagccataaccatgagtgataacactggttttagagctagaaatagcaagttaaaataaggctagtccgttatcaacttgaaaaagtggcaccgagtcggtgctttttttctcggtaccaaattccagaaaagaggcctcccgaaaggggggccttttttcgttttggtccctcaatgatc

SEQ ID NO: 236 - DNA, 10298 bp, Artificial Sequence
  Description of Artificial Sequence Synthetic polynucleotide
tccttatccggttaagatggcttttaaatcaattttcagctcctgtatacaattaccaaagtttttctgaatgaagccatgtgttttgacacattctatactcacaaggaggtgagacacatgagtaaatcctggggcaaattcatcgaggaggaagaggcggaaatggcttcccgtcgtaatctaatgattgtcgatggaactaacttaggctttcgcttcaaacataacaatagtaaaaaaccatttgcctcaagttatgtttcaactattcaatctctggcaaaatcctactctgccagaactacgattgttctaggtgataagggcaaaagcgtgttcagactagaacatctaccagagtataaaggtaatcgtgatgaaaagtacgcacaacgtacggaagaggagaaagcgctagatgagcagttcttcgaatacctcaaagatgctttcgagttgtgtaaaactacattcccaacttttaccattcgtggtgtagaagcagatgacatggcggcgtatattgttaagctcatcgggcatctttatgatcacgtttggctaatatctacagatggtgactgggatactttattaacggataaagtttctcgtttttctttcacaacacgtcgtgagtatcatcttcgtgatatgtatgagcaccataacgtggacgacgtagaacagtttatctccctgaaagcaattatgggagatctaggagataatattcgtggtgttgaaggaataggagcaaaacgcggatataatattattcgtgagtttggtaacgtactggatattattgatcagcttccactgcctggaaagcagaaatatatacagaacctgaatgcatcggaagaactgcttttccgaaacttgattctggttgatttacctacctactgtgtggatgctattgctgctgtaggtcaagatgtgttagataagttcacgaaagacatccttgagatcgcggaacaatgataagctcttaaagggggttttagataccagagatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactgataatcaaatgtcagacgaaaatgccaattattgaagcggctaacgccgctttttttgtttctggtctcccttgcttgtactttacagtatagcttctagagatctgcagtgactggagcttctagagatcaggtcagacgcgatttcctgggtgctcgagccatgggaaagcggtggtggaaaaaacgccgaatgaagcggatgaaattgctcatcgtctcgtgaaaaagccgaagaaggttaagcctgggtataaaaagaaaatgagctatgagatggagaaattgaagtcatgcgccggttagttttagagctagaaatagcaagttaaaataaggctagtccgttatcaacttgaaaaagtggcaccgagtcggtgctttttttctcggtaccaaattccagaaaagaggcctcccgaaaggggggccttttttcgttttggtccctcaatgatccgacaggaagtggtattagcgactcctacaaggaataggtgtctgggtcatgcagtgactgggaaaaccctggcgactagtcttggactcctgttgatagatccagtaatgacctcagaactccatctggatttgttcagaacgctcggttgccgccgggcgttttttattggtgagaatccaggggtccccaataattacgatttaaatccttcaaacttcccaaaggcgagccctagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatcccgtttgttgaagacttttgtccttttccgctgcataaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttccaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagcgtctgtacgttccttaaggaattattccttagtgctttctaggttaatgtcatgataataatggtttcttagacgtcaggtggcacttttcggggaaatgtccgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggaggtttttcgaaggttctctgagctaccaactctttgaaccgaggtaactggcttgcaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcagggggcggagcctatggaaaaacgctttgcccgagcctatggaaaaacgctttgccccacacaggctcttgaacaagggataacaatttgtcatagctgtattcgttacaaggaggattttcgaaaaaacaccctaacgggtgtttttttatagctggtctccccaagtgctgtaaaaaacgccgaatgaagcggatgaaattgctcatcgtctcgtgaaaaagccgaagaaggttaagcctgggtataaaaagaaaatgagctatgagatggagaaagccataaccatgagtgataacactggttttagagctagaaatagcaagttaaaataaggctagtccgttatcaacttgaaaaagtggcaccgagtcggtgctttttttctcggtaccaaattccagaaaagaggcctcccgaaaggggggccttttttcgttttggtccctcaatgatc

SEQ ID NO: 237 - DNA, 9819 bp, Artificial Sequence
  Description of Artificial Sequence Synthetic polynucleotide
gagtaaatcctggggcaaattcatcgaggaggaagaggcggaaatggcttcccgtcgtaatctaatgattgtcgatggaactaacttaggctttcgcttcaaacataacaatagtaaaaaaccatttgcctcaagttatgtttcaactattcaatctctggcaaaatcctactctgccagaactacgattgttctaggtgataagggcaaaagcgtgttcagactagaacatctaccagagtataaaggtaatcgtgatgaaaagtacgcacaacgtacggaagaggagaaagcgctagatgagcagttcttcgaatacctcaaagatgctttcgagttgtgtaaaactacattcccaacttttaccattcgtggtgtagaagcagatgacatggcggcgtatattgttaagctcatcgggcatctttatgatcacgtttggctaatatctacagatggtgactgggatactttattaacggataaagtttctcgtttttctttcacaacacgtcgtgagtatcatcttcgtgatatgtatgagcaccataacgtggacgacgtagaacagtttatctccctgaaagcaattatgggagatctaggagataatattcgtggtgttgaaggaataggagcaaaacgcggatataatattattcgtgagtttggtaacgtactggatattattgatcagcttccactgcctggaaagcagaaatatatacagaacctgaatgcatcggaagaactgcttttccgaaacttgattctggttgatttacctacctactgtgtggatgctattgctgctgtaggtcaagatgtgttagataagttcacgaaagacatccttgagatcgcggaacaatgataagctcttaaagggggttttagataccagagatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactgataatcaaatgtcagacgaaaatgccaattattgaagcggctaacgccgctttttttgtttctggtctcccttgcttgtactttacagtatagcttctagagatctgcagtgactggagcttctagagatctgcagtgactgggaaaaccctggcgactagtcttggactcctgttgatagatccagtaatgacctcagaactccatctggatttgttcagaacgctcggttgccgccgggcgttttttattggtgagaatccaggggtccccaataattacgatttaaatccttcaaacttcccaaaggcgagccctagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatcccgtttgttgaagacttttgtccttttccgctgcataaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttccaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagcgtctgtacgttccttaaggaattattccttagtgctttctaggttaatgtcatgataataatggtttcttagacgtcaggtggcacttttcggggaaatgtccgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggaggtttttcgaaggttctctgagctaccaactctttgaaccgaggtaactggcttgcaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcagggggcggagcctatggaaaaacgctttgcccgagcctatggaaaaacgctttgccccacacaggctcttgaacaagggataacaatttgtcatagctgtattcgttacaaggaggattttcgaaaaaacaccctaacgggtgtttttttatagctggtctccccaagtgctgtaaaaaacgccgaatgaagcggatgaaattgctcatcgtctcgtgaaaaagccgaagaaggttaagcctgggtataaaaagaaaatgagctatgagatggagaaagccataaccatgagtgataacactggttttagagctagaaatagcaagttaaaataaggctagtccgttatcaacttgaaaaagtggcaccgagtcggtgctttttttctcggtaccaaattccagaaaagaggcctcccgaaaggggggccttttttcgttttggtccctcaatgatctccttatccggttaagatggc

SEQ ID NO: 238 - DNA, 10197 bp, Artificial Sequence
  Description of Artificial Sequence Synthetic polynucleotide
gagtaaatcctggggcaaattcatcgaggaggaagaggcggaaatggcttcccgtcgtaatctaatgattgtcgatggaactaacttaggctttcgcttcaaacataacaatagtaaaaaaccatttgcctcaagttatgtttcaactattcaatctctggcaaaatcctactctgccagaactacgattgttctaggtgataagggcaaaagcgtgttcagactagaacatctaccagagtataaaggtaatcgtgatgaaaagtacgcacaacgtacggaagaggagaaagcgctagatgagcagttcttcgaatacctcaaagatgctttcgagttgtgtaaaactacattcccaacttttaccattcgtggtgtagaagcagatgacatggcggcgtatattgttaagctcatcgggcatctttatgatcacgtttggctaatatctacagatggtgactgggatactttattaacggataaagtttctcgtttttctttcacaacacgtcgtgagtatcatcttcgtgatatgtatgagcaccataacgtggacgacgtagaacagtttatctccctgaaagcaattatgggagatctaggagataatattcgtggtgttgaaggaataggagcaaaacgcggatataatattattcgtgagtttggtaacgtactggatattattgatcagcttccactgcctggaaagcagaaatatatacagaacctgaatgcatcggaagaactgcttttccgaaacttgattctggttgatttacctacctactgtgtggatgctattgctgctgtaggtcaagatgtgttagataagttcacgaaagacatccttgagatcgcggaacaatgataagctcttaaagggggttttagataccagagatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactgataatcaaatgtcagacgaaaatgccaattattgaagcggctaacgccgctttttttgtttctggtctcccttgcttgtactttacagtatagcttctagagatctgcagtgactggagcttctagagatcaggtcagacgcgatttcctgggtgctcgagccatgggaaagcggtggtggaaaaaacgccgaatgaagcggatgaaattgctcatcgtctcgtgaaaaagccgaagaaggttaagcctgggtataaaaagaaaatgagctatgagatggagaaattgaagtcatgcgccggttagttttagagctagaaatagcaagttaaaataaggctagtccgttatcaacttgaaaaagtggcaccgagtcggtgctttttttctcggtaccaaattccagaaaagaggcctcccgaaaggggggccttttttcgttttggtccctcaatgatccgacaggaagtggtattagcgactcctacaaggaataggtgtctgggtcatgcagtgactgggaaaaccctggcgactagtcttggactcctgttgatagatccagtaatgacctcagaactccatctggatttgttcagaacgctcggttgccgccgggcgttttttattggtgagaatccaggggtccccaataattacgatttaaatccttcaaacttcccaaaggcgagccctagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatcccgtttgttgaagacttttgtccttttccgctgcataaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttccaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagcgtctgtacgttccttaaggaattattccttagtgctttctaggttaatgtcatgataataatggtttcttagacgtcaggtggcacttttcggggaaatgtccgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggaggtttttcgaaggttctctgagctaccaactctttgaaccgaggtaactggcttgcaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcagggggcggagcctatggaaaaacgctttgcccgagcctatggaaaaacgctttgccccacacaggctcttgaacaagggataacaatttgtcatagctgtattcgttacaaggaggattttcgaaaaaacaccctaacgggtgtttttttatagctggtctccccaagtgctgtaaaaaacgccgaatgaagcggatgaaattgctcatcgtctcgtgaaaaagccgaagaaggttaagcctgggtataaaaagaaaatgagctatgagatggagaaagccataaccatgagtgataacactggttttagagctagaaatagcaagttaaaataaggctagtccgttatcaacttgaaaaagtggcaccgagtcggtgctttttttctcggtaccaaattccagaaaagaggcctcccgaaaggggggccttttttcgttttggtccctcaatgatctccttatccggttaagatggc

SEQ ID NO: 239 - DNA, 11978 bp, Artificial Sequence
  Description of Artificial Sequence Synthetic polynucleotide
gtgactgggaaaaccctggcgactagtcttggactcctgttgatagatccagtaatgacctcagaactccatctggatttgttcagaacgctcggttgccgccgggcgttttttattggtgagaatccaggggtccccaataattacgatttaaatccttcaaacttcccaaaggcgagccctagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatcccgtttgttgaagacttttgtccttttccgctgcataaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttccaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagcgtctgtacgttccttaaggaattattccttagtgctttctaggttaatgtcatgataataatggtttcttagacgtcaggtggcacttttcggggaaatgtccgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggaggtttttcgaaggttctctgagctaccaactctttgaaccgaggtaactggcttgcaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcagggggcggagcctatggaaaaacgctttgcccgagcctatggaaaaacgctttgccccacacaggctcttgaacaagggataacaatttgtcatagctgtattcgttacaaggaggattttcgaaaaaacaccctaacgggtgtttttttatagctggtctccccaagtgctgtaaaaaacgccgaatgaagcggatgaaattgctcatcgtctcgtgaaaaagccgaagaaggttaagcctgggtataaaaagaaaatgagctatgagatggagaaagccataaccatgagtgataacactggttttagagctagaaatagcaagttaaaataaggctagtccgttatcaacttgaaaaagtggcaccgagtcggtgctttttttctcggtaccaaattccagaaaagaggcctcccgaaaggggggccttttttcgttttggtccctcaatgatctccttatccggttaagatggcttttaaatcaattttcagctcctgtatacaattaccaaagtttttctgaatgaagccatgtgttttgacacattctatactcacaaggaggtgagacacatgagtaaatcctggggcaaattcatcgaggaggaagaggcggaaatggcttcccgtcgtaatctaatgattgtcgatggaactaacttaggctttcgcttcaaacataacaatagtaaaaaaccatttgcctcaagttatgtttcaactattcaatctctggcaaaatcctactctgccagaactacgattgttctaggtgataagggcaaaagcgtgttcagactagaacatctaccagagtataaaggtaatcgtgatgaaaagtacgcacaacgtacggaagaggagaaagcgctagatgagcagttcttcgaatacctcaaagatgctttcgagttgtgtaaaactacattcccaacttttaccattcgtggtgtagaagcagatgacatggcggcgtatattgttaagctcatcgggcatctttatgatcacgtttggctaatatctacagatggtgactgggatactttattaacggataaagtttctcgtttttctttcacaacacgtcgtgagtatcatcttcgtgatatgtatgagcaccataacgtggacgacgtagaacagtttatctccctgaaagcaattatgggagatctaggagataatattcgtggtgttgaaggaataggagcaaaacgcggatataatattattcgtgagtttggtaacgtactggatattattgatcagcttccactgcctggaaagcagaaatatatacagaacctgaatgcatcggaagaactgcttttccgaaacttgattctggttgatttacctacctactgtgtggatgctattgctgctgtaggtcaagatgtgttagataagttcacgaaagacatccttgagatcgcggaacaatgataagctcttaaagggggttttagataccagagatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactgataatcaaatgtcagacgaaaatgccaattattgaagcggctaacgccgctttttttgtttctggtctcccttgcttgtactttacagtatagcttcctcaatgatctccttatccggttaagatggcaagcttgacaagtatttcttcagaatttacaatgaagtttactagaggattaactaataaggagggcaaactactagagatgtctaaacgtcgtatgaagtaccacagcaacaatgagatcagctattacaatttcctacatagcatgaaggacaaaatcgtgacggtgtatagaggcggccctgagagcaagaagggcaagctgacggcagtgaagagcgactacatcgcgctgcaggcggagaagaagatcatctactatcaacttgaacacgttaagtcaatcacggaagacacaaacaactcaacaacgacgatcgaaacggaagagatgttagacgcggacgacttccactcactgatcggccacctgatcaatcagagcgtgcagttcaatcagggaggaccagaaagcaagaaaggccgccttgtatggttaggcgacgactatgcggcactgaatacgaacgaagacggagtggtatacttcaacattcaccacattaagtccatctcaaagcacgaaccggaccttaagatcgaggaacaaacaccggtgggcgtgcttgaggcggacgacctgtcagaagtgttcaaatcattaacgcacaagtgggtgagcatcaacagaggcggccctgaggcaatcgaaggcattttagtggacaacgcagatggacactacacgatcgttaagaaccaggaagtattgagaatttacccgttccacatcaaatcaatttcactgggcccgaagggaagttataaaaaggaagaccagaagaacgagcagaatcaagaggataacaacgacaaagattcaaacagctttatcagcagcaaaagctattcaagcagtaaaagctcaaagcggagcttgaagagctctgacgaccagagcagtaagtcaggcagaagttccagaagcaagagtagctccaagagtagtaagaggagtttgaagtccagcgactaccagagcagtaaaagtggaagaagttccaggagcaagtcctcaagtaagtccagtaagagaagcctgaagagtagcgactaccagagcagcaaaagctcaaaaaggagcccgcgcagtagcgactaccagagtagtcgtagcccgggatatagctcctccatcaagtctagtggcaagcagaaagaggactattcatacgagacaatcgttcgcacaatcgattatcattggaagaggaaattcggtttcaaactcgggggggacggaggtgaaatgtctaagggcgaagagttattcacgggcgtagttccgattcttgtggagctcgacggtgatgtaaacgggcacaagttttctgtttctggggaaggtgaaggcgacgcgacctatgggaaactcactcttaagttcatctgcaccacggggaagttaccagtcccgtggccaacactggtgactaccttcgcatacggactgcagtgttttgcgcgttatcctgatcacatgaaacaacacgattttttcaagagcgccatgccagaagggtacgtccaagaacgcacgatttttttcaaagatgatgggaattacaaaacgcgtgcagaagtaaaatttgagggcgacaccctggtaaaccgtattgaactcaaaggaatcgactttaaggaggacggaaacattctgggccacaaactggaatataactacaactcacataacgtttatataatggctgataagcaaaaaaatggtatcaaggtgaactttaagataagacataacattgaggacggcagtgtccaacttgcagaccattaccagcagaacacaccaattggcgacggtccagtacttttaccggacaaccactatctctcaactcagagtgctctgtcaaaggaccctaatgaaaaacgcgaccacatggtactgctcgagtttgtgactgcggccggaatcacccacgggatggacgagctttacaagtaatcaaatgtcagacgaaaatgccaattattgaagcggctaacgccgctttttttgtttctggtctcccaactttgctaaagagcgtctcagcttctagag

SEQ ID NO: 240 - DNA, 12394 bp, Artificial Sequence
  Description of Artificial Sequence Synthetic polynucleotide
tagagatctgcagtgactggagcttctagagatcaggtcagacgcgatttcctgggtgctcgagccatgggaaagcggtggtggaaaaaacgccgaatgaagcggatgaaattgctcatcgtctcgtgaaaaagccgaagaaggttaagcctgggtataaaaagaaaatgagctatgagatggagaaattgaagtcatgcgccggttagttttagagctagaaatagcaagttaaaataaggctagtccgttatcaacttgaaaaagtggcaccgagtcggtgctttttttctcggtaccaaattccagaaaagaggcctcccgaaaggggggccttttttcgttttggtccctcaatgatccgacaggaagtggtattagcgactcctacaaggaataggtgtctgggtcatgcagtgactgggaaaaccctggcgactagtcttggactcctgttgatagatccagtaatgacctcagaactccatctggatttgttcagaacgctcggttgccgccgggcgttttttattggtgagaatccaggggtccccaataattacgatttaaatccttcaaacttcccaaaggcgagccctagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatcccgtttgttgaagacttttgtccttttccgctgcataaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttccaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagcgtctgtacgttccttaaggaattattccttagtgctttctaggttaatgtcatgataataatggtttcttagacgtcaggtggcacttttcggggaaatgtccgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggaggtttttcgaaggttctctgagctaccaactctttgaaccgaggtaactggcttgcaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcagggggcggagcctatggaaaaacgctttgcccgagcctatggaaaaacgctttgccccacacaggctcttgaacaagggataacaatttgtcatagctgtattcgttacaaggaggattttcgaaaaaacaccctaacgggtgtttttttatagctggtctccccaagtgctgtaaaaaacgccgaatgaagcggatgaaattgctcatcgtctcgtgaaaaagccgaagaaggttaagcctgggtataaaaagaaaatgagctatgagatggagaaagccataaccatgagtgataacactggttttagagctagaaatagcaagttaaaataaggctagtccgttatcaacttgaaaaagtggcaccgagtcggtgctttttttctcggtaccaaattccagaaaagaggcctcccgaaaggggggccttttttcgttttggtccctcaatgatctccttatccggttaagatggcttttaaatcaattttcagctcctgtatacaattaccaaagtttttctgaatgaagccatgtgttttgacacattctatactcacaaggaggtgagacacatgagtaaatcctggggcaaattcatcgaggaggaagaggcggaaatggcttcccgtcgtaatctaatgattgtcgatggaactaacttaggctttcgcttcaaacataacaatagtaaaaaaccatttgcctcaagttatgtttcaactattcaatctctggcaaaatcctactctgccagaactacgattgttctaggtgataagggcaaaagcgtgttcagactagaacatctaccagagtataaaggtaatcgtgatgaaaagtacgcacaacgtacggaagaggagaaagcgctagatgagcagttcttcgaatacctcaaagatgctttcgagttgtgtaaaactacattcccaacttttaccattcgtggtgtagaagcagatgacatggcggcgtatattgttaagctcatcgggcatctttatgatcacgtttggctaatatctacagatggtgactgggatactttattaacggataaagtttctcgtttttctttcacaacacgtcgtgagtatcatcttcgtgatatgtatgagcaccataacgtggacgacgtagaacagtttatctccctgaaagcaattatgggagatctaggagataatattcgtggtgttgaaggaataggagcaaaacgcggatataatattattcgtgagtttggtaacgtactggatattattgatcagcttccactgcctggaaagcagaaatatatacagaacctgaatgcatcggaagaactgcttttccgaaacttgattctggttgatttacctacctactgtgtggatgctattgctgctgtaggtcaagatgtgttagataagttcacgaaagacatccttgagatcgcggaacaatgataagctcttaaagggggttttagataccagagatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactgataatcaaatgtcagacgaaaatgccaattattgaagcggctaacgccgctttttttgtttctggtctcccttgcttgtactttacagtatagcttcctcaatgatctccttatccggttaagatggcaagcttgacaagtatttcttcagaatttacaatgaagtttactagaggattaactaataaggagggcaaactactagagatgtctaaacgtcgtatgaagtaccacagcaacaatgagatcagctattacaatttcctacatagcatgaaggacaaaatcgtgacggtgtatagaggcggccctgagagcaagaagggcaagctgacggcagtgaagagcgactacatcgcgctgcaggcggagaagaagatcatctactatcaacttgaacacgttaagtcaatcacggaagacacaaacaactcaacaacgacgatcgaaacggaagagatgttagacgcggacgacttccactcactgatcggccacctgatcaatcagagcgtgcagttcaatcagggaggaccagaaagcaagaaaggccgccttgtatggttaggcgacgactatgcggcactgaatacgaacgaagacggagtggtatacttcaacattcaccacattaagtccatctcaaagcacgaaccggaccttaagatcgaggaacaaacaccggtgggcgtgcttgaggcggacgacctgtcagaagtgttcaaatcattaacgcacaagtgggtgagcatcaacagaggcggccctgaggcaatcgaaggcattttagtggacaacgcagatggacactacacgatcgttaagaaccaggaagtattgagaatttacccgttccacatcaaatcaatttcactgggcccgaagggaagttataaaaaggaagaccagaagaacgagcagaatcaagaggataacaacgacaaagattcaaacagctttatcagcagcaaaagctattcaagcagtaaaagctcaaagcggagcttgaagagctctgacgaccagagcagtaagtcaggcagaagttccagaagcaagagtagctccaagagtagtaagaggagtttgaagtccagcgactaccagagcagtaaaagtggaagaagttccaggagcaagtcctcaagtaagtccagtaagagaagcctgaagagtagcgactaccagagcagcaaaagctcaaaaaggagcccgcgcagtagcgactaccagagtagtcgtagcccgggatatagctcctccatcaagtctagtggcaagcagaaagaggactattcatacgagacaatcgttcgcacaatcgattatcattggaagaggaaattcggtttcaaactcgggggggacggaggtgaaatgtctaagggcgaagagttattcacgggcgtagttccgattcttgtggagctcgacggtgatgtaaacgggcacaagttttctgtttctggggaaggtgaaggcgacgcgacctatgggaaactcactcttaagttcatctgcaccacggggaagttaccagtcccgtggccaacactggtgactaccttcgcatacggactgcagtgttttgcgcgttatcctgatcacatgaaacaacacgattttttcaagagcgccatgccagaagggtacgtccaagaacgcacgatttttttcaaagatgatgggaattacaaaacgcgtgcagaagtaaaatttgagggcgacaccctggtaaaccgtattgaactcaaaggaatcgactttaaggaggacggaaacattctgggccacaaactggaatataactacaactcacataacgtttatataatggctgataagcaaaaaaatggtatcaaggtgaactttaagataagacataacattgaggacggcagtgtccaacttgcagaccattaccagcagaacacaccaattggcgacggtccagtacttttaccggacaaccactatctctcaactcagagtgctctgtcaaaggaccctaatgaaaaacgcgaccacatggtactgctcgagtttgtgactgcggccggaatcacccacgggatggacgagctttacaagtaatcaaatgtcagacgaaaatgccaattattgaagcggctaacgccgctttttttgtttctggtctcccaactttgctaaagagcgtctcagcttctagag

SEQ ID NO: 241 - DNA, 12014 bp, Artificial Sequence
  Description of Artificial Sequence Synthetic polynucleotide
ttaagatggcaagcttgacagccgaattcggattcccgcgtatatcttgaattaaaattaaaatctgccaagatcgaaaaataaaaaggattttttgtgtcattggcgaattatgatctattgaagcaaccgttaagcttacataaggaggaactactatggaacaggatacgcagcatgttaaaccacttcaaacaaaaaccgatattcatgcagtcttggcctctaatggacgcatcatttatatatctgccaactccaaactgcatttgggctatctccaaggagagatgatcggatcattcctcaaaacgtttctgcatgaggaagaccaatttttggttgaaagctatttttataatgaacatcatctgatgccgtgcacctttcgttttattaaaaaagatcatacgattgtgtgggtggaggctgcggtagaaattgttacgacaagagctgagcggacagaacgggaaatcattttgaaaatgaaggttcttgaagaagaaacaggccatcaatccctaaactgcgaaaaacatgaaatcgaacctgcaagcccggaatcgactacatatataacggatgattatgaacggttggttgaaaatctcccgagtccgctatgcatcagtgtcaaaggcaagatcgtctatgtaaacagcgcgatgctttcaatgctgggagccaaaagcaaggatgctattattggtaaatcgtcctatgaatttattgaagaagaatatcatgatatcgtgaaaaacaggattatacgaatgcaaaaaggaatggaagtcggaatgattgaacagacgtggaaaaggcttgatggcacacctgttcatttagaagtgaaagcatccccgaccgtctacaaaaaccagcaggctgagctgctgctgctgatcgatatctcttcaaggaaaaaattccaaaccatcctgcaaaaaagccgtgaacgatatcagctgctgattcaaaattccattgataccattgcggtgattcacaatggaaaatgggtatttatgaatgaatcgggaatttccctgtttgaagcggctacatatgaggatttaattggcaaaaacatatacgatcagctgcatccttgcgatcacgaggatgtaaaagagagaatccaaaacattgccgagcaaaaaacagaatctgaaattgtcaagcaatcctggttcacctttcagaacagggtcatctatacggagatggtctgcattccgacgaccttttttggtgaagcggccgtccaggtcattcttcgggacatctcagagagaaaacaaacagaagaattgatgctgaaatcggaaaaattatcaatcgcagggcagctcgcggcgggaatcgcccatgagatccgcaaccctcttacagcgatcaaaggatttttacagctgatgaaaccgacaatggaaggcaacgaacattactttgatattgtgttttctgaactcagccgtatcgaattaatactcagtgaactgctcatgctggcgaaacctcagcaaaatgctgtcaaagaatatttgaacttgaaaaaattaattggtgaggtttcagccctgttagaaacgcaggcgaatttaaatggcatttttatcagaacaagttatgaaaaagacagcatttatataaacggggatcaaaaccaattaaagcaggtattcattaatttaatcaaaaatgcagttgaatcaatgcctgatgggggaacagtagacattatcataaccgaagatgagcattctgttcatgttactgtcaaagacgaaggggaaggtatacctgaaaaggtactaaaccggattggagagccatttttaacaacaaaagaaaaaggtacggggcttggattaatggtgacatttaatatcattgaaaaccatcagggagttatacatgtggacagccatcctgaaaaaggcacagcgtttaaaatttcatttccaaaaaaataagcatgcccggggatggacgagctttacaagtaatcaaatgtcagacgaaaatgccaattattgaagcggctaacgccgctttttttgtttctggtctcccaactttgctaaagagcgtctcagcttctagaggtgactgggaaaaccctggcgactagtcttggactcctgttgatagatccagtaatgacctcagaactccatctggatttgttcagaacgctcggttgccgccgggcgttttttattggtgagaatccaggggtccccaataattacgatttaaatccttcaaacttcccaaaggcgagccctagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatcccgtttgttgaagacttttgtccttttccgctgcataaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttccaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagcgtctgtacgttccttaaggaattattccttagtgctttctaggttaatgtcatgataataatggtttcttagacgtcaggtggcacttttcggggaaatgtccgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggaggtttttcgaaggttctctgagctaccaactctttgaaccgaggtaactggcttgcaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcagggggcggagcctatggaaaaacgctttgcccgagcctatggaaaaacgctttgccccacacaggctcttgaacaagggataacaatttgtcatagctgtattcgttacaaggaggattttcgaaaaaacaccctaacgggtgtttttttatagctggtctccccaagtgctgtaaaaaacgccgaatgaagcggatgaaattgctcatcgtctcgtgaaaaagccgaagaaggttaagcctgggtataaaaagaaaatgagctatgagatggagaaagccataaccatgagtgataacactggttttagagctagaaatagcaagttaaaataaggctagtccgttatcaacttgaaaaagtggcaccgagtcggtgctttttttctcggtaccaaattccagaaaagaggcctcccgaaaggggggccttttttcgttttggtccctcaatgatctccttatccggttaagatggcttttaaatcaattttcagctcctgtatacaattaccaaagtttttctgaatgaagccatgtgttttgacacattctatactcacaaggaggtgagacacatgagtaaatcctggggcaaattcatcgaggaggaagaggcggaaatggcttcccgtcgtaatctaatgattgtcgatggaactaacttaggctttcgcttcaaacataacaatagtaaaaaaccatttgcctcaagttatgtttcaactattcaatctctggcaaaatcctactctgccagaactacgattgttctaggtgataagggcaaaagcgtgttcagactagaacatctaccagagtataaaggtaatcgtgatgaaaagtacgcacaacgtacggaagaggagaaagcgctagatgagcagttcttcgaatacctcaaagatgctttcgagttgtgtaaaactacattcccaacttttaccattcgtggtgtagaagcagatgacatggcggcgtatattgttaagctcatcgggcatctttatgatcacgtttggctaatatctacagatggtgactgggatactttattaacggataaagtttctcgtttttctttcacaacacgtcgtgagtatcatcttcgtgatatgtatgagcaccataacgtggacgacgtagaacagtttatctccctgaaagcaattatgggagatctaggagataatattcgtggtgttgaaggaataggagcaaaacgcggatataatattattcgtgagtttggtaacgtactggatattattgatcagcttccactgcctggaaagcagaaatatatacagaacctgaatgcatcggaagaactgcttttccgaaacttgattctggttgatttacctacctactgtgtggatgctattgctgctgtaggtcaagatgtgttagataagttcacgaaagacatccttgagatcgcggaacaatgataagctcttaaagggggttttagataccagagatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactgataatcaaatgtcagacgaaaatgccaattattgaagcggctaacgccgctttttttgtttctggtctcccttgcttgtactttacagtatagcttcctcaatgatctccttatccgg

SEQ ID NO: 242 - DNA, 12430 bp, Artificial Sequence
  Description of Artificial Sequence Synthetic polynucleotide
ttaagatggcaagcttgacagccgaattcggattcccgcgtatatcttgaattaaaattaaaatctgccaagatcgaaaaataaaaaggattttttgtgtcattggcgaattatgatctattgaagcaaccgttaagcttacataaggaggaactactatggaacaggatacgcagcatgttaaaccacttcaaacaaaaaccgatattcatgcagtcttggcctctaatggacgcatcatttatatatctgccaactccaaactgcatttgggctatctccaaggagagatgatcggatcattcctcaaaacgtttctgcatgaggaagaccaatttttggttgaaagctatttttataatgaacatcatctgatgccgtgcacctttcgttttattaaaaaagatcatacgattgtgtgggtggaggctgcggtagaaattgttacgacaagagctgagcggacagaacgggaaatcattttgaaaatgaaggttcttgaagaagaaacaggccatcaatccctaaactgcgaaaaacatgaaatcgaacctgcaagcccggaatcgactacatatataacggatgattatgaacggttggttgaaaatctcccgagtccgctatgcatcagtgtcaaaggcaagatcgtctatgtaaacagcgcgatgctttcaatgctgggagccaaaagcaaggatgctattattggtaaatcgtcctatgaatttattgaagaagaatatcatgatatcgtgaaaaacaggattatacgaatgcaaaaaggaatggaagtcggaatgattgaacagacgtggaaaaggcttgatggcacacctgttcatttagaagtgaaagcatccccgaccgtctacaaaaaccagcaggctgagctgctgctgctgatcgatatctcttcaaggaaaaaattccaaaccatcctgcaaaaaagccgtgaacgatatcagctgctgattcaaaattccattgataccattgcggtgattcacaatggaaaatgggtatttatgaatgaatcgggaatttccctgtttgaagcggctacatatgaggatttaattggcaaaaacatatacgatcagctgcatccttgcgatcacgaggatgtaaaagagagaatccaaaacattgccgagcaaaaaacagaatctgaaattgtcaagcaatcctggttcacctttcagaacagggtcatctatacggagatggtctgcattccgacgaccttttttggtgaagcggccgtccaggtcattcttcgggacatctcagagagaaaacaaacagaagaattgatgctgaaatcggaaaaattatcaatcgcagggcagctcgcggcgggaatcgcccatgagatccgcaaccctcttacagcgatcaaaggatttttacagctgatgaaaccgacaatggaaggcaacgaacattactttgatattgtgttttctgaactcagccgtatcgaattaatactcagtgaactgctcatgctggcgaaacctcagcaaaatgctgtcaaagaatatttgaacttgaaaaaattaattggtgaggtttcagccctgttagaaacgcaggcgaatttaaatggcatttttatcagaacaagttatgaaaaagacagcatttatataaacggggatcaaaaccaattaaagcaggtattcattaatttaatcaaaaatgcagttgaatcaatgcctgatgggggaacagtagacattatcataaccgaagatgagcattctgttcatgttactgtcaaagacgaaggggaaggtatacctgaaaaggtactaaaccggattggagagccatttttaacaacaaaagaaaaaggtacggggcttggattaatggtgacatttaatatcattgaaaaccatcagggagttatacatgtggacagccatcctgaaaaaggcacagcgtttaaaatttcatttccaaaaaaataagcatgcccggggatggacgagctttacaagtaatcaaatgtcagacgaaaatgccaattattgaagcggctaacgccgctttttttgtttctggtctcccaactttgctaaagagcgtctcagcttctagagtagagatctgcagtgactggagcttctagagatcaggtcagacgcgatttcctgggtgctcgagccatgggaaagcggtggtggaaaaaacgccgaatgaagcggatgaaattgctcatcgtctcgtgaaaaagccgaagaaggttaagcctgggtataaaaagaaaatgagctatgagatggagaaattgaagtcatgcgccggttagttttagagctagaaatagcaagttaaaataaggctagtccgttatcaacttgaaaaagtggcaccgagtcggtgctttttttctcggtaccaaattccagaaaagaggcctcccgaaaggggggccttttttcgttttggtccctcaatgatccgacaggaagtggtattagcgactcctacaaggaataggtgtctgggtcatgcagtgactgggaaaaccctggcgactagtcttggactcctgttgatagatccagtaatgacctcagaactccatctggatttgttcagaacgctcggttgccgccgggcgttttttattggtgagaatccaggggtccccaataattacgatttaaatccttcaaacttcccaaaggcgagccctagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatcccgtttgttgaagacttttgtccttttccgctgcataaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttccaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagcgtctgtacgttccttaaggaattattccttagtgctttctaggttaatgtcatgataataatggtttcttagacgtcaggtggcacttttcggggaaatgtccgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggaggtttttcgaaggttctctgagctaccaactctttgaaccgaggtaactggcttgcaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcagggggcggagcctatggaaaaacgctttgcccgagcctatggaaaaacgctttgccccacacaggctcttgaacaagggataacaatttgtcatagctgtattcgttacaaggaggattttcgaaaaaacaccctaacgggtgtttttttatagctggtctccccaagtgctgtaaaaaacgccgaatgaagcggatgaaattgctcatcgtctcgtgaaaaagccgaagaaggttaagcctgggtataaaaagaaaatgagctatgagatggagaaagccataaccatgagtgataacactggttttagagctagaaatagcaagttaaaataaggctagtccgttatcaacttgaaaaagtggcaccgagtcggtgctttttttctcggtaccaaattccagaaaagaggcctcccgaaaggggggccttttttcgttttggtccctcaatgatctccttatccggttaagatggcttttaaatcaattttcagctcctgtatacaattaccaaagtttttctgaatgaagccatgtgttttgacacattctatactcacaaggaggtgagacacatgagtaaatcctggggcaaattcatcgaggaggaagaggcggaaatggcttcccgtcgtaatctaatgattgtcgatggaactaacttaggctttcgcttcaaacataacaatagtaaaaaaccatttgcctcaagttatgtttcaactattcaatctctggcaaaatcctactctgccagaactacgattgttctaggtgataagggcaaaagcgtgttcagactagaacatctaccagagtataaaggtaatcgtgatgaaaagtacgcacaacgtacggaagaggagaaagcgctagatgagcagttcttcgaatacctcaaagatgctttcgagttgtgtaaaactacattcccaacttttaccattcgtggtgtagaagcagatgacatggcggcgtatattgttaagctcatcgggcatctttatgatcacgtttggctaatatctacagatggtgactgggatactttattaacggataaagtttctcgtttttctttcacaacacgtcgtgagtatcatcttcgtgatatgtatgagcaccataacgtggacgacgtagaacagtttatctccctgaaagcaattatgggagatctaggagataatattcgtggtgttgaaggaataggagcaaaacgcggatataatattattcgtgagtttggtaacgtactggatattattgatcagcttccactgcctggaaagcagaaatatatacagaacctgaatgcatcggaagaactgcttttccgaaacttgattctggttgatttacctacctactgtgtggatgctattgctgctgtaggtcaagatgtgttagataagttcacgaaagacatccttgagatcgcggaacaatgataagctcttaaagggggttttagataccagagatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactgataatcaaatgtcagacgaaaatgccaattattgaagcggctaacgccgctttttttgtttctggtctcccttgcttgtactttacagtatagcttcctcaatgatctccttatccgg

SEQ ID NO: 243 - DNA, 22 bp, Artificial Sequence
  Description of Artificial Sequence Synthetic primer
actgcccgttactggtataaag

SEQ ID NO: 244 - DNA, 21 bp, Artificial Sequence
  Description of Artificial Sequence Synthetic primer
tgcaccttgctttggtttatg

SEQ ID NO: 245 - DNA, 21 bp, Artificial Sequence
  Description of Artificial Sequence Synthetic primer
ggcggtgatcactgatgaata

SEQ ID NO: 246 - DNA, 22 bp, Artificial Sequence
  Description of Artificial Sequence Synthetic primer
gctgtctctccactgtcaaata